SEQUENCE LISTING (278 sequences)

SEQ ID NO: 1 - DNA, 23 bp, Artificial Sequence
  BRAF primer
tcataatgcttgctctgatagga

SEQ ID NO: 2 - DNA, 22 bp, Artificial Sequence
  BRAF primer 5′ biotinylated
ggccaaaaatttaatcagtgga

SEQ ID NO: 3 - DNA, 16 bp, Artificial Sequence
  BRAF sequencing primer
ccactccatcgagatt

SEQ ID NO: 4 - DNA, 24 bp, Artificial Sequence
  KRAS primer 5′ biotinylated
gtgtgacatgttctaatatagtca

SEQ ID NO: 5 - DNA, 20 bp, Artificial Sequence
  KRAS primer
gaatggtcctgcaccagtaa

SEQ ID NO: 6 - DNA, 15 bp, Artificial Sequence
  KRAS sequencing primer
gcactcttgcctacg

SEQ ID NO: 7 - DNA, 21 bp, Artificial Sequence
  TP53 primer
gttctggtaaggacaagggtt

SEQ ID NO: 8 - DNA, 20 bp, Artificial Sequence
  TP53 reverse primer
ccaggcattgaagtctcatg

SEQ ID NO: 9 - DNA, 18 bp, Artificial Sequence
  TP53 forward primer
ggttgcaggaggtgctta

SEQ ID NO: 10 - DNA, 21 bp, Artificial Sequence
  TP53 primer 4 reverse
ccactgacaaccacccttaac

SEQ ID NO: 11 - DNA, 19 bp, Artificial Sequence
  TP53 primer 5 forward
cctgcttgccacaggtctc

SEQ ID NO: 12 - DNA, 22 bp, Artificial Sequence
  TP53 primer 6 reverse
tgaatctgaggcataactgcac

SEQ ID NO: 13 - DNA, 18 bp, Artificial Sequence
  SFRP1 rprimer 1 forward
gaattcgttcgcgaggga

SEQ ID NO: 14 - DNA, 21 bp, Artificial Sequence
  SFRP1 primer 2 reverse
aaacgaaccgcactcgttacc

SEQ ID NO: 15 - DNA, 23 bp, Artificial Sequence
  SFRP1 probe sequence 5′ 6FAM and 3′ BHQ-1
ccgtcaccgacgcgaaaaccaat

SEQ ID NO: 16 - DNA, 25 bp, Artificial Sequence
  SFRP1 primer 3 forward
gttaaattcgcgtatgatttcgaga

SEQ ID NO: 17 - DNA, 16 bp, Artificial Sequence
  SFRP1 primer 4 reverse
ttcccgcgtctccgac

SEQ ID NO: 18 - DNA, 24 bp, Artificial Sequence
  SFRP1 probe sequence 2 5′ 6FAM and 3′ BHQ-1
aacgaacgaccctctcgctccgaa

SEQ ID NO: 19 - DNA, 28 bp, Artificial Sequence
  LMOD1 forward primer
ttttaaagataaggggttacgtaatgag

SEQ ID NO: 20 - DNA, 22 bp, Artificial Sequence
  LMOD1 reverse primer
ccgaactaacgaattcaccgac

SEQ ID NO: 21 - DNA, 27 bp, Artificial Sequence
  LMOD1 probe sequence 5′ 6FAM and 3′ MGBNFQ
tcgtccctacttatctaactctccgta

SEQ ID NO: 22 - DNA, 50 bp, Artificial Sequence
  KCNK13 source sequence
gtaggtgcctccccaggtagatcgacgatggtgcctcctagttgtggtcg

SEQ ID NO: 23 - DNA, 50 bp, Artificial Sequence
  KCNK13 Unmethylated AlleleA Probe Sequence
ataaatacctccccaaataaatcaacaataatacctcctaattataatca

SEQ ID NO: 24 - DNA, 50 bp, Artificial Sequence
  KCNK13 Methylated AlleleB Probe Sequence
ataaatacctccccaaataaatcgacgataatacctcctaattataatcg

SEQ ID NO: 25 - DNA, 3001 bp, Homo sapiens
aatgcactcgccagagctgtgtttctttattcgaaatcacacggttttactcaacagggctcctagcccttctgtctctgctcctggtgcgtaagggcggtgtgtggggctctgcgcctgaaaccgctctctcggaaggtgggaagatgcccacgtatccattcctctccgcgtacagattcgctcccacacgcccgggcgggtgattccggagggccgcgcggggctgtgtgcgcgggcgggggcgggatgtgtctggagggggcagagcgccctctcccggccccggagggcccacgcggaccggctgagcgtcccaccagccggttctgcatccactacccagatctgcgcgccctggtgggagcaccgcagccccgggtcggcccttcgaacgccgagcgtctacccgcggagaccagcgcaggaggcgggcccgcgcgcaaggaggcgggcgacagggtggagtcgccgcagctggcagccgcacggctctacctgttgcagcgtcggagcgcgcccgcctgagaaggcagagcgcggtgcggacctgtgctccgcgcgccggctggcagggcagggccgcggctgccaggaggcccgggaacagtcccgacggagcggcgcggagagggcgctccccggaaggtgagatctgagcgcggcagcggcgccagcgcccgctacaaggcggggtctcggggagggggccccggcggtttgctggagagccttgctactgttctgcatgaggtgggggatgccttctgcgcggaccccgcgtccaggcaaaactctggaacgcactaggcagccaagcccggggggaagagatggaacgctccaaggtcaaggtctcctggccgcggagcccttccagcccccagtgtttggcctcctctccggaagcggggctggctcgcggtgcgcgcttggtccgggcggagcctactagtaggcgcagaggacgcccccgcgcaggatccagagaccaagggcgctctggcatggtcaaggcgagggaggctgcccgggggagaagcccctaacttcctttgctccctgttccaacctccctttctggtctcctctctccagcccgtccgcccaacggatgcgcagcgttggggcccggccggaggtcggagccctcggacgccgcagccctcggacgccgctcagcacagcccctctctccgtgcgcccgccctggaccccctccatccctaagccccggcagccgattcggagactcgggaggccacaggctcagcgcgacaccacgaccacaactaggaggcaccatcgtcgatctacctggggaggcacctacaaagccagcagactgacacccgtgtcctagccgtcggcggcctgcgaagcgatagtcaaaggaaccaggacccaggaccccggcgctctgcttacccgcagacctccggaccgcgccccccccccgccgccccgccccgccccgccccgccccgcgaccgtctctttgcgggaaactcgagttggctcgagcttgccctgaagacgcgccacgccccctcccctatctgagccggacagggagccggggtggaaactgcgcccgcatcccagcgccaagcagacgtgaactctgcgcccgttccccgggaccttcagcgttaaggagaactccgaggagagcagggggagtggaggggaacggcggcgacaaagcggatttgaaactaggagtcaaggaggacgtggggagctggcgccacaggagctaccgaggcggcggccgggggagcctcgcggcctgcgggagagcccggcggtcatgggcgagccggcggtggggcgcccgggagctggctgagcgccggggcccttatttcccgggggtgtgggcgagactccgccgacgcccggtgccgtgggcctgggggctgcccccgggggcccggccatggctggccggggtttcagctggggcccgggccacctgaacgaggacaacgcgcgctttctgctgctggccgcgctcatcgtgctctacctgctgggcggcgccgccgtcttctccgcgctggagctggcgcacgagcgccaggccaagcagcgctgggaggagcgcctggccaacttcagccgcggccacaacctgagccgcgacgagctgcgcggcttcctccgccactacgaggaggccactcgggccggcatccgcgtggacaacgtccgcccgcgctgggacttcaccggcgccttctacttcgtgggcaccgtcgtttccaccataggtaagtgtgctggccggactcgctgacaacctccgggcggcctccacttcctccggggggcaggaccgaccctctcatcctttcattcatccatctgggcgcccagccagactccactgacatgagctgtcgcctgatcaacaggtggtattgcctccccgctgctctaatgtggtccaggcaccggcagcttcagcgtcacctggcagcttgttagaaatgcagtctcagtcccctctgcagacctgccgagtcagaatctgcatgttaacaaagtccccaaaggattggaatgatccttaaattggagacgctctgctctgggagattttcagtctaatgaaggagacacggcttgagttcatggggaagaaggcttttacagatagggacaggaaactaaacatttactgagtcagtcagaggggaagtgcagattttaaaagattctgccctcgacttcacacttaccgagaaaaaatcagaacagcagactgaaagtgaggaagcaaaagtgccaatgacggttcaaaagatggaaaattttccaccaggaagaaccagggaagtgacctttgggctgaatcaattcgtgttaggcataacttgggtttgggaactgaaagcgcggttgcactctagagtaggagagaatcgcactcaggggccccatgagctggttggagtggtggaaggaagactaggaaggtctgctggctaatggg

SEQ ID NO: 26 - DNA, 2255 bp, Homo sapiens
cgtaagggcggtgtgtggggctctgcgcctgaaaccgctctctcggaaggtgggaagatgcccacgtatccattcctctccgcgtacagattcgctcccacacgcccgggcgggtgattccggagggccgcgcggggctgtgtgcgcgggcgggggcgggatgtgtctggagggggcagagcgccctctcccggccccggagggcccacgcggaccggctgagcgtcccaccagccggttctgcatccactacccagatctgcgcgccctggtgggagcaccgcagccccgggtcggcccttcgaacgccgagcgtctacccgcggagaccagcgcaggaggcgggcccgcgcgcaaggaggcgggcgacagggtggagtcgccgcagctggcagccgcacggctctacctgttgcagcgtcggagcgcgcccgcctgagaaggcagagcgcggtgcggacctgtgctccgcgcgccggctggcagggcagggccgcggctgccaggaggcccgggaacagtcccgacggagcggcgcggagagggcgctccccggaaggtgagatctgagcgcggcagcggcgccagcgcccgctacaaggcggggtctcggggagggggccccggcggtttgctggagagccttgctactgttctgcatgaggtgggggatgccttctgcgcggaccccgcgtccaggcaaaactctggaacgcactaggcagccaagcccggggggaagagatggaacgctccaaggtcaaggtctcctggccgcggagcccttccagcccccagtgtttggcctcctctccggaagcggggctggctcgcggtgcgcgcttggtccgggcggagcctactagtaggcgcagaggacgcccccgcgcaggatccagagaccaagggcgctctggcatggtcaaggcgagggaggctgcccgggggagaagcccctaacttcctttgctccctgttccaacctccctttctggtctcctctctccagcccgtccgcccaacggatgcgcagcgttggggcccggccggaggtcggagccctcggacgccgcagccctcggacgccgctcagcacagcccctctctccgtgcgcccgccctggaccccctccatccctaagccccggcagccgattcggagactcgggaggccacaggctcagcgcgacaccacgaccacaactaggaggcaccatcgtcgatctacctggggaggcacctacaaagccagcagactgacacccgtgtcctagccgtcggcggcctgcgaagcgatagtcaaaggaaccaggacccaggaccccggcgctctgcttacccgcagacctccggaccgcgccccccccccgccgccccgccccgccccgccccgccccgcgaccgtctctttgcgggaaactcgagttggctcgagcttgccctgaagacgcgccacgccccctcccctatctgagccggacagggagccggggtggaaactgcgcccgcatcccagcgccaagcagacgtgaactctgcgcccgttccccgggaccttcagcgttaaggagaactccgaggagagcagggggagtggaggggaacggcggcgacaaagcggatttgaaactaggagtcaaggaggacgtggggagctggcgccacaggagctaccgaggcggcggccgggggagcctcgcggcctgcgggagagcccggcggtcatgggcgagccggcggtggggcgcccgggagctggctgagcgccggggcccttatttcccgggggtgtgggcgagactccgccgacgcccggtgccgtgggcctgggggctgcccccgggggcccggccatggctggccggggtttcagctggggcccgggccacctgaacgaggacaacgcgcgctttctgctgctggccgcgctcatcgtgctctacctgctgggcggcgccgccgtcttctccgcgctggagctggcgcacgagcgccaggccaagcagcgctgggaggagcgcctggccaacttcagccgcggccacaacctgagccgcgacgagctgcgcggcttcctccgccactacgaggaggccactcgggccggcatccgcgtggacaacgtccgcccgcgctgggacttcaccggcgccttctacttcgtgggcaccgtcgtttccaccataggtaagtgtgctggccggactcgctgacaacctccgggcggcctccacttcctccggggggcaggaccg

SEQ ID NO: 27 - DNA, 50 bp, Artificial Sequence
  SLIT1 source sequence
cggtggactgccacggcacggggctgcaggccattcccaagaatatacct

SEQ ID NO: 28 - DNA, 50 bp, Artificial Sequence
  SLIT 1 Unmethylated AlleleA Probe Sequence
aaatatattcttaaaaataacctacaaccccataccataacaatccacca

SEQ ID NO: 29 - DNA, 50 bp, Artificial Sequence
  SLIT1 Methylated AlleleB Probe Sequence
aaatatattcttaaaaataacctacaaccccgtaccgtaacaatccaccg

SEQ ID NO: 30 - DNA, 3001 bp, Homo sapiens
caaaatctacacacacacagcccattacggggactctcatggcctcacgctattaaggtggccccttaatactaggaattcacacagaaacacattctgcaggagagtcacagaaaacaaagtataaaggtctgtaaatacacacactcaaacacacccaaacacacacacacacattctactagcttactgggtctagaaaagaaaaggatcaagtttcctcaagaattcacaagtgagacagaacaggggaagagaaatgctatttttttattacttgtcatggtcaatatctttaaccttgggcccccctctccaagctcctgacttcttggcattatccaccgggttctccatccaagacacacctgccccttttcgggtgctgaatgttacataccccaagccccatacccaaagccctcaggtcccattgtctgcctgtgttggggatggctgggtgtggagggaggcgggtgagtaggggaccaacgcgtggcacctgcaggggaagaattctcaaggctctggggcaaacaatggagaagaaggggcagaccagaaacttcgcctccatcccaccctgctgcccttcgtcaggagcaagctctgggagcgggaatctgcgcagctggctcagaacgcggccgtagccccatgtgcccaaaaggagacattgtgcttggcgagtgtgggaagtgtccctcctgggtgcaaggaaggctttctgacaccagttcgcaggcaccctggaggaataaagcgctggcgcagtgaggagggcctgagggtgcggagtgcagggtgcagggtgcaggtgtgaggacggaagctgacaaccccgccgagggccctttgaatcccagggatggggaaaccacggcgctcctggctcctggctcgggaaagaccccggcaggcactgacccgtctgtgggctgcggagcccggacgggccccaatggtcctgcccccacccccaagtccggaattgcgtctgggtgggagggcaacctcggagccagggagccaggggcggggaccatactcacaggcgctcggtgttccgaggtatattcttgggaatggcctgcagccccgtgccgtggcagtccaccgtggttccggtgcaggtgcagagggcggggcacgccgaggcacccaggcgccacgcggctgcccacagcagcagccagagctccggccggaccggccccgccgaggacccccacccgggagtcagcgccatggtgccctcacagcgtcccgctcgcgagccagacggcagcagccgctgaccatccccgtccggggccgcctccaggtgcagtcccggggcagagccaccgaagagcccgcgggcttgcgcgcggcgcccctgcgggctgggaggcaccttgctcctccaagcgacggcgcctgtgcgcggacggagggagggcgccttgggcggagggggctcggctcctctgccgtttcgccgcctgcgtctccctctccctccctccagctcccaactgactgctgcgaggaggaaaatgggcaggccccgcgcgcgcacgcaccctgcgcacacaccacacacacacacacgatcacacacacacccgcgcgctcgctcccacccggcagtcatccatcaatcgaaaagcacacatccagggccaggctcctccccgccctcccccgccgcagcccccctctcccgacgaccactcagcggctgcaagccgcctacagtttcaaaggtggaacctccgaggcgccgtcagcgggcacggggctgcgggggatgggagaagagggaagggggtgtgactcgacccgccaagtcctggctgtacacacacacacacacacacacacacacacacacacacacacacggcgaggatgcgcacatttagacgcacgcaccccggcacccgcggccccagctgctagtccagggtgggagggagatcttattttgccgcttggacgccttcccggggagtcagacccgtttccccccatcccccgacggcagcagcggcctctcggccccacccgactcgctgtgtcaattcttggttaagggaaaccctgtgcctgtgccccaggtagcatcctcaagccaccaaccttacttagcctggactttgaggatggagctttgtcgggggggagggtccacgaaagcgctaggcggagtacagtctccttcctacctcaatgcacccacttctcagaggggaacaagctagccgcatgcccaggatgagaatggctctgattggaacacttgctcagcagggcgtatttgtaccatacagttacacgggcttcttcccccgttgtagaagagagggacagacattaaggggacttggaaatctggcaaacgtcttcctccactcacaccagcgttcctccatttctgacaggtggaaacagatgaaaatgaatattccatcttgacatcaaaggagacagagagggctggcatagcgctgggcggcctgcttgggagagaaccccacaggccagccccgtcagacccaggcctcacacccggctgggtagggaccttcgtcaccgtaaggcatgtgtggacgtgggtgtgaacgggacatccaggtggggagagaattctcctcaaccccagaaaagacgaaagatctcctcaacgccaaggacagctgtggctcgctggggcagtgtccagcagtcggggccttgacgtgaggctgcttggcaagaggacacagctgggtagcccgagaagcacctcccgtctcaggagcggcctgggtccagacacagcatggacgcatttggtggggagagtgtcgggcagggtggggataggggaggttgatagactccacaaggttgaagccttatcacccaccaggtacccaaggctcaggcgttttctttgcttttcttttccatgttgatgagaattttgcagtctctctactgtcatagcacagtatgttacaatttaggtggctga

SEQ ID NO: 31 - DNA, 1177 bp, Homo sapiens
cggcgctcctggctcctggctcgggaaagaccccggcaggcactgacccgtctgtgggctgcggagcccggacgggccccaatggtcctgcccccacccccaagtccggaattgcgtctgggtgggagggcaacctcggagccagggagccaggggcggggaccatactcacaggcgctcggtgttccgaggtatattcttgggaatggcctgcagccccgtgccgtggcagtccaccgtggttccggtgcaggtgcagagggcggggcacgccgaggcacccaggcgccacgcggctgcccacagcagcagccagagctccggccggaccggccccgccgaggacccccacccgggagtcagcgccatggtgccctcacagcgtcccgctcgcgagccagacggcagcagccgctgaccatccccgtccggggccgcctccaggtgcagtcccggggcagagccaccgaagagcccgcgggcttgcgcgcggcgcccctgcgggctgggaggcaccttgctcctccaagcgacggcgcctgtgcgcggacggagggagggcgccttgggcggagggggctcggctcctctgccgtttcgccgcctgcgtctccctctccctccctccagctcccaactgactgctgcgaggaggaaaatgggcaggccccgcgcgcgcacgcaccctgcgcacacaccacacacacacacacgatcacacacacacccgcgcgctcgctcccacccggcagtcatccatcaatcgaaaagcacacatccagggccaggctcctccccgccctcccccgccgcagcccccctctcccgacgaccactcagcggctgcaagccgcctacagtttcaaaggtggaacctccgaggcgccgtcagcgggcacggggctgcgggggatgggagaagagggaagggggtgtgactcgacccgccaagtcctggctgtacacacacacacacacacacacacacacacacacacacacacacggcgaggatgcgcacatttagacgcacgcaccccggcacccgcggccccagctgctagtccagggtgggagggagatcttattttgccgcttggacgccttcccggggagtcagacccgtttccccccatcccccgacggcagcagcggcctctcggccccacccgactcg

SEQ ID NO: 32 - DNA, 50 bp, Artificial Sequence
  RAB31 source sequence
cggccaggactcaccccgagaaggcacactttgagctcccgtatcgccat

SEQ ID NO: 33 - DNA, 50 bp, Artificial Sequence
  RAB31 Unmethylated AlleleA Probe Sequence
ataacaatacaaaaactcaaaatataccttctcaaaataaatcctaacca

SEQ ID NO: 34 - DNA, 50 bp, Artificial Sequence
  RAB31 Methylated AlleleB Probe Sequence
ataacgatacgaaaactcaaaatataccttctcgaaataaatcctaaccg

SEQ ID NO: 35 - DNA, 3001 bp, Homo sapiens
agctggaaagccaaaaggacatgtttcactgtggaggtgccctcagggctggcgaaggccttgggggaggtgtctttgcaactgtgtaggggtggttttctgattagcaaatctgagggcagggagatattgtgatcaggactgtttgattctggggaagcctctttttaccctgcccacatagaagactctgctttggatacagagtcaaggacggatactgatacaatggacctgtaacttgtccatggctgccttttgtacagtttagtccatttgagtaatctctacaagcctgcacctcgagggcaaacttcctgctcttctggggaatttaattgtgaattgtctgtttcggggcccattttgactcctgtattccaggacttatctctttggaggccttgatttggaatttcccattggatggggctacttgggtactgggtagcttcaacctggcttccgtaagaacaaagccagatgatcacggtgggtggtctgctaggagaatggaaatgccattgattaaaattggaaggttaagagagaggcctgagagtttgggataggagatgaagtttcaaaaaatcagtttttaaaaaaacatttcccatctgaaatgccagtgaattgttcagatagattacattgaaaaggctgacatttctgtagcagcaacgaatgaagtcatccgagtggatgaggtgctctaggccatacatggagaagagaacacagttacccaagcctccccgactgtgtgcggccacagactctgctcatttcccagttaggaacgatgcaggggtctgctgggatctccacgtttctacctgtacctctgaggactgggtttctgcccacacgagttgcaggatttgctctactgagaaaaagacttttgctccagaaagcaagtttcccgtttcagacacggacagcgagttttgcttctagaatcattttcagctgcatacagaaaaatgaacttggctgagctgaagagccacacatatgttaaacggaagctcgggtattaggaacgtcgagaaaggcaggccgaaaagttagcccccttctcctcacccctgtcctcacagcggccccttcagtggcctgactttttctgcgtgggacccagaatgtcgcttgaggatgggacccccgcagacctccacgcgctgccacgcaaacggcctcggccgagagcattcgctttccagatgagaatggcatcttcagggacccgctggtagtggggccaggtctccacagggccggtccagtccttcctacccacccgggccctgcggccaggcggggccgggaggggcggatgccggagggcgctgggggaggccgacccgcgcggggctggtccgagggcggaggcaatccgccggcgacgccgcaggtcccgggtggctgacgccgtccgggggcgttcctgggagctgggggccggcgaggctggacccgacgcgcccgggaggagccggccggcggccacgtgacccaccgcggcggggccggcggcccagaaatagcagcggcggcggttccgcccgcgggcggcgcgagcgaggggcagaggcgagagacgccggcggggcgcgggcgcggcggccccggaggatgctgctgagccccggcactgcctggctgcgagcacatgatggcgatacgggagctcaaagtgtgccttctcggggtgagtcctggccgccacccgccggcggaccccggcccgcgctctcgcgccccttcgctcccctattccctgcgcgctcagtccccgtgatcccctcgctctccgcacccctctcgtagcccccgtccccctcgtccgcgcgccccctggttccccgggtccccctggctcccctagtccgtgcgcccctcgctctccgcgcccctcgctctccgcaccccgcctgttctccgcctccccgccgtccgtgcgcccctctggtccgcccccgctctcaccctgccggggtccgggtccgagcctgccccgggcttactccgctctttcctcccggcccgcgagtccccggatccgcggcgacctcgcggggacccccagcggggacggggcagtggcggcgagtctggggccccgagggctttctcctccgcttttgatagtttccttccggcagaaagtttaagttgctcagccaagtcgctgagtgctcttttgcctcctgggggcaattgatttactcacgtaaaacagaaaaataaaaccaaaagcgaacccagccaagggggtgaaagccgagggcgcggccggggtggaaggtgaggcagtgacctgtgcggagcgcgccctgcaaacttccctgctgcgggtgaaagaagctgcctgggaaagtcctccccaccatcactcccctaaagtggggttttctccgcccgaagaagccttaaagcagccagaaacaaaaaagcagtcaatccgtgcggcccggggactgcagacaggggacccggcgaggacgcagggagccgctggcgaaccgcccctgggaattcagataaacgtagggtcgcttcctttcgggggcccactccgcattccagaattccttcttctaaaagaagagccggttctaagggcgttttcagcagccattagcatctggtgtacccgcccgtgtcattggcaaagctggtctcatgaaaaccatttcctaaaaaagggcagcgaaagggtcgcattcttgtgcgaactgatctgtcaagatgggggtacaagaactgtagaaagaggaaatgatttcggtgaggactgaattgtgccagcgtctctaggcttcctctaggctcttgtggaacctccggcaggctcccatcatcggctcccatcaccggcctcttccaaaaccatcccttacgctaactcgattgtccggttggcttagaattccagaggatctgggaaggccctgccctccctgctagcccctggcacagatccgcacagatcccc

SEQ ID NO: 36 - DNA, 1559 bp, Homo sapiens
cggaagctcgggtattaggaacgtcgagaaaggcaggccgaaaagttagcccccttctcctcacccctgtcctcacagcggccccttcagtggcctgactttttctgcgtgggacccagaatgtcgcttgaggatgggacccccgcagacctccacgcgctgccacgcaaacggcctcggccgagagcattcgctttccagatgagaatggcatcttcagggacccgctggtagtggggccaggtctccacagggccggtccagtccttcctacccacccgggccctgcggccaggcggggccgggaggggcggatgccggagggcgctgggggaggccgacccgcgcggggctggtccgagggcggaggcaatccgccggcgacgccgcaggtcccgggtggctgacgccgtccgggggcgttcctgggagctgggggccggcgaggctggacccgacgcgcccgggaggagccggccggcggccacgtgacccaccgcggcggggccggcggcccagaaatagcagcggcggcggttccgcccgcgggcggcgcgagcgaggggcagaggcgagagacgccggcggggcgcgggcgcggcggccccggaggatgctgctgagccccggcactgcctggctgcgagcacatgatggcgatacgggagctcaaagtgtgccttctcggggtgagtcctggccgccacccgccggcggaccccggcccgcgctctcgcgccccttcgctcccctattccctgcgcgctcagtccccgtgatcccctcgctctccgcacccctctcgtagcccccgtccccctcgtccgcgcgccccctggttccccgggtccccctggctcccctagtccgtgcgcccctcgctctccgcgcccctcgctctccgcaccccgcctgttctccgcctccccgccgtccgtgcgcccctctggtccgcccccgctctcaccctgccggggtccgggtccgagcctgccccgggcttactccgctctttcctcccggcccgcgagtccccggatccgcggcgacctcgcggggacccccagcggggacggggcagtggcggcgagtctggggccccgagggctttctcctccgcttttgatagtttccttccggcagaaagtttaagttgctcagccaagtcgctgagtgctcttttgcctcctgggggcaattgatttactcacgtaaaacagaaaaataaaaccaaaagcgaacccagccaagggggtgaaagccgagggcgcggccggggtggaaggtgaggcagtgacctgtgcggagcgcgccctgcaaacttccctgctgcgggtgaaagaagctgcctgggaaagtcctccccaccatcactcccctaaagtggggttttctccgcccgaagaagccttaaagcagccagaaacaaaaaagcagtcaatccgtgcggcccggggactgcagacaggggacccggcgaggacgcagggagccgctggcgaaccgcccctgggaattcagataaacgtagggtcgcttcctttcgggggcccactccg

SEQ ID NO: 37 - DNA, 50 bp, Artificial Sequence
  FOXL2 source sequence
cgggcgagttcatctccaagtcactttttgtaaacgccccgcacagcctg

SEQ ID NO: 38 - DNA, 50 bp, Artificial Sequence
  FOXL2 Unmethylated AlleleA Probe Sequence
caaactatacaaaacatttacaaaaaataacttaaaaataaactcaccca

SEQ ID NO: 39 - DNA, 50 bp, Artificial Sequence
  FOXL2 Methylated AlleleB Probe Sequence
caaactatacgaaacgtttacaaaaaataacttaaaaataaactcgcccg

SEQ ID NO: 40 - DNA, 3001 bp, Homo sapiens
ccagtaagagcaatgcatcatggcgagctcgggctgccgggcacaagcgaactgcaggcccggcgcactggtgggcgcgggcgccgggggcgcggcggtggctgggctggcagggctgagctggcccggcggcggcgcggcggccccgtggtgcggtggggcaggcggcggtgcggcggccgcgtgcagatggtgtgcgtgcggatgcgggtgggggtgcggcggaggcgggggtgcggccggcgggcctcccaggccattgtacgagttcactacgccggggggcagcgccatgctctgcacgcgtgtgtacggcccgtacgaggcggccgggcccgccagccccttgaccacagcggccgcgccagggctaccggggcccgcggctgcagccgcagctgctgcagccgctgcggctgccgccatctggcaggaggcatagggcatgggtgagggaggctgcggtagcggccacgagttgttgaggaagccagactgcaggtacttggggggcgccaggtagccgtagccgtcggccccggcgcccgccacgccgcacccgcctgcggcgcctccggccccgaagagccccttgccgggctggaagtgcgcgggcggcggccggaagggcctcttcatgcggcggcggcgccggtagttgcccttctcgaacatgtcttcgcaggccgggtccagcgtccagtagttgcccttgcgctcgccgccgccctcgcgcggcaccttgatgaagcactcgttgaggctgaggttgtggcggatgctattttgccagcccttcttattcttctcgtagaacgggaacttcgcgatgatgtactggtagatgccggacagcgtgagcctcttctccgcgctctcgcggatcgccatggcgatgagcgccacgtacgagtacgggggcttctgcgccgggtccggcttctccggggctgtcccgccgccacccccaccgcccttgcctgggctcggcggcggcccttctggctccttgactgtgcgaccggtctctggggccagcagggcccccgccgcgtcctcgggctcggggtagctggccatcatgacaaagccggcgcgccgcggccgggccgcctctgctctccgctccaggcgctggcgcggcaaagagttggggcgcacgagtccgcttacggccaagtctcaaacttctggagactgcggatgccgcccgcgcttgcttgctggaggcctgtcgctgctctcccctctccttccccttcccctagggagcggccggcgggagtggagctcagcctctggccatggggagtccgcccaacagagaggggctccggcctcgccgcccctccccgctcaggccagtccccgccttggtgggttttcttttctgcgctcttcccctccccccgccccccggtttcccgaagcacgacccgcgtctctggcggagctgcctcctggagtccctagtgcgccaggagcctcgctctgttctgattcgtatgggctccaccgagttccgcttgcgtcaggcgccttcgcccctatagcggggcggccagccgcgcacgggcgagttcatctccaagtcactttttgtaaacgccccgcacagcctggaccggcctgcccccgcccagcgagcctcaggggcccagccgacagccaggctcacgcgcccttgaaatctgccggtactcgctctgcgggctgggctgggagatgacgaggaccccggtggggtctgcccgcacccggccaaagcccaggaagctcgggccccagcgaggaaaggcgctccaagcctcctcgcggctttcaggtgaaagaaaacgactcctttgctctgccgtttgctgccgtcttgaggctgaacttctagctcggggctggggaggggcgagacggcgagggggctggacggggtagggtggggagagctgctctgaggctttgggaaagtcagcccagaaacgggtgtgactgtacgaagaagcctcggcctggcctgtccctcgcgctctcagagtgactgggctggaatggggcaggggagaggatctctggaaatagtcgtcaggggcgccgcctgaatcacctctgcctctccctgcgttaccagtggatctaggaaccaggaatccgtgtactaatcctacggggttggagtgaaggttggatgtgtgctttaacagcaccaagtagatgcccctcagctattgcactaacacaggcggggctgttgcccgggactttgcgggactgtgtatgtgtgtgcacaggggtcacatatggaagtcatggagaggagctgtccttgagatgggtgagataccaggtgcatgtgtgctgctcagaaacacaggtatgcacatgtacaaggtgtgagatttgtatttacgaggtaaaacacacttatactgatagttatgtaatatgttcatcaagtattagagtaaatgtagctggtggatatagcatcttctctttttctcctttcttacttctttccttcccccctttcttcctttctttcacaattcttcgatgcctctttccctcccagtgtatgtattttgccacagactgggaagacaaacatcaggctgagggagggacttgttcaaatctctctcctgatgagacatttgttctgatctttctccaaagaatcaggagttagttgtcacctaccttaagcagaattctgatgggaatccaattatttcagcatgtacagagattgtttcagggaaaatcaggtgtgccttttaacagtgtgctccttaacagtgccttttaacttggcctaaacaaaaagtctgcagtcataaagtatgagagagatggccagtgttttgaatcctcaagccccaaactccttggtgctgaggaggggggcaaggccaccttatggaggaaggtggactgaatggatgtcatatagctgtgcattagtgattggcatgtctggagtggctgactcaatgcctgtcac

SEQ ID NO: 41 - DNA, 2628 bp, Homo sapiens
cgccggggcccggaggtgcccgaagcgacgggactgatagcggaggaaacgcagcctccctccgcgcccgcccgcggtgtaaaccgagtacaggccgtggagccaggctgccccggctcccgctgggtcccaacccccgccccgcctagtgggccccgcggcaagcggcttctgaacagcttcaagagggttcgcggagcaaacacacgtattggtccgtccctttctcgggcagcgcggtcccgccatcagtcctgtccggcgcgtctagccatggactgcacggcagtcgggcggggaacgcggagagcgagcgcaccgacctgtgagagaaggccaagaggtctgcgctgccgacgcccggtcgcacctccgccccgggccctttccgcggtgaatttgggcaggagacgctggggctccggaaagagacgagcccagtagaaagcgcgcagagaggcagcttcaggccaggggagtgcaaggtcacagaggtcagggaggtgagcacaggaggacataaactgaggggacaaagaggagcgacaggagcttaggaaagcgaaaaagcacagagggaccctgggcgctggctccagaggcgggcccagagggtgtgaggtcaggctggcggcggcgtcgtcggctgcgaccggggccggcgtcgcgcgtccctgcatcctcgcatccgtctgcaccggcatgcggtgggctctcagagatcgaggcgcgaatgcagcgcgccggtcttgctgtcgtggtcccagtaagagcaatgcatcatggcgagctcgggctgccgggcacaagcgaactgcaggcccggcgcactggtgggcgcgggcgccgggggcgcggcggtggctgggctggcagggctgagctggcccggcggcggcgcggcggccccgtggtgcggtggggcaggcggcggtgcggcggccgcgtgcagatggtgtgcgtgcggatgcgggtgggggtgcggcggaggcgggggtgcggccggcgggcctcccaggccattgtacgagttcactacgccggggggcagcgccatgctctgcacgcgtgtgtacggcccgtacgaggcggccgggcccgccagccccttgaccacagcggccgcgccagggctaccggggcccgcggctgcagccgcagctgctgcagccgctgcggctgccgccatctggcaggaggcatagggcatgggtgagggaggctgcggtagcggccacgagttgttgaggaagccagactgcaggtacttggggggcgccaggtagccgtagccgtcggccccggcgcccgccacgccgcacccgcctgcggcgcctccggccccgaagagccccttgccgggctggaagtgcgcgggcggcggccggaagggcctcttcatgcggcggcggcgccggtagttgcccttctcgaacatgtcttcgcaggccgggtccagcgtccagtagttgcccttgcgctcgccgccgccctcgcgcggcaccttgatgaagcactcgttgaggctgaggttgtggcggatgctattttgccagcccttcttattcttctcgtagaacgggaacttcgcgatgatgtactggtagatgccggacagcgtgagcctcttctccgcgctctcgcggatcgccatggcgatgagcgccacgtacgagtacgggggcttctgcgccgggtccggcttctccggggctgtcccgccgccacccccaccgcccttgcctgggctcggcggcggcccttctggctccttgactgtgcgaccggtctctggggccagcagggcccccgccgcgtcctcgggctcggggtagctggccatcatgacaaagccggcgcgccgcggccgggccgcctctgctctccgctccaggcgctggcgcggcaaagagttggggcgcacgagtccgcttacggccaagtctcaaacttctggagactgcggatgccgcccgcgcttgcttgctggaggcctgtcgctgctctcccctctccttccccttcccctagggagcggccggcgggagtggagctcagcctctggccatggggagtccgcccaacagagaggggctccggcctcgccgcccctccccgctcaggccagtccccgccttggtgggttttcttttctgcgctcttcccctccccccgccccccggtttcccgaagcacgacccgcgtctctggcggagctgcctcctggagtccctagtgcgccaggagcctcgctctgttctgattcgtatgggctccaccgagttccgcttgcgtcaggcgccttcgcccctatagcggggcggccagccgcgcacgggcgagttcatctccaagtcactttttgtaaacgccccgcacagcctggaccggcctgcccccgcccagcgagcctcaggggcccagccgacagccaggctcacgcgcccttgaaatctgccggtactcgctctgcgggctgggctgggagatgacgaggaccccggtggggtctgcccgcacccggccaaagcccaggaagctcgggccccagcgaggaaaggcgctccaagcctcctcgcggctttcaggtgaaagaaaacgactcctttgctctgccgtttgctgccg

SEQ ID NO: 42 - DNA, 50 bp, Artificial Sequence
  B3GAT2 source sequence
gatgggtgcgctgtccatggggccgagggcgctgcagagacctggagccg

SEQ ID NO: 43 - DNA, 50 bp, Artificial Sequence
  B3GAT2 Unmethylated AlleleA Probe Sequence
aataaatacactatccataaaaccaaaaacactacaaaaacctaaaacca

SEQ ID NO: 44 - DNA, 50 bp, Artificial Sequence
  B3GAT2 Methylated AlleleB Probe Sequence
aataaatacgctatccataaaaccgaaaacgctacaaaaacctaaaaccg

SEQ ID NO: 45 - DNA, 3001 bp, Homo sapiens
cagaaaagccccagggcagactcccgccccttaagccacagtgatcctctcacacacagcccctccacccccacggccagcggaggctgcggagtgccagcaaggagcggctccactcggtgccccgaatgcgcgcacggagaactgagaactcaaaagtgcacccggagtgcaaggggcgtgtgactgcagcccggccggcccggaggcccactcccgccctcgcccacccagcggggcaggctggcctttacctcctggaagagctccagactataggtgttgtcgtcgtcagcgaagaagagcacgccgggctgcgcgcgctggtgctggtgcctctggcgcagccaggcgaggcccgcgttgcgctgctcagtggcgcgcggcagcccgggccgcttgtagcgccgcggcgtgggcacgtgcaggtgagtgctgggcagcccggcccgcgccaggaagcggctcaccagctcgctgcgcgccgccgcgtcctccaccaggatccagtgcagctgcgccacctggcggaacgtgttggccaggcgggtcagctccgctttctgcaccgggcggctgtaggtgggcgtgatggcatagatggtgggcagctgcggctccggctgtggctgcggccgagactggttgcgcttttgggtcccgtgagccgggccgcccctgcggagcgggagtcgggcgcccccgcggcccaccgcgtagggagagaagtaggggcgcggggtgagcgggggcactggcctgcgcgtgtccacgtcgagcatgatgatgacaattaggatccagggcaggaggataaagaagcgggtgaaaagcgcggacttcatggtgcacgctccctggcctctcggacaccccagagaggggcgagccgagggaccccagtgcgcagcttggacagcggcggcgccagcacttagggagtggtgatgggtgcgctgtccatggggccgagggcgctgcagagacctggagccgcggggctcactacctgggcgtggaggagcggcaggttcgcgcaagctagagcgacaaggggtgcaggcgggcgggcaggggctgcccccgactccaggtgagctggcgggaagcgggactcggtccagccgcgcgccgccggtcccggagttgtgccgagtgcgggaaaggcgctgatccccaccgcgctctttctgaagagtggaagccgagaagcggcacccggtgcgcctcgccgctccagtccggcggtgctgcgggcacaagggctccagccgcgggcccccaggacgctctctgggacgccttcgagggcgggcggcggcgctgggggctttccttcctcacattcccgccggggtggcgaggagcgggtggagacgctgggggttgtgtcccggctgtgttcgcgcgccgcagcggaagcctgctctcagtcccttgctcttgtcttctcagaacctctccggatccagcagcaagatcccaaagcaaggaaagaaagaaaaagcgagggggcgggggcgctgcagaatccagcgtcctcccaagtcctcctgtgggaagacagcagcgctgctactggtggagaggggctgcgagagggaggcgcggagggaccccttggctctccgccgggtctggcgctaacggccccgcgagcaagctcgggaggcggccgctgcaccgcgggtggaggagcgggaggagcgtccccgcggggctgcacgacctcctggagggggtgatgctcgttcacctctcttcagctttgtgaaataccgcaaggtctttggagatgagccaggaaaatccattcacgtgcggaaatttgccgggctacgcgcgttgctgctgatgcctagggttcggagtgaaaggaggggagaaagcgaagggattcctcctcctgctcccagctgctgcgttgatgtgctggggtgtatcgaacagcggcaacaaaaagcgctttcactgcaaggcagacatcaataggagcccacggagatggaggggtgctgcacataggtgtttggcatgtgagacagtccataagaaagaatcaattcggagggctgctgcctgactgcagtttgatcatggcgggtgggggcgggtggaattcctaaaactccagccgagcatgtttaacttatttttcttcagtaatttggagtcacggtccctcccagttcttcgctgtcaccggaattactccgtttggctctcccaaggctcctgtcaagaaggcctaagaagtagaatggccttcattttcaggataaggaagctgaggttcagggtttaaaatgatttctctgaggtcagtggcaagtggcatatggaaatagaacgtgggtctcctgcgtgtgtctatagctagtggcgtcctcactgcatggcctgcagaaatcccacccaacacagccgggccggtttggccttcctacatataaagatggtgcgattgaaatcaaacagagtgtaccatgacgaatagaaccatgatagggtctggaagaccccaagtaaacagttggcacttagtttagacactttttgctgtctagctggcctactagctgagtgcctgaccttctgtaacttaccaggaagaagggctgtgcttcccaggcaggtcccagggaagtgcttgcaaagcagaaggaaagctccagactgttctgccctagcctatgataaataatttctgagcccatcttgtctgggttgtctgcctttctcgggcttgttcacaaataaatctctctccgactgtgcgttctggtgcaactgagagctctggggcgaatggctctccagcttttctgacagctcctcttctcccagtcctacccctgaaccccccacctgttaaaaggcattggctcatctgtgaatttccctaacttcctcttcagattctgaaacgtgagagtgaaatttcagactaacacccagccaccccaacaactccctagagctagccttctgtcccaacttc

SEQ ID NO: 46 - DNA, 1772 bp, Homo sapiens
cggccagcggaggctgcggagtgccagcaaggagcggctccactcggtgccccgaatgcgcgcacggagaactgagaactcaaaagtgcacccggagtgcaaggggcgtgtgactgcagcccggccggcccggaggcccactcccgccctcgcccacccagcggggcaggctggcctttacctcctggaagagctccagactataggtgttgtcgtcgtcagcgaagaagagcacgccgggctgcgcgcgctggtgctggtgcctctggcgcagccaggcgaggcccgcgttgcgctgctcagtggcgcgcggcagcccgggccgcttgtagcgccgcggcgtgggcacgtgcaggtgagtgctgggcagcccggcccgcgccaggaagcggctcaccagctcgctgcgcgccgccgcgtcctccaccaggatccagtgcagctgcgccacctggcggaacgtgttggccaggcgggtcagctccgctttctgcaccgggcggctgtaggtgggcgtgatggcatagatggtgggcagctgcggctccggctgtggctgcggccgagactggttgcgcttttgggtcccgtgagccgggccgcccctgcggagcgggagtcgggcgcccccgcggcccaccgcgtagggagagaagtaggggcgcggggtgagcgggggcactggcctgcgcgtgtccacgtcgagcatgatgatgacaattaggatccagggcaggaggataaagaagcgggtgaaaagcgcggacttcatggtgcacgctccctggcctctcggacaccccagagaggggcgagccgagggaccccagtgcgcagcttggacagcggcggcgccagcacttagggagtggtgatgggtgcgctgtccatggggccgagggcgctgcagagacctggagccgcggggctcactacctgggcgtggaggagcggcaggttcgcgcaagctagagcgacaaggggtgcaggcgggcgggcaggggctgcccccgactccaggtgagctggcgggaagcgggactcggtccagccgcgcgccgccggtcccggagttgtgccgagtgcgggaaaggcgctgatccccaccgcgctctttctgaagagtggaagccgagaagcggcacccggtgcgcctcgccgctccagtccggcggtgctgcgggcacaagggctccagccgcgggcccccaggacgctctctgggacgccttcgagggcgggcggcggcgctgggggctttccttcctcacattcccgccggggtggcgaggagcgggtggagacgctgggggttgtgtcccggctgtgttcgcgcgccgcagcggaagcctgctctcagtcccttgctcttgtcttctcagaacctctccggatccagcagcaagatcccaaagcaaggaaagaaagaaaaagcgagggggcgggggcgctgcagaatccagcgtcctcccaagtcctcctgtgggaagacagcagcgctgctactggtggagaggggctgcgagagggaggcgcggagggaccccttggctctccgccgggtctggcgctaacggccccgcgagcaagctcgggaggcggccgctgcaccgcgggtggaggagcgggaggagcgtccccgcggggctgcacgacctcctggagggggtgatgctcgttcacctctcttcagctttgtgaaataccgcaaggtctttggagatgagccaggaaaatccattcacgtgcggaaatttgccgggctacgcgcg

SEQ ID NO: 47 - DNA, 50 bp, Artificial Sequence
  FAM78A source sequence
ggacggtatcagcggagatgtcacgggcggctattattcgctggtgcgcg

SEQ ID NO: 48 - DNA, 50 bp, Artificial Sequence
  FAM78A Unmethylated AlleleA Probe Sequence
aaacaatatcaacaaaaatatcacaaacaactattattcactaatacaca

SEQ ID NO: 49 - DNA, 50 bp, Artificial Sequence
  FAM78A Methylated AlleleB Probe Sequence
aaacgatatcaacgaaaatatcacgaacgactattattcgctaatacgcg

SEQ ID NO: 50 - DNA, 3001 bp, Homo sapiens
aactctgcctaaatgcagctcttttcaccctggggacggccttctctggggtcaggaagcaggccaggaaaggagccccagggtcccagggtcctcaccgggacccccaataccctaggcacactctggagaccgggctttggggctgcagaggagaggctcctctttgctgttcctgttagggcctcgccagagggacactggacttgtctgcataaagcggtcttctgccttggagacagaaagcgatacactgtgaagaaatcgggcttctaaaactaactgtgctctcaaacacagtgccgtttggaacggggaagcactcccccagctcctggcatacactggtaaaagccagaaagataattgaggcccaatcgcaaagccctgaacacaccggtctgggagttgcagagtgcctgaatgtctgccatgggcccgcacagggccagaggcctggagggcagccctgcagcctctctggtgacccctgatggcacagacactcctcaggcggacggcacacttgctctgccaggaaacactgggagtaatgtctctctgctttctcggcatcttgcagggaaggacacaggaacccggggggaacagtggcagggtagattgcaggaccgtgactccaatctggatccacaggcaaacttttcagggagccaccgggccaatgaggccacctgcatacctgcctaaagcttccctctggcctccgtcctgtcttcatggtatctccaccttccccctatccgcggccccccaccaggcctccaagctcggccatccctagcagttcccagagctggctctcggccgtactcacatgccctgctcgccgtactggttgtagaactccatgtggctgcacgcctggatccagccaactacccaagtctccttcttggggatgggcggcatgaccacctgggccgaggcccggaagtggggtgtccggtagcggagcaccacgctggaggactcatcgatgctagtggggacggggtcgatggaggctttcacatcaatcaccgtgatcccttcccggaagactctggctttgcctccgatgctctgaatacagcccatggcatacaggagcgctctgatctccagggaaggccagcagtcacagaaaaaaccaggcattgaaaggacagaggctgcaggacccagtacagacggcgctgctctccaatctcaactctcaagaccgatatccataggatagaaaactcactgagtagactggggttgcatatatcactaccgcggcctgtttataaataaggattctgctgcatttcatgagccctgggctctctcttcttctcctcgcagtggacaaaaatcaccgatattctttgggttaaaaaaagtttgtagtttaatgaataattatgcggttctgacatccagcccttctgtgcctcacacgcggggacggcagctcgcagactctccctgaagtcttcggaggaagcaggcgagcgccggcagactcataaataaggaaggctctgtccccgcgcggccgcgccaccctcgcggcagaagcctgacttcctgccctccggccttccgcacgcgctccctccgggccccgcacccgcacccgcccggcgcggtcgaatcaattgcagagcagtttgtgttcacccggccgaggtcccggcgccctcggggcgagcgccgacccgcggcttccaggaggcgagctcagggctccggccgctcgccggtgacgggggagcgcggcgcgggtccccgtcggcgcccactcgcgggcgcgggccggacgtccccacgcaggggcgccgcgtcccttcgccgctgtcgctgccggccgccgtgcagggtcccggggctgcgctccggccgccgccgcctgcttcgccgcggccgccgaggacctggtgggaggtggaagggacagcgcgtcaaggccagcctcaaagttaagccgaactttgctgcggggggcgcgcgggggcggcgacgcgtgtgcgctgggcgcgggctgcggagctgacggcgcagctctgggcggtctccccggaggcggtggcccccgccccccgcgcccgccccgtcagtggcggccgcacgaccgcgcgcaccagcgaataatagccgcccgtgacatctccgctgataccgtcccggacgggcggggtggggggcgagcggctgccgctgcagggggcggcgagcacttgccctgctctgtccccaccgccgcgccgccagcgccaggtgccccttccccaccccggtccctccggctccctgggcgcagggccacccaccagtgccgtcggatccggcgggcacccctttcccggctggggtccctcggccctcggagggcatagtgcccccgctccgccgcgtcaagcgcctctgtcgcccctctccagccgcgagcagccccgggtccccacgcgtcgggcgcgctcgccgctgccggggaactttggcgcgcacgcccccactcgggcggcgccgccgactcacctgccccgggggccactccgctggcccgggcagccgctctccggacgcccctgacccggccgcgggcacgttcttggggcgcccccacccccttccccgggcactgtcccttggctctactggagtctggggccggcgtgggcgcccccggcccccaccctcctcccggcttggggcttcgggggggcggccgggggcggagggctgaggctcggggctctctcgcccccaccccgcgcgcctcctttattttatgatcattgtgagcgcgctgtcgccgctcagccccgccgccgcgccgctgccgcctggcgcgccccgcgctgcctcgctctgctgcagcatcggaaaggcaaccgggtcaaactttgcagaaactcggcccctgcgccggcccggcccccgcgtccgctcgccgccgccgccggccgagcgcagccggcgcacgcagggaccgccgcccgctcgccgg

SEQ ID NO: 51 - DNA, 1162 bp, Homo sapiens
cgcggggacggcagctcgcagactctccctgaagtcttcggaggaagcaggcgagcgccggcagactcataaataaggaaggctctgtccccgcgcggccgcgccaccctcgcggcagaagcctgacttcctgccctccggccttccgcacgcgctccctccgggccccgcacccgcacccgcccggcgcggtcgaatcaattgcagagcagtttgtgttcacccggccgaggtcccggcgccctcggggcgagcgccgacccgcggcttccaggaggcgagctcagggctccggccgctcgccggtgacgggggagcgcggcgcgggtccccgtcggcgcccactcgcgggcgcgggccggacgtccccacgcaggggcgccgcgtcccttcgccgctgtcgctgccggccgccgtgcagggtcccggggctgcgctccggccgccgccgcctgcttcgccgcggccgccgaggacctggtgggaggtggaagggacagcgcgtcaaggccagcctcaaagttaagccgaactttgctgcggggggcgcgcgggggcggcgacgcgtgtgcgctgggcgcgggctgcggagctgacggcgcagctctgggcggtctccccggaggcggtggcccccgccccccgcgcccgccccgtcagtggcggccgcacgaccgcgcgcaccagcgaataatagccgcccgtgacatctccgctgataccgtcccggacgggcggggtggggggcgagcggctgccgctgcagggggcggcgagcacttgccctgctctgtccccaccgccgcgccgccagcgccaggtgccccttccccaccccggtccctccggctccctgggcgcagggccacccaccagtgccgtcggatccggcgggcacccctttcccggctggggtccctcggccctcggagggcatagtgcccccgctccgccgcgtcaagcgcctctgtcgcccctctccagccgcgagcagccccgggtccccacgcgtcgggcgcgctcgccgctgccggggaactttggcgcgcacgcccccactcgggcggcgccgccgactcacctgccccgggggccactccgctggcccgggcagccgctctccggacgcccctgacccggccgcgggcacgttcttggggcg

SEQ ID NO: 52 - DNA, 50 bp, Artificial Sequence
  MYOCD source sequence
cgcctgtcagtagtaaagggtatcagatggcaaagttgggaccttcataa

SEQ ID NO: 53 - DNA, 50 bp, Artificial Sequence
  MYOCD Unmethylated AlleleA Probe Sequence
ttataaaaatcccaactttaccatctaataccctttactactaacaaaca

SEQ ID NO: 54 - DNA, 50 bp, Artificial Sequence
  MYOCD Methylated AlleleB Probe Sequence
ttataaaaatcccaactttaccatctaataccctttactactaacaaacg

SEQ ID NO: 55 - DNA, 3001 bp, Homo sapiens
tgtgtgctgttttaagccactacattcgtggtaatttgttatagcaggaatgggaaaatgatgttacgtaatcgctgagatgctcctgcacacgaaatcagcctatagacaggcgcccaggccaggactagatccccaaagggaacatggatggattgttcttcctcgtctctgacacatcagcaagaaggtgtctatccctgaagaccttatttacaaaatcgccccgctcagatccagagagtaagctctagggctaacccctctaatgtgttttcagcaacaggggatggggcagaggccgggaccaggacattagaggttgtccccatgagctattgtcacctcgggtctccatctgtcaccgactgatcttttaaggactcgtagtatgcaggcaggcttgtgtgtaatagacgacacccggggtccacgtgcatatgaagttgcaaataagccctaattatatacgtatacagagtggtttttatgttctgagccaccaactatttaacgggcttcttgagagatgggtctttgttttatttcattgatccatgtgcctataacacctcggccagatgcacaaataactctgggtcggttacggaatggattttcccctaaggagagactcattcgcaggcaaagtttttgagccttcccaagctaagcccagcccgagctctgacttgccccctgctccagagcccctccctatttagagcccagtaggtttttcactccaactcaggtatcttttcccattccctcggggttgtgaggtgggagcgttgtgtcgcagctggatctccagatgcgccgtgtcacatttgtccccccaaaaagcatatcctagaaaatgaaagcctttccatttccccgtggaaatgcatctaagtccagtattggagctgcgagatacagaatcgtctctgccagtgccctgagcccctcaaaccgaggggaatgcgtgggcttcggtccgtcggaagcttttcttctcaggggtccagcttatttccaaattgccccgcgtgaactgtcccgcgggcgcgcacgccctttcgcgcagcctttgcagccggctggcgggagatcccggccgcttccctttcgccggcagaagcccggccggggaagccccggctgcgcacgcccatccccaggacctcggagctcccagcggctgccgcagccccgcgaaccacctgcaagccccgcggcgtcgcagcccttccttctttcctccgggcatccgctctttagctcgcgcccctctcccagattcccgacccctggccctgctcccgccctctgtgcctcggcggctgcggggcccgccgcaaagagttaagagccggttcccgagacggcttcggcggctccgggtccccagaccccgctcgccgctcctgattggctgagcgcctgtcagtagtaaagggtatcagatggcaaagttgggaccttcataaaggcgtggtggcgattctccgcaatcgccggcagcctatgacatcagacaggaacgcctgggatgccgcgctgctcctggccaacctccgaggaggaggagggtcccgccggctaagagttaattagccccgcacggcgaggggggaggcgccagttttctggggacactggctgccactgtactcctacccaggggagctcacggagagttggatgaattctgggttgttagctgcggtcagctgggctcccgggagcctgttgctggtggagaacagggggcgcctggccaagggaccagcggcttgctgagactcaacatgacactcctggggtctgagcattccttgctgattaggagcaagttcagatcaggtagggctaaggcatttagcattccttcttaaactttcctcttctgcaattctcaactctagaactgctttccaataaatgtttcaaagcctgccaggtctacctcaactttgttccccaaaagcacattgtggaagcgaagagttttgtttgactttgggggcctttcgtgtcttccgttgtaagtgctttgggtggtcgagtgtggttgttcatctgtgaaaaggccaatccttttattttattttctctgcatctatgaatagaaattttggaggggtacattcagagggagcaactttggaaatgaacaaataagggatttcaagtttggaagctgcgtagtatcggttataatcgctgagaagatgaacctgatgttgtgtgttaggtacaggggtcacttaaaatgtagctaatccaaacaaatgtatttcccaaaaaaagtcacttgggggaagtaaaaatagcaaatttggagcacaccaagtatttaaaatgacatcactgtagaaaaaaaaagaaagcccagtgccacaaatattttttcccaaaccatctgatatttataaatagagaatttcccccaaaatttctgtgttagtggcacatggagaaacttctcaaaggacgctgagtggaggtgagtggaagggagagacgggagctctaaccgcttctccagaagggcttgtacacgcgcccatggaaggtgtctgtgtgggtctccacgtttagacaaagcaacacttgacttctgaagttatttttcagtagctctgtgtataatgttggttttagaaaactggttaacaacacactcaacaatgtaggacttctacttagattgatgacttttaggagaactctgatgtgaacattagacataacaggtgtcagaaagatgctggcataggttgctaagctaaagtttccttagcccttctacatggggccaactcagttgtttcacttgggatggatggttcaaaaaggaaaaatgtagatgctgggaggtaaggttcatctgtgactttcttagatgcccttggtttttagatgcctgtggagtcaactggagattttcttaggatcttcattgttgatgcaagttgaaaactcaagagcttaggcggtcacaagtctc

SEQ ID NO: 56 - DNA, 668 bp, Homo sapiens
cgaggggaatgcgtgggcttcggtccgtcggaagcttttcttctcaggggtccagcttatttccaaattgccccgcgtgaactgtcccgcgggcgcgcacgccctttcgcgcagcctttgcagccggctggcgggagatcccggccgcttccctttcgccggcagaagcccggccggggaagccccggctgcgcacgcccatccccaggacctcggagctcccagcggctgccgcagccccgcgaaccacctgcaagccccgcggcgtcgcagcccttccttctttcctccgggcatccgctctttagctcgcgcccctctcccagattcccgacccctggccctgctcccgccctctgtgcctcggcggctgcggggcccgccgcaaagagttaagagccggttcccgagacggcttcggcggctccgggtccccagaccccgctcgccgctcctgattggctgagcgcctgtcagtagtaaagggtatcagatggcaaagttgggaccttcataaaggcgtggtggcgattctccgcaatcgccggcagcctatgacatcagacaggaacgcctgggatgccgcgctgctcctggccaacctccgaggaggaggagggtcccgccggctaagagttaattagccccgcacggcgaggggggaggcg

SEQ ID NO: 57 - DNA, 50 bp, Artificial Sequence
  KCNC1 source sequence
ctggaggagatggcgggccccctgggcaggggcacccggggtgttgctcg

SEQ ID NO: 58 - DNA, 50 bp, Artificial Sequence
  KCNC1 Unmethylated AlleleA Probe Sequence
ctaaaaaaaataacaaaccccctaaacaaaaacacccaaaatattactca

SEQ ID NO: 59 - DNA, 50 bp, Artificial Sequence
  KCNC1 Methylated AlleleB Probe Sequence
ctaaaaaaaataacgaaccccctaaacaaaaacacccgaaatattactcg

SEQ ID NO: 60 - DNA, 3001 bp, Homo sapiens
gatggtccctggtttggaaggcaaggggagggtgccaacgcccttctcacctcagtctgacccgggggcgcgggcccagacgtctctcggagcccccgcccctggagtggcgttccccgagggcggaccgtgggcgggtcccccgggagcagcaggagccgcgcggggcgggcgcccccagcgccgaggttgagggaggccgggctcggtgcggggctgggaggggggcgcggaatcagggctttttaggacccagcgcggcgccttcctccgggggcgagtgcggctgcgcgcgccgtgtgcccgtgtccggagagcggcgggctggacgcggaccgcgcgaacgagcaggcgacgggcgagcagcaagcggagcagcagcccgggcggcagcagcgtcgcggcggcggcggcagcggccgctccgcgcctcgcctcaccggcctcgctggcctcacctcgccgcgcccggacccgccacccccgcctccccgcccgacctccgcagcccccgtctcggccggcagcgcccaccgcctgcccgaagcgaggagcggagtgcggggcgcagaggcaccggagccagtgcccgccggccccgcaaatcaaacccagccaggagaacccccgcctggccccgtgcagctccgcgaccgccgggagctgtcccttcagcaccgccgcgggagcccaagtccgagcgcagcccagcggaaccccagctcgagcccgggctcacggagagcagcgctcggcgttagccgcacgagcaacaccccgggtgcccctgcccagggggcccgccatctcctccaggaggcggggagaaggaacgaggagggcgggcgggcaggcgggcccgccgccggggggagcaggcagccaagcaaggagagagggagggggccggagcccgccctgcgtcccgagctgcagcccggccacgcagggaggaaggcaggcggcatccatctcccccgcgccgagagcgcgccgcggcgaccacctcacagaggagcgcgaatttcccggccctgtctcctcccctccccctcctgggggctggcctctagtccggccccgggagcggctgacctttcccccaaaagctttgtgccgatttctccattttcccgcggagatggggacgggagcccggccccccaacccattttccccacctcccggggctcgctgctgagcccgcccccctccctctttccccctcactccctccctccctccctttctccctccctttctccctcccttctttcctcctctgcctcctccgctgccggaccagctccctcccacatctggctcctagagacccctgggatcccgcgcacattcccctggaccggcacccgacaaagcgcccggagaggcttggctcgctcgttggggtggccagagccgcaggcctctgttccccccgacggctggggggaggggggaagagggcgcgcgcccccctccccggcgccaactccccctggcggccgctcccatgggtgtcgctgggccgcgccatgcctaagggggcgccgcgatgggccaaggggacgagagcgagcgcatcgtgatcaacgtgggcggcacgcgccaccagacgtaccgctcgaccctgcgcacgctgcccggcacgcggctcgcctggctggcggagcccgacgcccacagccacttcgactatgacccgcgtgctgacgagttcttcttcgaccgccaccccggcgtcttcgcgcacatcctgaactactaccgcacgggcaagctgcactgcccagccgacgtgtgcgggccgctctacgaggaggagctggccttctggggcatcgacgagaccgacgtggagccctgctgctggatgacgtaccgccagcaccgcgacgccgaggaggctctggacagcttcggcggcgctcctctggacaacagcgccgacgacgcggacgccgacggccctggcgactcgggcgacggcgaggacgagctggagatgaccaagcgcctggcgctcagtgactccccggatggccggcctggcggcttttggcgccgctggcagccgcgcatctgggcgctcttcgaggacccgtactcgtcccgctacgcgcgggtaagtgacaatttacccatcagaagagcggggcgggaaggcagcgtcctgtgcctccccgcgggcaggggtggaccggagaactggcgcctagggagttcagaatcgaaagggggtgtgtgcgcatgtgtgcacgtaccagggtaagagaggaagggtgtccgccggggttctggttttctatgtatgtcagtctgtgtgttgccaagtttaatatgtatgagtatgaatgggtgtgtttgtgtgtatgagtagattgtgtgtgcatatttatgtaagtgtggtggtgtgtatttgggtgggtaaagtgtggagtgggaatgagtgagcatgtgtgtatgttcgagtgtgcatgagcgcctgcccgtgaacatttgtgtctttgcagaggtgcactgtcaaagtatggactgctctcgagatttcagtgtggggagggaagctgggagcccttcctcctgtccaggtaaaggcgcatctggagccagccatggtcccccaaaaaggcctgaggcctgctgggcagcttgagacagacaccccctcctcagggagcagcctccggcaattttgaggaagtggtcaagagggcatctccatggctaatctcggtggcttggaagttggtggagggctgggctgtaaggtaggggtggtccgtctgagcccccgatcctgagatcacagcctgtccttcctccttgtcgaaagcggagggtgttggagtgggtcactgggcttatgccagtgcttcctctgggacatcccgtggtggggagagggggtcggtccttaagggtccttaagggggaacctgcaaggagaaggtgtgtgagaggtgggcgtgtcttcatgggccactcccctatagccata

SEQ ID NO: 61 - DNA, 2230 bp, Homo sapiens
cgggggcgcgggcccagacgtctctcggagcccccgcccctggagtggcgttccccgagggcggaccgtgggcgggtcccccgggagcagcaggagccgcgcggggcgggcgcccccagcgccgaggttgagggaggccgggctcggtgcggggctgggaggggggcgcggaatcagggctttttaggacccagcgcggcgccttcctccgggggcgagtgcggctgcgcgcgccgtgtgcccgtgtccggagagcggcgggctggacgcggaccgcgcgaacgagcaggcgacgggcgagcagcaagcggagcagcagcccgggcggcagcagcgtcgcggcggcggcggcagcggccgctccgcgcctcgcctcaccggcctcgctggcctcacctcgccgcgcccggacccgccacccccgcctccccgcccgacctccgcagcccccgtctcggccggcagcgcccaccgcctgcccgaagcgaggagcggagtgcggggcgcagaggcaccggagccagtgcccgccggccccgcaaatcaaacccagccaggagaacccccgcctggccccgtgcagctccgcgaccgccgggagctgtcccttcagcaccgccgcgggagcccaagtccgagcgcagcccagcggaaccccagctcgagcccgggctcacggagagcagcgctcggcgttagccgcacgagcaacaccccgggtgcccctgcccagggggcccgccatctcctccaggaggcggggagaaggaacgaggagggcgggcgggcaggcgggcccgccgccggggggagcaggcagccaagcaaggagagagggagggggccggagcccgccctgcgtcccgagctgcagcccggccacgcagggaggaaggcaggcggcatccatctcccccgcgccgagagcgcgccgcggcgaccacctcacagaggagcgcgaatttcccggccctgtctcctcccctccccctcctgggggctggcctctagtccggccccgggagcggctgacctttcccccaaaagctttgtgccgatttctccattttcccgcggagatggggacgggagcccggccccccaacccattttccccacctcccggggctcgctgctgagcccgcccccctccctctttccccctcactccctccctccctccctttctccctccctttctccctcccttctttcctcctctgcctcctccgctgccggaccagctccctcccacatctggctcctagagacccctgggatcccgcgcacattcccctggaccggcacccgacaaagcgcccggagaggcttggctcgctcgttggggtggccagagccgcaggcctctgttccccccgacggctggggggaggggggaagagggcgcgcgcccccctccccggcgccaactccccctggcggccgctcccatgggtgtcgctgggccgcgccatgcctaagggggcgccgcgatgggccaaggggacgagagcgagcgcatcgtgatcaacgtgggcggcacgcgccaccagacgtaccgctcgaccctgcgcacgctgcccggcacgcggctcgcctggctggcggagcccgacgcccacagccacttcgactatgacccgcgtgctgacgagttcttcttcgaccgccaccccggcgtcttcgcgcacatcctgaactactaccgcacgggcaagctgcactgcccagccgacgtgtgcgggccgctctacgaggaggagctggccttctggggcatcgacgagaccgacgtggagccctgctgctggatgacgtaccgccagcaccgcgacgccgaggaggctctggacagcttcggcggcgctcctctggacaacagcgccgacgacgcggacgccgacggccctggcgactcgggcgacggcgaggacgagctggagatgaccaagcgcctggcgctcagtgactccccggatggccggcctggcggcttttggcgccgctggcagccgcgcatctgggcgctcttcgaggacccgtactcgtcccgctacgcgcgggtaagtgacaatttacccatcagaagagcggggcgggaaggcagcgtcctgtgcctccccgcgggcaggggtggaccggagaactggcgcctagggagttcagaatcgaaagggggtgtgtgcgcatgtgtgcacgtaccagggtaagagaggaagggtgtccgccg

SEQ ID NO: 62 - DNA, 50 bp, Artificial Sequence
  FSTL1 source sequence
tcccgcttacggcccgaactacttttcctgctttaaagatttaagtttcg

SEQ ID NO: 63 - DNA, 50 bp, Artificial Sequence
  FSTL1 Unmethylated AlleleA Probe Sequence
tcccacttacaacccaaactacttttcctactttaaaaatttaaatttca

SEQ ID NO: 64 - DNA, 50 bp, Artificial Sequence
  FSTL1 Methylated AlleleB Probe Sequence
tcccgcttacgacccgaactacttttcctactttaaaaatttaaatttcg

SEQ ID NO: 65 - DNA, 3001 bp, Homo sapiens
taaaaacagttgattgagtttccacctcaatcaacaacgtagtagtgaattaaatgagaattcagtacaatgcgtattttttaaaaattgtgaagaccttgtatgcatttatgacttttttgggcagagtcaagctgcgatgctaagggtgtggttaaactgtaatatgaaggaaaaaattcaaacagggagcataaaccacccaccagggagctatttcttttgtaatgtgcattccatttcttctccctcatgctttttaattccttccatatttcctgccataattatcaaatgtttagtttctgacctcactttcctggagactggagcccggggaatttagaattgtttattttctgtccataaacctttgagttaatggagcggcttctgagcaaagagtttgttggtttgtttgtagtgtctcaggaaggtcgttagagttgaaaaaattggtacaaaaaaacaagcgctgaaatgctttaactggaaactcgagcccccaaagagcagagcaatggatcttctcttacacttgcgcaacgttacagggaacacttgggaaaaggccgttacattgcccagatgataacacaaaacccgaggacacttcacaaagaagaaaatacatttcagagaatcatgatctcactttaaaagagagaaaggaaaattaattttgccccaaaagaagaatcccgattcctctgcccccgcccccaggagacagacttgggagggaaaagtccccaactactctcagtctcctctgaagtgaaggacggaactgaggcgcctttttgaactcgctgctgctccagtcgccgcctcgtccccgtcggcccagcccgtcccctctcctcctccccgctctccctccatgttagccgcggcggggctcccgcttacggcccgaactacttttcctgctttaaagatttaagtttcgtaagcagcattactgccccagctccaccccagcacaccccaaggaccgaaactcccagcgccaccccgggagagcatccccaggacgcgcgcccacccgcccagcgcgcagacccaagaggccccggggaccgagttcggactcctcggcccctcgcctacctcggcgcggacccaggcgaccgccaccagcgcgagcgcgagcgcgagccagcgtttccacatctgcggaagagagaagagagcgagtctgaaggcgccgggccccgcgctgacctgggaaacttgctcgggtcccgcaggctcgctccggccgcccaagcacccccggccgcccgaagagtccggcctccgcgccgtgcgccctgcgctcaccgtggtctggtccaggtctcctgggggcgcggggcaggacggcggcagcgagctgtaagcggaggtgggagctccgccgatcgccagcctcggaggaaatgcgacctcccctcgccgccccttaagacctctccttccccgcctgcctgcccccccagctcctccccattctcctcccccttctgctgcagcggcccccgcggctcccgctccccctccttccgacccctgctccggggagcccgcgggagtcctggcgccgccgaacccctctctccgcacggtccctcgtccggcgccccttcggccccctggacgtggagcggaggcagagggggtgtcccgtgggtccctgccacaaaggggtagggggaagggaaaccccaccccgggaggggctgctggctggcggcggcccgggcgggctgcggcggtggccaggtgggcggtgccgtggggggcccgggaaggtccgggactggaacctggaggaaaagacgtcccgcggcctaggaagcggagccgccccccatggctgaagggggaggcccagggaggcagaggagaccccgcatcccggggtcacggcagcagggcagcggggcaccgggaagtgggcgcagggccgggaagaccggccggcgtggggaaggctgctgtgggttctacagtgcattgtcccgggtgaggtggcggccgccctgctctgctgggaagatcctgagtccagcgtttgccggcagcctcatcccctccttccctcgggccggggagcggtgtccccctcctctcttccgcccactcactcatcacctgtgtccctcctcccatccctgtcatcccaagtagcctctgtgttcaggattgccatcttcaccaccttcttcccagcccttggccttcgaaagtgtgctacctctttgacagggttaactccctaaaatgctcctaattaaggcgcctttcctgccataactagtaaggagagcagatccacttagcaactcagatttcacatgcccaagactaagcctcacttcccccagcttcagggcctgcttatctacctctttcagggtggttgccttcaaacctatctccttttctcagagccggcttgtacccatagcctggcagtttagccgctgagatctcatatgcatcccctcgcctccgttcacatgtccactttcctaggccagaccttcatcctccctcactcagaaattctggaaacctcctttcgttgtcctctgactccaggctctcccctgtagtctccactgcaatccattgtcatattctttcccttattttaaacattgtcttcctcgtgttcatgttcttcctctactcagaaacttcagcgttcttctacccacatcaaatccaaactctgcctttcttataagccctgcattactgggcccaccctgtccatctaacttctcttcaactacatcagcatacttcctccgctcaaagcaggcccatctcctcgttgtcctctgaacttgtcatactcactctgctgccaaggtttgttgagtgcccactgttgtggctttagggggcttcaagataatacccatggtccccatgtaagttcagaatccactgagatagcaacaggagtcagacttatggagagcaaaatgtcagctttgtctctgct

SEQ ID NO: 66 - DNA, 1317 bp, Homo sapiens
cggaactgaggcgcctttttgaactcgctgctgctccagtcgccgcctcgtccccgtcggcccagcccgtcccctctcctcctccccgctctccctccatgttagccgcggcggggctcccgcttacggcccgaactacttttcctgctttaaagatttaagtttcgtaagcagcattactgccccagctccaccccagcacaccccaaggaccgaaactcccagcgccaccccgggagagcatccccaggacgcgcgcccacccgcccagcgcgcagacccaagaggccccggggaccgagttcggactcctcggcccctcgcctacctcggcgcggacccaggcgaccgccaccagcgcgagcgcgagcgcgagccagcgtttccacatctgcggaagagagaagagagcgagtctgaaggcgccgggccccgcgctgacctgggaaacttgctcgggtcccgcaggctcgctccggccgcccaagcacccccggccgcccgaagagtccggcctccgcgccgtgcgccctgcgctcaccgtggtctggtccaggtctcctgggggcgcggggcaggacggcggcagcgagctgtaagcggaggtgggagctccgccgatcgccagcctcggaggaaatgcgacctcccctcgccgccccttaagacctctccttccccgcctgcctgcccccccagctcctccccattctcctcccccttctgctgcagcggcccccgcggctcccgctccccctccttccgacccctgctccggggagcccgcgggagtcctggcgccgccgaacccctctctccgcacggtccctcgtccggcgccccttcggccccctggacgtggagcggaggcagagggggtgtcccgtgggtccctgccacaaaggggtagggggaagggaaaccccaccccgggaggggctgctggctggcggcggcccgggcgggctgcggcggtggccaggtgggcggtgccgtggggggcccgggaaggtccgggactggaacctggaggaaaagacgtcccgcggcctaggaagcggagccgccccccatggctgaagggggaggcccagggaggcagaggagaccccgcatcccggggtcacggcagcagggcagcggggcaccgggaagtgggcgcagggccgggaagaccggccggcgtggggaaggctgctgtgggttctacagtgcattgtcccgggtgaggtggcggccgccctgctctgctgggaagatcctgagtccagcgtttgccggcagcctcatcccctccttccctcgggccggggagcg

SEQ ID NO: 67 - DNA, 50 bp, Artificial Sequence
  SLC6A4 source sequence
cgcaaaaattcttcaagagctctttggcggcggctatctagagatcagac

SEQ ID NO: 68 - DNA, 50 bp, Artificial Sequence
  SLC6A4 Unmethylated AlleleA Probe Sequence
atctaatctctaaataaccaccaccaaaaaactcttaaaaaatttttaca

SEQ ID NO: 69 - DNA, 50 bp, Artificial Sequence
  SLC6A4 Methylated AlleleB Probe Sequence
atctaatctctaaataaccgccgccaaaaaactcttaaaaaatttttacg

SEQ ID NO: 70 - DNA, 3001 bp, Homo sapiens
atccatattggaacggtcactgccaccccagagtgcgtggcaggaaaacccgaaccctggggcctcagtggcttcacacagaagcaagtaatgggacaaagagtcaaagctgtgtgctccctacgctcctaaaaacccaacactgacgctgcccacagttctctgtttcattaaacacaagtcccaatgtcaccttcttagacatttttcctatgtaactgaaggcaaaacacattaaatatttgctctcgtcttcctatgatgagagttagctagcaggctcataaataatccattgaccaggttcacttttctggacaaaactgatggtaaaaatacatacacaaagtctttgtaatgtctgattcagaaattctacccctctcaaattcttcagagtcccaaatggaaaaggaacaattcaatatgcaaaagccccagttcagaagaaaatctgctccttagcccaagacccaaatccactcttgagctccagagagctttttttcaagaacaagtttgaatgtacacaattttcaagcaaatgatggttatatccttcaaccacgtagcacagtcgttggagttggagtttcacatacattctgaatggcctgtgaggtgtctgagagcgcctcagctctgacagtgaaaccacccaagcgcacggagactcggcccacttaggcgccactcagagatttctctagctgtctgggcatttgtgtcaaccagaactctccctttgcataaagccacctgcacgcgatgacagcaaagtaaagatcaaacataaaccatgggttgaaacgaaagcaagcaactcttcccaaagcgcaggacagcactttgctcaagaccctctttaaggggtctttcacgggtcctcaagaggttgcaaagcccctgcaacaatagacaaaggagccccctacgcccgcccacttcgagcactccacgttcctcgtctcccactctggccggtcagcttcagctctggctcttgctgagcgtggagggcgcaggggcaggatccggcgcccaccgctggggcgcatgcacctcctcgcctcctcgcagggcggtggggcgcagcggttctatccccctccccgaggcggggaagaaggtctggaaagaaacgtgggttcgaggcggagaggaaaagcggacccacctgccaggctgcgcggggaggctggtcccgggctgggcaggcgggctggcctcgcgccctcgaggcacccggcggcgctggctgtgcggaggggcgccggcgcggccgtatttgtacccgcgggccctcacatggtctgatctctagatagccgccgccaaagagctcttgaagaatttttgcgtcactttgaggcgaataaacttaatgcttccccgcggccgcggctccgcgctcccgctggatggggttgcgctcgccagggaggggccgcgctacggggcggggtgcgcgcccgaccccagagccaggaggggagggacccccgacacacacacacgctcgcagggaggagcggagcgcggagcagccggcagggcagggccgagcgaggaatcctgactttcctgctctttaactttgcgggaggggggaggctgcacaaaggagcaggtgtgcgcctcccctgccgccccgcgcccacaggacggcacacaggggtctgctcgtgccgctttctcttgacctcggacacctccccatcgtccccatcccgaaagctgctctctctttcttcctgggacctagcagtactcgatacccatgcgtttatctgactgaccggctgggatgtgagctcagcttagaggcccttgtctaattcctctccgtccccagagtctcacaataaagacttccacaatgaaggaatgaagaactggttctcattttgttaaccagctacagttcaaagctgcgtcaaaaaggagcacagtgctttgggaggctgaggcgggaggattgcttgagcccaggaattcaagaccagcctgggcaacacagcaagaccccatctctagaaaaaaaatttttttaattagccgggcgtggttgctcgcgcttgtgttcccagctactcaggaggctgaggtggaggatcagctgagcccgggaggtcgaggctgcagtgagccgtgatcgaaccactgcactccaacctggtgacaatttgaattatctgaaagggcccagcaggagcctattgttttgaaccatgtgcatgtagttttgataattttttcaaaaagtttaaaaattgatttaatgttaatatcctttttgctaaaattatttaaaactttaaaaagggcacatgcagtacttgctttgggttgtccctgaggcggaaaggagagggctctacctctccccaagaggaggccaggaacgataggaagtagaagaccgaaaggaaatagcagtgacaagtttgcagctccagagaagccaccgccccttgtacttggaggaactgacccctgaaaactgtgcggccggttgggctgagcgtctagagggactgagctggacaaccacgggcaagcgagggcagctcccagcgggtggagtccgcgcgggattctggtgccacctagacgccagggcggggaccgcaaggtgggcgggaggcttggaggccgggatgcgggggaatactggtagggtgcaaggagaatgctggaggggtgcaggggggatgccgggggtgcatggggggatgctggggggtgcaggggggatactgcgaggggtgcaggggggataatgggggttgcaggggagatcctgggagaggtgcagggggatgctggaagggctgcaggggggatgctgggggtgcaggggagatgctgggggggctgcaggggggatgctgggggtgcaggggggatgccgcgaggggtgaagggggggataatgggggatgcaggagcgatcctaggaggggtacagggagttgctgggaggttgcaggggggatgc

SEQ ID NO: 71 - DNA, 799 bp, Homo sapiens
cgcccgcccacttcgagcactccacgttcctcgtctcccactctggccggtcagcttcagctctggctcttgctgagcgtggagggcgcaggggcaggatccggcgcccaccgctggggcgcatgcacctcctcgcctcctcgcagggcggtggggcgcagcggttctatccccctccccgaggcggggaagaaggtctggaaagaaacgtgggttcgaggcggagaggaaaagcggacccacctgccaggctgcgcggggaggctggtcccgggctgggcaggcgggctggcctcgcgccctcgaggcacccggcggcgctggctgtgcggaggggcgccggcgcggccgtatttgtacccgcgggccctcacatggtctgatctctagatagccgccgccaaagagctcttgaagaatttttgcgtcactttgaggcgaataaacttaatgcttccccgcggccgcggctccgcgctcccgctggatggggttgcgctcgccagggaggggccgcgctacggggcggggtgcgcgcccgaccccagagccaggaggggagggacccccgacacacacacacgctcgcagggaggagcggagcgcggagcagccggcagggcagggccgagcgaggaatcctgactttcctgctctttaactttgcgggaggggggaggctgcacaaaggagcaggtgtgcgcctcccctgccgccccgcgcccacaggacggcacacaggggtctgctcgtgccgctttctcttgacctcggacacctccccatcgtccccatcccg

SEQ ID NO: 72 - DNA, 50 bp, Artificial Sequence
  NPTX1 source sequence
cgacctgggtccttgggcacccagcccggctccaaacgaccccggcccaa

SEQ ID NO: 73 - DNA, 50 bp, Artificial Sequence
  NPTX1 Unmethylated AlleleA Probe Sequence
ttaaaccaaaatcatttaaaaccaaactaaatacccaaaaacccaaatca

SEQ ID NO: 74 - DNA, 50 bp, Artificial Sequence
  NPTX1 Methylated AlleleB Probe Sequence
ttaaaccgaaatcgtttaaaaccgaactaaatacccaaaaacccaaatcg

SEQ ID NO: 75 - DNA, 3001 bp, Homo sapiens
gcacccagctgtacgctgaggctggggaccagggagagacaggctttcttaagcagggagggtttgagccagcgacagccactgtgcaaagttactgaggattaaacaaatcagacttgaaaaatccagcgagtgtgtgagtgtgtctggatgggggagggggcactatgcaatcccctttcccagagacagccccagcccaaggcacccagccctgatagaaacaccgactagaagcccagaacctgggaaggggtgcggggaatgagaaagcggtgcaggggttgggggtagcggagcggcttgaggcttgcacagtgcagagctgggctgttagggatcgggaccgaggcagggtcgggcaagcaggtgcaggcaggcagcacggctccccctggccccagtacctttctcgagctcgctgatccgctggtgcagggaggtcagggcggtctcgatcttgaccctctcctcggtgtcgttcctggggccccccttgccctcctccagggtgttcacccgggacagcacctgcctctccagctcatcgatcttgctctgcagcagatccttgaggctgttggtctggctggaggaattgaggcggctgtactgctgcggggtgcaagggcgggggaaaccacaccgttaggcgaggcgcggggaccgtgctggaaggcccgcggcgcggtcccctctgggcgactgcgggggcggcctggcagggagctggggcgagtggccgccgcggggcctcgcaggcgcggggaggcaccggccgggcacccgcgcggctgaggcgagggcgggggatgcctggccgggtagggaacggggtgggggagggcagaggggcgagcgagccggagggggaaccggagccgagggcgcgcgcggacctcgaggttctccaggcgggttttgagcgattgcaaagtttgcccgagttggctgagcgtctcggcggccggtgtccgggacaggtcgcccatggtgttcttgcccgagccgggctgcttgcggccgccgcccgcccgggcctcgccggctccggggtccagcgtgctctggctctcgcagcggcccagcttggcggtcagctcgcggatggtctccttctggctcaggatggtctccttctgctgcagcaccgtctcgcggagctgcagcacgctgctccggagctcctcggcgccgccggcggccacggacgcggcgcacatgtcggcgtccacgggcaccgaggtgcagatgaagcgcgtcggcccgaaatcctgggccccggcgcccaggaggcagagggcgagcagcgcacaggtgcgcgcggcgcggccggccggcatggctgcgggcaccgggcgctccgggcccggctcggctcggctggggctcggctccggctgggacccggctcgggctgtggctccgcgagcggcccgcgctctgggcgccgcgctcttcggccgcgcggtccacaccgccgcgctatcaggcccccactgccgcctgcgctgcggagcccgcgccttttatgccgccgcgggaggggcctcgagccgccgacgtcagcgggggaggaatcccgctttaattgtccgcggcccgggcccgcggcgcggcgggggtctcacgagcgcgccaccggggctgcctccgctcgcgtcccccccgcccttctgggagctgccgcggccccctcctcccccgcccccgcctgggccggcgctgctgccccgaaggctccgagagctcagggggaaggaagtcgggctccggccccggactcgaggatggaccacctgcgtggtgtccccgaacctccgacggggctccaggcgggcgggaaaccaccgcgtccgggagatagaattcggttcggccctcctggctgcactgaacacccttgggccggggtcgtttggagccgggctgggtgcccaaggacccaggtcgtcaacacccgagtgcccggtggtcaacggctcggagtggggcctcgcggcgccactaagtccagagcgggggtcaccgggccgccccgcgtttcttagatgtccgtgtgctattgagaagccgccgggcagcctgggtacagggacttcccacgggtcggggcccccggcgcctcccctcccccagctcccgggctctgtggacccggccccgtccccgggggactgtcagtccccaccccgtgcgggcggggcgctggggacttctcggaggcagacctagctgatcgagcctttcctgtccgcggcgccggcccgatcgcgggcaggcggcgtcgccggggctggacgttcgcagcggcgcttcggaagggggccccgcgggagcagccgcccgcgtctccagcagcttccccttgccaggcgccgcgcgcgcccggtatccccgggtgtccacctgtgcgtggggggctgtttcccgtctgtccagccgcgcccacttctcaggcccaaaggccagcaggaagggtcccggaggtggctgggggcgtccacctgagaagctccgctctcgctcagacaccccacccggcacccgcgcactcgcgccttcatctggaccgcggggctcggctcccgagatcgcggtccgggttccggcgccgggctgggggctcaggaccagggctggcgcagccgccccgccccgccccgccccggggctatcagagcggtcaaagcctcggaaatccctccgtccacgctcccgtggtacagagaaggaaactgaggctccgagactgcggggccgggctacagccgtggccccccgacccctccccgccgcccccggcttctctggtctctgcgttctgcagcctttccctgcgcgcagccgggggctgggggtccccagccagcagggacccacgggcagtcccgggtgccgggatcgggcgtccgcccctcgggcgggagcagaggccggctcgatggagacctttttgagaaatggcggcctctacccgccccccagcccccgagcgccc

SEQ ID NO: 76 - DNA, 3276 bp, Homo sapiens
cggctgtactgctgcggggtgcaagggcgggggaaaccacaccgttaggcgaggcgcggggaccgtgctggaaggcccgcggcgcggtcccctctgggcgactgcgggggcggcctggcagggagctggggcgagtggccgccgcggggcctcgcaggcgcggggaggcaccggccgggcacccgcgcggctgaggcgagggcgggggatgcctggccgggtagggaacggggtgggggagggcagaggggcgagcgagccggagggggaaccggagccgagggcgcgcgcggacctcgaggttctccaggcgggttttgagcgattgcaaagtttgcccgagttggctgagcgtctcggcggccggtgtccgggacaggtcgcccatggtgttcttgcccgagccgggctgcttgcggccgccgcccgcccgggcctcgccggctccggggtccagcgtgctctggctctcgcagcggcccagcttggcggtcagctcgcggatggtctccttctggctcaggatggtctccttctgctgcagcaccgtctcgcggagctgcagcacgctgctccggagctcctcggcgccgccggcggccacggacgcggcgcacatgtcggcgtccacgggcaccgaggtgcagatgaagcgcgtcggcccgaaatcctgggccccggcgcccaggaggcagagggcgagcagcgcacaggtgcgcgcggcgcggccggccggcatggctgcgggcaccgggcgctccgggcccggctcggctcggctggggctcggctccggctgggacccggctcgggctgtggctccgcgagcggcccgcgctctgggcgccgcgctcttcggccgcgcggtccacaccgccgcgctatcaggcccccactgccgcctgcgctgcggagcccgcgccttttatgccgccgcgggaggggcctcgagccgccgacgtcagcgggggaggaatcccgctttaattgtccgcggcccgggcccgcggcgcggcgggggtctcacgagcgcgccaccggggctgcctccgctcgcgtcccccccgcccttctgggagctgccgcggccccctcctcccccgcccccgcctgggccggcgctgctgccccgaaggctccgagagctcagggggaaggaagtcgggctccggccccggactcgaggatggaccacctgcgtggtgtccccgaacctccgacggggctccaggcgggcgggaaaccaccgcgtccgggagatagaattcggttcggccctcctggctgcactgaacacccttgggccggggtcgtttggagccgggctgggtgcccaaggacccaggtcgtcaacacccgagtgcccggtggtcaacggctcggagtggggcctcgcggcgccactaagtccagagcgggggtcaccgggccgccccgcgtttcttagatgtccgtgtgctattgagaagccgccgggcagcctgggtacagggacttcccacgggtcggggcccccggcgcctcccctcccccagctcccgggctctgtggacccggccccgtccccgggggactgtcagtccccaccccgtgcgggcggggcgctggggacttctcggaggcagacctagctgatcgagcctttcctgtccgcggcgccggcccgatcgcgggcaggcggcgtcgccggggctggacgttcgcagcggcgcttcggaagggggccccgcgggagcagccgcccgcgtctccagcagcttccccttgccaggcgccgcgcgcgcccggtatccccgggtgtccacctgtgcgtggggggctgtttcccgtctgtccagccgcgcccacttctcaggcccaaaggccagcaggaagggtcccggaggtggctgggggcgtccacctgagaagctccgctctcgctcagacaccccacccggcacccgcgcactcgcgccttcatctggaccgcggggctcggctcccgagatcgcggtccgggttccggcgccgggctgggggctcaggaccagggctggcgcagccgccccgccccgccccgccccggggctatcagagcggtcaaagcctcggaaatccctccgtccacgctcccgtggtacagagaaggaaactgaggctccgagactgcggggccgggctacagccgtggccccccgacccctccccgccgcccccggcttctctggtctctgcgttctgcagcctttccctgcgcgcagccgggggctgggggtccccagccagcagggacccacgggcagtcccgggtgccgggatcgggcgtccgcccctcgggcgggagcagaggccggctcgatggagacctttttgagaaatggcggcctctacccgccccccagcccccgagcgcccgccgtctgcgttcagcatctcagcttttaagcctccctgagccggggctgcgttctcgttgtctccttcggccgccgcttcctggcgccagagggaccccgcggcccgcacgggcctgtcctcggctccgctcggagggtgtgcgggttcccgctccgctggccccgcttgcccaccacggcgcgcccaaacccacgcggcccgcggccccttgcgtgctctgccgaccccgggccgcaggaggggttagcctcgccggtccccacgcggggtctgcgccggcagcgccacgggctggaacgcgcctcgggagaaattacccgcgcggggtcgcggcgtcttctgccccaactcccgtagggagccgctaggcgggtgccctcctggccccagcgctttggcctcgccgccctcccgcacgcgtccagtggggaggccgcgcccgtcaggcctggagccggaccgtgcccagccgagagcgcgcgggcgcgggagaggagccctgccctgcccagggcaggctgtttagacggggcgggaacccttagcctggccccgcgtctctatccccagcagccgtggagccttcctagccgcccaaggccagggaccctgcctggcttttcctccagggcagcagatctcagcggcctggcacctcggcacagaggagcgtgtcccccttccgcagaggaagggagtggacggcgtcagaaagggaggggagcctggggcggtgtctggggctagcgggacctctgggcactgtggggccccaggagaggagaccctcgggtccagcctgcaactgttgtgcaggaggcgctgggcgggacacctgcaggcgctgcacacacacgcgagggccaccatgccgcg

SEQ ID NO: 77 - DNA, 50 bp, Artificial Sequence
  SNCB source sequence
cgtccccacagccgcccgggcagctgcaaacaccggagcatactcacata

SEQ ID NO: 78 - DNA, 50 bp, Artificial Sequence
  SNCB Unmethylated AlleleA Probe Sequence
tatataaatatactccaatatttacaactacccaaacaactataaaaaca

SEQ ID NO: 79 - DNA, 50 bp, Artificial Sequence
  SNCB Methylated AlleleB Probe Sequence
tatataaatatactccgatatttacaactacccgaacgactataaaaacg

SEQ ID NO: 80 - DNA, 3001 bp, Homo sapiens
agctccgttcaaaccccagcttcagcatttaccagctgctgaccttgggcaaggtacttagatctctgggaagcccagtttcccttacctgtagaatgggccgaagcattacatgaggaaatggatgttatgacattttacccagtacttggcatacagaaagtgttcaacaccagataactattaataatggtaaaaaagataagaaaaaggaggctgtctgctttcatcactgcactggtccctggcctttcagctggagccccccacctcatcagcccgccccgtgtccccatttccggatacagacccaggcttctatgggctggctatgtcccctatgacccctgctgacctcgccccatctgctgactcatattctcctgcccagaacccccctccccgggacccggccccagctccacactgtcgggggacccccagccctgcagccccagaaaccccgccccttacccaccgacgtagaggacgccctccttggtcttctccgccgcctcggtgaccccctgcttggttttctccgcggctgccacaacgccctccttggccatggacaggcccttcatgaacacgtccatcctggcggcctggggagggcgatacacgggcaccggtgcactggccccgcactctcaccccagcccctcccgcgggacgcagatgcccccctactcccgagaccgcggcgcccttctggaccctgagccccctcccgctttccccccatcccaccccactccccagtgcgaagcctcagggccgcggagagtcctagcgtccttgaaacctggggacgcgggaggggccactgcctcggttatccgggccctgcaaactgcagccccgtcgaaccggagtgctgggttcggcgcgaatatccaggacccgcctgtacacgcacggcacagtcacacggtcatgcacacaaacatacaccacggacaagctcacgtgcacacacaggacacgcagacgctccaacgcgcacgaataccccgacccacatgggcgcggacacacgtccccacagccgcccgggcagctgcaaacaccggagcatactcacatactccggggtgcacactgacgcgcatcccccgggtctccctccattttccatccccttccccagctccctcctgtcgccccagtcccctctccatcctcatcccgttccccatccccggcgcgcggccgcctcacctggatgcggggccggggctggggatggagcggcggctgcggcgggcggacgcgggggctccgctaggccaggccggggtggaccaggggccggaggggggatagggccgggatcaggcgctcgggtcgcgggtcctgagcaggagcgcagcagtgcccgggtctcgggtgcgtagccagccaccgctcgttcctcgcgccctgcgagctcgcgcgctccggctccggctccggctccggcgctgcggcagctctgagctcagcgcccccgctggcggccgcgccgccccttcggcctgactgacagggggcggggagggtgacagcgcgtagccgatgagcctcccggggcgggcccgggacgcgcccctgggggccccggccgtaagactcgcgtagtctgttgggagttgagggagggggacaacgcggcgtgtgaagagtggggaagaaatcacgcgactggatttggagttctgctgggctcgaatctcagctgtgctttttattaaccgtgggaccttgagctactcggctctcctttctgaatgacagtttcctcctctgtaaaacaggaatggttaattcaacgaatgtttactgagcgcttagtccagtggtgcaggtttaaggctggaggcagcggaaaaggaaaacaaggttcctggtctcgggaagcttaccatgggggtgtctgtacgcgtgtgtgttcgtgcgctcagtggtggaagacaggacagaaaataaacggatcaacatccaaagcaacaaggtcattacagcttgtaagttgtgaaggtaacggtgatgtaacacggggactagaggaggggtgcttgagacagggtgctcagggaaggcctctaccagcaggtgaattgtagcagagactccagagaggcaccatgacaccacgatgggtgaggacaagatccatagagagtgcaagtgcaaaggctccaaggcaggactgtgctgacaaaagacagcgcagctggagagccaggatggagggaggtggagaacaaaaccacttttttgtgtgcttacggtggggactgtcaaaatgatcacagaatgccaggcacacagtaggccctcagttggttctatgatagaattatgcccaagaagctgtagtatcccagaggaaagggactctgacaacttaggggagtcaggaaaggacataaaaaagaggtgacaactgaactgtggcctgacaggaaggggactatttcatcgagcaggctagggagaaagagaattttaggctgtgggtacagcatgggcaaaggcactgtggccagaaatgcagggtgtgtaagtgacaggtaattaggcgagggggccaccagcagccagactgcaaaaaaaaaaaaaaaggaaaaaacccaaccaaccaaacaaaaaacattcacaggccgctggtagaactgcaaattggcgcagacattttggaaggcaatctggcaatgtttaccaagcataaatgcatgcgcctttggagccaacagctccccttccaggaattgatcccgtgcgactctagccatacatgcacacaaagatacctgtgcaaggatgttcactgtagcatgtttttttgtagcaaaaaactgaaaatgatctaaatgatcatctgtacaggactggctagctaactgtggcacatccacccagtgcaatgccacactatggttggcaaagatgagctagacccatgtgtgctgacatgggagtgtgccaagatgtggtggtaagtgtgagaagcaag

SEQ ID NO: 81 - DNA, 974 bp, Homo sapiens
cgccccttacccaccgacgtagaggacgccctccttggtcttctccgccgcctcggtgaccccctgcttggttttctccgcggctgccacaacgccctccttggccatggacaggcccttcatgaacacgtccatcctggcggcctggggagggcgatacacgggcaccggtgcactggccccgcactctcaccccagcccctcccgcgggacgcagatgcccccctactcccgagaccgcggcgcccttctggaccctgagccccctcccgctttccccccatcccaccccactccccagtgcgaagcctcagggccgcggagagtcctagcgtccttgaaacctggggacgcgggaggggccactgcctcggttatccgggccctgcaaactgcagccccgtcgaaccggagtgctgggttcggcgcgaatatccaggacccgcctgtacacgcacggcacagtcacacggtcatgcacacaaacatacaccacggacaagctcacgtgcacacacaggacacgcagacgctccaacgcgcacgaataccccgacccacatgggcgcggacacacgtccccacagccgcccgggcagctgcaaacaccggagcatactcacatactccggggtgcacactgacgcgcatcccccgggtctccctccattttccatccccttccccagctccctcctgtcgccccagtcccctctccatcctcatcccgttccccatccccggcgcgcggccgcctcacctggatgcggggccggggctggggatggagcggcggctgcggcgggcggacgcgggggctccgctaggccaggccggggtggaccaggggccggaggggggatagggccgggatcaggcgctcgggtcgcgggtcctgagcaggagcgcagcagtgcccgggtctcgggtgcgtagccagccaccgctcgttcctcgcgccctgcgagctcgcg

SEQ ID NO: 82 - DNA, 50 bp, Artificial Sequence
  ARHGEF7 source sequence
cgtggctcatcactctgggggtgctggagtcgcccaaaaaaaccatctcg

SEQ ID NO: 83 - DNA, 50 bp, Artificial Sequence
  ARHGEF7 Unmethylated AlleleA Probe Sequence
caaaataatttttttaaacaactccaacacccccaaaataataaaccaca

SEQ ID NO: 84 - DNA, 50 bp, Artificial Sequence
  ARHGEF7 Methylated AlleleB Probe Sequence
cgaaataatttttttaaacgactccaacacccccaaaataataaaccacg

SEQ ID NO: 85 - DNA, 3001 bp, Homo sapiens
aagtatcaaaccgaatttagtaggattcgaaatggtaaggactgcattaaattatttctctaattcagcagtaaggaaagtgaacagaagtctcggtgtacaaccacgccccgggttgacatctttccttccacgaagacactgggtacaagccttgatgtagctgttaattgccctcctgtttcctagaaagcttatagggcagcagtaggaaacgggttaggtatgcctttcctggtaggagccgagctttgggtcttatctctgccacttgttcggcatctgatcagtaaaaggggaccatgatgggaggcgaggcagtagacagcactttgtgacggttcctaaccagtcgctgttatcactgagcacattgtcctcgtaatccagaattcatactgccatttgaatattttttgtatttgcaaagaaaattttaaccacttaactcaaggctagaaataaaagcaattagtgattgcaagagaaatccaccggcagaaggcagggggagacgacttaagcctctgattatttttggaagctttatatgctttctagcctttgtcattgaatggattaatgtatgtgcctggcatagactaagtgctttgcttgttaaactgcccgcgggtcatagcgagcaaactgggggagcgcccagggacagtggatggagtgagtggagactcgtgtccttttaagaagaggctccatttcttcctcgcatgtcaatgtgtgcgtgtcatcagttgttcgaggaagctcgcgcatgacgcgacacttgccgtctgggctctccaggttcggcctgctgctttcacagcccagatcttcggcgtccccgagacgcagccaggcgcccggcccggcccggcccggccctctgcacagccccacttcaggccacgccgtttccttgaccttcccccggcagggtctgaagcgtcaccgtccgtctagagcagagtcgcgcaggcccctgccctgccggtgaggtcggcgccgcgctccggagagtcggttcctcccgattcagacccatatggcttcagatggaaccatcatttctgttccgtcatgaccggcttgcaaggtagcaaagcgaaacaaacgtctcgttttcagcctgtccccgccaccttgaaataaaaccggcaaaaataaaaagagcattctgggaagtacttcatcatttcaacgctcacgaattcaaaacataaacaaaggcttccgagtgccccggccaggggcgcggggcgcacggcgggcccggggcaggtaagcgcaggtgcgcgcccgcccccacccccggctccctccccatccgctccccgctccccttcccctttcccttccccgcccgctcccagccgccgccgccgccccgcgcacggcctggagcggaggctgcgcagggcgcggggcggcgcgggccgggcgcgggccgggcggacggccgcgtctttcttctcctggcggtgatgtcattgggcgacggcggccgaggccggggggcggcggcgggcgcccgcaggttcccgagccgctcctgagaaggcgcctgacagcgggccggggcgcacggagaagcgggccgggccggacctgctgggccgcgccgagccaatcgccggcgccggccgctcgatgggcgaggcggcggcggcggcggcgggggccgcgggccgggccgccgctccgaggtgaaggcgcgcgcccctccccgcctgcctcccgggccgcagcgatgaattccgccgagcaaaccgttacgtggctcatcactctgggggtgctggagtcgcccaaaaaaaccatctcggacccggagggctttctgcaggcgtcgctgaaggatggggtggtcctctgcaggctgctggagcgcctgctccccgggaccatcgagaaagtaagtcccggcccgcgcccccgcccgcgccccccggtccggcccgctgcggcccgggatgcgcgcggagcacctgaggccggccgcgctcgggaaacccgtgcgccctcctttgtgcgcggagcggcggcgcggccggcgccaggaccgtggcgggagctcggggggcgccattgtgtgcggggcagggggagggggccggggagcggggtcggggggaggggcggcgcgggcggcgggggtccccgggaaggagagtgcaccgcagcgaggagacggggcttctgcgccgcccgaacgctaagttgcccctccgcgtggggggccggccccgctccctggcagcagccgcatcggaaaccgcgttccgacgcggtgcggctcactccgctggctttgtgtggacagttcttgccggtggcgttgcgtgagtttcgcctcccaggttaattgaccggttgctctgtatttttaaagaggcacgcatctgtagctgagggctgcgctgtgaaacgatctactactttcccctcgagaaaattccccacctatccgagccgagaggacggcagacacagggtctaagtcctggcactcttcaaggcttgttaaaattctagatttcgttctcaggtccctccctcgttctgaacagaacctttagtcctgcaggcactgcttctcccttccggctggtggttcccaggctggagcgtgtgaaccctgacccgaatctctggaagtgattaaagtttgttttcatcgcccacaagccggccagcagggccgaggaggcaggagagtttctaaaaacgatgtctccctgtagctcaaaatactaaaatcttattcccagggtgtttcttcggggggtggggtgggggtgggggtggccaatatatagcgttaagatcaacagttatttaaaaggtgttaaatatagaaattgaagtgtaccataattaggaagaaggatttcagtttttaagtggggtgtattggcccccatacaggatgacaacaacttgtaaataaaatgccacgtaagtgacttgaaaaagtaaaatgtttaatttttttcattca

SEQ ID NO: 86 - DNA, 50 bp, Artificial Sequence
  ABCC8 source sequence
aagcaaaacttggtgatcccatgggtcacaaacgtccgctgtgtttggcg

SEQ ID NO: 87 - DNA, 50 bp, Artificial Sequence
  ABCC8 Unmethylated AlleleA Probe Sequence
aaacaaaacttaataatcccataaatcacaaacatccactatatttaaca

SEQ ID NO: 88 - DNA, 50 bp, Artificial Sequence
  ABCC8 Methylated AlleleB Probe Sequence
aaacaaaacttaataatcccataaatcacaaacgtccgctatatttaacg

SEQ ID NO: 89 - DNA, 3001 bp, Homo sapiens
aaagggcggggggggtctcactatgttgcccaggctggtctcgaactcctggactcaagcagtcttcctgtcacagcccaccaaaatgctgagattacagatgtaagccactgtgccctgccaaggtaccccattatacatactaacttcttgtgtgacaagtcatcaggttaataaaccgtttggctgagaacagaagtgtaaatgctgttcttttttgatttttgatgcacatatgagcacatattttgtatgttttatttctatttctatctaactgtctttgttaataactagcaactctgcttctgcagcaacctcttgtaaatctaattttgtgacaatctttcctttcttccccaaagctgtatatgaattttaagatttgcttgttttcttaaagtgaatgaacacgttttggggatgcaggggacaccctatgagttgatagacttgactggtctggaagagtcaaggtcttggagagagatgggggggacctcagtggagaggcacgcacgattgtcccggccgtggcgcccaactttgcaagcaagggcgccggggtcgctgcgtcggcgagtcggagctactttgcgcagagcctgagaagttgcgctctggagacacatgcccgggtggggtggcgggggaagggaatccaagtccctagctctcgcctctcctcgcccagggcgcgggcctgggactgtcagagatcacggaccactgcgcggctcagcccggtgaaagcataggggttagctgggcctccatcccagactccaaagcacaggcttccctcttccctaggcccagctgcggctgacagggaggctgccttgcgggaggggtgacccggctccctaagctgcgccacggagggtgaggcccgacccctctccagtttcccactgcgtgctgagacctcctctttcccttttccccatcccagctcttggccctgcagaagcaaaacttggtgatcccatgggtcacaaacgtccgctgtgtttggcgcgggatcgggggcaccgggggagtgaagggatgagctggtgtgcgagtgcggggaccggcgggcaggacacagggcaggggaccccgggaacgaggcggacggcggtcgcgggccggaaggggacgccgagcggtgcggcgcgcagcgcctcctcctccctccctgctctcccgtcccctcctccgcggctcgctgcgcgcactcaccaatgaagaggatggggaaggtgatgaagagtaggaagacgtgcggcaccacgttgagcgcgtccacaaagcagccgttgttgaggaccccctggtccacccggtaggcggccgagtggttctcgctgccgcagaaggccaggggcatggcggcgcgggcgcgggctgggctcgggctcagctggctccgctggctccgcgcgcctgccgcgcctctgtcccttgcagctccgccgcccggcccggccgtcaggcccccgccccccgggccccgcccagccccgctggcctccccctccctcaccccgccctgctccaccacctgcggggccggggggcggcgaggaacccgaccggccctgctacgcttgcgcccaccttccagatgtggaggcgcgtgctcccagatgttgccggggtggcttcgtgagtgccctgatgcacgcgtgcgctgggggagaggggaaaggcaggctaagatggggagtggggtgtgggcacactggagtctgggtgggaaaggagggtggggcgtgcagaagaggagagaagggcccgcaaggcctggagtgggcgggttcagggtcccgagtgtgagttcacctgggcgtggtgcggatggatgtagagagccgagggatggcgcaagtctccacccaacatggatgagaaaagtggggcagaacgttaccttgtggtggtggtggtggtggggtggggcttggacattttcaaaagtggtcccaggtggggcactcctaggtactgctttacagcgtatctggcactttcacacaagggatgtcccagaactcttgctaagtacaggttctaatcgcttccaattttctgatggggaaacccaagctgcgagggagctgaagggaggccagtaacatgacttcccccaaggtctcagtgccagcaagcagggaagcatagcttgaacccagctcctcaggtcccccgtccccacccccatcatttgtactcatgcctctggtacatgacaaccttacagcgactggcagaccaatgggtagaatgggtgacagccccaggctgataaagaggagtttaagaaacgcatctggccagatggatgtgaggcagtggagagggactgaaattcttaacctcacctgggacaggcttaacagttggaggacaccgtgcaattgtaaagacaacattatgtcttcctatacgttttggagagattctcacaagactgtggcccaaagaagtttatattgtatgtagaaagaacagaagatgggatttgggggttagatttaaggagaactttcctagttagggtatgaagcataaatgcacatgttcttaggaacagtcaggggaacacaaaagaaagaccccttcagcccctttgcctacaagcttcactgagcctgccctgaggctgctgagcctggtgagcctctccagcacgctgtcagcaaggccgaggctctgcccaggccctgaggcagagggcagatggggttgggtgaaaagccttgggaaagcaggagttcagggtctgaatgcctcagtctctgactttggagaatttctcagaagccaaaagatgtttcttgcagcatcagccaaaatagtgaaaacttggtggcaagaaatatccagcaccagggacaggggttaatcaaccttgaaaaatgcaactgctgggacggtgctagctgagtctgtgtagtcaatagaaaaactttacaagatagtgtcaggtgaaaaacggcaaacaaaattgtgc

SEQ ID NO: 90 - DNA, 1163 bp, Homo sapiens
cgcacgattgtcccggccgtggcgcccaactttgcaagcaagggcgccggggtcgctgcgtcggcgagtcggagctactttgcgcagagcctgagaagttgcgctctggagacacatgcccgggtggggtggcgggggaagggaatccaagtccctagctctcgcctctcctcgcccagggcgcgggcctgggactgtcagagatcacggaccactgcgcggctcagcccggtgaaagcataggggttagctgggcctccatcccagactccaaagcacaggcttccctcttccctaggcccagctgcggctgacagggaggctgccttgcgggaggggtgacccggctccctaagctgcgccacggagggtgaggcccgacccctctccagtttcccactgcgtgctgagacctcctctttcccttttccccatcccagctcttggccctgcagaagcaaaacttggtgatcccatgggtcacaaacgtccgctgtgtttggcgcgggatcgggggcaccgggggagtgaagggatgagctggtgtgcgagtgcggggaccggcgggcaggacacagggcaggggaccccgggaacgaggcggacggcggtcgcgggccggaaggggacgccgagcggtgcggcgcgcagcgcctcctcctccctccctgctctcccgtcccctcctccgcggctcgctgcgcgcactcaccaatgaagaggatggggaaggtgatgaagagtaggaagacgtgcggcaccacgttgagcgcgtccacaaagcagccgttgttgaggaccccctggtccacccggtaggcggccgagtggttctcgctgccgcagaaggccaggggcatggcggcgcgggcgcgggctgggctcgggctcagctggctccgctggctccgcgcgcctgccgcgcctctgtcccttgcagctccgccgcccggcccggccgtcaggcccccgccccccgggccccgcccagccccgctggcctccccctccctcaccccgccctgctccaccacctgcggggccggggggcggcgaggaacccgaccggccctgctacgcttgcgcccaccttccagatgtggaggcgcgtgctcccagatgttgccggggtggcttcgtgagtgccctgatgcacgcgtgcg

SEQ ID NO: 91 - DNA, 50 bp, Artificial Sequence
  SIRPA source sequence
tttgcgcaaacttgtttttctaaggtcagcgctgcgagctggctacatcg

SEQ ID NO: 92 - DNA, 50 bp, Artificial Sequence
  SIRPA Unmethylated AlleleA Probe Sequence
tttacacaaacttatttttctaaaatcaacactacaaactaactacatca

SEQ ID NO: 93 - DNA, 50 bp, Artificial Sequence
  SIRPA Methylated AlleleB Probe Sequence
tttacgcaaacttatttttctaaaatcaacgctacgaactaactacatcg

SEQ ID NO: 94 - DNA, 3001 bp, Homo sapiens
cactttccagtccacccagctcataacagtagccgggggatttgaacccacggagttcagcccatgttctcaagtgtggcaccttccctgcccccctgcacaacagcagggccatgccttcaccaggaggctcggggcaggaggggaggcagtggtgttactttgcacaggtctttaacctgggaatcaggaattcaaatcttgtttctaccccaagctttgctgtgtggccttaagcaaacctattccctctccgggcctcaactttctgtccatttacagggttgggccttctctcaccttgtgtcattcctgtcctcttaaccgccactgtgtgaaccaaaattctctcccattagcagcggggtcttgaacttgtgttcctcccacagtgcggagccttctgccaggtgaccacgacactggcactccccatcactgacctggctactacaggaacggcgttccctccgggaaaaccggggttctgtcctctccaactccaggcctctggccccggctccccttcccctcatcttcctcagggcttgtccttctcccggaacccaggccttcagggccccgctctcccacacacgccccggcctgggcctcccacccgcccagtccgccctccaccaccgagacacctggccagggggcctccacctccctgggaaggccccgacccttcccctctggttgcgcgccacagcccccttctcgctccctctcctccccgcgcctcttttccaggcggcaccgaagtccttcctacacacacgctatttttgtaccatcatctcttcttcctctccccttccccctgcgttcacttacttccttcttttccttcacctcggcccggctctgaaaggatcgggcccctccggcccgcacccacccccaagaggggccttcagctttggggctcagaggcacgacctcctggggagggttaaaaggcagacgcccccccgccccccgcgcccccgcgccccgactccttcgccgcctccagcctctcgccagtgggaagcggggagcagccgcgcggccggagtccggaggcgaggggaggtcggccgcaacttccccggtccaccttaagaggacgatgtagccagctcgcagcgctgaccttagaaaaacaagtttgcgcaaagtggagcggggacccggcctctgggcagccccggcggcgcttccagtgccttccagccctcgcgggcggcgcaggtccgggcccggcagggacggggaggggcagagcgggaccggtcggggctggggaccagccgcgcgggcttctgaacgggcacgctgtcggagccggaggctcgtctagtcccgagccccagcgggccgcggcggctcgcggggccgccttcccgggacagcagccggggcccgggcgccggcgcgggggaggacgctgccctccccggccgggcgcgcagctcgcagccctcgctccctcccccggggcgcgcaggcgggcgctcgctcgcagagaaggtgccggcggggccggagccccgaagcctccccgggcggcgggaagacgggaggaggggaaagggaaggagggagggggtcgggctgccgggagggcgggggcagcggccgggactgcgggccgggagcgagcccagccgcccgcgccgtgtggagcccgggcggggcaggtgggcagcggcgggggcggctccgcgcggccgggctcggccgtgctgcccctcgcccctgctcgcgcggtcctctctcccgcgcggtctgtcggtctgcgcgccgcctcgctcctcgctcctcgctctccccctctttctccccctctcgcctctccgtgtctctggctctctgcctggctcccttgggtctgtttctctctcctgccgcctctctctggccgcccctggctttatttctcgcgcgcttggggtctctcccagtctccgtctctccatttctcctggggggcggggagggggggtctccaaaaaccgcggcggcggcggcggccgctccaggcgcccgttccggagtcggggggaggcccagccgggaggggggaagggggggagccttagtcatttccccgctccagcctgctcccgcccgagcgcgcactcacggccgctctccctcctcgctccgcagccgcggcccatggagcccgccggcccggcccccggccgcctcgggccgctgctctgcctgctgctcgccgcgtcctgcgcctggtcaggtaagcacccccccgctccccaccgctgcactccccaaactgcgggctcaggcctctcagactggcactgggaccccttcggctaatgccgagcagtaatagggggagagacgcctgtaaccagggttatagatgcagaaactgaggcccagagagggccagcgacatgcccaaggtcacacagccaagggtagaacccaagtctcttctatgagagtttgtctctgtggactctgactctgtgcactgtgctgggcactgagacaaaagagccgagttgcttcactcagcccctgcccggtccaatggggaaggctgactgacagtggctttcagagcagggccgtccggcttccccagaatctgtccccatggacatacgactttccacatgtttgacttcagggtgtgaggggaggaggtgagggcagaggtcctcccgatgctgcctggaatggaacagagacaagctgttgcaattagggaagagtgagccctccctctggtgcgcagagtaaaccggtgttagtgtgtgcctgggagagagggagccgggaggccgcggtaaccccagagcgagctgctgcgagcaacagatccttatgtaaaaacacggtggattgtccctgtggttgccaaagcctctccctggtggtcttgctccttgaggcgctcccagctcccccttcctcctgccttccccgagcttccaggcagctccctgcagttcaggtgctccgcggggatgttacc

SEQ ID NO: 95 - DNA, 785 bp, Homo sapiens
cgccgcctccagcctctcgccagtgggaagcggggagcagccgcgcggccggagtccggaggcgaggggaggtcggccgcaacttccccggtccaccttaagaggacgatgtagccagctcgcagcgctgaccttagaaaaacaagtttgcgcaaagtggagcggggacccggcctctgggcagccccggcggcgcttccagtgccttccagccctcgcgggcggcgcaggtccgggcccggcagggacggggaggggcagagcgggaccggtcggggctggggaccagccgcgcgggcttctgaacgggcacgctgtcggagccggaggctcgtctagtcccgagccccagcgggccgcggcggctcgcggggccgccttcccgggacagcagccggggcccgggcgccggcgcgggggaggacgctgccctccccggccgggcgcgcagctcgcagccctcgctccctcccccggggcgcgcaggcgggcgctcgctcgcagagaaggtgccggcggggccggagccccgaagcctccccgggcggcgggaagacgggaggaggggaaagggaaggagggagggggtcgggctgccgggagggcgggggcagcggccgggactgcgggccgggagcgagcccagccgcccgcgccgtgtggagcccgggcggggcaggtgggcagcggcgggggcggctccgcgcggccgggctcggccgtgctgcccctcgcccctgctcgcgcggtcctctctcccgcgcggtctgtcggtctgcgcgccg

SEQ ID NO: 96 - DNA, 50 bp, Artificial Sequence
  BHLHE22 source sequence
cgttaacctgattgggtagaaagcgctgtcccaacaggcgagtcttcttc

SEQ ID NO: 97 - DNA, 50 bp, Artificial Sequence
  BHLHE22 Unmethylated AlleleA Probe Sequence
aaaaaaaactcacctattaaaacaacactttctacccaatcaaattaaca

SEQ ID NO: 98 - DNA, 50 bp, Artificial Sequence
  BHLHE22 Methylated AlleleB Probe Sequence
aaaaaaaactcgcctattaaaacaacgctttctacccaatcaaattaacg

SEQ ID NO: 99 - DNA, 3001 bp, Homo sapiens
tccccggaagactggatctcagggaattattatggaagatactgctgagcccctttcatcctttccaggtgaaaatacagacatttttgtgtcgccctcacccttcctttatgaaataaggtgcccaactgttctgatattaccattcaaaaacaggttctgtggcgaacctcatttgtgaatctattacagagattaatagattatttctcctttttcaactaattctcagtggggaaatttaaccatatggtaaggagagaattagaatttcatcacattagagcaaaatgtaatgaaaagagtccaacacctggggccaactccgaaagccacaattaaaaggtttttaatgaaaccagagaaaccaaaaattgcatagtcttcagtacttctgccatataagaaagagttattggaaggtgttgggaaatattgtttttacttatgtcataaggatgaaaaccagctgctaaataccatgtgtacccagagaccgaactcaggagtgagatgaccgcgtgtaccagtgcccatccggagaggagcacttgtttttacattctcccttcccgaaccctccaagaacaaccgaggctgatccagatgtccttaaatggcttccgggtaaataaatatgcataaatgcctcacttcgtgtgggagcggatttgactcctggtgaagttgactcttttgaagcaatcagattttcatcttaaggaaagtttgagaaaccggtttgtttgtttgttttcttcctgtttaggaaattgtgtacttcacaattaccatcactgtaacagttatttggagaccactgcaaaatcactgccaccccaccttaaaaaaaaatgggctgcatgatcacttgctcttcattttgcttttttctttctttttttccaatctgggtaggaaatgggcagtggcgggtgtggaaacgaggcagagtgttcggggggacgactgctttgctctctgaccagctgaaaacctagagtgaattttgggcaagccagctgggacaccaccttctctcggaaagtcccatccccaaatccagaccagtcatcctacgaacaggggtggagtataattctcgcccggaagggtaatttagcacaagctgagacagcagtggcgagggaagggcagtggggggtggggtgcggtgggtgggggcgtctgctttccacaggactcccaggcttcgccgccgatctacaatttgctgaaggagcaaagaacatcctcggctctaagtagggcttttagtgtgctcattgatgagtgaaagtcgccacacatgtcaagctaaaggcagttgttgggttactaacaggacccagcgccttgcaaacatatgcgctaagctgtgtatacagatggcaggcagaataatggagcaggcgccttttataaagctctagctgctgcctgtcttcagacctgggaaatgaaactattcagacttgcggccagatagcgcctgcgattgtttgttaccgttttaatcctattaattaaaacgttaacctgattgggtagaaagcgctgtcccaacaggcgagtcttcttcataataacctactcagagataatgatgtaaaagactcccccgtctgtggcggcggctgtttgatgggtccggaaatctcttgaaggtgaatccaagcaagataaacggtgcggagaggaggcgcggggctgggctcagagcggcggcggcggcggctccactccctccgcgcccaccctcccaccatgcggggccgcggcccatggtgagccccagcagccagcaccatcggctggagacgaagaagaagaagaagaggaggcggcgagcgcgggggaaggcgaaaaagaaaaagaaagaaggggagagggctcccggcagcaccaggaccgacgcgcgcaccagctccggagcccagctcgcgcgcgtctgtggggccgcctgactccggggccgaggcggcggcggcggcagcgggcgcggcggcccgggctgcgcgccggcgcgggaccatggagcgcgggatgcacctcggtgcagcggccgccggcgaggacgacctcttcctgcacaagagcctgagcgcctccacctccaagcgcttggaagcggctttccgctccacgcccccgggcatggacctgtccctggcgccgccgcctcgggaacgcccggcgtcctcctcctcgtcgcccctgggctgcttcgagccggctgaccccgagggggcagggctgctgttgccgccgcctggaggaggcggcggcggcagcgcgggaagtggcggcggcggcggcggcggggtgggtgtccccgggctgctagtaggttcagccggcgttgggggcgaccctagcctaagcagcctgccggccggggccgccctttgcctcaagtacggcgaaagcgcgagccggggctcggtggccgagagcagcggcggcgagcagagccccgacgacgacagcgacggtcgctgcgagctcgtgctgcgggccggagtagccgacccgcgggcctccccgggagcgggaggtggtggcgcgaaggcagccgagggctgctccaatgcccacctccacggcggcgccagcgtccccccggggggcctgggcggcggcggcggcgggggtagcagcagcggtagcagtggcggcggtggcggtagcggtagcggcagcggcggcagcagcagcagcagcagcagcagcagcaagaaatccaaagagcaaaaggcgctgcggcttaacatcaatgcccgagagcgccggcggatgcacgacctgaacgacgcgctggacgagctgcgcgcggtgatcccctacgcgcacagcccctcggtgcgaaagctctccaagatcgccacgctgctgctcgccaagaactacatcctcatgcaggcgcaggccctggaggagatgcggcgcctagtcgcctacctcaaccagggccaggccatctcggctgcctccctgccca

SEQ ID NO: 100 - DNA, 1517 bp, Homo sapiens
cgtctgtggcggcggctgtttgatgggtccggaaatctcttgaaggtgaatccaagcaagataaacggtgcggagaggaggcgcggggctgggctcagagcggcggcggcggcggctccactccctccgcgcccaccctcccaccatgcggggccgcggcccatggtgagccccagcagccagcaccatcggctggagacgaagaagaagaagaagaggaggcggcgagcgcgggggaaggcgaaaaagaaaaagaaagaaggggagagggctcccggcagcaccaggaccgacgcgcgcaccagctccggagcccagctcgcgcgcgtctgtggggccgcctgactccggggccgaggcggcggcggcggcagcgggcgcggcggcccgggctgcgcgccggcgcgggaccatggagcgcgggatgcacctcggtgcagcggccgccggcgaggacgacctcttcctgcacaagagcctgagcgcctccacctccaagcgcttggaagcggctttccgctccacgcccccgggcatggacctgtccctggcgccgccgcctcgggaacgcccggcgtcctcctcctcgtcgcccctgggctgcttcgagccggctgaccccgagggggcagggctgctgttgccgccgcctggaggaggcggcggcggcagcgcgggaagtggcggcggcggcggcggcggggtgggtgtccccgggctgctagtaggttcagccggcgttgggggcgaccctagcctaagcagcctgccggccggggccgccctttgcctcaagtacggcgaaagcgcgagccggggctcggtggccgagagcagcggcggcgagcagagccccgacgacgacagcgacggtcgctgcgagctcgtgctgcgggccggagtagccgacccgcgggcctccccgggagcgggaggtggtggcgcgaaggcagccgagggctgctccaatgcccacctccacggcggcgccagcgtccccccggggggcctgggcggcggcggcggcgggggtagcagcagcggtagcagtggcggcggtggcggtagcggtagcggcagcggcggcagcagcagcagcagcagcagcagcagcaagaaatccaaagagcaaaaggcgctgcggcttaacatcaatgcccgagagcgccggcggatgcacgacctgaacgacgcgctggacgagctgcgcgcggtgatcccctacgcgcacagcccctcggtgcgaaagctctccaagatcgccacgctgctgctcgccaagaactacatcctcatgcaggcgcaggccctggaggagatgcggcgcctagtcgcctacctcaaccagggccaggccatctcggctgcctccctgcccagctcggcggctgcagcggcagcagctgctgccctgcacccggcgctcggcgcctacgagcaggcagccggctacccgttcagcgccggactgcccccggctgcctcctgcccggagaagtgcgccctgtttaacagcg

SEQ ID NO: 101 - DNA, 50 bp, Artificial Sequence
  COL2A1 source sequence
cgcagaagttcaccaagaagagttctgccaatcaaggactctgtcccagg

SEQ ID NO: 102 - DNA, 50 bp, Artificial Sequence
  COL2A1 Unmethylated AlleleA Probe Sequence
cctaaaacaaaatccttaattaacaaaactcttcttaataaacttctaca

SEQ ID NO: 103 - DNA, 50 bp, Artificial Sequence
  COL2A1 Methylated AlleleB Probe Sequence
cctaaaacaaaatccttaattaacaaaactcttcttaataaacttctacg

SEQ ID NO: 104 - DNA, 3001 bp, Homo sapiens
ccccccgccgaagagtttccctctgcgattgatttacaatctgtaacatacaaagacagggctggaatcccagccgtcggaaacgtgcccaaacacgtggaaaaaaaaatcctctctacctaatacactttccaggcttcgtttgtcctgaatataatcccagacagtggaccttaccaaaggaggggtagaaaggtgcagtccgaacagcatttggacccgactacaagagccatctctaactataccggttgaaatagttgtagcaaccgtagcaatcgaccagcctgctccccttccctgacacacacatacacacacacacacgcacacacacacacaccacgcagcctggctcggtgagttccagaatttagaaaagtaactcattgtagtacctcggggcggcggggaatggggtaagaccgcggggaagggggcgctattttcgggggcatttgaaaagcacggtctggttggaatgttatcgtctttcagatggcctctcctactggaagtttctgtaccctttatcagaagagaggcgcaggcggacggagggaagtcgagatgagcgctagggctgcaccttgctccagggccagctgggagggggagggtaccctaaagaggcgctgaggtctcagagggcctatcggaagccgttctttgtggcctgggatttcagcccaccagtgtcagagtgttagtcccctttgtctaactctgctttgagcccaccagccaccaagccttggacaggctgtgaagtcaaccctttggcgtttgcttgctccctcgctcgcgctctctctttcctacttcacctaagttcgatgagttgagatctgtttttgctgaactcgaagtgcttcgaagaggcgagctgtgtgcatgtgcgcctaattatcaaagctggtggaaaagagcccatttatatctgtgcgcaaacttctccgagccaacactttgcaagttcagtattctagcgcaacgtagggacctgcaaatacttgcaactgcgatgggcaccgaggaccctgggacagagtccttgattggcagaactcttcttggtgaacttctgcgtgtcctgtgcctaagtcggcgcgctacgaccccgggagccgttttagcccggagccgcgggctccagagctggagcgggccggggagaaggatgctgagggacgcatggaaagcaggcaggcaggcaggggcgggggaagacttacggacatcctggccctgacaccgaaggacagcggcgacgagcagcgtcagcagcaccagcgtctggggagccccgaggcgaatcatggctcaccgcggggcctggctgagccgggcccgggcggagcgcagcgaaacggcaggagcacggcgcgggtccgggtctctaccgcgccctcatgcaggaggcccttggagcaggagggggaagcgggagacccggcagcccagcagcgctctgcgtcttctccccgccgcggcgcccgttatatgcgcccggcccctttcgaggctggcgaactcgcccaaaccgcgggccgccccctgccccccgctgggctgtaacctgaaccgcccgcccccggagcccgcctgggccctgccagtgcccgcacctgcccaagccggacccccctctctgggagtcacgcttcccgccctccacccctagtgcgccgagtggcctgatcgggcggggcgcagagatggcccctgccccccaaacccggggctgcggcgctgcccccctctgaccacaggcgggaagggtggcctccggcccctcccctcggagttctgccggagttggaggggagggggtgtttgcagaggcgcaggccgagagccctagaccaaggacggaaaagacatgggagggaggagggaaagcggtagaaaggagcagcggagggcgagggggcaccgcgtcaggcgtttgggagtggggacagggaggagcccacagagattcagagtcctaaatcttggagacaggctagctgagccagatctgaagggttaccccgacctatcttttagcccttccctgcagtacctggtccccagactcccacctggacgttcacagcgactgccctccacccctcccccatccagctctgcgttgaaggcttcctctggagcacaggccgaagttggcctgagccgacagccaggaaaagagctggtgtgttactgcacagaaggagacctgtgtgaaggtgtggggggcaggggcaggccctgggaaggggactgaaagagccacagaggacggagggagccgagactaaacggtgcaagctgcctgctgtggggggactgtctagctccaggctcagacgtggtgtctgctcttcctggctggggtccgggagtcccccggggagtgggcactgccggccgtcgtagagcagctttggccagggacggttggtcactgctgcggccctggccagggatcggggaggccgatccagctggcgagttctgtgagccggggagaagcgccagaatttcccagtcggccccctagccctccgcttgccgcctcacctcaccttccccggtgagcgccctctctcaccttagccaaactgccttcttcccagggcttagctggtctcttctcttggtctccggtgcctcctttcataccagggaggaaacgtccccaaacagaccttccttttgagcgatatggtcccccaaagaactataggcaaaatgaaactcagaatttggggtctgacttaaccagaacttctgccctcctgggcagttttgtttctctggagctttccccctagaagctgagagcacttgaaagaagctacttctgggccttaggcttccaccaggggtcctgggaccccatccctagcctatcatgctttgacacttgtcctggtagaggctggagttccaagtgctctggttggaaagggaaagaactcattgttgaaggaaatgcttctaggctttgtcc

SEQ ID NO: 105 - DNA, 842 bp, Homo sapiens
cggcgcgctacgaccccgggagccgttttagcccggagccgcgggctccagagctggagcgggccggggagaaggatgctgagggacgcatggaaagcaggcaggcaggcaggggcgggggaagacttacggacatcctggccctgacaccgaaggacagcggcgacgagcagcgtcagcagcaccagcgtctggggagccccgaggcgaatcatggctcaccgcggggcctggctgagccgggcccgggcggagcgcagcgaaacggcaggagcacggcgcgggtccgggtctctaccgcgccctcatgcaggaggcccttggagcaggagggggaagcgggagacccggcagcccagcagcgctctgcgtcttctccccgccgcggcgcccgttatatgcgcccggcccctttcgaggctggcgaactcgcccaaaccgcgggccgccccctgccccccgctgggctgtaacctgaaccgcccgcccccggagcccgcctgggccctgccagtgcccgcacctgcccaagccggacccccctctctgggagtcacgcttcccgccctccacccctagtgcgccgagtggcctgatcgggcggggcgcagagatggcccctgccccccaaacccggggctgcggcgctgcccccctctgaccacaggcgggaagggtggcctccggcccctcccctcggagttctgccggagttggaggggagggggtgtttgcagaggcgcaggccgagagccctagaccaaggacggaaaagacatgggagggaggagggaaagcggtagaaaggagcagcggagggcgagggggcaccgcgtcaggcg

SEQ ID NO: 106 - DNA, 50 bp, Artificial Sequence
  PTEN source sequence
gatggaaatggctctggacttggcggtagctgatgcccctcgctctgccg

SEQ ID NO: 107 - DNA, 50 bp, Artificial Sequence
  PTEN Unmethylated AlleleA Probe Sequence
aataaaaataactctaaacttaacaataactaatacccctcactctacca

SEQ ID NO: 108 - DNA, 50 bp, Artificial Sequence
  PTEN Methylated AlleleB Probe Sequence
aataaaaataactctaaacttaacgataactaatacccctcgctctaccg

SEQ ID NO: 109 - DNA, 3001 bp, Homo sapiens
tgagtctcaggtctcagtcctttggcttgctcttagggtagcaggcgaggagtggcaccagtttggggactctctccccgcgttctgtaagaatcggcggcagccagcaggcggggaggcgggggcacgtgtttggatgtgggtgcttgtgtaaccagttccccaagcgccagccccgacagcgctccttcgggaggctggtccgagcccctgtttccgccgcggcgcaggaagggttggggttccgctgcctgcaccaggcaagagcaccccgagcaaaggaagaagacgacttgcctccggagctatcactggggagtgggaatttggaaagttccccaactagggacacacgtgacctccttcggaaagtagttccgactgtggcccgtgtatccttccacctccttttgaaccctcctaggtctcctcgccccgcccactcgctgggctgcagcttcctaccgttccgtactttccactcaacccggtaaccccaaacgtgcacggtccggccggggcgcgcggagcctggccccgggcgatccatcctgccgggttttcacggcggccaagggggggcggggctaggtggtctctgagaaccgagcttgactccgacgccgcgaaccgacctggagcccgaggggaaagatgctcgactctcttgggggcaccggagcgggcgcaggagaggcctgcggggtgcgtcccactcacagggatcctctttcagttcatttagataggtgccctttgggcccttgaaattcaacggctatgtgttcacgttcagcacgctcggctgagagctttcatttttagggcaaacgagccgagttaccggggaagcgagaggtggggcgctgcaagggagccggatgaggtgatacacgctggcgacacaatagcaggttgctctttgtgctaagactgacaccatgaggacacagatttgggggaagggggaatctctaggcaaaggctgttacagtcaaatctctgcgaacgattgtgatccgacagcggtgcaaaaggaaagagcgaatgcagtccacgccgcggaaatctaggggtagaggcaaggggggagggtattccccttgcagggaccgtccctgcatttccctctacactgagcagcgtggtcacctggtccttttcacctgtgcacaggtaacctcagactcgagtcagtgacactgctcaacgcacccatctcagctttcatcatcagtcctccacccccgccccacaacagcctaccctgcctccggctgggtttctgggcagaggccgaggcttagctcgttatcctcgcctcgcgttgctgcaaaagccgcagcaagtgcagctgcaggctggcggctgggaaccggcccgagcaagccccaggcagctacactgggcatgctcagtagagcctgcggcttggggactctgcgctcgcacccagagctaccgctctgccccctcctaccgccccctgccctgccctgccctcccctcgcccggcgcggtcccgtccgcctctcgctcgcctcccgcctcccctcggtcttccgaggcgcccgggctcccggcgcggcggcggagggggcgggcaggccggcgggcggtgatgtggcgggactctttatgcgctgcggcaggatacgcgctcggcgctgggacgcgactgcgctcagttctctcctctcggaagctgcagccatgatggaagtttgagagttgagccgctgtgaggcgaggccgggctcaggcgagggagatgagagacggcggcggccgcggcccggagcccctctcagcgcctgtgagcagccgcgggggcagcgccctcggggagccggccggcctgcggcggcggcagcggcggcgtttctcgcctcctcttcgtcttttctaaccgtgcagcctcttcctcggcttctcctgaaagggaaggtggaagccgtgggctcgggcgggagccggctgaggcgcggcggcggcggcggcacctcccgctcctggagcgggggggagaagcggcggcggcggcggccgcggcggctgcagctccagggagggggtctgagtcgcctgtcaccatttccagggctgggaacgccggagagttggtctctccccttctactgcctccaacacggcggcggcggcggctggcacatccagggacccgggccggttttaaacctcccgtgcgccgccgccgcaccccccgtggcccgggctccggaggccgccggcggaggcagccgttcggaggattattcgtcttctccccattccgctgccgccgctgccaggcctctggctgctgaggagaagcaggcccagtcgctgcaaccatccagcagccgccgcagcagccattacccggctgcggtccagagccaagcggcggcagagcgaggggcatcagctaccgccaagtccagagccatttccatcctgcagaagaagccccgccaccagcagcttctgccatctctctcctcctttttcttcagccacaggctcccagacatgacagccatcatcaaagagatcgttagcagaaacaaaaggagatatcaagaggatggattcgacttagacttgacctgtatccatttctgcggctgctcctctttacctttctgtcactctcttagaacgtgggagtagacggatgcgaaaatgtccgtagtttgggtgactataacatttaaccctggtcaggttgctaggtcatatattttgtgtttcctttctgtgtattcaacctagggtgtgtttggctagacggaactcttgcctggttgcaagtgtcaagccaccgattgctttcttaggctatctatatggtctcttcctgagggctattgtccgttaatacagaatacagtacactgttagtggattagcgagctcggtaatccggtctcctaaatgaacaaaaaagtagacgctttttgaggttgagcatatttcgattaaatcttggcttaggccc

SEQ ID NO: 110 - DNA, 2356 bp, Homo sapiens
cgcgttctgtaagaatcggcggcagccagcaggcggggaggcgggggcacgtgtttggatgtgggtgcttgtgtaaccagttccccaagcgccagccccgacagcgctccttcgggaggctggtccgagcccctgtttccgccgcggcgcaggaagggttggggttccgctgcctgcaccaggcaagagcaccccgagcaaaggaagaagacgacttgcctccggagctatcactggggagtgggaatttggaaagttccccaactagggacacacgtgacctccttcggaaagtagttccgactgtggcccgtgtatccttccacctccttttgaaccctcctaggtctcctcgccccgcccactcgctgggctgcagcttcctaccgttccgtactttccactcaacccggtaaccccaaacgtgcacggtccggccggggcgcgcggagcctggccccgggcgatccatcctgccgggttttcacggcggccaagggggggcggggctaggtggtctctgagaaccgagcttgactccgacgccgcgaaccgacctggagcccgaggggaaagatgctcgactctcttgggggcaccggagcgggcgcaggagaggcctgcggggtgcgtcccactcacagggatcctctttcagttcatttagataggtgccctttgggcccttgaaattcaacggctatgtgttcacgttcagcacgctcggctgagagctttcatttttagggcaaacgagccgagttaccggggaagcgagaggtggggcgctgcaagggagccggatgaggtgatacacgctggcgacacaatagcaggttgctctttgtgctaagactgacaccatgaggacacagatttgggggaagggggaatctctaggcaaaggctgttacagtcaaatctctgcgaacgattgtgatccgacagcggtgcaaaaggaaagagcgaatgcagtccacgccgcggaaatctaggggtagaggcaaggggggagggtattccccttgcagggaccgtccctgcatttccctctacactgagcagcgtggtcacctggtccttttcacctgtgcacaggtaacctcagactcgagtcagtgacactgctcaacgcacccatctcagctttcatcatcagtcctccacccccgccccacaacagcctaccctgcctccggctgggtttctgggcagaggccgaggcttagctcgttatcctcgcctcgcgttgctgcaaaagccgcagcaagtgcagctgcaggctggcggctgggaaccggcccgagcaagccccaggcagctacactgggcatgctcagtagagcctgcggcttggggactctgcgctcgcacccagagctaccgctctgccccctcctaccgccccctgccctgccctgccctcccctcgcccggcgcggtcccgtccgcctctcgctcgcctcccgcctcccctcggtcttccgaggcgcccgggctcccggcgcggcggcggagggggcgggcaggccggcgggcggtgatgtggcgggactctttatgcgctgcggcaggatacgcgctcggcgctgggacgcgactgcgctcagttctctcctctcggaagctgcagccatgatggaagtttgagagttgagccgctgtgaggcgaggccgggctcaggcgagggagatgagagacggcggcggccgcggcccggagcccctctcagcgcctgtgagcagccgcgggggcagcgccctcggggagccggccggcctgcggcggcggcagcggcggcgtttctcgcctcctcttcgtcttttctaaccgtgcagcctcttcctcggcttctcctgaaagggaaggtggaagccgtgggctcgggcgggagccggctgaggcgcggcggcggcggcggcacctcccgctcctggagcgggggggagaagcggcggcggcggcggccgcggcggctgcagctccagggagggggtctgagtcgcctgtcaccatttccagggctgggaacgccggagagttggtctctccccttctactgcctccaacacggcggcggcggcggctggcacatccagggacccgggccggttttaaacctcccgtgcgccgccgccgcaccccccgtggcccgggctccggaggccgccggcggaggcagccgttcggaggattattcgtcttctccccattccgctgccgccgctgccaggcctctggctgctgaggagaagcaggcccagtcgctgcaaccatccagcagccgccgcagcagccattacccggctgcggtccagagccaagcggcggcagagcgaggggcatcagctaccg

SEQ ID NO: 111 - DNA, 50 bp, Artificial Sequence
  AUTS2 source sequence
agtgtggggctccccacagcaccgagggtcggagatgcctgggagcagcg

SEQ ID NO: 112 - DNA, 50 bp, Artificial Sequence
  AUTS2 Unmethylated AlleleA Probe Sequence
aatataaaactccccacaacaccaaaaatcaaaaatacctaaaaacaaca

SEQ ID NO: 113 - DNA, 50 bp, Artificial Sequence
  AUSTS2 Methylated AlleleB Probe Sequence
aatataaaactccccacaacaccgaaaatcgaaaatacctaaaaacaacg

SEQ ID NO: 114 - DNA, 3001 bp, Homo sapiens
acgggtcctcgagctggggcggcggtcccggccccttcggccccgcccctcgcccgcggccgcctccgaggttcaagttcggcccccggctgttaggcctcgcgctcccggatctgaagccgcccgtgcgtccctaggtcccccgctggaagacgaggcgggaatcccaggggtcatccgggcgcggggtcgctcgtccacgcactgtggtgccacgtggaggaaaagtttccaactctgccagttggtgctcccactgcgcccaggcggagcaaaggcccgcgctttggcacccgctccccgttctgtctcctccagtcgcgccccctcccagggacgccccccttgtaccccttctctgaattcccttctctcctcgcagcgcaggtagccaagggaaaacacccttgggggctggtggggcttcccgagaaggcggcctcgggccggccaggggtagaggatccagggacaagtgtcccgaggaggatgtggggggcgcgcgcgcgaagtggcctgggagcaggggcccacacgcgtccccgctgcgacaggcgggatcctccggcggcttccacgccctggcgcgccaactctgcccggccgcggccgaccccacgcgggcgccccctccacgcccccgcccccgcccccctcacagctccccaccgcccccagtgcgcaggcccggccgccccagcgcgcatgccctgggctcgcgagcgcggccagcccccagccttttgctttctacacactctacaactggggagggggcgggggaggagggagcccagccgtccacgtgatcccgccggccggggccgcgggggcgggagcgcgcgccgagagaggcgcggggagggcgggtgcgcgggagcgcgcgcactcgagcgagcgagcgaaagcacgaacgagcgagcgagcgagcgagcgagcgggcgggcgcggggttgggctgaggcgggcggcggcggcggcggcggcggtgggctggccgcggagggggaggagcgggagctgcgcgaggcaggcgggcgggcggaggatggggcagtgcccgggctcccgctgcagccgcccggggacacgccgtgcaccctccggctcggggctttctcggcggcggcggcggcagcagcagcagcgttagcggcggcggcgagagcagcgttcccggctgcgcttctccctcaggcggggcggcgagagaagcggcggcggcggcggcggcacaccggtgtctctcccgctggagatttctttctgctttcctcatcgactgactgactcaccccctcccttttttgaggaagggtgacctcttctctgggactggaaaaaatatttttgtgaggaggggggcgggtggcagcgacagggtttgcttcttctcttctttcatctccctctcctcccccctcgaagaccgaaaagcaaaccccacaactgctccagcagcatcctccttcccttcctccgcctcccttctcctccgccgctcgcagtttcgccctctcttccgctaatgattgcattattatgctcccctctctggggggtctcgcccctcttgggtcgctccggagccccggcctcccctggctgcatttcttaaaaatttgggagcctgggagtgagttttctccgaggcgtgtgtgagaggcggcgggggtgttttcctgcgcgaggggcgggtgaagttcattgcccccacttttcccgcgacctttttcggacccgattttggatcgagttgaggggggcgcgggcgttttcggggggcggggggcgcggcggagaatggccgcggggagggctccccggagcctcccagtctcttgatcaaagcattccgctattctgatttattgcttgcttggtgagttatttttttttcctctaaaggagacctgtgtgttcagccattactttgctcggcgctgctcccaggcatctccgaccctcggtgctgtggggagccccacacttgggctcctcgcctctcgccctcgctccccgtccctcctcccctctctccgccccttcccccttttctttctcctctctttcttcccctctctcccttctttcggccgccgtctcccccgcgccctcctcggggcggagggaagccgtgaagggggagggagggctcggtgtcaatttttttttgtgtggctgcggccgtagcctgtggcgggcaagcggggagaccccggcgcagcagaaccatggatggcccgacgcggggccatggactccgcaaaaagcggcggtcgcggtcgcagcgagaccgggagaggcgctcccggggcgggctgggggccggcgcggccggcggcggcggggctggccggacccgggcgctctcactcgcctcgtcgtcgggctccgacaaggaagacaatgggaagcccccgtcctccgccccgtcccggcccagacccccgcggaggaagcggagagagtccacctcggcagaagaggacatcattgatggatttgccatgaccagctttgtcacttttgaagcgctggaggtaagggggaccccccttcccccgggttccctttatgcacgaccccactcggctgcgcccggctctcctgcctccctcgccgcgctccgggctgtctgtctgtctgtctgtctgtctgtctgtctaggctgaggtcttattgttggtggggggagctgggggcgcgggcagacaggcagcaggagggagtcgcaaagccgtgcctggtctcgtttcctcgctcgcctttcccctgcgccccctccctgcaccccctccacagcagatgggcagaaagaaaggggttcgggcatcacaacaatgcgccccctctccacagagagctctgcccccactccttcctcctctccctccccaatcatggcttcttggctggtggaagtctctttgctgggtttggggcgcctgtggacggttctgtgtgctttctacagagtcatatgttcgtgtgtgtgtgt

SEQ ID NO: 115 - DNA, 2663 bp, Homo sapiens
cgcggggcagggactgcggtttgccggtctaacgggtcctcgagctggggcggcggtcccggccccttcggccccgcccctcgcccgcggccgcctccgaggttcaagttcggcccccggctgttaggcctcgcgctcccggatctgaagccgcccgtgcgtccctaggtcccccgctggaagacgaggcgggaatcccaggggtcatccgggcgcggggtcgctcgtccacgcactgtggtgccacgtggaggaaaagtttccaactctgccagttggtgctcccactgcgcccaggcggagcaaaggcccgcgctttggcacccgctccccgttctgtctcctccagtcgcgccccctcccagggacgccccccttgtaccccttctctgaattcccttctctcctcgcagcgcaggtagccaagggaaaacacccttgggggctggtggggcttcccgagaaggcggcctcgggccggccaggggtagaggatccagggacaagtgtcccgaggaggatgtggggggcgcgcgcgcgaagtggcctgggagcaggggcccacacgcgtccccgctgcgacaggcgggatcctccggcggcttccacgccctggcgcgccaactctgcccggccgcggccgaccccacgcgggcgccccctccacgcccccgcccccgcccccctcacagctccccaccgcccccagtgcgcaggcccggccgccccagcgcgcatgccctgggctcgcgagcgcggccagcccccagccttttgctttctacacactctacaactggggagggggcgggggaggagggagcccagccgtccacgtgatcccgccggccggggccgcgggggcgggagcgcgcgccgagagaggcgcggggagggcgggtgcgcgggagcgcgcgcactcgagcgagcgagcgaaagcacgaacgagcgagcgagcgagcgagcgagcgggcgggcgcggggttgggctgaggcgggcggcggcggcggcggcggcggtgggctggccgcggagggggaggagcgggagctgcgcgaggcaggcgggcgggcggaggatggggcagtgcccgggctcccgctgcagccgcccggggacacgccgtgcaccctccggctcggggctttctcggcggcggcggcggcagcagcagcagcgttagcggcggcggcgagagcagcgttcccggctgcgcttctccctcaggcggggcggcgagagaagcggcggcggcggcggcggcacaccggtgtctctcccgctggagatttctttctgctttcctcatcgactgactgactcaccccctcccttttttgaggaagggtgacctcttctctgggactggaaaaaatatttttgtgaggaggggggcgggtggcagcgacagggtttgcttcttctcttctttcatctccctctcctcccccctcgaagaccgaaaagcaaaccccacaactgctccagcagcatcctccttcccttcctccgcctcccttctcctccgccgctcgcagtttcgccctctcttccgctaatgattgcattattatgctcccctctctggggggtctcgcccctcttgggtcgctccggagccccggcctcccctggctgcatttcttaaaaatttgggagcctgggagtgagttttctccgaggcgtgtgtgagaggcggcgggggtgttttcctgcgcgaggggcgggtgaagttcattgcccccacttttcccgcgacctttttcggacccgattttggatcgagttgaggggggcgcgggcgttttcggggggcggggggcgcggcggagaatggccgcggggagggctccccggagcctcccagtctcttgatcaaagcattccgctattctgatttattgcttgcttggtgagttatttttttttcctctaaaggagacctgtgtgttcagccattactttgctcggcgctgctcccaggcatctccgaccctcggtgctgtggggagccccacacttgggctcctcgcctctcgccctcgctccccgtccctcctcccctctctccgccccttcccccttttctttctcctctctttcttcccctctctcccttctttcggccgccgtctcccccgcgccctcctcggggcggagggaagccgtgaagggggagggagggctcggtgtcaatttttttttgtgtggctgcggccgtagcctgtggcgggcaagcggggagaccccggcgcagcagaaccatggatggcccgacgcggggccatggactccgcaaaaagcggcggtcgcggtcgcagcgagaccgggagaggcgctcccggggcgggctgggggccggcgcggccggcggcggcggggctggccggacccgggcgctctcactcgcctcgtcgtcgggctccgacaaggaagacaatgggaagcccccgtcctccgccccgtcccggcccagacccccgcggaggaagcggagagagtccacctcggcagaagaggacatcattgatggatttgccatgaccagctttgtcacttttgaagcgctggaggtaagggggaccccccttcccccgggttccctttatgcacgaccccactcggctgcgcccggctctcctgcctccctcgccgcgctccg

SEQ ID NO: 116 - DNA, 50 bp, Artificial Sequence
  KIF1A source sequence
cttgcgccagggatgctggctccgggtgtaacaggtgcgcggtgaaatcg

SEQ ID NO: 117 - DNA, 50 bp, Artificial Sequence
  KIF1A Unmethylated AlleleA Probe Sequence
cttacaccaaaaatactaactccaaatataacaaatacacaataaaatca

SEQ ID NO: 118 - DNA, 50 bp, Artificial Sequence
  KIF1A Methylated AlleleB Probe Sequence
cttacgccaaaaatactaactccgaatataacaaatacgcgataaaatcg

SEQ ID NO: 119 - DNA, 3001 bp, Homo sapiens
cttccctggcaaaggccccgccagcctgagtgcgacctggtgtcccgcagagccgagcacggtgtcgcgctgtgcgtccccagggtgaggccagtgccagccctgcgaggcaggggcctctccgcaccgcctcctctccgcagccgcaagatccaatccgcttgccctggcaggcctttaattccagcaatgcaggcaccccttggaaagggctgacgcttggggaaggggtcctccagtctctgggggaaagtccctattcccggggacgtgggcgcgcatcgggtccgcgccgggcgacgtcgcgctcgcgcccagcaggccccatgacttggcattccctccttcccctccccctcccgcccctgcggccggcagggcccccccaaccccacctaccggcgcccagcgccccgctcctgcggaccctccctcccggcggggatgctgccccggcccttcccagcgagagggcagcgcggctggtttgggggaagtcagcgtgaggagcggcttaaagcgggagtgcgaaggtcttcgcggccactgttccttcgtgtcatccgagcccgggcgccgggggcgcgccgccagcctagaccctgcggccccgcagcccctccctcgccgccgggccacacctgttacaggggcgctggctgctgagacgcaggcagtgctcgccggcggcccctcccaggtgctgggtctttctggaagggagctggggtctctggcgtgggagggggtgatgggcaggccggccgtgcaacgaagcgggtggccaggccccaacgccgcagcccccgccccgcccgcctgttttgctcccggctccagcatccgccctcccccaccccgggaaccggagcagctgcccgcgcccggccgccctgcgcgctctcccctcgggccgaggcgctgcagccgcgcaccctcccaggtgcggcgtcagcgtcctcagccccgctcaccatccctcccctcccccgccgtctgggcgcagcccctcccgcagccccgccggtggctcctctccagggaagcggaatagccggggaagggcgggggtatcccgatcccgccggccgggcgagccctcccgagggcgacccccgcccgcctaagccgagggtcccgctgcagccgagatcgcggcaaagggaccttttccagcggcacctgaagggcggacacccctgaatttcccccattgttctctgcggagatggaggaaggtctggaagtgaaggggcctcgcagtgcctgcgctttgggcggcaacaagggcgccgggccggagctccccgccctgggcgcagtgggtgcaggtgcgggctgcgggcgcgggctgccgggcgcaggtgcggggcgaggggcgcgggcgcgggaggccgggcgggcggcggccttacctctccggcgtcactggcggcggccccgcatgggcactggcatgggcgcgggctctcgagcccggagctgctgccgctcgccggctgctctgcgctgtgacggcgccgccgcgtgacgggctgcgcgcccccggcccggaccgccccgcgcccctcccgaggcgctggttggctgcgcgcggcgtccgagctccagtcgccagctggtttgtcgcgcggggagagggacgggcgacgcggccgccgcggggatggggctggggacggagggggccggggccgggctgtgacgtcaccgctgacaccgcggcgggccccgcggagggtgggggaccgacacccggctcggacgcagcgtcccctcccccggcgtgcccctgagacctcgcgggggagggacccggcggcgccccgtcccgaagccccgcgtgcacctgaacttgcccgcggacccctcgccgggccgtcccccaccctggcccctttcgagtgaaatcgtaaaacgtccacctgcccccgctctgagccagcgacagacgcatcgtggggggcgggcggggctggggagggagggctccttgcgccagggatgctggctccgggtgtaacaggtgcgcggtgaaatcgcatcttgtgtcggggccggggcgccgggggaagagcagcagtgacccaggccctccccgccggatgatgcaacccgcccgcggacccgccgcctcgcctctaccgcggctcgtggggaccccccgccctcctctcgccccacctctccaggcccccattgtcctcaccccagcctgtccagtttaacttccttggacccccaagtcacacgccgctcgggggccaccccggcaaaccccagcctgcgtcacccgcccccagccgccttcctagttccaggggctgactggtatctctgtgcgcgccaccgtcctccctccaccgtcccagggctgggcaacttcgacaaagcctctgggcagctcgcccatgggaacctccccgcactgcggtgcgggggtgcacggatttcgggccaccctgaaccccggggctgggtcccctccagcccagcccctgccatcgagcccctgccatgggcatcctggcgtcgcctggctaagcctccctggcgcccagcaggggcctgcgctgccctagcggcctcgccgttaaccatttcatccacactcctgggagagccggcttcacctcagtcccgacagctgcgtgcgagcgccttgcaggtggctgggggagattcacatccacttctgcaagacacgggccccccactcccagccccagccccgccgaaaccagctagacagctcctcctccttcactatttcctggaaccgccggcatgaaagagaaatcaattgttcttcctaaccctggcagaaacaataaaaccatctgggcttctggggctggggtctttatcacctccgttttcctcatgctttttaatctggcaagttctctatttctcgaggcaatagacatttttctaggaattaatctatttcacgttaatatttaaatgactagcatatctttttttctttatattctgg

SEQ ID NO: 120 - DNA, 2642 bp, Homo sapiens
cgccagcctgagtgcgacctggtgtcccgcagagccgagcacggtgtcgcgctgtgcgtccccagggtgaggccagtgccagccctgcgaggcaggggcctctccgcaccgcctcctctccgcagccgcaagatccaatccgcttgccctggcaggcctttaattccagcaatgcaggcaccccttggaaagggctgacgcttggggaaggggtcctccagtctctgggggaaagtccctattcccggggacgtgggcgcgcatcgggtccgcgccgggcgacgtcgcgctcgcgcccagcaggccccatgacttggcattccctccttcccctccccctcccgcccctgcggccggcagggcccccccaaccccacctaccggcgcccagcgccccgctcctgcggaccctccctcccggcggggatgctgccccggcccttcccagcgagagggcagcgcggctggtttgggggaagtcagcgtgaggagcggcttaaagcgggagtgcgaaggtcttcgcggccactgttccttcgtgtcatccgagcccgggcgccgggggcgcgccgccagcctagaccctgcggccccgcagcccctccctcgccgccgggccacacctgttacaggggcgctggctgctgagacgcaggcagtgctcgccggcggcccctcccaggtgctgggtctttctggaagggagctggggtctctggcgtgggagggggtgatgggcaggccggccgtgcaacgaagcgggtggccaggccccaacgccgcagcccccgccccgcccgcctgttttgctcccggctccagcatccgccctcccccaccccgggaaccggagcagctgcccgcgcccggccgccctgcgcgctctcccctcgggccgaggcgctgcagccgcgcaccctcccaggtgcggcgtcagcgtcctcagccccgctcaccatccctcccctcccccgccgtctgggcgcagcccctcccgcagccccgccggtggctcctctccagggaagcggaatagccggggaagggcgggggtatcccgatcccgccggccgggcgagccctcccgagggcgacccccgcccgcctaagccgagggtcccgctgcagccgagatcgcggcaaagggaccttttccagcggcacctgaagggcggacacccctgaatttcccccattgttctctgcggagatggaggaaggtctggaagtgaaggggcctcgcagtgcctgcgctttgggcggcaacaagggcgccgggccggagctccccgccctgggcgcagtgggtgcaggtgcgggctgcgggcgcgggctgccgggcgcaggtgcggggcgaggggcgcgggcgcgggaggccgggcgggcggcggccttacctctccggcgtcactggcggcggccccgcatgggcactggcatgggcgcgggctctcgagcccggagctgctgccgctcgccggctgctctgcgctgtgacggcgccgccgcgtgacgggctgcgcgcccccggcccggaccgccccgcgcccctcccgaggcgctggttggctgcgcgcggcgtccgagctccagtcgccagctggtttgtcgcgcggggagagggacgggcgacgcggccgccgcggggatggggctggggacggagggggccggggccgggctgtgacgtcaccgctgacaccgcggcgggccccgcggagggtgggggaccgacacccggctcggacgcagcgtcccctcccccggcgtgcccctgagacctcgcgggggagggacccggcggcgccccgtcccgaagccccgcgtgcacctgaacttgcccgcggacccctcgccgggccgtcccccaccctggcccctttcgagtgaaatcgtaaaacgtccacctgcccccgctctgagccagcgacagacgcatcgtggggggcgggcggggctggggagggagggctccttgcgccagggatgctggctccgggtgtaacaggtgcgcggtgaaatcgcatcttgtgtcggggccggggcgccgggggaagagcagcagtgacccaggccctccccgccggatgatgcaacccgcccgcggacccgccgcctcgcctctaccgcggctcgtggggaccccccgccctcctctcgccccacctctccaggcccccattgtcctcaccccagcctgtccagtttaacttccttggacccccaagtcacacgccgctcgggggccaccccggcaaaccccagcctgcgtcacccgcccccagccgccttcctagttccaggggctgactggtatctctgtgcgcgccaccgtcctccctccaccgtcccagggctgggcaacttcgacaaagcctctgggcagctcgcccatgggaacctccccgcactgcggtgcgggggtgcacggatttcgggccaccctgaaccccggggctgggtcccctccagcccagcccctgccatcgagcccctgccatgggcatcctggcgtcgcctggctaagcctccctggcgcccagcaggggcctgcgctgccctagcggcctcgccgttaaccatttcatccacactcctgggagagccggcttcacctcagtcccgacagctgcgtgcgagcg

SEQ ID NO: 121 - DNA, 50 bp, Artificial Sequence
  COL6A2 source sequence
gacctggctggggctgtgtccgcctccggggccctgggtgttggggaccg

SEQ ID NO: 122 - DNA, 50 bp, Artificial Sequence
  COL6A2 Unmethylated AlleleA Probe Sequence
aacctaactaaaactatatccacctccaaaaccctaaatattaaaaacca

SEQ ID NO: 123 - DNA, 50 bp, Artificial Sequence
  COL6A2 Methylated AlleleB Probe Sequence
gacctaactaaaactatatccgcctccgaaaccctaaatattaaaaaccg

SEQ ID NO: 124 - DNA, 3001 bp, Homo sapiens
gctgggggggtggccaaagaggacggaagcctcagaccacaggtgagacccctgtgaaggagatacggggagccttggccagcacgtgcccctagacagtgctggccaggccgagggagaacttctggagccaaggtaacccctaaaggattctggtcttgtgaggaggggcctggcttccccctgggagctgctggcttggaagcaggaggcattgcccaggtgagagttcggcatggacccaggggagtggcacacggtgcccgccgtcagctgtgctcaggagcagcaggcaggagccctggactcatggctaccatgtggctgccacactcaggcacacttaggcacatacactcacactcaggcacacttagacacacacattcacactcaacctcagacgcagacacacaccacacactcacagatgcacacagtctcagacacagacacacaccacacacagatacacacaccacagactcaaactcagtctcagacacagacacacaccacacactcagatgcacacaccacacacactcagtctcagacacacacacacaccacatttgcaaacctgctcatcagaagagggacctcgcccccagccgctccgccaccttccaggacagctctgaagggagccctgtcccggagcccagggacctctccccaacccggctgcgcctcacctcaaggaggaaccccggccctggctgggggactgtgaccagggtggggagtggggaccccagacagagccctaccagggacccctgtcactctgtccccggctgggctcaggtggggacctcacggtggtcccagggcccagcaccaagcccacctgtggtttccagcgggaaaggggtggcaggggtggctcgccgcatgcccaggctctgccccaacctccgcgcccaggctctgctgtccctgccctcccggctccccaccctcaggccccaggagcggcagtttctgcaggagctcctgacccggggcctctcgcgggaggcctgagcaagccggacacaggacacggggtaggggaggggtcggggggctgatggggggaaccctgcaccccccagggcagctgctaccaaggggcgagtcccagggcccccgtcggccctgcgtgcggggcgcggtccccaacacccagggccccggaggcggacacagccccagccaggtcgtccgggaaatggggcgggggcgacgggcggccgggcccgggacgcgaagtccgagcagcagcgggcaggggctggcgggggagctcggcccgggctgcaggggggtccccaccctctccacctcctccagcctcccgccctcgagggtccccgcttccctcccatccccctcccgtgcccccggccccctcctcccatccgcggggccgcagcgcttcctggcggcggggcgggtcaggccggcggggcggggtataaagggggcggcgccggccgcggttccctccctgctgcttactcggcgcccgcgcctcgggccgtcgggagcggagcctcctcgggaccaggtgagcgcctcccggaccccgcaccctggaagccgctcggcccgcggggggtgaccccgagtcctgggaaggcggcggcggcggctccgtccctcgggtccccgggaagggggactccagccccagggacggcggggggctcggcgggttcggggctcctcctcgcggggctggggccgcgcctgcccctgtggctccgcgtctctgggtccgaccctcgggcgcgcgacttggggccacctccccgcggcctcctctggggcggagccggcctgggcggggtgggggggtccctgtctgcgcccgagctcggtgctgggacccccgctcccgagacgaccccggcaccgcacgccccgccaggccccgcgtctgcgagcggttcgggtccggctccggcccgcggggaagacgccccggctggctgggacctccgggggcgcagggcctctccccgggccggacggaaggggcggcggggcggggggaggaggggctttcggtgcccgagggcgggactgggcggggaggggacgcgggtggccccgacgccccatcgctgcgcccctcccggcctggagcccaccagggccccgccaggcccaggagaagctgcagacggaggcggctccccagggcggcgggacccgggctgacagcgacccgcagcccctgccgggccgcccacacccgccctgggactccgccggggcgctggtggaaccgctgggcctgggtctccactgctggcaaccgaacggatcggccctctgtggagccgcaggtgtgcgggcgagcggcgcccatccgggctgtgccagcagaaccccggtgcccgcgcctaggacgcccctggagaagggaccttccctttggggtcggaacccagaaaggaggggcctgcgatccgcggagctccttgttcttgggataacacagctctggcttggaggcccccttgcacttcgactctggtgattatttcaagaaaggccagaccgggcacggtggctcacgcctgtaatcccaacacttggggaggccgaggcgggcagatcacctgaggtcaggagttcgagaccagcctggccaacagggtgaaaccccgtctctactaaaaatacaaaaaaaaattagccgggcgtggtggcaggcacctgtaatcccagctaatcgggaggctgaggcaggagaaaatcacttgaacctgggaggcggaggctgcagtgagctgagatcgcgccactgcactccagcctgggtgagggagcgagactgtctcaaaaaaaaaaaaaaaaaaaaaaaaaaaggaaagaaaggcccggtgagatgctttctcttaaacacggccctgcacgttgagttgctgcctcctgtggcctatttcacgtttatgcaaagtcgggcgcctgatgcggggctcacccgccacaagcaggggtcctg

SEQ ID NO: 125 - DNA, 1348 bp, Homo sapiens
cgagtcccagggcccccgtcggccctgcgtgcggggcgcggtccccaacacccagggccccggaggcggacacagccccagccaggtcgtccgggaaatggggcgggggcgacgggcggccgggcccgggacgcgaagtccgagcagcagcgggcaggggctggcgggggagctcggcccgggctgcaggggggtccccaccctctccacctcctccagcctcccgccctcgagggtccccgcttccctcccatccccctcccgtgcccccggccccctcctcccatccgcggggccgcagcgcttcctggcggcggggcgggtcaggccggcggggcggggtataaagggggcggcgccggccgcggttccctccctgctgcttactcggcgcccgcgcctcgggccgtcgggagcggagcctcctcgggaccaggtgagcgcctcccggaccccgcaccctggaagccgctcggcccgcggggggtgaccccgagtcctgggaaggcggcggcggcggctccgtccctcgggtccccgggaagggggactccagccccagggacggcggggggctcggcgggttcggggctcctcctcgcggggctggggccgcgcctgcccctgtggctccgcgtctctgggtccgaccctcgggcgcgcgacttggggccacctccccgcggcctcctctggggcggagccggcctgggcggggtgggggggtccctgtctgcgcccgagctcggtgctgggacccccgctcccgagacgaccccggcaccgcacgccccgccaggccccgcgtctgcgagcggttcgggtccggctccggcccgcggggaagacgccccggctggctgggacctccgggggcgcagggcctctccccgggccggacggaaggggcggcggggcggggggaggaggggctttcggtgcccgagggcgggactgggcggggaggggacgcgggtggccccgacgccccatcgctgcgcccctcccggcctggagcccaccagggccccgccaggcccaggagaagctgcagacggaggcggctccccagggcggcgggacccgggctgacagcgacccgcagcccctgccgggccgcccacacccgccctgggactccgccggggcgctggtggaaccgctgggcctgggtctccactgctggcaaccgaacggatcggccctctgtggagccgcaggtgtgcgggcgagcggcgcccatccgggctgtgccagcagaaccccggtgcccgcgcctaggacgcccctggagaagggaccttccctttggggtcggaacccagaaaggaggggcctgcgatccgcg

SEQ ID NO: 126 - DNA, 50 bp, Artificial Sequence
  SFRP5 source sequence
cggcagccagctgctcgcctgctgcttcacttcggccaggctctcgtgct

SEQ ID NO: 127 - DNA, 50 bp, Artificial Sequence
  SFRP5 Unmethylated AlleleA Probe Sequence
aacacaaaaacctaaccaaaataaaacaacaaacaaacaactaactacca

SEQ ID NO: 128 - DNA, 50 bp, Artificial Sequence
  SFRP5 Methylated AlleleB Probe Sequence
aacacgaaaacctaaccgaaataaaacaacaaacgaacaactaactaccg

SEQ ID NO: 129 - DNA, 3001 bp, Homo sapiens
gggaggctgagaccagggagaaattagggttcaggggactgaggggtgggtgggaggagggacttaggacctcataggtttcatgagcctgggatggcttcctattttatggatggggaaactgaggctcagtgaagtcaagcaacttgcctaagggcttctcttcaggggcagagcctaatctgctcttccttccgcactgagccttacccgaatgactcccagccaggggaactggggaagagggtggagatcttagggttccaggtacagaaaacggggacgcagatcatagtccagaacattcttagtccccagtctttctccacatcccttccctcacctctcctctcttcccaaggtcccaacacggagtggggatgatgcttgctttggtgcctcgggatccaggtgaaaggcattgtttaacgaccccaggcattcttgggctgagaggggtcataaggcatttatcaggacgtcccaggattggggggacggcattgggggattctcagcagcttggttggatgggtactgagaggggtgccaggggtttccagggaacgagagccagagggaaggtctggagccgccgggaggcactatgctgccactccctggggaggtgggaaggcgggaaggctgcgggggataattccggggacgctgggctgagctaggacagcgtgtgtgtgtgtgtgtgggcggagggggggagctgcacctcttggggggttcgcagagctgtgctaggggagctgcagggccgtcggagcgcgcggggacggcggcggcggcgctacctggaggcgcggtggcgggcaggtgcccgaactgcacggcgatgcagaggtcgttgtccagggggaacttgtggcagtgcagcatctcaggccaggggaagccgtaggcctccatgagcggcgcgcagccggcgcgcacggcctcgcacagcgagcggcacgggtagatgggccggtcgagacagacgggcgcaaagagcgagcacaggaagacctgcgtatccgagtggcagcgcttggccagcagcggcagccagctgctcgcctgctgcttcacttcggccaggctctcgtgctccagcaggttgggcagccgcatgcgcttgtagcccaccgtgtggcagagcggcaggtcggcagggatgtcaaggcactgcggcggcttggagtaggagcggccgtgcagcggctcggcctgccagccatagtagtcgtactcctcgcagcgcgccggcgcccagtgcagcgcccccagcagcagcgccagcgcggccgtccgcacgcccccccccgccgccgccgcccgcatggctgcgccctctccaggtgcgcgccgcgcagccccccgacgctcggtgcccggcggcccaggtgttccggcgtgcgcccccggccctgactctacccagccgccgccgccgccgccgcggaggtcgcccaggtgggtggcagcctgcgctgcgggcgccccgactgatcctggcgcctcccacctcggggctccagcccgggcctcgccgtgcgccccagccaatctccggccgcctggccccagctccaaggcggggaggcagtgggggcggccctcagcccctcccgcaccctgcccgagcgtcccgcccagcgccaggtcttgcgcccctctcccggctcggctcccctgccgcagccctggcctcgcccagcgtttctccccagggctccctgcctccctgggcattttcctcgaccctcgccttctccatccttcctccttatctctctccgccacagcctctcctgttccttttctcactcacttcacttgcttatctctctgccttggcttttctccctctgctctgttttctcttccccgacacctcggttcctcttctccctggtgggaggctcgtgccaccccacaccctggatcttctggtcctcctggcaggtcttcattgagcttggtgccaggccccatgctggctgctggggaatcaaagatggatatgacccagaacccacctccagggccaggatagtctagtgggggtgaaggtaatcatagcttctatttagtgagctcttactacctgccagggacggtgcaaggtgcaaggtgcggtgcaaggtgcggtgcaaggtacgttccaagcttatcagctattgctaatgatagccaatacttagtgggtgctaattgtgtgccaagtctgatctgagcacattgcataaactccctcagctaattctcacaataaggctagatggtgggtaggtactattattgtccccaccttacagataaagatcctgcggcagagaggttaagcacagattagagctgggatttgaacccagatagtctttttcgagagtcagagcccagaaacaaccaggaggcctggattctgagtctgtaggcctaatgaccaaggggatagggagttcagagaagtggagtaactggcccttggccacacagctcatatgtgaaggagctaggatttgaatccagatgttgggctacaaaggccagggcttaaccactctgtggtaagataacttccaacccatggtagggaacacagtgggaagagggggaattgatggtgtgtctgagcagggcacccaggcaagcatcagaggggaggggaccttcaggagccccatgcttggtgtctttctctcctgtctgagtcctgcctttcattggctctcccttgagaggaatttggactggaggcttgaagggaggggaattccttcctgagagcctagcctgacacctgacctcccagtctttggtggggagggtaccttggggaggtgtctcctaggagggcgtggggagctgggggctggctctgctggaaggcaggagtactgggcactttccccctctgctcctccccttagtcccactaatggatgtttccagggctcctgtcttcctctgggtctgtccttgcagtggtagtggagaggggtga

SEQ ID NO: 130 - DNA, 943 bp, Homo sapiens
cgtcggagcgcgcggggacggcggcggcggcgctacctggaggcgcggtggcgggcaggtgcccgaactgcacggcgatgcagaggtcgttgtccagggggaacttgtggcagtgcagcatctcaggccaggggaagccgtaggcctccatgagcggcgcgcagccggcgcgcacggcctcgcacagcgagcggcacgggtagatgggccggtcgagacagacgggcgcaaagagcgagcacaggaagacctgcgtatccgagtggcagcgcttggccagcagcggcagccagctgctcgcctgctgcttcacttcggccaggctctcgtgctccagcaggttgggcagccgcatgcgcttgtagcccaccgtgtggcagagcggcaggtcggcagggatgtcaaggcactgcggcggcttggagtaggagcggccgtgcagcggctcggcctgccagccatagtagtcgtactcctcgcagcgcgccggcgcccagtgcagcgcccccagcagcagcgccagcgcggccgtccgcacgcccccccccgccgccgccgcccgcatggctgcgccctctccaggtgcgcgccgcgcagccccccgacgctcggtgcccggcggcccaggtgttccggcgtgcgcccccggccctgactctacccagccgccgccgccgccgccgcggaggtcgcccaggtgggtggcagcctgcgctgcgggcgccccgactgatcctggcgcctcccacctcggggctccagcccgggcctcgccgtgcgccccagccaatctccggccgcctggccccagctccaaggcggggaggcagtgggggcggccctcagcccctcccgcaccctgcccgagcgtcccgcccagcgccaggtcttgcgcccctctcccggctcggctcccctgccgcagccctggcctcgcccagcg

SEQ ID NO: 131 - DNA, 50 bp, Artificial Sequence
  FOXL2 source sequence 2
cgagaagaataagaagggctggcaaaatagcatccgccacaacctcagcc

SEQ ID NO: 132 - DNA, 50 bp, Artificial Sequence
  FOXL2 Unmethylated AlleleA Probe Sequence 2
aactaaaattataacaaatactattttaccaacccttcttattcttctca

SEQ ID NO: 133 - DNA, 50 bp, Artificial Sequence
  FOXL2 Methylated AlleleB Probe Sequence 2
aactaaaattataacgaatactattttaccaacccttcttattcttctcg

SEQ ID NO: 134 - DNA, 3001 bp, Homo sapiens; identical to SEQ ID NO: 40

SEQ ID NO: 135 - DNA, 2628 bp, Homo sapiens; identical to SEQ ID NO: 41

SEQ ID NO: 136 - DNA, 50 bp, Artificial Sequence
  RCSD1 source sequence
cgcaaatctaaacagatactgtccctcccagggcaggactcaagagcggg

SEQ ID NO: 137 - DNA, 50 bp, Artificial Sequence
  RCSD1 Unmethylated AlleleA Probe Sequence
cccactcttaaatcctaccctaaaaaaaacaatatctatttaaatttaca

SEQ ID NO: 138 - DNA, 50 bp, Artificial Sequence
  RCSD1 Methylated AlleleB Probe Sequence
cccgctcttaaatcctaccctaaaaaaaacaatatctatttaaatttacg

SEQ ID NO: 139 - DNA, 3001 bp, Homo sapiens
aatcagagcagttaagtgacttgtctaaaaaatacaactagaaagtagcagagctggaattcagccctcaatctgtctgttcctaaaaccaggcgtcttcctctatgcttagctaagctaaagaactagaagccactctggcattagggctgccagtgttttagaaagttgggccttagaaagttctaggacaggaaggaggccatatttctaagactggtacccaaagcagcaatctaagtgcctgaaagtgcaaagatggtgccgggataaccagaatcagggtagtctttgtagcaggagcagactctcagccttttttctctactgtttctccacatgtctcttcacagtgctcaccatcacctgtcccattgcccacctctgatggggctcccctcccctggttgagctggggttgggcgggtgagaatgctgtgggataacccagaggcgtcatatacccagccaataccctggcactcacaggtctctcaaggcattttcctgtttctctctgccgaagggtctgaagggggccgggatccgggtagcagggagtgaccctggtgcagattccagtccccgaatccctcccatgcggtctctgctgcccagccgggatggccctaaaggaaggttgctatgctgggccctatgggaatcagatgttgagcatcttgccctttgctatatttaacaggaatggagacgaggccctttagcaagcagcttgtgggagccaaaattcccacaaaaccgaatgcatcagtcaaagcaaggtttgaagaaaagatttaccacttcagggagcttggaaaactttgcgtggattcctgattttcctcctcctcctcctccttctactcgtatacagaagatagatggatagatggatagatagtggtttcctggaaggtggagaggaaaagaggtgagaagggaaaggtggtcccgtcggcgccacgctgagctctgaagcctcggtcagaagggctcctcttttggtagaacacaaacgccttccaccagggagggtcctcgaactccacccaagaactgcagggaaaccaggtatgacagtaatgcaattttaaaaagagggagaggaagaaggaaagcaggaaggatggacacaaaagaaggaaatcgaaaagagaggaaacaaaaagaattaaaggaccactctaattcttgaagggacaggaaaaagaaagaaggcgcattaagggagggagacgttgagaaggactgagaccgtggcaaagcccagggtcctgagaaacttcgcgagacagaggggcccggtggctgcccatccccatgtttgggcacatggagtcgtggctcccgctcttgagtcctgccctgggagggacagtatctgtttagatttgcgtctaaatttaaaccctgatcgtcggcctccctccccgctccccaccctcccctcccctccccttcctccctccctccctcctcctccttcctcgttctctcgcgcagggcccccgcggccggggcagtcccgcagccgagcgcagccgggcgcgcgccaccgcccactcgccctgtgcccgccgcagcccgaaactggccacggccgggagcggaggggacagcggggatcgtgagctccggcccgggcgagcgggtgcgtctgccgcagagtcggcacctgaaggacatggaggtaaaggaccccggagggagacgcggggctgagcggtgagcggtgtatcgggcgccccttccccgggcggctgcccctgccctgagcatggagcacgcggctcatcgctgctgccaactagaagggcaaatgcctgctgttccgacgcctccttgaccaactccgaaggatcgtccctggtcccacctaggacacccgggttgggaggctggggttggtaaggaaagatctcagagggagagaaaagggctttcctagaggcccgggtattttggctaggaacttaaatgctaaaaaaaaaaaaaaaaaaaaaaaaaaaaaaaagttaggtgcagccaaggtttgtaaatgggtaaacgtgtcagcaaaaactgcaagaaggcagaagcccttcaatctgaagagatgggtattgaaagagaaacagaactggcaacacgaggccagcacgtcttcgtgggtctgtgggcttcccttttcgctttcggagtggcattttagagagtccagttgtcttcttgcttctgaagtccttcctgtgtctgtcgcatcttcttccataactttctgaaagctccccttactggtcagtcgaatgacattatttctgataaaacttaagactcccttggacacccttgcctctgaagctgtgtgtatggaggtatgctggccacatctgcaggctttgccagagggaggccccgcacttctctgcctgctgagaggaggtccggggactttccttgttctgttccaacctcagcagaggagtggttgggtgtattccccacatccctctcttctgcacatcttgaggcttccctgtgtttcctccttgaaaatgagacctgatatgtagccctaagtcaatcttagtaggacctggggcttttgagaagatgcaaatgttatatagttttaaaggtgattcccactcatattcaaaaggaaacatgtgttgctcactagactgtggtcagtgcaatcaggattcacagttaactccgctatctcttccccgctccaggctctgtgcctgggccctggtggtctttttatttttttcttttgagacagggtctcattctgttacccaggctggagtgcagtggcacaatctcagctcactgcaacctctgcctcccaggctcaaaggatcctcccacctcagcctcccaagtggctgggactaccagtgtgtaccaccatgcctggctagttttaaaattttttgtagagacaagttctcactatattacccatgctggtcttgaactcctgggctcaagtgatcctctaagcctt

SEQ ID NO: 140 - DNA, 375 bp, Homo sapiens
cgttctctcgcgcagggcccccgcggccggggcagtcccgcagccgagcgcagccgggcgcgcgccaccgcccactcgccctgtgcccgccgcagcccgaaactggccacggccgggagcggaggggacagcggggatcgtgagctccggcccgggcgagcgggtgcgtctgccgcagagtcggcacctgaaggacatggaggtaaaggaccccggagggagacgcggggctgagcggtgagcggtgtatcgggcgccccttccccgggcggctgcccctgccctgagcatggagcacgcggctcatcgctgctgccaactagaagggcaaatgcctgctgttccgacgcctccttgaccaactccgaaggatcg

SEQ ID NO: 141 - DNA, 50 bp, Artificial Sequence
  SLC47A1 source sequence
cgggtcttgcaaagaccaaggaactcctgcacttggaaggcaaagtttga

SEQ ID NO: 142 - DNA, 50 bp, Artificial Sequence
  SLC47A1 Unmethylated AlleleA Probe Sequence
tcaaactttaccttccaaatacaaaaattccttaatctttacaaaaccca

SEQ ID NO: 143 - DNA, 50 bp, Artificial Sequence
  SLC47A1 Methylated AlleleB Probe Sequence
tcaaactttaccttccaaatacaaaaattccttaatctttacaaaacccg

SEQ ID NO: 144 - DNA, 3001 bp, Homo sapiens
gcgattgttccagaggtgggcaagaaacctaagcagggccaaccagattttctcccagttttttgtttgttagtttggaaccagaagagaggaggggagggccttactttctggtttaacagacgggaaagataggaacgagggtatcagaggccaccactgtcacttgcacaggaagagaaaggctgccggcagaggaggaagagaaactacggaaagaatcccaaggggcgaggagaagggaggagagggacagagatgggaagagggacaacaagaatgatctggttttgttttttttttttttttgagtggagtctccctctgccaccaggctggagtgcagtggcgagatctcggctcactgcaacctctgcctcccgggttcaagcaattctcctgcctcagcctccctagtagctgggattatggcgcgcgccaccatgcacagctaattttcggtacagacggtgtttcaccatgttggccaggatggtctcgatctcttgaccttgtgatccgcccgcctaggcctcccaaagtgctgggattacaggcgtgagccaccgcgcctggcctaatgaccttgttttcaaatccctgaatccaaacttcctgattatgtgagccaaaaaatttgttttccattttctctacttacattggatcattacaacggaaggaattttgactattagagaaatcaatcttgtcctggtaactgacacttattaggtaacggctggtgccaggctgagggcaggtggcactgactccggtcctccgcccaggcaaccccagtcttcacagctggcgacgagtgttctttcacgtttgcagatgaggaagctgaggctctgggaggaaagcgcgctgctcctgcccttctgggccgcagcctctcgtactcccacagcaggcttgatggataaacttccaacacgaccagctggcacaaactgtgctacttggcaaatgggagcatgttgctctatcctggtagattctaagttctctaatggaacaactgatgagaggagaggagtatgcaacaccctaaccagcatcaaagggtgtacccaactcaatctgcacagcaaatgcgcccaaccccacacaccccgcggggtgctaaccgagtcacggctcgaggcctctgggtccttccacgccaggtcattcagaacctgaaacccccagcggaggtggagagccggggtgagtcctggcgtccgagacgtggaggacccagcccccggagcgcgttccgttagtttacagagctttttcaaactttgccttccaagtgcaggagttccttggtctttgcaagacccgcaggcgtccgcagtggtgcagagagaggtgcaaggggcgcgcccaaggtcgcaggctgcctggctgcgcgctcagtcgggcccccgggcgccaggcgcggggcgggctctgggcgggcggccggggcggggactgcagagcaggctgcactgcgcggtacccactgccggcctgcgcggtactcactgccggcctccgcggtacccactgccggcctccgcgctacccggccgcagcgcgcgagtcacatggaagctcctgaggagcccgcgccagtgcgcggaggcccggaggccacccttgaggtccgtgggtcgcgctgcttgcggctgtccgccttccgagaagagctgcgggcgctcttggtcctggctggccccgcggtgagtaaggtggcctcagtggcaggccggtaccggcgggctggggacctggtgatttctgcctccgcgggtgactttggcgggctttggggaccgagcgagctgtccgccggcgggctccggggagcatcgtgccaggagccgggcgggcaccccagctcctctgcctgcgtcccggccgctttccgctccgcaccacccgactgggggccccgcggggcactgcgggaacggctggtacgcgcccgggcgcatgttggctcggagaggccggcggctccagcctcctgcgccaggagacaccggagagctgggacctttgcccctaacggggctctaccaggtccaggctgcatttgcatccgggggtgccttctctgggagctgattccggccaataaaacgcaagttgcctgaacttcaaaacacaagggaacttgtttctttgaaaagtagcggtcaaaagttcgggagctctgcgatctaaataatcccgaggcacggaggagggggtgcgtggtctgtcgtggactcagagctgcgaaatgatttcttcagaattcagtcaacctctaccgaggaaaccagaggcatcctgtaccaccccctccccgccagtaattatgtatgtgaaagaaaaaaggggttttaatgtggttctttgtggggtcgaggtgttcttatctgtgggattcttttctttttaatcattaggttagggctgtttctgccttgtctcctaaaaataatgtggttttaaaaaccacaaaaacacaatcctatccacgttctcctgccagcttgggtggttttcaaaccgagagggtaggacgcggcacaagaagcaactggtatgcacttaaggcgtgaataagcttttgactttgaggcccacattctgcacctgcgcgggctgaggggggccgctgtgcctctggagtgggggctcggtagtttgtgggatattgtggcacccgctgtggtgtgcaggacagcaagggtaatgccgaggtcaaagccatggaataagatccatcagccatgctttctcacagaccagaaagaactcatgatttggggctgggcgcggtggttcacgcctgtaatcccagcactttgggaggctgaggcagatggatcacttgaggtcaggagttcgagaccagcctggccaatatggtgaaactccgtctctactaaaaatacaaaaattagccagacgtggtggcgggcgcctgtagtgccagctactccagaggctgaggcacgagaatcgcttg

SEQ ID NO: 145 - DNA, 904 bp, Homo sapiens
cgcggggtgctaaccgagtcacggctcgaggcctctgggtccttccacgccaggtcattcagaacctgaaacccccagcggaggtggagagccggggtgagtcctggcgtccgagacgtggaggacccagcccccggagcgcgttccgttagtttacagagctttttcaaactttgccttccaagtgcaggagttccttggtctttgcaagacccgcaggcgtccgcagtggtgcagagagaggtgcaaggggcgcgcccaaggtcgcaggctgcctggctgcgcgctcagtcgggcccccgggcgccaggcgcggggcgggctctgggcgggcggccggggcggggactgcagagcaggctgcactgcgcggtacccactgccggcctgcgcggtactcactgccggcctccgcggtacccactgccggcctccgcgctacccggccgcagcgcgcgagtcacatggaagctcctgaggagcccgcgccagtgcgcggaggcccggaggccacccttgaggtccgtgggtcgcgctgcttgcggctgtccgccttccgagaagagctgcgggcgctcttggtcctggctggccccgcggtgagtaaggtggcctcagtggcaggccggtaccggcgggctggggacctggtgatttctgcctccgcgggtgactttggcgggctttggggaccgagcgagctgtccgccggcgggctccggggagcatcgtgccaggagccgggcgggcaccccagctcctctgcctgcgtcccggccgctttccgctccgcaccacccgactgggggccccgcggggcactgcgggaacggctggtacgcgcccgggcgcatgttggctcggagaggccggcggctccagcctcctgcgccaggagacaccg

SEQ ID NO: 146 - DNA, 50 bp, Artificial Sequence
  HTR7 source sequence
ctcgcagcctgggttttatggctgggcaggctcgaatggcagccgggtcg

SEQ ID NO: 147 - DNA, 50 bp, Artificial Sequence
  HTR7 Unmethylated AlleleA Probe Sequence
aaaatacccccactaaactataactaactaatatacaaaaactaaaatca

SEQ ID NO: 148 - DNA, 50 bp, Artificial Sequence
  HTR7 Methylated AlleleB Probe Sequence
aaaatacccccactaaactataactaactaatatacgaaaactaaaatcg

SEQ ID NO: 149 - DNA, 3001 bp, Homo sapiens
ttcccctagcatccctgacagatggtcagcctctgtttcaattactccagtgggggagttctcaagagaaggttcccaagggctatgatgaaaggctagaaccagccattcagacctggaggaaggctccagtcagcaagctcgcaagatacagacagcattaccaaacattgaaaccagacttccacaagcaagaatgcgattccgaactgaccatcataaagatgcaatgtgtaagacatacaaaccccaattctagaaccatccaaggtccttttactggcctagggagtagctcccacaatggggtaagcaaggtggcttttctccgacttattattattattttttaatgactctttaaggctcacagcaaggcttgatcaagtcagggagcaccaaatcccttccttccaaaaaccaaggcaagggacccgctggtggaaatgctaaggcttaaaatactcttaactttggtcttaaattcgtcactacagtaaaccagaacatttccggtttttcttttaaagatgtacataggctatgccgtaaccgaagtttggttggtgtagggtggattggggggagcggtgttttaagcgcagcccttcatcccgccttgaagtctagcttgatcctcccaggaaaggcgagcgcgcggggctgagctgccagccggtccccagccggagcctgggacggggcggtccggcccttacctgtcaatgctgatcacgcacagggtcatgatcgaggccgtgcagcacatgacgtccatggcgatgaagacattacagaaaaagtgtccaaagatccacttgcccccgatgaggtcggtgacgctgacgaagggcatgaccgccacagccaccgagaggtcggccagcgccagggacacgatcaggtagttggagggctggcggagcttcttgacgaagcacacggagatcaccaccaggcagttgcccgcgatcgtcagcagcgtgatgagcgtcaggatggagccgatcacaactttctcgactctgccgtagttgatctgttccccacagccggaggcattgtccgggggcgcgtcccaggtgggcgccgggctggctgtcacctcgctcagcaggtgcggcgcccaggagcccgcgaccgggtcggcgccaccgtcggggctcaagtcgggcagcccgcgccccacttctggcagaaggaaagagcggaggtgcccgtagaggtccgggcggccgctgctgttaacgtccatcatcgcgccgccgtgtgccgctgcccatggagccggcgccccggccacgcgcctccggctgccggccccggggcttcacctcaccggttccgctccgcccggcccagccatggggcccgcgccgaccgctggggggcgcctggctctgtctcggagccccgcactccccggacccccggccgctgcgggtaacgcggcagcgcggcctcacggggactccctgcgggaggcgcttcggcccccgacggacgcctgggacgcgcggagtcgagggagctcgggctgggctccgctggcagctccacagttcgcagcagcagctcggctgcgccgagagcgcccgggcggcagcggcagaagttgcggagtgcgccccgcccctcgcgcccgccgccgccgccgccgccgccgccgctcccctgcggcagccgcgccgcgtccccgccgccgccgcctccggccgctgcccgctccctcgcgctcctgggacgggatacccgggctgggcaggcaccgccgcctctcccgcgcctccctactgtgagggggcggcgctgcacgcgcctcccgggatcccgggtcccctcgcccgggaagctgtccttgccgcatgatagatacttagaacccctccagccgaccccagcctccgcacactagtcagccacagttcagtgggggcatttttcttttgttaagaggcctaatgagccagcggatgtttgggctcattgcgaaatgaagaacctagagggggattttatttttcttaattaaacaacaaccaaaaaaacgtctctgagcactacactaagcaacaaataggtacttccgttttctattttgaccgcttatgggaagaagcgaagatggaaaagaagcagccgaatggggtgagggtgggagctccgagaagaggaagaaagaaaaaaaggagggagggggattccttaagcccctggcacccaggagatgaggatgctctggggacaaggcctccaagggggttacacccatttggactaatccttcctggatgtctagaaacagtcctctccgctctgaaaatagaccctgattgcctttatatttagacttcaaagcctaatatattgaaaagagagttaattttcccgtagcattctggaagtcttacacataagcaaagaaatgaccggttatactcttctataaaggaatcctggaggtgtatgttgatttaatacctattggctaaaattccccttggtatccaaaccctgtcagagcggctgatttttattatttgtgagacttgcttcattccattttgtttgaaggtttcattggaagccagcagtgatttctgtagtttaagactctaaaaataatcaaatctgttttttcttgttaggccctagcggattataaataagaagaaaaggaataaaaacctcaaagtatattgatcagagtgctgaatagtactttcattcttcaaggtgggtattagctctgtttttccagatagggaaggttgttagctcttgcaatcagctggtaaggaatagttatgtttcaaacccaagtttatctgattactatgcccatattcttccctgtacacattcagaaaaagttttaatggaaaaaaaaaaaagccctaatgttcacagctcttttgctgtgatcaacatttcagaggctcctctctgcctaggataagcaaaccaatccattgagtcagaggaagagttgaatataata

SEQ ID NO: 150 - DNA, 1214 bp, Homo sapiens
cgagcgcgcggggctgagctgccagccggtccccagccggagcctgggacggggcggtccggcccttacctgtcaatgctgatcacgcacagggtcatgatcgaggccgtgcagcacatgacgtccatggcgatgaagacattacagaaaaagtgtccaaagatccacttgcccccgatgaggtcggtgacgctgacgaagggcatgaccgccacagccaccgagaggtcggccagcgccagggacacgatcaggtagttggagggctggcggagcttcttgacgaagcacacggagatcaccaccaggcagttgcccgcgatcgtcagcagcgtgatgagcgtcaggatggagccgatcacaactttctcgactctgccgtagttgatctgttccccacagccggaggcattgtccgggggcgcgtcccaggtgggcgccgggctggctgtcacctcgctcagcaggtgcggcgcccaggagcccgcgaccgggtcggcgccaccgtcggggctcaagtcgggcagcccgcgccccacttctggcagaaggaaagagcggaggtgcccgtagaggtccgggcggccgctgctgttaacgtccatcatcgcgccgccgtgtgccgctgcccatggagccggcgccccggccacgcgcctccggctgccggccccggggcttcacctcaccggttccgctccgcccggcccagccatggggcccgcgccgaccgctggggggcgcctggctctgtctcggagccccgcactccccggacccccggccgctgcgggtaacgcggcagcgcggcctcacggggactccctgcgggaggcgcttcggcccccgacggacgcctgggacgcgcggagtcgagggagctcgggctgggctccgctggcagctccacagttcgcagcagcagctcggctgcgccgagagcgcccgggcggcagcggcagaagttgcggagtgcgccccgcccctcgcgcccgccgccgccgccgccgccgccgccgctcccctgcggcagccgcgccgcgtccccgccgccgccgcctccggccgctgcccgctccctcgcgctcctgggacgggatacccgggctgggcaggcaccgccgcctctcccgcgcctccctactgtgagggggcggcgctgcacgcgcctcccgggatcccgggtcccctcgcccgggaagctgtccttgccg

SEQ ID NO: 151 - DNA, 50 bp, Artificial Sequence; identical to SEQ ID NO: 146
  ACSL6 source sequence

SEQ ID NO: 152 - DNA, 50 bp, Artificial Sequence
  ACSL6 Unmethylated AlleleA Probe Sequence
ctcacaacctaaattttataactaaacaaactcaaataacaaccaaatca

SEQ ID NO: 153 - DNA, 50 bp, Artificial Sequence
  ACSL6 Methylated AlleleB Probe Sequence
ctcgcaacctaaattttataactaaacaaactcgaataacaaccgaatcg

SEQ ID NO: 154 - DNA, 3001 bp, Homo sapiens
aaggagcttagggagctgaggggattagggaggcttcactcactctcccagctcaggggaagtgtccctgtggcctaacttaaggaaatggtgggggtgaaggaagtgattactaggcccaccaaccctccaggttcaggcctgatccagccaagcacttagcattggttgatcaattaaaacctcgaatggaatcccagtctgccaaatatccatgtgacactaggcaagtcaattgacctctgaactcagtaaaaaaagggatggtgccaccgacctcctagggttttccatgaatattaaataacacgtacaaagcctcaggtaaagatttcaagagggtcaacttcctccccttccttggctcagaaaaagctttattaccacttctgatggtgctgtttgaatgtgagataataataaacactaacattcatagagcatttgctgtggcctaggacaggtttaaggccaatttaaggatcaagcactgcacacaaattatcaggctgaatctgcacatgggggttggggtggaggtggagatcctttagtggtattatactctggaggaccccaggctcagagaggggaagtagccggtgcaggtggtagaactggcagataaaagggcctgtggtgagactccaggtgtgggtgtatggggggttgagggaggtaagcgcggaggcgggatcgagcaggggtccttgtagccgcctaagaagtgcagtggtgaagctgactcctgtgaggtggaggggaggggtctggaaacagtggagacacagcagccctgggcagagcagaggagccaggtgaaccctaccttacagaaatcttgtactctggctgaaggacgggcagggaggggtcgtgaggaagcccctcgccgggatcaggaagcctaggtcagtccgggttacatagctgacctgctgtgggacctcggggaccaacaccctcggtttctggtcccaggagatggacaaggacgcaatgtctgttcctggccttggctcagggcctaatctgatccgcggatggtccttgccatcagggaagggggacgcaagaactcggcgggggtttgtggtggggtcgcagagagcaagccctatatctccctccgcagacccaggtgctccccaaacccggcccggagcccgcgagaactgggggcggagggtgtacttaggcggccctggggaccttgacgggacagctcagcagcaggggatgggggctcggcggccgcggagatgtaacacctccacctcgggcgaagacctcatagcctgcgggagatgggagtccgggacgcggacaggacgggcacttacctacgaatagccagagggagccccccgacacgaggaagaaggtcagcatggcggtcagcggggcccggcccggcccggcccggcctccccgacccgcagccccgcagccccgcagcccagcagccccagcagtagccgcgccgccgccgccgctgccgggtatttttagcccccgccctccggccccgcagctcccgcctccccgcgccgctgcggagacggctcaagggggagggcgcggcgcgcactcgcaaccgagccttactccctgccctgcagcctggccggccgggtctggggcttgccccgcactgaccgcggtgtcggaaccgccaagctcccgggcgggggaggggcccggccgcagagcgaaccagccctctccggccccgctctccaacgtcagtacctggcattctgcggaaaccggctggaagggggagcacgggcgacgagcgccagagcgtgacattgagcccaccccgccgccacccaccccatcaacacgcgaccctgcccccgcgcgactcgcagcctgggttttatggctgggcaggctcgaatggcagccgggtcgcggttacctgtcctaggaggtgcacactcctgtgggctgttgcccgctgacaccaacgccccctaggacccgagtttgcagacatggggagcctccgggtgccacctctgcaaaccagtgaggggagggagtggcaagtgttcacctaggctctgacaaatcaggcagctagaccagatagtgcccagatcatctggggacgcacgagttcaggtctgaggctacgcaaggtcacaggagactcagtgtgatcacaggctgccatatgtgttcaccagtagtaacatggtcacaggcagccacatggaatcacaggtgtctcatgaggaaatcatgatcttcgtacacaactgtatgtggcacacgaggtctcaagaagcccctcatagccacaataaatggccatatagccacacacagccacagatgcccacactgctgggctgagggcttggacacctgttgcagtgtctgccattgaaaggaggaagcccagttgcacattttctgaaataataagggtaggcatcctggtgggcaccccatgaacatctgcacctccaagcaagatcacctcttcccaatgcccttgcttgttttcctcaagcatgcagagcccaccaggactccagcaccgaggacagcgctcaggcagccgggccttctctatacgcagccctcccaggccaggagctggctgagccacagcccagaaagaagaggatagaaaaccagagagcagaagtcaggaagcagagagtggaagagcatgagatctcttcaggcccaccagagaccatcagccatgagtgcatgtgggctcacagctcagcagctgggtgtgtgggggggcctgtatctgtagtacgagtacatggcatgtgtgtgcccaggcctgtgtgcaccttgtagtggtgcaagtgtgcagatgtgtgcctctgagtcacttccaatctttacaataggatcctacaccttctccatcataggatctggactcctgggagagctgctccagcttgaactcagccacagcctggcagggactttgctgggatctcaagcttggcagggctcctcc

SEQ ID NO: 155 - DNA, 884 bp, Homo sapiens
cgcggatggtccttgccatcagggaagggggacgcaagaactcggcgggggtttgtggtggggtcgcagagagcaagccctatatctccctccgcagacccaggtgctccccaaacccggcccggagcccgcgagaactgggggcggagggtgtacttaggcggccctggggaccttgacgggacagctcagcagcaggggatgggggctcggcggccgcggagatgtaacacctccacctcgggcgaagacctcatagcctgcgggagatgggagtccgggacgcggacaggacgggcacttacctacgaatagccagagggagccccccgacacgaggaagaaggtcagcatggcggtcagcggggcccggcccggcccggcccggcctccccgacccgcagccccgcagccccgcagcccagcagccccagcagtagccgcgccgccgccgccgctgccgggtatttttagcccccgccctccggccccgcagctcccgcctccccgcgccgctgcggagacggctcaagggggagggcgcggcgcgcactcgcaaccgagccttactccctgccctgcagcctggccggccgggtctggggcttgccccgcactgaccgcggtgtcggaaccgccaagctcccgggcgggggaggggcccggccgcagagcgaaccagccctctccggccccgctctccaacgtcagtacctggcattctgcggaaaccggctggaagggggagcacgggcgacgagcgccagagcgtgacattgagcccaccccgccgccacccaccccatcaacacgcgaccctgcccccgcgcgactcgcagcctgggttttatggctgggcaggctcgaatggcagccgggtcgcg

SEQ ID NO: 156 - DNA, 50 bp, Artificial Sequence
  SCG3 source sequence
ctcctttgttccattcccgggggattggagtagcgttggagtcaccgacg

SEQ ID NO: 157 - DNA, 50 bp, Artificial Sequence
  SCG3 Unmethylated AlleleA Probe Sequence
ctcctttattccattcccaaaaaattaaaataacattaaaatcaccaaca

SEQ ID NO: 158 - DNA, 50 bp, Artificial Sequence
  SCG3 Methylated AlleleB Probe Sequence
ctcctttattccattcccgaaaaattaaaataacgttaaaatcaccgacg

SEQ ID NO: 159 - DNA, 3001 bp, Homo sapiens
catcctgaatctctcccaattgcaaacatgtattgaggagataaatgagtcaggaatgggacaaaggacaaagtgaagctgagggattaggtcagtgagtctcaggatcagtgaatggctatttcagatagcgttgggtaatgtttcataaaagctgaaaacgtttcatgacttattttcttaggctgtatcaaaatattcttggtatcaaaatgactagttcaaaacatgtacccatttgatcttcataataacagtgtgaggtaaacagagcctgtcatttccatcataaagaaaaggaaactaaggaaatatacccttcattcagctgggattttaaaatatgacttaattttttaatgcagtatactgggataataatgctatgttattactgccaataaagagttttttgttttgaaggagctgttagatttagaatactctttagatcctcaaaggaagtatttgagtgacttccaaactcaaaaacctccggagcaagggagatttgtttgtcagcttggttgtttgtttgtttttaggtacttgaataacttcctccttgaaaaggagggtgcagaaatcaaaggcctgattgtgctgaatccttctctttggtcattattcagactggttcccagcatgccgcagtccagaggaaaaggtccaagttctgtttaaaaatcagagactaaagagcttactcagcatcctgctcacattaatgttgcttgttttcatcatttaaagtcttttgatagttccttgtatacaataaccttaggcttctgggaatattctcagaacataggaagcacctccctgacttagtaaagatgaaaaatgcttttcatgactatcccctctcttgaaggaccccctggaacagccaggtctttgggcataattaaattctgggcaagattttacagaaatgcagcttcttttttctaatttagttgtgtggtgtgtgtgtgtaaagtgggggaggttccacttggtggggaaaggagaatttgccattgctgctcgtctactcaggactgtttctgttgttgttgtgtttcagcatcagagagtgagagtgtattgcagcaatctgactatttggaagactgttccttgaatttcccaattcaaaagcctcggtagagctgagggatgcttgatacgtcaacacagaccacaaaaggcagggcttttctaaagagattataattatatctaccttttgggtacaggaggtgaatggaaggaagggattctggagcagatatcccaaaagaagaatcccgaagcagaactcctcgcacaaggttatctaaatctccttgacaggtgcacaggcagagaaggcatttggcccttgaagtaacatttacttgagaggttgggacaattctgtcacgcttaggacaagccagctgaccctgagcccaggagcaccctaggactgcagcacagaaaatacaccagctggccggtcgcccctcctttgttccattcccgggggattggagtagcgttggagtcaccgacgccatcccctcccgcctctggcgtgcatggagcatgcgcttccttcctcacttcctctgcaggagggagcgagagtaaagctacgccctggcgcgcagtctccgcgtcacaggaacttcagcacccacagggcggacagcgctcccctctacctggagacttgactcccgcgcgccccaaccctgcttatcccttgaccgtcgagtgtcagagatcctgcagccgcccagtcccggcccctctcccgccccacacccaccctcctggctcttcctgtttttactcctccttttcattcataacaaaagctacagctccaggagcccagcgccgggctgtgacccaagccgagcgtggaagaatggggttcctcgggaccggcacttggattctggtgttagtgctcccgattcaagctttccccaaacctggaggaagccaaggtatgtgaacacttttcttcttcctaccttccttttatttcgccacgaaaaggtaaagtttggcataatacgtgagctgtaaatcaccctgacgcgttttctgatcaaatcatatccatgaatacggacagagaaatcagttcgaatttagagacagaagacagattttttttcttcacttttaaaatgatagagcaataatatgggttgtttttaaagatcttattttgaaaagggaagggaacctttttcacctaaagtggcttggattgttttctagttgccttacaacctttctcagacagtctattcattatatatgcagtatatgatgaaagagcttttagtgtgccaataataccaatccaaattatgctctctctagctgagaatagctgataaccctagtgttagaactatatgttaaatttctggtcagaaaaaaaaaaaagtgtgattctgtcagctcaagaaatacaccaagataataaacaaggcaatattgataattactattattgtataatcttgataatctttgttaatatgcatttgtttcatagtcatccaaaattatttctcctaatatttttcctttttataaaaattttatttaataagaggcatattttgtccttcaacgcacaattaaatttatattcacatgtgtttattctagacaaatctctacataatagagaattaagtgcagaaagacctttgaatgaacaggtaggtcaaaagtaacattatgaatgctatttcattttgatttagttattattatttaatgtaattatgctgtgacattttggtcattttgaatttacaaacatcaggaacttaatagttaattgacagaaattaggacgggaaatctagttttggtaaattctatgccctagcagttatttacattgagttctgtataatgaatacatagagtaactatataataacccagtaagctcagcatcacatccactctgttgtaagattgcctaatttaccagaga

SEQ ID NO: 160 - DNA, 305 bp, Homo sapiens
cggtcgcccctcctttgttccattcccgggggattggagtagcgttggagtcaccgacgccatcccctcccgcctctggcgtgcatggagcatgcgcttccttcctcacttcctctgcaggagggagcgagagtaaagctacgccctggcgcgcagtctccgcgtcacaggaacttcagcacccacagggcggacagcgctcccctctacctggagacttgactcccgcgcgccccaaccctgcttatcccttgaccgtcgagtgtcagagatcctgcagccgcccagtcccggcccctctcccg

SEQ ID NO: 161 - DNA, 50 bp, Artificial Sequence
  LOX source sequence
gggcaggtgttcagcttgctgagcctgggctcacagtaccagcctcagcg

SEQ ID NO: 162 - DNA, 50 bp, Artificial Sequence
  LOX Unmethylated AlleleA Probe Sequence
aaacaaatattcaacttactaaacctaaactcacaataccaacctcaaca

SEQ ID NO: 163 - DNA, 50 bp, Artificial Sequence
  LOX Methylated AlleleB Probe Sequence
gaacaaatattcaacttactaaacctaaactcacaataccaacctcaacg

SEQ ID NO: 164 - DNA, 3001 bp, Homo sapiens
ggggagcggggcctcagacatatcagcccgtacctggccagacagttttcctccgccgcgcatctcaggttgtacatggacatcttctgcacgtacgtggacgcctggatgtagtaggggtcggccaccaggtctgggagacctaaacgtcagcaggcgacgggcgcagcagtgaaacaacccggcgccccccgctccaactccctacccctctaggtcccttactcctcacccttcgcccaccgggactgcagagtggagcggaggacagcaagagaactggggacgcccgggactgcaaagcaatgtgaaaaggaagcaggaggggccagacgcgcggtttgcactggattccagggctgccactgcaggcgcgtgggggagggatcggatctgcgaggaccggggcccgccgcgcccaggcagccacgtcgagaagccacatagctggggaccaggtgcacgggtgcttccagcggacttgggggtacttaccgtactggaagtagccagtgccgtatccgggccggtacctgcccccaggtctgggcctttcataagtatcgtagtagttgtaataagggttgtcgtcagagtacttgtaggggttgtaagggtcgtcgcccaccatgccgtccacgcggctgggcggccgcaggttactgagcgcaggaacttctcccggcgctgtctggttctccgcgcgcgaggcgccagcttcgcgggctctagatgtcgagtagccagcttggaaccagtgacgggcggtgggcctggggcggccagcggtgactccagatgagccggccgtccgcgttcgcgccgcggcggtgcggttgtcgcggatcagcaggatcggagtgcggggctgctgggcggaggcgttggctgcaccagggacggcggcgcccgggtcccggcggcgctgaggctggtactgtgagcccaggctcagcaagctgaacacctgcccgttgttctcccattggatctgctggcgccaggcgcccggagccgccggcggctcgcgcgggggctgctgttggccggcggcgggaggggcgcagtgcactagcgcgcagagctgcaaaggcccgagcaggagcacggtccaggcgaagcgcatcactccttttgccagattgaccccgctcgaggaggacgtggctcacagaaaataaaaacggggctcaaatcacgtgagggaaggagaaatcttcaaccaaggaggcgagcggagcacgggtatctcagtctccaccaagcaatgccaagggtgggattcagacccttccccagtcaaggcggctgctcggacgtggcacccttccctttcccctttctcagtcctggaaggcaacggggagcggcgcggcagtcccggagagcgggcagtgtctggagtgaaggaaggaggagagattttaaactttctggcacgtttgcaaagttacacaagccgttctggcccggccgcccctcagctatttgttcacgtaatgcgattggaaacgtgcaaggcggacagctcctcgcctctgcccctccctctctcccgtccctgccctcctccccccagacacgttgcacagggagcgttaaggggaaagaacaaaacggaacacacatcctgagacacactggcttaatctggggcgaacatgcaggaatttcaaaaaactcagacaggcgaaggcagccaggccatggggcgacgccaaaatatgcacgaagaaaaatgctattaggtcaccagcccttgagaggcgggaagtcgggaggttgggagggtgctgggggtgagaggggggcgggggaggattgtgactaaagtttatccataaaagagttaaccagacacatttcccaacgcacactctctcgcttttatgggaagcgccctcagcaaataaaccccaggatatcaaattttgtttttctgtgtgtaggtaatatgagaaatgggttacaaatttatttcctacagtgaatattttaataaccaaaagtgaaattgtacagggaacaaagccagctaacgctgcagctatgaataaacctgtctctttttcaaaatgccggcctgatttgtactttgagtagaagtggagaaccatctttcatctccacagtgtctacaaggaagattcattaaagactagaacaagtctgcatgagaaagcactcagaaggcatcttcacatatgcattttataagacatttttctccttcatcttataaagaagtttgactttggatttaaaaaaaaattaaatcacctctgttaaatactgaaagtcagtaaaacatcgcatgctgtttaatttatcttccaatctaaacaaagaagttgctgaagaaacagccttagagtttagattttggtaaagagaaggaagtaggagtggtcagaaaagaatataggtgggggataaattatgctgactggaaagagatacataatggacgagttattcatataccactcagggtttgggcagtgctagaaactttgcaacaaaaaatggttactaaatggtctgatttaacactactactcaaaaccagattttggtaaccattttattggtctcatcattgggctattgtccagacatacttcaatattcacatacctctgttagtctctgtctctccatatttaatcaccacattttggtggtgtggtggacctctgctgattttgactgcctcacatccattttccttttctttctagtaagaacaaccggattttcctttgaggggcttcccacccaccccactcagttgcatagtggaaggactgagcatttatgagacttacccacttcaagaagtggtcatgagatccaggtttggccaatcaagccatctctctagatggtttaattttatctgggatctgaatcttgagcagaaggacactgtcaaaaatgttgctcctcaaactactaaagaaagtgcccaggcctggcgcagtggctcacacctgtaatc

SEQ ID NO: 165 - DNA, 1577 bp, Homo sapiens
cgcgaagcagtagcagtcagaaccaggcaccagagcgccccctgaaggtagaccggggagcggggcctcagacatatcagcccgtacctggccagacagttttcctccgccgcgcatctcaggttgtacatggacatcttctgcacgtacgtggacgcctggatgtagtaggggtcggccaccaggtctgggagacctaaacgtcagcaggcgacgggcgcagcagtgaaacaacccggcgccccccgctccaactccctacccctctaggtcccttactcctcacccttcgcccaccgggactgcagagtggagcggaggacagcaagagaactggggacgcccgggactgcaaagcaatgtgaaaaggaagcaggaggggccagacgcgcggtttgcactggattccagggctgccactgcaggcgcgtgggggagggatcggatctgcgaggaccggggcccgccgcgcccaggcagccacgtcgagaagccacatagctggggaccaggtgcacgggtgcttccagcggacttgggggtacttaccgtactggaagtagccagtgccgtatccgggccggtacctgcccccaggtctgggcctttcataagtatcgtagtagttgtaataagggttgtcgtcagagtacttgtaggggttgtaagggtcgtcgcccaccatgccgtccacgcggctgggcggccgcaggttactgagcgcaggaacttctcccggcgctgtctggttctccgcgcgcgaggcgccagcttcgcgggctctagatgtcgagtagccagcttggaaccagtgacgggcggtgggcctggggcggccagcggtgactccagatgagccggccgtccgcgttcgcgccgcggcggtgcggttgtcgcggatcagcaggatcggagtgcggggctgctgggcggaggcgttggctgcaccagggacggcggcgcccgggtcccggcggcgctgaggctggtactgtgagcccaggctcagcaagctgaacacctgcccgttgttctcccattggatctgctggcgccaggcgcccggagccgccggcggctcgcgcgggggctgctgttggccggcggcgggaggggcgcagtgcactagcgcgcagagctgcaaaggcccgagcaggagcacggtccaggcgaagcgcatcactccttttgccagattgaccccgctcgaggaggacgtggctcacagaaaataaaaacggggctcaaatcacgtgagggaaggagaaatcttcaaccaaggaggcgagcggagcacgggtatctcagtctccaccaagcaatgccaagggtgggattcagacccttccccagtcaaggcggctgctcggacgtggcacccttccctttcccctttctcagtcctggaaggcaacggggagcggcgcggcagtcccggagagcgggcagtgtctggagtgaaggaaggaggagagattttaaactttctggcacgtttgcaaagttacacaagccgttctggcccggccgcccctcagctatttgttcacgtaatgcgattggaaacgtgcaaggcg

SEQ ID NO: 166 - DNA, 50 bp, Artificial Sequence
  OGDHL source sequence
gcagcgaggtccggaggctgcaggtcagggggctgcgcggaaggggtgcg

SEQ ID NO: 167 - DNA, 50 bp, Artificial Sequence
  OGDHL Unmethylated AlleleA Probe Sequence
acaacaaaatccaaaaactacaaatcaaaaaactacacaaaaaaaataca

SEQ ID NO: 168 - DNA, 50 bp, Artificial Sequence
  OGDHL Methylated AlleleB Probe Sequence
acaacgaaatccgaaaactacaaatcaaaaaactacgcgaaaaaaatacg

SEQ ID NO: 169 - DNA, 3001 bp, Homo sapiens
ccactcccacccccacaggggaatggggcagtttgggccagatagggacaaagtctggctgtgtccagaatggcccggtgcaggaacactgttttataaacactaaacagtctgggttggccactgggcactgttgtgaccttgacccagcctggcaggagggcagcctgccacccacccccacctgcatcccagcagggcatgagggctcagtatctagagccagagcccaagccagccctactagttcctgcccatacagtcaaggacagcaacaaggagtgagagtcaggtgccctgtgcaaccccctgcccagtccagactccagcagagttgccctgggtatctcttgggggtctcttctctgaggacaaggtcacatagcatccccaacccaacctcagaacctcaacccaaccccagaaaactgccgacaccatggccactaagactcccacacacacaaccctgtctggggtgccctagtgaggactactccctaatgcacccattgcagagatcagtaaacagaggccgggaagggttctccccagagggccctccaagctgggcagcagcagagcctggattctctcagcagcaggtctactctgaccccactttctgagaagcagatgagcttttgctcagcaacaaaccgcaattctatttagttggggcctgttccaagacactggggaggggctgctagggcctgcttgagccaggtggccgaggggcgtcaaagggtctctccggttcggcgaatgcgtactgcagcctgctgtggcctcgggattaccccgtccccctcccacatctcccggggggcggaagaagcgcatcccatgcaagcagcgacctcaggccatgccgccctctctcaggcctcagttttctcatctgtaatcggagaccgcggttaaaaccgtctattaggctccttggtagtccgaagctgcctatgggtcccgccaggtgcacagcgctgaccctccccatcccccgcacagccctcgcagctccgggccctcccctggccacggaaggcgcggggcagtctgaagtcctcgggctggagaaacccaccgcgagcacccgctcctctgcgcaggaccctggactagccgggtggtacgcgcgcgatgggctgggggtcccgggggaggccacggccagccaaggtcagagccaaggtcacagtctgttcctcctccccctccccccggcggggccagactcagggctgcgggcagcggcgccccaggacccaggagagaggagctgtggggccggtccccaggacggcaggagaagcagcggcgcagggaccgggagggggcgggccgccgcgctcaccgggcgggcgggccgggtccgcgctgcagcgaggtccggaggctgcaggtcagggggctgcgcggaaggggtgcgcgcgcgccgtgccaattacctgggtcacgtgacgcagccgcccacgggcgcgcgcccctgagactccgcgcgtcgtgacgtctccatggatgccccgccccgctcctggcgctgtccacggtgctggcggcccaggcccgaggtcgcccacggccgaaggggggactcctctctgagaccgcagaacggggctgaggccctgaaccccgtgtgctcgtcctaaaactaggacgaatgtaccagacgacaagcagcctttgtgtgtaggagaggataggttttgttttgtcgtctgtaaaatgggaatgctatgaaaagttccactatcttagggatgttttgaaaaaaagatgagataatgcgtgaaaagtgcttaccccaaatctgatacacattttacccattcatcctcccactgatggtgacccaggctgtcctcaccaccccgtggccacaaacaaggctgcaatgaacttcttcatacacattcccttaggatcctgcgtggaactttctctgggatagagatcaactttctaattattatcagaatagcagatgcaatgtcctgttattgttttaatctgcatttctctggttatcaaccatttgcatctcccgttgtttgttagtttggggttttctgttgtatttttttcagctttctgttgggttttgttctcatttaaatgcagggatttcttgtataggaacataacatacctgtggttcaagttgccatcaaatctctcctggattatcgcagcagcctcctaactggcttctctgcttctacacttacactcttgaattttaatctcaaaactacagtatgcacttcacaagaatatattcaagtacatatcaaaccaattaatatggttgcttgtcgagggaggaggatggggaatgtggatatgaggtaattaatcaattacccagatcactgctgacagagtgccacgaagaaaggaatacaactaacttctgcacctgaagtctcccaaaagttctttatttggagacagttagggatacatgtggtcaagaatcttcagcacttaacacagcactatcatggtatttttcagtaaatatttgttgaatgaatgactgagtgagtaaatggacacttgacttggatcctgaaggatgagtaagattttatctggtgcagaagggcagggttcattgggaattctttggacacaagcaccaaaaatgacctggctagcttcagccgaaaacgagttgatatgaaggttgtatataaggcaactcacagaatcagaggaaagctgaaaatttacttcaggaagcagggctgagccatagaggaactgtctcttcaaggctcttctgtttttcaaaattcaaattccgggtgagaggcagtaatgtacttgcagtagagacggagtaaggtggccagatttgagagacttacggggatcagctccacggggtttagtggttgaccagatatgagggtggagacatagataactcacaggtgtctggctttagtgacttgggcacagtggaacttctcagtgagctagggaa

SEQ ID NO: 170 - DNA, 947 bp, Homo sapiens
cgaggggcgtcaaagggtctctccggttcggcgaatgcgtactgcagcctgctgtggcctcgggattaccccgtccccctcccacatctcccggggggcggaagaagcgcatcccatgcaagcagcgacctcaggccatgccgccctctctcaggcctcagttttctcatctgtaatcggagaccgcggttaaaaccgtctattaggctccttggtagtccgaagctgcctatgggtcccgccaggtgcacagcgctgaccctccccatcccccgcacagccctcgcagctccgggccctcccctggccacggaaggcgcggggcagtctgaagtcctcgggctggagaaacccaccgcgagcacccgctcctctgcgcaggaccctggactagccgggtggtacgcgcgcgatgggctgggggtcccgggggaggccacggccagccaaggtcagagccaaggtcacagtctgttcctcctccccctccccccggcggggccagactcagggctgcgggcagcggcgccccaggacccaggagagaggagctgtggggccggtccccaggacggcaggagaagcagcggcgcagggaccgggagggggcgggccgccgcgctcaccgggcgggcgggccgggtccgcgctgcagcgaggtccggaggctgcaggtcagggggctgcgcggaaggggtgcgcgcgcgccgtgccaattacctgggtcacgtgacgcagccgcccacgggcgcgcgcccctgagactccgcgcgtcgtgacgtctccatggatgccccgccccgctcctggcgctgtccacggtgctggcggcccaggcccgaggtcgcccacggccgaaggggggactcctctctgagaccgcagaacggggctgaggccctgaaccccgtgtgctcgtcctaaaactaggacgaatgtaccagacg

SEQ ID NO: 171 - DNA, 50 bp, Artificial Sequence
  SYNM source sequence
gaaaagccaccaatctccagggcctcgggtaagaagtcgctccaaaatcg

SEQ ID NO: 172 - DNA, 50 bp, Artificial Sequence
  SYNM Unmethylated AlleleA Probe Sequence
aaaaaaccaccaatctccaaaacctcaaataaaaaatcactccaaaatca

SEQ ID NO: 173 - DNA, 50 bp, Artificial Sequence
  SYNM Methylated AlleleB Probe Sequence
aaaaaaccaccaatctccaaaacctcgaataaaaaatcgctccaaaatcg

SEQ ID NO: 174 - DNA, 3001 bp, Homo sapiens
gcaccccaaccaagggtcagctggtttgtccctgtgagccttaatgacacttatactgagaactgtcattcataaataactgccatgaaaactaatgaaatgcaagtgtacaaaaaagagctgtggtttccatggcaattaaactgaatactttcttagggaaatgctggaaagaaacagctgtcacattaggggtgtgtgtgtgagatttgtaaaagttcggggagatgttagatgtctagaaagttctgtacttggagcactttatggtctctaagttttcactccaaatgaaagaaattcgttttggaaaatgtaatgtactatgagggtagcttgtataagaagggcaactgggactcctgtcaactgacatcaagagaagtattttgcacattaaaatactggtaaatggatatatatttatgttttaagttaaaaagattatttaggctgggcacggtagctcacgccagtaatcccaggactttgggaggccgaggtgggcggatcacctgaggtcgtgagttcgagaccagcctgaccaacatggagaaaccccgtctctcccaaaaatccaaaaaaaaaaaaaaaaaaaaaaaaaaaaaattagccaggcgtggtggcgctggcctgtaatcccggttactcgggaggctgaggcaggagaatcgcttgaacccgggaggcggaggctgtggtgagccgagatcgtgccattgcactccagcctgggcaacaagagcgacactctgtctcaaaaaaaaaaaaaaaaaaattaatgcacatgtatgtgtaatgtatatatatgagatgaggcctctctctgttgcccaggctggagcgcagtgccacaatcaggttcactgcagccctgaactcctgggttcgagtaatcctcccgcctcagcctcccaagcagcgcgtgtgacaccaccacgccaggctccatatatatatatatatatatatatatttttttttttttttttttttttgggagagacagggtctctctatgttgctcaggctggtctcgaactcctgggctcaagcgagcctcccacctcggcttcccaaaatgctgggattataggcctgagccaccgcgcccggcccatatcgtttttttaaaaatagaaaagccaccaatctccagggcctcgggtaagaagtcgctccaaaatcgccagcaccacagcaactcaataggcttctatttccatccaccaaggccaggacactcgtatgctcccggacgccctctccggcgtcccttcgctgagcccggcctggctagcccgccaccccgcccgctgttacccgactgcccgcaccgaccacctgcggccgccaaggtgcggcagggggcgctgtgcggccgcagaggccgcgccgggccgcgctctgattggctggaggccgcggccctcgcccaggccgcccgcccagtctgcccgccccgtccgctaagtgcctgggctctcccgctcgcgtcccagtctgcgggcctccggggcagcggcgaggccggagcgtcgcggcggagaggacgagaccgggacaagaccagggcaggagggagccggccagccgcgagaaccccgcacgcccggcaagatgctgtcctggcggctgcagacgggccccgagaaggccgagctccaggagctcaacgcccggctctatgactacgtgtgtcgggtgcgggagctggagcgcgaaaacctactcctggaggaggagctgcgcggccggcgcgggcgagagggcctgtgggccgaggggcaggcccgctgcgccgaggaggcgcgcagcttgcggcagcagctggacgagctgagctgggccactgcgctggcggagggcgagcgggacgctctgcggcgcgagctgcgggagctgcagcgcctggatgcggaggagcgcgccgcccgcggccgcctggacgccgagctgggtgcgcagcagcgcgagctgcaggaggcgctgggcgcgcgcgccgccctcgaggcgctgctgggccggctgcaggccgagcgccgaggcctcgacgcggcccacgaacgcgacgtgagggagctgcgcgcgcgcgccgccagccttaccatgcatttccgcgcccgcgccaccggccccgccgcgccgccgccacgcctgcgggaggtgcacgacagctacgcactgctggtggccgagtcgtggcgggagacggtgcagctgtacgaggacgaggtgcgcgagctggaggaggcgctgcggcgcggccaggagagcagactccaggcggaggaagagacgcggctgtgcgcgcaggaggcaaggcgctgcggcgcgaggcgctcgggttggagcagctgcgcgcgcggctggaggacgcgctgctgcggatgcgcgaggagtacgggatacaggccgaggagcggcaggtccgtgcgcggggatggcgcgctgaccccatacccgctgccgtcgccccagcaccctgcccttgacggcgtggggcagcggccccttcaccaggggcgcggcgtcgcggaccggtagggcccgcccagagggtgcccgaatggcatggggacccggactcggtccgctggtccatcgactcagcgggcgagcgcggggcggcaggtcctagcggatagccccgtcctcgtcatggggttaggtggcagctgctttaactcttgacaagcgtgactgggcgcatcctgataggccaccctgttagctctaggttttccttccattttccgtgctccgtagactcagtagacccagatctaagagtcaaccgagaaacctctctcacttgcagaagtgacggcttatggtagccccgctgtctatagcgctcctcaggggaaaactcaaattaatttccaaaacggtttttattttgccaatatgatttcccagttggaagccgcagttgtggctctctgggcggctgccttgtttgatcttgggaggggcagcaaccctcatgcagaacaga

SEQ ID NO: 175 - DNA, 1414 bp, Homo sapiens
cgtatgctcccggacgccctctccggcgtcccttcgctgagcccggcctggctagcccgccaccccgcccgctgttacccgactgcccgcaccgaccacctgcggccgccaaggtgcggcagggggcgctgtgcggccgcagaggccgcgccgggccgcgctctgattggctggaggccgcggccctcgcccaggccgcccgcccagtctgcccgccccgtccgctaagtgcctgggctctcccgctcgcgtcccagtctgcgggcctccggggcagcggcgaggccggagcgtcgcggcggagaggacgagaccgggacaagaccagggcaggagggagccggccagccgcgagaaccccgcacgcccggcaagatgctgtcctggcggctgcagacgggccccgagaaggccgagctccaggagctcaacgcccggctctatgactacgtgtgtcgggtgcgggagctggagcgcgaaaacctactcctggaggaggagctgcgcggccggcgcgggcgagagggcctgtgggccgaggggcaggcccgctgcgccgaggaggcgcgcagcttgcggcagcagctggacgagctgagctgggccactgcgctggcggagggcgagcgggacgctctgcggcgcgagctgcgggagctgcagcgcctggatgcggaggagcgcgccgcccgcggccgcctggacgccgagctgggtgcgcagcagcgcgagctgcaggaggcgctgggcgcgcgcgccgccctcgaggcgctgctgggccggctgcaggccgagcgccgaggcctcgacgcggcccacgaacgcgacgtgagggagctgcgcgcgcgcgccgccagccttaccatgcatttccgcgcccgcgccaccggccccgccgcgccgccgccacgcctgcgggaggtgcacgacagctacgcactgctggtggccgagtcgtggcgggagacggtgcagctgtacgaggacgaggtgcgcgagctggaggaggcgctgcggcgcggccaggagagcagactccaggcggaggaagagacgcggctgtgcgcgcaggaggcaaggcgctgcggcgcgaggcgctcgggttggagcagctgcgcgcgcggctggaggacgcgctgctgcggatgcgcgaggagtacgggatacaggccgaggagcggcaggtccgtgcgcggggatggcgcgctgaccccatacccgctgccgtcgccccagcaccctgcccttgacggcgtggggcagcggccccttcaccaggggcgcggcgtcgcggaccggtagggcccgcccagagggtgcccgaatggcatggggacccggactcggtccgctggtccatcgactcagcgggcgagcgcggggcggcaggtcctagcggatagccccgtcctcg

SEQ ID NO: 176 - DNA, 50 bp, Artificial Sequence
  PCOLCE2 source sequence
ctgctgccgcgagagctgggtggcggcagccagcagcaggcagagtggcg

SEQ ID NO: 177 - DNA, 50 bp, Artificial Sequence
  PCOLCE2 Unmethylated AlleleA Probe Sequence
ctactaccacaaaaactaaataacaacaaccaacaacaaacaaaataaca

SEQ ID NO: 178 - DNA, 50 bp, Artificial Sequence
  PCOLCE2 Methylated AlleleB Probe Sequence
ctactaccgcgaaaactaaataacgacaaccaacaacaaacaaaataacg

SEQ ID NO: 179 - DNA, 3001 bp, Homo sapiens
tacactccaggaaaaccttcactgccaataaatccagactctccagtaagaatgccaccacatgtgaaaacaggtctgggaacataaaagaagaaaagataatgtaatttgaaacatggtctaacaatctgatgttaccttcagaaaggtgtagcctattaatataaataaaagttaatgttgattaatcaatgcttgcaccatataagggatctgaaaatttgctagctcctagaatatttaaattatagcctgacaaagcattcagtccagcgccatctgtctccatcataataacacaagattatatatatgtataatcaagtcctcggcattgtggcaaagtcttgtaggaaaatggtaaatacatcaatttaactgggggagtgggagagaaggtaagagccaggctgaatgagaagcatggttttaaagaaagacaggcaagcaattggttaatataaaaaaagtttgtcgttcccatatttggagagatgcccagttctcctccccctttctgaaaatcactcgccattggcagagcttcccatcgaaagggaggctgatgttgatggaggctgaagaggtgatgccaaagttggccaccgggcaggtcccacagcccagctcctggggccttccagaggttggcgaagagttaactttccggagcggctccctaggcgccgcgccgctagggttgttccctagtttacgtctctgggcaaagcctctcggggaaggaaaaaaaacagcggtaaaccaaaatccacatatttgggccccagtgcttccctctggccccatagacacgaggagaggggagacacaagtcagattaagccccgccacgtggtggccaagccagctgcagggccatccggcaggccgggctggggacccaggagggagccccgcgagcaaggatgggtcccgaagtcccgggcttagcctacccgagggtggcggacgcctgcaccgcgcgggagcgcagggtcgagcgctgggtcacccaggcgcgccgaagcgggttgagtaaagcagcaggcgaggctgcaggggtggaggaagggaaaggagagaaagggagcccgggcaggggtcgcgttacctctctggggactgctgccgcgagagctgggtggcggcagccagcagcaggcagagtggcgcccaggcgttcgcgcccctcatggcagcgtagacgctcggggtttgcaccccacggcgcgcgcgccggcacacacgcccctccgcacccaccgcgctcacaccgccgctcacactggcagcagcgctggctcacaccggcgctcggctgcccgcgcgctccctctcacgcgcgcaccgccgcggggcggcccaggtagccgggggatacgcggccggcagggcggaggcacttttaaaggcgattatggtgccgtctgactcgacgcttagtttcccctgaggagctgctctggcagccacaggccactgctgaatatcccgtttgttctccggcggcaggaggggcggctccgaggccagagccgccccctctcgtggggcgggcgaacgtctcctcccggcaccccgggaccgacgggtcctctctcgtccagccctcagggaccccggggagaagaggaaccccttgcggagtggcaccccagcggcccgggccccgacgcccagagacgattctcagggagggcttctggttttgctttggggaatgtttttcccagttgcttttaaataaaaagtaatagcgcttgctggctgctggaaagcctgggggtccttctttgtctccttcaaaacccagatccaaggaaagaaagggaaaagggattttattgtattttaacttctgtgcttattctcctctctctctctctagatacttttcttttttttactggtatgacaatgacaaggaagacagtctctgtgccgggatttaaacacgggttttcaggcgagcgatttctgtgtgaactgtcattaaatgtcagataatgaagacggaaccaaatcagcaaatctcaccatcccatgtcatgttctgaaaatggtgtttctctgcttgcatctctctgttcttagacctttccaacagcatctttctgtggtattaacgggttaaaatttacggagaattcgtttttcttagacgaacaattctagccttgtggaatcaatactctcttttacttaattacagaaatataattttaaacatttattttgttaagagcttcccaataaccagggattaataaaagaatgacaggaacagactagattcatagagatttttaataatcaccagctatgggagatacagcttaactttgatgcctcacgttgttccttctaaattgttttcacctttctaagcaagaaaattaagtattaaagtagttcttatcgagactttatttctaataatgagctgttaagaatctaaacacgggtgggcgcgatggcccacgcctgtaatcccagcacttttggaggccgaggcgggcggatcacgaggtcaggagttcgaaaccagtctggccaagatggtgaaaccccgcctcgactaaaaatacaaaaaattagccaggtgttgcggtgtgcgtctgtaatcccgggtactcgggaagctgaggcagaagtatcgcgtgaacccgggaggcggaggttgcagtgagccgagatcacgccattgcactccagccccgggcgacagagtgagactccatctcaaaaaagaaaaaaaaaaaaaaaagaacctaaacactcctggcaaggaagtaagtaaaactggctacaggaaatattctgacttttcatagtgagtgcaccctgaagcctatactaggaaccagaatatacggacaggacttagattatcttaccatttttttaaaaagtgaaggcaaaaacatcataaagtacaactttcaaaacttctctacaggcaccacaccctttatgacattaaaaaaaagggtgacagaat

SEQ ID NO: 180 - DNA, 1034 bp, Homo sapiens
cgaagagttaactttccggagcggctccctaggcgccgcgccgctagggttgttccctagtttacgtctctgggcaaagcctctcggggaaggaaaaaaaacagcggtaaaccaaaatccacatatttgggccccagtgcttccctctggccccatagacacgaggagaggggagacacaagtcagattaagccccgccacgtggtggccaagccagctgcagggccatccggcaggccgggctggggacccaggagggagccccgcgagcaaggatgggtcccgaagtcccgggcttagcctacccgagggtggcggacgcctgcaccgcgcgggagcgcagggtcgagcgctgggtcacccaggcgcgccgaagcgggttgagtaaagcagcaggcgaggctgcaggggtggaggaagggaaaggagagaaagggagcccgggcaggggtcgcgttacctctctggggactgctgccgcgagagctgggtggcggcagccagcagcaggcagagtggcgcccaggcgttcgcgcccctcatggcagcgtagacgctcggggtttgcaccccacggcgcgcgcgccggcacacacgcccctccgcacccaccgcgctcacaccgccgctcacactggcagcagcgctggctcacaccggcgctcggctgcccgcgcgctccctctcacgcgcgcaccgccgcggggcggcccaggtagccgggggatacgcggccggcagggcggaggcacttttaaaggcgattatggtgccgtctgactcgacgcttagtttcccctgaggagctgctctggcagccacaggccactgctgaatatcccgtttgttctccggcggcaggaggggcggctccgaggccagagccgccccctctcgtggggcgggcgaacgtctcctcccggcaccccgggaccgacgggtcctctctcgtccagccctcagggaccccggggagaagaggaaccccttgcggagtggcaccccagcggcccgggccccgacgcccagagacg

SEQ ID NO: 181 - DNA, 50 bp, Artificial Sequence
  No overlapping gene source sequence
ctttgctgtgtaaagtgctatgcagaggcaacttcacgtcccctagctcg

SEQ ID NO: 182 - DNA, 50 bp, Artificial Sequence
  No overlapping gene Unmethylated AlleleA Probe Sequence
ctttactatataaaatactatacaaaaacaacttcacatcccctaactca

SEQ ID NO: 183 - DNA, 50 bp, Artificial Sequence
  No overlapping gene Methylated AlleleB Probe Sequence
ctttactatataaaatactatacaaaaacaacttcacgtcccctaactcg

SEQ ID NO: 184 - DNA, 3001 bp, Homo sapiens
gttctgatgatcagagaagcctttccagtagagatgacatttacctccttgggatttcagcagacacaggagagtgagaatgtcactctggcagaggaaacagaatttacagattaggagtacatggctggaatgaaagagttggggatggagtggataagaaatagcagtgactacagtttgagtgatacatttatttgcatattcattgattcagcaaacttttcattggtgctgagggaggtaggcccaaggtatgtgctgggtacagaggctgagaaaacacgggacagagtggtggaagatgggcctgggattgtggaaaaaacagaaagcaccagccctgtttgatccttctagaaggatttttttgtagtgtcccaatgatacctttcattgataatggttaccatttcattgagcacctcctaagtgctgtgctgaatagctgtgttactgctagttccagcaatcctgcatggtaggtgttttctctatttacacataggaagagaagctctgagtgcctgagtgcccagcttgtcatatacgatgcttggatttgtgcaagcattgatctcaatggcctggtaattataaaagctgaagaacagctgaaatcaagaacttctccaccccagcccctgaggacaataccacctccaaatgccttgggcccactctggtctctggtgtggtgggacagcgctgtccggtctgcgcaggtgatttatgtctgcctgcttttcggagaggccagagtttattgccttgctgggcagaactgcagctggggcctgccctgtcccatccccatccccccgaaagcaaagatttccaattactgttctgtagaaaatgtcaaaagcattattcaactattcattatcccaggttttgcaatttttcatatgtttttactcttatctgccagcacttgaaaaagaccagctttttcctcttaagagagcttaatttgcagagctgtgcagtcttcctattatgttcttatcatcctctccctttggtaggcagaatgacaaggatgacaaaaaaagaaccaaaaatatttgaaggccttcctgaaaatccaccggaatgaaggatcccaaaagcctgaggctcggttcctcgagtgacctggacgggagccaagggcggctggcattagctctcacagtccactcggcaggagctcgtggctagtgctcatgggaccaaaaccaccaccccagggccatgggattcccccccaggggcttaactgggggattaagcagctctgaggtgccaagaggtcccctctccccgaaatcctcgtaccagtgctaaattacctttggtatcccaaggaccagaggctaagggtagcttggggtgagtggttagctcttcccccatctcctgtggcccctccgccccattctttgataaggtaactgacatttttctctttgctgtgtaaagtgctatgcagaggcaacttcacgtcccctagctcgctctgttccacctagatggcgccctaagccccgcctgtatggagattcggagcagccatctccctggatgccttcctcccacgatccccaggtttctgaagcgggcgcaccaggactcctggctcacccagagagacgccttgcccgcggcggggctcgctcggggaaatccccgcccaccttgttattcctgcaggggaatccccgccccccgtccctgtcactcggggaagggagttcccgcccctcgaatcaaccagggggagggaatccccgcccatcctggaggctcggcggatcccctgcgcagcgaggcgaggagcggaccccagcgccggtgcgtgccggccccgggcagcgggacgcggcggggcggcggctgcaggcagccgaggagccgcaggccgaacccaaggcaccgggattgcgcctcccgcggctgccggcgaaccgcggctctgcagctcggggcaggcgcggcggcggcaccggtggtggccgcggtggcggcagctgcgcggggacccgccgggcggcgcctgggtctggacgcgcgaggaagccgcgggagcctcggccaagccgcgagcaggtgaagcgaccgtcccgctgcagccgggacgcgcggctccgggtgggcaggggatgggggaggcccggcacccccgagaccgtgtgtgcccaggaaagtttagctacaaatccgggtgcgggaatcccagccctggcagggcggggagtgaggacgccgaaccggggtcccacgttgggcggcgcaaactcctctagcatccggccggggacggggagggcgcagcccaggggagggggctagacagagcgggaccgagacagcgggacaaccggagacgcactgccgggggtactcaagacagggccgggacctcctgctcgggcgcgcatcccgggtccccatccctgggccgtcttctggtcccttcctccgacaggagtggaggtactcaggtactccggcctcaggtaatgtcttggggaccatcctccctcgggcttgtttcatctctcaacccccgcctctttgcccagatgagacgttagacgcggggcgagggaatgcagggggcgcgcaaagatcaagggcatcttagggccggttattggcgtctgccggggagatggggaacatagtgatagggaaatcctgctctctctctgaagtcgagtttctcctaaaatctggtttctccctccctctgttcctctctccctccctcgggtttgcgtttgtgttttaagtctttgagctgcctgtggtccccgctatttggaaaatccattaaagcgcaaggctgaattcagcagcaggagggtccaggacccctggcaggagtttgccagaagctgtcctcggcagcccaaatctatgcagagaccttgataagggacactggagtgggaagaggacaaggtctggcatgggctggtcgcagagactggaatggcaggcacgagggcat

SEQ ID NO: 185 - DNA, 1051 bp, Homo sapiens
cgggcgcaccaggactcctggctcacccagagagacgccttgcccgcggcggggctcgctcggggaaatccccgcccaccttgttattcctgcaggggaatccccgccccccgtccctgtcactcggggaagggagttcccgcccctcgaatcaaccagggggagggaatccccgcccatcctggaggctcggcggatcccctgcgcagcgaggcgaggagcggaccccagcgccggtgcgtgccggccccgggcagcgggacgcggcggggcggcggctgcaggcagccgaggagccgcaggccgaacccaaggcaccgggattgcgcctcccgcggctgccggcgaaccgcggctctgcagctcggggcaggcgcggcggcggcaccggtggtggccgcggtggcggcagctgcgcggggacccgccgggcggcgcctgggtctggacgcgcgaggaagccgcgggagcctcggccaagccgcgagcaggtgaagcgaccgtcccgctgcagccgggacgcgcggctccgggtgggcaggggatgggggaggcccggcacccccgagaccgtgtgtgcccaggaaagtttagctacaaatccgggtgcgggaatcccagccctggcagggcggggagtgaggacgccgaaccggggtcccacgttgggcggcgcaaactcctctagcatccggccggggacggggagggcgcagcccaggggagggggctagacagagcgggaccgagacagcgggacaaccggagacgcactgccgggggtactcaagacagggccgggacctcctgctcgggcgcgcatcccgggtccccatccctgggccgtcttctggtcccttcctccgacaggagtggaggtactcaggtactccggcctcaggtaatgtcttggggaccatcctccctcgggcttgtttcatctctcaacccccgcctctttgcccagatgagacgttagacgcggggcgagggaatgcagggggcgcgcaaagatcaagggcatcttagggccggttattggcgtctgccg

SEQ ID NO: 186 - DNA, 50 bp, Artificial Sequence
  UBE2E2 source sequence
attgctgcacatggtgtgtctcctggcgagtgccgggtttggctccttcg

SEQ ID NO: 187 - DNA, 50 bp, Artificial Sequence
  UBE2E2 Unmethylated AlleleA Probe Sequence
attactacacataatatatctcctaacaaataccaaatttaactccttca

SEQ ID NO: 188 - DNA, 50 bp, Artificial Sequence
  UBE2E2 Methylated AlleleB Probe Sequence
attactacacataatatatctcctaacgaataccgaatttaactccttcg

SEQ ID NO: 189 - DNA, 3001 bp, Homo sapiens
caacagaatttgctgacgaatgtgatgctgagtgtacaacagagaaggttcagacatagggaagttctgaggacaaggatttttttcttttattcaatgtcatatatctaccacctagacagtgcctggtatcaataaatattccttgaataaatgaaaagggtaactgatttgtattaaacccattttgaacttagtaaatagcagttaaaacatctacagtcaaatgaagtactgtagcaaccataatttcagcaattaagcagtgaaagatcttccaatcctttcctccacaaagatagaagttaattttgctgcatccctggacaccccgttgccaacgacccagcattccgttggtaagtgaggctggaggttggagtgggagaaatttcgtggaagctaagttcgcaaagcaggtacgttttttttttttttttaaacggttatttgggatgaggagtacaacggaacctaatgttccctcatgccacctacagcactgctactgttcccatatccttcaactgggcatgaaggcagcttcccactcactctgacagtggtgcaccctgtacccccacccctctcttcaataccccagggctaaacggcccgaagctcctctctaccggctggtgcgcattctgggccagggcgagccaaaaggcagaagggccttcgtgctgcagtgtctggtgaagactcaggcttagcctggaagaagtttccaggccaacgtgaagttttcctgtcttgggtacatgcgagccccacgcgtctgcgagtttgggttagtgtgtcaacagggtcagtccccgtatctactttgcgaaagcttcgaggcgagcgtgaagtcaagggctgcggtggatgggggtaaaaggcctcctcgtcccactgcctgcaccgtcttggggtaacccctaacccccagccggcgtttccctttaatgcgcgtgcccggcaaggtcctcgcgtcccctcccctcaagccaccccgtcagcctcggtttcccccacttctccccgtccttgtcgttccttccccgtgggacgcccctcgctcacccccgtcagagcccagtctgcgcgcgggacccagctgtcactttacttttcctcgccgcctccccaccccctctcccgaccggcggggcagcccgcccgggcgcgctcccgcccccgccccctcgcggccgcgcgccggaggaggaggggcggggggaggagggaggcgcgcggcgccgcatggccgacgtggggctcgcgctctcgcggtctctcccgggcgggcgcgcaggcacgcgcactcgcgggtgcgcgcgcggacggccgggcggcggcggctcccggaggtggtggcttcactttccaggactcaggggcagccacagcgacagccgcgggcagcagcctcaggagccggagctggaacggccgggggcggcggcagcggcggcgctgagggtgagtccgggcgggcgcgagcgcggcggggacaggcgtggtcgggtgcgtggtgcgtgggtccggctttcggtgactagacggtccgcaggggacatcccgtccctggggcctccccagtctccctccccctcgcgcctgggcagctctctcccagggcttcggctcgagcctgcgacctgcacggacacccccccctcaggtattcgctcgggccgcgccggtgcctcccctcctcgggcgtcctccctctcgctgacgtcctcctcactcccccgacctcccctccgcgtcgcaggtctctgcttacaccgctcgtgccctagttccctccttcctcgctccccgtgcccggttccctacgccccctcccgcgagcctcctgcccccgtttctcgccctttctacccctcccccttcttcccatcccccccttctgcctcttctctccctgtcctccttctacccccttccgttctcagttccccatccttctcaaccctcccagcgctttccttctcctagccgcatcctggaactaagtcttttcaaaagtacaggataaatgtcacggtggaaagtgcccggcttgatcttttgctattttccctaattgcataattgctgcacatggtgtgtctcctggcgagtgccgggtttggctccttcgccgccgcaccccggtggcccgcccggagaagaccttgtggggtatatttgtgtgtgtcctggtgaggggtgtgtgttgtattggtcttttgtgatgcctggcgcttgtcacagtttggaaccgaagcccatttttctctggtctcttctctgcagcccccactggcctggtttgaaactggattccctctgtcctctcccccttcccgcccaccccacttctccccctcttttctccgatctctctcataatgttttgatctttttgccctttaccatttctaatgaagataaagcgtggcttcttgtggcttttttcccccacgaccaaccagaaatgtccaatccatcttcccttaaggaaattaaggctgctttagaaagacatcatcctgtgctcggtggctgttagacccatttgcatgttttcaagattcatcgttttgtattgtgtattcttgagagtctactaaaatatttttatttaaaagtacgaaaggaagtgctagggtttaccctcaaagtatctcgcggttctaaaggattttacgctcttcttagtttttaatccctgatttctttaaaaagtatgtttctagatatgtgataattcagtctggactttgtgaatctaaatatttgcctttactacggcaatttggatgctccttggctctttggaattctgtatgtgggctgacgctgaaatagagtgttttcatataatgccataccccattctctttcccctgcagcaccttttacccccaaatcaggtgttccactgttattttctgcataaatgtctttgcagttaaatttaaagctggatattgctgtgtgcatatactatatatttatt

SEQ ID NO: 190 - DNA, 1021 bp, Homo sapiens
cgagccccacgcgtctgcgagtttgggttagtgtgtcaacagggtcagtccccgtatctactttgcgaaagcttcgaggcgagcgtgaagtcaagggctgcggtggatgggggtaaaaggcctcctcgtcccactgcctgcaccgtcttggggtaacccctaacccccagccggcgtttccctttaatgcgcgtgcccggcaaggtcctcgcgtcccctcccctcaagccaccccgtcagcctcggtttcccccacttctccccgtccttgtcgttccttccccgtgggacgcccctcgctcacccccgtcagagcccagtctgcgcgcgggacccagctgtcactttacttttcctcgccgcctccccaccccctctcccgaccggcggggcagcccgcccgggcgcgctcccgcccccgccccctcgcggccgcgcgccggaggaggaggggcggggggaggagggaggcgcgcggcgccgcatggccgacgtggggctcgcgctctcgcggtctctcccgggcgggcgcgcaggcacgcgcactcgcgggtgcgcgcgcggacggccgggcggcggcggctcccggaggtggtggcttcactttccaggactcaggggcagccacagcgacagccgcgggcagcagcctcaggagccggagctggaacggccgggggcggcggcagcggcggcgctgagggtgagtccgggcgggcgcgagcgcggcggggacaggcgtggtcgggtgcgtggtgcgtgggtccggctttcggtgactagacggtccgcaggggacatcccgtccctggggcctccccagtctccctccccctcgcgcctgggcagctctctcccagggcttcggctcgagcctgcgacctgcacggacacccccccctcaggtattcgctcgggccgcgccggtgcctcccctcctcgggcgtcctccctctcgctgacgtcctcctcactcccccgacctcccctccgcgtcgcaggtctctgcttacaccgctcg

SEQ ID NO: 191 - DNA, 50 bp, Artificial Sequence
  OXTR source sequence
ttttaaaccactgcaaaataaacccatttgttaaggctctgggaccaacg

SEQ ID NO: 192 - DNA, 50 bp, Artificial Sequence
  OXTR Unmethylated AlleleA Probe Sequence
ttttaaaccactacaaaataaacccatttattaaaactctaaaaccaaca

SEQ ID NO: 193 - DNA, 50 bp, Artificial Sequence
  OXTR Methylated AlleleB Probe Sequence
ttttaaaccactacaaaataaacccatttattaaaactctaaaaccaacg

SEQ ID NO: 194 - DNA, 3001 bp, Homo sapiens
ccctcggcccccggcggcgcggcgctggcgttggctgcctcggcgctccagttggctgcgagcgcgccctccatgaccctggcggcagcggtgcgccccggccttcgagccctttacggcttggcgcggctgggccggatccggcgtgtcggagggtgtaggggcgcccggggctccactcctggagactccacggacggatctgctgggtccaccctgaaacaaaccgggagggccgtgaggagaccgccgcgtttctcttccgacgcgggtagggcgtgcttgtcccattcccaggaacccaactcatctgaaacaacagggcacaaccgccggcctcgggttgctcagccgccaccccagaaatccccgttggaggtacctcctctgagccactgcaaatgagcgggaatcctctaccggccacaagcccagagcccccagcgtgccagttcctcgggatgttcagcggcttccactgggggagaagggagggtcaaaatcagcaacgttcctccgggagtgggaatctaaaaccaaatcacctccccgagggaatctccaaactacctactcgccccaggctccgaccctcagcatatccacctgccgcgggacccccaaaccagcccctctcctagtaaacattaattcaagtacttcctccggaaatccccaaatagccacttgcctgggaaaccccagctctggtccttccccgtctaacccccttttctaagacggtcggccgtcactccctgaacttccacagcacctgcttcggtggaaacccggttctgcagtccctgctgggggaacccctgcctcaaagcccaagatgcccaagcgctgttcccagaccctggcatagacacctcccgcggggctcctccccagcccggcagcctcgaaagttctggaacccctgacttgcgctttggacccagataatcaaggactctgagtctcaatcccgagaagtcactttacaatctctcggaacaccccgagctcctccaggaacaaggagtgcgaggcaccccaccacctccactcgggtcccgatggctccaccggccctagccatcccgtccacccctgcttagcacccagctacctgcaccgagtccgcaggcgaacctaaagttgactccccccggggaagttgcacggcgactgacggccccagtgacgcgtgcgctcccggcccgagttgggatggcctgccggctgcacctaatgtgatgctaagctgaggtctctcactggagcctcggttggaatcccctcgccgcagcctggctggtcgctggatgggtacaggaggcgagcgaggagcgcctcccaggctgcgcgcgcggacagcgtctggatgcggcgctgtgcgctggggctgaggctgcactatcgcacgggtccgctagggggcgggcgaggccagccgcgcagacccctccggagcggagctggttcgcccagcgttggtcccagagccttaacaaatgggtttattttgcagtggtttaaaactgcgagagggagggaactcgtcaataacccgcccgtttcttcctttcttggtttagaagctcttgactccgagacacataggaggcttgaataataatgttcttccccgggtagatagtgatgaagttacaaaagcatttaaactgattatttcccaaaaatgatgttaattttcaggcttttctctccccccacacctccagcttgatgtagcggctttagcagtgaactcaaataagtctttgctttggaatacttttgttcctattcccgttaatgaggaattagaaatggcctctattctaagcagtgctgggagaggcgttattttttcggtagttttagaggaaattctgaagcacaacactgtcttcatttaaagtgattctgcttgtctgcgcccaagagccgtttctgcctccttgtcgcgcttcggaattaaaattctaaatgatgccaaaggaagataaaatgttaaagacagcttctcagcctccctgccccttctctattctaaaatcattttttgaagtaatttacagtggccaagaattaagtcagaaaaaaaggccaggattcaaaaataaacagggctctttaaaatgcattaaatgtgtatttcctcctgacatttcacttctccgtctttttattatgtggagagacttgagttagccaaggactgggtttccttaatccacaattacagccccatgccgagatatcccaggggccaaccttacagcacctggatcccagggttctagctgagtaatcattccagaatggggctgggaatcttgacagcattgccaaggggtggcaggcagtattaatcaatattagtcaatagtacctatcggcctcgaaaattacagattaaagttacagacgactcaggtgagaatgaagtgaccttgggcaagccccttctctttgggcctcagtctcctcatatgtaaaatgagagcactggtcaagatagctaatgtctgaggtcctgtccctctggttttctgaaactgtataatctgtgcaatctgagggttcaatcctagcaggtaaaacacccctgcctcactccacggctggatgaaggcagatttttccctatgagcccctgaccggtgaatgtgagaaggaaaacactggcctgtagaaccaagcctgcccttctttctcctggaagacagctgctttctagggagcaagtttactgagccacttcaccaaggacagactcttctaccctttgtagggaagcctgcctacctgtgtggtcctcgcgtagcccgtgagacagcctaattctggattcacctaacagggctatttagtctcatgcaaaaggtaaattctctaacttagttttaatctaaacaacaacaataacaacaacagtaatagtgtgatccccatttcctgtcttttgttttatttctcattgtgtgcagaaaccaggcaggaaata

SEQ ID NO: 195 - DNA, 2319 bp, Homo sapiens
cgcgttggcatcccagacgctccacatctgcacgaagaagaaaggcgtccagcacacgatgaaggccagcacgatgatgaaagtcatcttgaccgtgcggatcttggccttggagatgagcttgacgctgctgacacgcgccagggccacgcgccccccatcgccagccgccgcgccctctggcgcctcggccgccgccgctgcagcggtcttgagccgcaagttctgccagatcttgaagctgataaggccgtagcaggcagcgagcacgatgaccggcacgatgtagacagctagcgtgatccatgtgatgtaggccttgggtccccagggctggatgaagacggcccagcagtcgaagacgccgtcagccacctcgcgcagagagaagatgtgcacctgcggcgcgctggccaccaggcagccgagccacgtggcgagcactgccaggcggtcggtgcggcggcgcagcgagcgcagcggctggcagatggccaggcagcggtccagggacatgagcagcagcaggtaggtggaggcgaacatgcccaccacctgcaagtacttgaccaggcggcacagcaggtcgggcccgtagaagcggaaggtgatgtcccacagcaactgcggcagcacctgaaacactgccaccaccaggtcggcgatgcttaggtgcttcatgaagaagaagaggcgcgagtgcttctggcgtgtggtgcgcagcgccagcagcacacacgcgttcccgctcagcgccaggagcaggatgagacacagcaccgccacctccacgcgcgccagggcctcgttgcgccgcgggggtccggcggtgcggttgccctcggcccccggcggcgcggcgctggcgttggctgcctcggcgctccagttggctgcgagcgcgccctccatgaccctggcggcagcggtgcgccccggccttcgagccctttacggcttggcgcggctgggccggatccggcgtgtcggagggtgtaggggcgcccggggctccactcctggagactccacggacggatctgctgggtccaccctgaaacaaaccgggagggccgtgaggagaccgccgcgtttctcttccgacgcgggtagggcgtgcttgtcccattcccaggaacccaactcatctgaaacaacagggcacaaccgccggcctcgggttgctcagccgccaccccagaaatccccgttggaggtacctcctctgagccactgcaaatgagcgggaatcctctaccggccacaagcccagagcccccagcgtgccagttcctcgggatgttcagcggcttccactgggggagaagggagggtcaaaatcagcaacgttcctccgggagtgggaatctaaaaccaaatcacctccccgagggaatctccaaactacctactcgccccaggctccgaccctcagcatatccacctgccgcgggacccccaaaccagcccctctcctagtaaacattaattcaagtacttcctccggaaatccccaaatagccacttgcctgggaaaccccagctctggtccttccccgtctaacccccttttctaagacggtcggccgtcactccctgaacttccacagcacctgcttcggtggaaacccggttctgcagtccctgctgggggaacccctgcctcaaagcccaagatgcccaagcgctgttcccagaccctggcatagacacctcccgcggggctcctccccagcccggcagcctcgaaagttctggaacccctgacttgcgctttggacccagataatcaaggactctgagtctcaatcccgagaagtcactttacaatctctcggaacaccccgagctcctccaggaacaaggagtgcgaggcaccccaccacctccactcgggtcccgatggctccaccggccctagccatcccgtccacccctgcttagcacccagctacctgcaccgagtccgcaggcgaacctaaagttgactccccccggggaagttgcacggcgactgacggccccagtgacgcgtgcgctcccggcccgagttgggatggcctgccggctgcacctaatgtgatgctaagctgaggtctctcactggagcctcggttggaatcccctcgccgcagcctggctggtcgctggatgggtacaggaggcgagcgaggagcgcctcccaggctgcgcgcgcggacagcgtctggatgcggcgctgtgcgctggggctgaggctgcactatcgcacgggtccgctagggggcgggcgaggccagccgcgcagacccctccggagcggagctggttcgcccagcg

SEQ ID NO: 196 - DNA, 50 bp, Artificial Sequence
  CELF6 source sequence
cgggctaaaccccggtcccgccgtacccatgaaggaccacgacgccatca

SEQ ID NO: 197 - DNA, 50 bp, Artificial Sequence
  CELF6 Unmethylated AlleleA Probe Sequence
taataacatcataatccttcataaatacaacaaaaccaaaatttaaccca

SEQ ID NO: 198 - DNA, 50 bp, Artificial Sequence
  CELF6 Methylated AlleleB Probe Sequence
taataacgtcgtaatccttcataaatacgacgaaaccgaaatttaacccg

SEQ ID NO: 199 - DNA, 3001 bp, Homo sapiens
aagatctccttctgtctcttcagcctgactcaccctgagaatgggcctggactgggaccagcctgtgaggggagggaaagaaagaggccataaagaggactggggaggagcaaagaagaggcacaggagtgaggggattagagaaggaaagaagtgtggatttagctgagagaaatccaagaagaaagggacacagggtggcaggagttcccagctgaggtcagggttctgaactgaaatcaaaagaggtagagtgcattgggattctggcgaagggcaggagagattggggctgttgttgaacatgagggggtgtctggaactgagagcctcctgggaattgaattggattttgtgttagacaggtctgggttctgaagaaggggagggtttgggtctgcggtgttttaggttggcagtctgagagtcaaaagaaggctcttgagggggatggccggactaggaggccagaggaggggtgcaagtccaagatatgcatgtgggtggttaggcctgagtgaggtgccaccagatggaggattcaggagtgtttgggctcagggtttgggaaattgagggtattcgagatctagaaggtggaataagttttcaaattccagaattttaggtctgatgggagaggtggtggaaaggtccaattgaaaggcagggactgctgggatgtgagaggtggaggccaggaatttggaggttagagtggagaacatgttaggggaccacagtgataatcagagggaggatgtcagagagaaaattctgtgatctgggaagggggctgggctgaggtggggctgatattggactggtgttggactggctctcggcagggctagggctggggctgggctggggtcgggccacactctaatcggattggggctgggctgggctgggctgaccccgcgcacctttgtggaggccggtgagccggtccttcagcaccgtcagctcgtagatgcggccgaactcctcgaacagcggcttgaggtcctgctcgtccaagccccgcgggatctgccccacgaagagcttgatggcgtcgtggtccttcatgggtacggcgggaccggggtttagcccgctcatgccgacgccgctgtccgcggtgctgaaacccaggcgcgggccggggccagcgggctgcgctgaccctcccggcgccgcggccatgtccccgccctgtcagccctcccgccggtcccactggtcccgcctgtcccgccgtcccctccctggaccggtggcgagggccagggggagggggcggagcccgggcggagagggcggggggctgcccagggggcggggtccgggtggaggggcgtagagggggtggggcgggcaggaaaggggcggggccggggcggggcgggcccgggccgaggtggcggctgaacgctggggtctggcttgaggtccccgtgcggctctctctgggctcccgcccgagctctcccagagccgagccccgagccccagcccccgtgcttggtgacgtcaggcagctggccgccgagtctacgcaaatgatttgcataatgaggaagcagccgccaatcagagtgggagttgactgggctcgccgggggcggggggggcggctgggctcacgcgctcctgctcgccatggcaaccggaccctcgaatgccctgtgtgtgtcgagtgtgtgcgggcgtggccgtggatcgcgcgtgtgcgccttgggccgcgtgaggggaagcagcttcgtcatgcgtgatggtgtctgccggagtcggggctaatccccaccgcacacacaggcacacacaggcaaacgcaggcacacgcaggcacacacacctccaaacgctggtgcgtttcctgcgggggtggatggggattcctcttctctattcccactatcgagccagtccctcggagccaatcccaccccgccccatcacacatctctgtcttcggtgtctttaaccactttcttacccctgagcctcagacacattcgtctcccttgcagccttaaaaagaaaggaagaataaaataaaaaccccttaaacactccttgaacacggattcctccttaggagagtgagggaacttagccgtatttactcagaacccattatgcaccaggtgcccagttcatcccagttctgtgaactcaggagtgttctctttttccggaaaaggaagctgaggttctgagggtcgaggatgttagggccaggcccaccccaacatttgttgggcccagcgcaaaataagaaatggaaatcgacatactgtgcatctaaacatttaaaagatacaaatcaaattacaatttgttaaatatgttcccctcttattttcacaaatacaggttcagaattacgaaaatgaaagacatatgtacagctacgctttctatatgactgaaagttggcaaaatatcagagctgatggaattttattactcctgcacatgtctaggtattctcttgatggatcagtgatgtttgcacaaagtagtaatacaatgtttggacaaagacacatacttcataaattatatatttattccataatatatttttcatgcctctttcagtaaaattgctaattattttaatagagatttcctttttttttttttgataggttatcattctgtcacccaggctgtagtgcagtggtgcgatctaagatcactgcagcctcaacctcctgggttcaagcgatcctcccacctaagcctcccaagtagctgggatgacaggtgcacgctaccatgcctggataattttttttctttttttttttttgtagagatggtggtttcaccattctgcccaggcttgtctagaacactcctgagctcaagtgatccacccacctcggtgccccagagtgctgggattacaggagtgtgccactgcacctggcctaatagagattttaacataacttgtgtccattgactattctaagaaactgctggagaccagcagtagcaactg

SEQ ID NO: 200 - DNA, 856 bp, Homo sapiens
cgcgcacctttgtggaggccggtgagccggtccttcagcaccgtcagctcgtagatgcggccgaactcctcgaacagcggcttgaggtcctgctcgtccaagccccgcgggatctgccccacgaagagcttgatggcgtcgtggtccttcatgggtacggcgggaccggggtttagcccgctcatgccgacgccgctgtccgcggtgctgaaacccaggcgcgggccggggccagcgggctgcgctgaccctcccggcgccgcggccatgtccccgccctgtcagccctcccgccggtcccactggtcccgcctgtcccgccgtcccctccctggaccggtggcgagggccagggggagggggcggagcccgggcggagagggcggggggctgcccagggggcggggtccgggtggaggggcgtagagggggtggggcgggcaggaaaggggcggggccggggcggggcgggcccgggccgaggtggcggctgaacgctggggtctggcttgaggtccccgtgcggctctctctgggctcccgcccgagctctcccagagccgagccccgagccccagcccccgtgcttggtgacgtcaggcagctggccgccgagtctacgcaaatgatttgcataatgaggaagcagccgccaatcagagtgggagttgactgggctcgccgggggcggggggggcggctgggctcacgcgctcctgctcgccatggcaaccggaccctcgaatgccctgtgtgtgtcgagtgtgtgcgggcgtggccgtggatcgcgcgtgtgcgccttgggccgcgtgaggggaagcagcttcgtcatgcgtgatggtgtctgccggagtcg

SEQ ID NO: 201 - DNA, 50 bp, Artificial Sequence
  RAB39 source sequence
tgagaccttgcttcattttccttggttgttgctgctgtcacgtcttcccg

SEQ ID NO: 202 - DNA, 50 bp, Artificial Sequence
  REAB39 Unmethylated AlleleA Probe Sequence
taaaaccttacttcattttccttaattattactactatcacatcttccca

SEQ ID NO: 203 - DNA, 50 bp, Artificial Sequence
  RAB39 Methylated AlleleB Probe Sequence
taaaaccttacttcattttccttaattattactactatcacgtcttcccg

SEQ ID NO: 204 - DNA, 3001 bp, Homo sapiens
gtagagacggggtttctccatgttgaggctggtctcgaactcctgactcaggtgatccacccgcctcagcctcccaaagtgctgggattacaggcgtgagccaccgcgcccggcctaacacacacttttaaactgtttcactttttaaagataaaccttgacgtcactattactcaatttgaggaaaggcacaagaaagtagtaagatctcaccagaatttaagttctgctttgtttacttacatcattaatcagacatgagctaagagaccacctgttcgcttttttctgacttaaacaatggagaccaaggagagctgaaaatttcatgagcttctcaacatttccccggcttttttttttttttttttttttgccactgtccccagggttggagggccccagagggtatggggatgatacttggcgtttctcactcttctgtggtactcatagcagtgaagaacagacagtggcagcacgcagcaacagccagagcccttgctagaagcacccagaactggctgtgcaaatttatttcaatcctgcccaaaactccttcaccttaaaatgccttggaggtggctctcatcaagacaagaaagcagggtgtggtggctcacacctgtaatcccagcactgggaggctgagcctggaggatcacttgaggtcaggagttcgagaccagcctgggcaacacagcaagaccccatctctacaaaaagttagaaaattagcgggatgtggtggcccccgcctgtagtcccagctactcaggaggctgaggtggatggatcgcttgaacctaggagtttgagaccagcctaagcaacatagcgagaccacatctctaaaaaaaaaatttttttttaacccagaagaaaattagccaggtgtgatggctcacacccgtggtccccgctgcttgggaggctgaggtgggcagatcgcttgaggctgcaatgagccgagatctcaccacatcaatccagcctgggggacaaaaaaaaaagaggaatcaaagcaatgacccaagttatggcctcagggacttccaaaatcagagccctaatctgaccaacccgccctgacgcctgtaagaaaacgactctcacttttcattggctgaccctcccggagttgagaccttgcttcattttccttggttgttgctgctgtcacgtcttcccgccacgcccactttcctgccggcgcccagtgagcgttgagggagagtgatttgcagccggtcaggggcgggagttcgcggcagcaaacctgcttccccctcccacgcgccaccgcccccgccccgcccccgtccagaggccccgcccctagccgggcctggccccgcctctctctcccgagagggcgggcaccaggcggcggccgcagccgcaggaatatgctggaaggcggcgggcgggcgcccgcgaggtgctgaaaggacagttcccgccgccgaacttagcccgcgggtggggcggcccgggagccagcggggcacgtgagcgatggagaccatctggatctaccagttccgcctcatcgtgatcggggactccaccgtgggcaagtcctgcctcctgcaccgcttcacccagggccgcttccccgggctgcgctcccccgcctgcgaccccaccgtcggcgtggacttcttctcccgcctgctggagatcgagccgggcaagaggatcaagctacagctctgggacacggcgggacaggagcggttcaggtagggaccccggggaccttgggcaccgcgccgccccctcagcccgcccggacgccccttccccaggcgtccgccccgccggccctggtcgggagaggctctggcccttccctctcgaaagtgcaaacactccattctcgcttcccctttgcccctcctcttccagggacgacttcctcctccgcaatttctcacttctttctttggcgcccatttcctgcgccccctttccatctttcctttcccgcctgaaccggggtcgtcctcgccgccccctcttccccaactctttcagcccctgtgggtatttttatcccctccggctccccggctgccttcttcccaaactcggcttccctggctgcgcacagcatgacccgaccaccctcctccaagcaggcgccccttttcctaccctaacccagcggctcccacgctgtgtggccggggagaaccgcgagggagggttcccagattcgtgatggatcctttagcttcacagcaaggagcgctccaacccaggttcctgcatgagctcactgggcatggttcacagagaggtggatgcggtaatggtttgggcacctcacagcactgaatgtggtcatcagaaatactgggtcatgacccgtgggaatgggtcttgtaggttaactagatgcaacatcagggacactggcacagctgagggtgtgtgtgtgtgtgtgtgtgtatgtgaagggttgagttgtctttagaagtttgtcatgtttggtgggaaaaagacatatattgagattcgccaggaactttaattttgcttatgatcagtacttaacgttccagggtgataatatttctaaagacagaaggagggatccaaatgaatagtctcctcctaaatatccgctggggacatgaagggtttcacaaaggcgacttgtgtaaataaaaagtaaatgaaattatatgcatttcctgaatatttgagatggaagcaggtttaaagcaaattttaaatgtctaggattcaaaggcacttcgttttagaggaagatggtgaaactgatgtattgtactgttctgtttcttaaagactgcattatccattggaactcatttaaggaagcattttgcgttagtcagcaagtactctgcttgtgaaatgcagtgttccatgaggggacttcccaaacatggcttcccctcgcccccagccagcatcactgcgaatgagatcagcctacatctgtgagaatggtccaacattcaaagtt

SEQ ID NO: 205 - DNA, 1022 bp, Homo sapiens
cgtcttcccgccacgcccactttcctgccggcgcccagtgagcgttgagggagagtgatttgcagccggtcaggggcgggagttcgcggcagcaaacctgcttccccctcccacgcgccaccgcccccgccccgcccccgtccagaggccccgcccctagccgggcctggccccgcctctctctcccgagagggcgggcaccaggcggcggccgcagccgcaggaatatgctggaaggcggcgggcgggcgcccgcgaggtgctgaaaggacagttcccgccgccgaacttagcccgcgggtggggcggcccgggagccagcggggcacgtgagcgatggagaccatctggatctaccagttccgcctcatcgtgatcggggactccaccgtgggcaagtcctgcctcctgcaccgcttcacccagggccgcttccccgggctgcgctcccccgcctgcgaccccaccgtcggcgtggacttcttctcccgcctgctggagatcgagccgggcaagaggatcaagctacagctctgggacacggcgggacaggagcggttcaggtagggaccccggggaccttgggcaccgcgccgccccctcagcccgcccggacgccccttccccaggcgtccgccccgccggccctggtcgggagaggctctggcccttccctctcgaaagtgcaaacactccattctcgcttcccctttgcccctcctcttccagggacgacttcctcctccgcaatttctcacttctttctttggcgcccatttcctgcgccccctttccatctttcctttcccgcctgaaccggggtcgtcctcgccgccccctcttccccaactctttcagcccctgtgggtatttttatcccctccggctccccggctgccttcttcccaaactcggcttccctggctgcgcacagcatgacccgaccaccctcctccaagcaggcgccccttttcctaccctaacccagcggctcccacgctgtgtggccggggagaaccgcg

SEQ ID NO: 206 - DNA, 50 bp, Artificial Sequence
  LOX source sequence 2
cggtaagtacccccaagtccgctggaagcacccgtgcacctggtccccag

SEQ ID NO: 207 - DNA, 50 bp, Artificial Sequence
  LOX unmethylated AlleleA Probe Sequence 2
ctaaaaaccaaatacacaaatacttccaacaaacttaaaaatacttacca

SEQ ID NO: 208 - DNA, 50 bp, Artificial Sequence
  LOX Methylated AlleleB Probe Sequence 2
ctaaaaaccaaatacacgaatacttccaacgaacttaaaaatacttaccg

SEQ ID NO: 209 - DNA, 3001 bp, Homo sapiens; identical to SEQ ID NO: 164

SEQ ID NO: 210 - DNA, 1577 bp, Homo sapiens; identical to SEQ ID NO: 165

SEQ ID NO: 211 - DNA, 50 bp, Artificial Sequence
  RUNX3 source sequence
cggccgctgttatgcgtattcccgtagacccaagcaccagccgccgcttc

SEQ ID NO: 212 - DNA, 50 bp, Artificial Sequence
  RUNX3 Unmethylated AlleleA Probe Sequence
aaaacaacaactaatacttaaatctacaaaaatacacataacaacaacca

SEQ ID NO: 213 - DNA, 50 bp, Artificial Sequence
  RUNX3 Methylated AlleleB Probe Sequence
aaaacgacgactaatacttaaatctacgaaaatacgcataacaacgaccg

SEQ ID NO: 214 - DNA, 3001 bp, Homo sapiens
gtcgtcttaatgggagcagggacggcctcagcttccagccacctcgggcagcaccacccccagccgccggcccttcctgccctgcccttttctcacggcagctgtgagaggtttaggggaaaaccgaggcgttttcgtttcatctcgctgcccccttaaaaaaatgaaaatgaaacagtcgcctactccctggcataaagaaaaaggtcctctaaatggctgggggctgccagggttaggggtcccccaatctcaactcgccattcgggacgcataatatccccgagcaaacgtctggagagcagtgccccgatcccggcctagcgccgtccggtaaaatttcggaagcccgagggtgtgagcaggaagcttttgcgaagcggcgcgggaggaggggtgctggaggcggagggtaggccctttcaccgttcgcaccccacccgcggtgtccttgcccctgtcccgggatcctcttctccgttacccgcagggctgtatctgagcgatccgggttaggggggcgcaaaaccccatccgcccatttccgcaccaacgtctctacgcaaggcgccccaaaacccaggtggagcggggcaaccccgttaaaagtcattcctgcagggcgcatccaaaacggaacgccgaggtcccggagccgagcgcgcagccagactgaaccgggtgcccgggtgtcgccgcggcgtctcgggcacctcccatccccactgctcccgaggctctggctcccgcagctcagacgcccggagccccagggccggcgccctcccgccccgggtcccgcactcaccttgaaggcgacgggcagcgtcttgttgcagcgccagtgcgagggcagcacggagcagaggaagttggggctgtcggtgcgcacgagctcgcctgcgtggtccgccagcacgtccaccatcgagcgcacctcgggccgggcgcgccctccgggccccacggccgcctgcgcgctcagcgcgccgctgttctcgcccatcttgccgccgccgccgccgcaggggaaggccggggagggaggtgtgaagcggcggctggtgcttgggtctacgggaatacgcataacagcggccgtcagggcgccgggcaggcggagacggcgcggcttcccccgggggcggccggcgcgggcgcctcctcggccgccgctgccgcgagaagcgggaaagcagaagcggcggggcccgggcctcagggcgcagggggcggcgcccggccactactcgccagggcccgcccgctgcgaggcctcgctggcccgacggccgcccgcagcctgcccggctagtcccgcatcctcggcgcgcggccccgcgtgcggccgcccctcgtggctgtcccggctgcctgggccgcggcggggcccgcgcggggctgtgccgctgccgccgcctcccgccccgaagctcgcccgcggccgccccgactccgcggccgcagccccagaacaaatcctccagaatcaagtggcggggccgcggccgcccgcgcggggttagtacccccggggcccgcggggcggggctggcggagcgacgcgtcgcacagccaatcggcggagcccccatcgcgggcacctcggtggcgttcgcggggaggaacggggcctgccggaggccgcccaacggggaggggcggaaggcgccaccccgcggaggaggccccagtgccacagcccagggcccccgagagctctgggagcccggggcaaatgctagaaatttgcttagaacgtccgggtcccacggaaggcgcccttgccgccctctctcgggtcgtagctccctgacgctggggcgcaaccccttcgctcctcctccccgctggccgcggccgggcttccccagctcttgctgcttcgggcctgtgacttctgcaaccccgggctgggggccgcggggtctcagggccggtgacgccgcactgggagccgccccaaagaggttactcacctccctcgtcccgcacattattctgacccaagagcctccaccccacacgggattttgcgcgtcgtccacgcccggccggcggcctttgctgctcccagccctgcgcggctttggtcccagcctcggtggcccctgtgccaaaccggggacaggcggaagggagtctcctagggaccctaagtagcctggggccaacaacccctttcctctctgctctcccctcaaaacaagtttcaggatcttgcaggcctcgcggcgtcgttcttcgttgtggcggcctgtggctctttgaaaaacacgacgaggcctgcaaaatgcgtttttctttttttcctttacgcatgtaaccacggtcctgcatcgtgaaacggtacgcgcgtcggtggcaaaagaaaaacagcagtggctgcaaagctaagggccctcgctttcagaggagagaattttctttctccatgcgggtggaaagtggcctctgcgggtccaaccccacttcttcttgggcccgtgcgctccggctgcgccgcagggaccgcggacagcttcgccaaggcactgcctgcccgcccggctccgggtccccgctcccactcccagccgcgtggcccaacctctcctgggcttcactgcaaatcaccccttcctctcccgcctcctaagtctgtcgagcagacctaggggccggctacagttgggagggcaacgggaaagatcaagccacaatcattccgaattatcgccccagacacctccctagactctggggaacgaacgcgtgctgagcctccccgccgctttggagacggggctagattttcgttgcctccggctctcgacaggtgcaaaacaatgaattccaagcctcggaagcaaagaagcttaggatccgacggtggccgcaagatctcatcatggatctgacccctgctcagcgcgcgccatttcgtcgttgccaaacgaaatcaagccccgcgtgcgctccaggggcgaaggactctggactcaccccgaccaccgggaga

SEQ ID NO: 215 - DNA, 3478 bp, Homo sapiens
cgccattcgggacgcataatatccccgagcaaacgtctggagagcagtgccccgatcccggcctagcgccgtccggtaaaatttcggaagcccgagggtgtgagcaggaagcttttgcgaagcggcgcgggaggaggggtgctggaggcggagggtaggccctttcaccgttcgcaccccacccgcggtgtccttgcccctgtcccgggatcctcttctccgttacccgcagggctgtatctgagcgatccgggttaggggggcgcaaaaccccatccgcccatttccgcaccaacgtctctacgcaaggcgccccaaaacccaggtggagcggggcaaccccgttaaaagtcattcctgcagggcgcatccaaaacggaacgccgaggtcccggagccgagcgcgcagccagactgaaccgggtgcccgggtgtcgccgcggcgtctcgggcacctcccatccccactgctcccgaggctctggctcccgcagctcagacgcccggagccccagggccggcgccctcccgccccgggtcccgcactcaccttgaaggcgacgggcagcgtcttgttgcagcgccagtgcgagggcagcacggagcagaggaagttggggctgtcggtgcgcacgagctcgcctgcgtggtccgccagcacgtccaccatcgagcgcacctcgggccgggcgcgccctccgggccccacggccgcctgcgcgctcagcgcgccgctgttctcgcccatcttgccgccgccgccgccgcaggggaaggccggggagggaggtgtgaagcggcggctggtgcttgggtctacgggaatacgcataacagcggccgtcagggcgccgggcaggcggagacggcgcggcttcccccgggggcggccggcgcgggcgcctcctcggccgccgctgccgcgagaagcgggaaagcagaagcggcggggcccgggcctcagggcgcagggggcggcgcccggccactactcgccagggcccgcccgctgcgaggcctcgctggcccgacggccgcccgcagcctgcccggctagtcccgcatcctcggcgcgcggccccgcgtgcggccgcccctcgtggctgtcccggctgcctgggccgcggcggggcccgcgcggggctgtgccgctgccgccgcctcccgccccgaagctcgcccgcggccgccccgactccgcggccgcagccccagaacaaatcctccagaatcaagtggcggggccgcggccgcccgcgcggggttagtacccccggggcccgcggggcggggctggcggagcgacgcgtcgcacagccaatcggcggagcccccatcgcgggcacctcggtggcgttcgcggggaggaacggggcctgccggaggccgcccaacggggaggggcggaaggcgccaccccgcggaggaggccccagtgccacagcccagggcccccgagagctctgggagcccggggcaaatgctagaaatttgcttagaacgtccgggtcccacggaaggcgcccttgccgccctctctcgggtcgtagctccctgacgctggggcgcaaccccttcgctcctcctccccgctggccgcggccgggcttccccagctcttgctgcttcgggcctgtgacttctgcaaccccgggctgggggccgcggggtctcagggccggtgacgccgcactgggagccgccccaaagaggttactcacctccctcgtcccgcacattattctgacccaagagcctccaccccacacgggattttgcgcgtcgtccacgcccggccggcggcctttgctgctcccagccctgcgcggctttggtcccagcctcggtggcccctgtgccaaaccggggacaggcggaagggagtctcctagggaccctaagtagcctggggccaacaacccctttcctctctgctctcccctcaaaacaagtttcaggatcttgcaggcctcgcggcgtcgttcttcgttgtggcggcctgtggctctttgaaaaacacgacgaggcctgcaaaatgcgtttttctttttttcctttacgcatgtaaccacggtcctgcatcgtgaaacggtacgcgcgtcggtggcaaaagaaaaacagcagtggctgcaaagctaagggccctcgctttcagaggagagaattttctttctccatgcgggtggaaagtggcctctgcgggtccaaccccacttcttcttgggcccgtgcgctccggctgcgccgcagggaccgcggacagcttcgccaaggcactgcctgcccgcccggctccgggtccccgctcccactcccagccgcgtggcccaacctctcctgggcttcactgcaaatcaccccttcctctcccgcctcctaagtctgtcgagcagacctaggggccggctacagttgggagggcaacgggaaagatcaagccacaatcattccgaattatcgccccagacacctccctagactctggggaacgaacgcgtgctgagcctccccgccgctttggagacggggctagattttcgttgcctccggctctcgacaggtgcaaaacaatgaattccaagcctcggaagcaaagaagcttaggatccgacggtggccgcaagatctcatcatggatctgacccctgctcagcgcgcgccatttcgtcgttgccaaacgaaatcaagccccgcgtgcgctccaggggcgaaggactctggactcaccccgaccaccgggagagctggcccctacccacctcgggacctcacagcacgccctcaggccgtgtcgaaaggaaggacggcaaaggtcccttactgaaccttttaagagagcctgcgcctggcagttgtcgattgcggacccaggcccgcgcgccctcggacgcgctggcacgagcagcagaactagaggaaagcgagtgatccagcctgggcgctcccacctccgggaacgtctccgagaaggcgcagcgcgtcgtggccaggtagggccctggccgggggcgggcaacacgtgctgccctcgagcaggttgcgggaccatgacccgctgtttcaggtggtggtaaattccatttgtcgaatggtttcggtttgcaccgtgccctttgcttgttcctccgcctgatttctccctctccgcttacgatgggttcacagacaagtttccagagaatgagggactcttgtgggccctggcacctggcgcagggcccggcacggctccggctctccgtagggcgctggctccccgtgggcaccagatccaagggaccagggcggcggggggagggggggcgggtgcaggcccttgggtccccagaccaaggtcgcggggccgcctggcaggcacagtggcgggagccgccgctagttggcgcccgcgccctgccagccgcggaggtgcgggcccggccgggctacagatgcgcgccagctgcggccccgggtgcaggcgcggcgaccgcccccg

SEQ ID NO: 216 - DNA, 50 bp, Artificial Sequence
  COL4A3 source sequence
cgccaggagctgccgccttgccaccccacgggacgcgcacctccagcccc

SEQ ID NO: 217 - DNA, 50 bp, Artificial Sequence
  COL4A3 Unmethylated AlleleA Probe Sequence
aaaactaaaaatacacatcccataaaataacaaaacaacaactcctaaca

SEQ ID NO: 218 - DNA, 50 bp, Artificial Sequence
  COL4A3 Methylated AlleleB Probe Sequence
aaaactaaaaatacgcgtcccgtaaaataacaaaacgacaactcctaacg

SEQ ID NO: 219 - DNA, 2881 bp, Homo sapiens
ttgcagcaaatatcagcaacactgttccaaagcaaagattcttaacctgaggatggaattcaggtgatctgtataggaaactcacataggaaaataagttgcatctttacttttagtaacatgtcacagaagttcaacatgtccttcccttatgagcacaggcaacagagcacagtagtattggcagttcttgataatttgcacccaccggtattttcatgtcacatttcagtttgggctattatctcaaaataccatttgcagacatcactactttgacattactgttgcttaatcggcctgctgttaaatcaagctatttaatgcaataatgatgatgcacctgtgttagttactatatcacgacttgaggacttccataattgcattccaataaaactgattctgatttaattgtattttatgcatttcacaacattcttctgagaaacagtctaattgctttaccagagccagtgtggtccgtagcacaggaaacggcgagagccggctggaggaccgtgccctggcacatttgcatcgcaggccacccccaggaagagcgcaccctgaccagagctgagtcttgttttgccctgcatttccgcagataagatttggcctgaaaagcagttcaactcagcggagaataagatgggctttctctttctcctcagctgcctctttctcttggcattttggagtgccccgactgccaggccggtaactcgagcctccaaattaatcaaaacacacagcacccagttaaagcccaggcagacaagcccaggagccagacgcgctctcctctggtgacccccgggccacaactccccagctgggcccgccaccaccccttgaatgccacgctccctccactgcccctcccaggcaggtgccggggccccgggacagcggttgccccacctatgggcgccttacctgtggggacgcccgcagcgccaggagctgccgccttgccaccccacgggacgcgcacctccagccccaatccacccgcgccccgctgcctcttcgcggcgctctcgcagccaagcccggcggccgcaagttggaggcgggctggaggcggggaacgcggaccgccgcgggcgcggggaacaagcggggcctcccgagtgcagacgcccggcgcgcctcccgcttaatctgggcagggccgctggccactccctccaccctgcgcagccacctccccaccgcgcagccacctccccaccgcacacccccaaacgccccacctccgaccgcaccccacttccccgcctgggcccccggaccttgggagcatcacctccttaaccccttaccctggatccgcgcccacctgcccctcaggcgcccagccctttctcgcctcctgggcacgatgcccgggtagaagggacactgcctggtaagttgggagggagggggtatgagggcgggacctgagccacgtcttccctcccttgaagccacaaccaaaaagcctgggtggggagggacgaaccgcgcgaccgagccctacaaaacccgccccggccgagtggcgaggcgagctttccagccgggctcccagagccgcgctgcgcaggagacgcggtggcctgagagcctgagggtccccggactcgcccaggctctgagcgcgcgcccaccatgagcgcccggaccgcccccaggccgcaggtgctcctgctgccgctcctgctggtgctcctggcggcggcgcccgcagccagcaaggtgagtgggggctgcgcgacccccacccccgcacttccatccctcctccacgcgtccgggggacgcgctggccccacccgcagcggcgcggtagtggagcggctgccgggctagtggcgaggctgagggcttcacgcaggtcccgggacaggcagcgagcggaagggagcaagcggggatgccccggaacaggtggaatgcgcggggctgggggaagaggcgaggagggggcttgtccagtgcctaggagtcagggatggcggggacaggcctccagaggtcagtaaattaaaaaatagacacatagataatcgccagggatgatacgtgagaacaccggatgtggagatgcacggtgtcacgctggtgaatcaaatcaagcagcttggcccagcgcacagagggctggcaggagacacctgcagggagtcccagccgccacttcctagctgggacgtcacactaaaccctgacctctctggggctcagtttcttccttgcaaaacttgccctccagttttcctttgctattgttgggaaaagcaaatgctatcactgaaaaattaagagcaccttagaaatcgaactcaggttagaatcatttaagcaatctgacactttgatttgatttttgacttggaatttgagtgaaactggatttatttaaatccatcccatatttcttattctttgccattaaatagaaattgattgtcctcaaaacgattgtgcctcctagagttgactcacttgattttaccttgtgaaagtaaactttaacttttcagatacttgttatttttacactttaaaaagttgatttgggtgataacgtggcataggaaatttcatttccaaggatgagcagatgtttttacaaatgttatcattgagattatgattggaaattaacggtcacataaatatttatatatgttacatgaaaagaattttagaacctatggtcacatataggttctatatgtttttcctatttaaattaaatgaagacattgcagacattataggtgaaactaaagccctctctgacaccccccatacctctccccatttccttgtcttccaagaggtgtccactttcaccactttcatatttcatgtagttttccaa

SEQ ID NO: 220 - DNA, 1054 bp, Homo sapiens
cggggccccgggacagcggttgccccacctatgggcgccttacctgtggggacgcccgcagcgccaggagctgccgccttgccaccccacgggacgcgcacctccagccccaatccacccgcgccccgctgcctcttcgcggcgctctcgcagccaagcccggcggccgcaagttggaggcgggctggaggcggggaacgcggaccgccgcgggcgcggggaacaagcggggcctcccgagtgcagacgcccggcgcgcctcccgcttaatctgggcagggccgctggccactccctccaccctgcgcagccacctccccaccgcgcagccacctccccaccgcacacccccaaacgccccacctccgaccgcaccccacttccccgcctgggcccccggaccttgggagcatcacctccttaaccccttaccctggatccgcgcccacctgcccctcaggcgcccagccctttctcgcctcctgggcacgatgcccgggtagaagggacactgcctggtaagttgggagggagggggtatgagggcgggacctgagccacgtcttccctcccttgaagccacaaccaaaaagcctgggtggggagggacgaaccgcgcgaccgagccctacaaaacccgccccggccgagtggcgaggcgagctttccagccgggctcccagagccgcgctgcgcaggagacgcggtggcctgagagcctgagggtccccggactcgcccaggctctgagcgcgcgcccaccatgagcgcccggaccgcccccaggccgcaggtgctcctgctgccgctcctgctggtgctcctggcggcggcgcccgcagccagcaaggtgagtgggggctgcgcgacccccacccccgcacttccatccctcctccacgcgtccgggggacgcgctggccccacccgcagcggcgcggtagtggagcggctgccgggctagtggcgaggctgagggcttcacgcaggtcccgggacaggcagcgagcggaagggagcaagcggggatgccccggaacaggtggaatgcgcg

SEQ ID NO: 221 - DNA, 26 bp, Artificial Sequence
  KCNK13 forward primer
tttatttttaagtttcggtagtcgat

SEQ ID NO: 222 - DNA, 30 bp, Artificial Sequence
  KCNK13 reverse primer
gacgataatacctcctaattataatcgtaa

SEQ ID NO: 223 - DNA, 28 bp, Artificial Sequence
  KCNK13 probe sequence 5′ 6FAM and 3′ BHQ-1
tcgcgctaaacctataacctcccgaatc

SEQ ID NO: 224 - DNA, 91 bp, Artificial Sequence
  KCNK13 amplicon sequence unconverted
tccatccctaagccccggcagccgattcggagactcgggaggccacaggctcagcgcgacaccacgaccacaactaggaggcaccatcgtc

SEQ ID NO: 225 - DNA, 23 bp, Artificial Sequence
  SLIT1 forward primer
aggatttttattcgggagttagc

SEQ ID NO: 226 - DNA, 24 bp, Artificial Sequence
  SLIT1 reverse primer
cgaacgaaaataatcaacgactac

SEQ ID NO: 227 - DNA, 30 bp, Artificial Sequence
  SLIT1 probe sequence 2 5′ 6FAM and 3′ BHQ-1
ccgtctaactcgcgaacgaaacgctataaa

SEQ ID NO: 228 - DNA, 90 bp, Artificial Sequence
  SLIT1 amplicon sequence unconverted
aggacccccacccgggagtcagcgccatggtgccctcacagcgtcccgctcgcgagccagacggcagcagccgctgaccatccccgtccg

SEQ ID NO: 229 - DNA, 22 bp, Artificial Sequence
  SLIT1 forward primer 2
aatggtttgtagtttcgtgtcg

SEQ ID NO: 230 - DNA, 20 bp, Artificial Sequence
  SLIT1 reverse primer 2
acgcctaaatacctcgacgt

SEQ ID NO: 231 - DNA, 27 bp, Artificial Sequence
  SLIT1 probe sequence 2 5′ 6FAM and 3′ BHQ- 2
ccctctacacctacaccgaaaccacga

SEQ ID NO: 232 - DNA, 85 bp, Artificial Sequence
  SLIT1 amplicion sequence unconverted 2
aatggcctgcagccccgtgccgtggcagtccaccgtggttccggtgcaggtgcagagggcggggcacgccgaggcacccaggcgc

SEQ ID NO: 233 - DNA, 20 bp, Artificial Sequence
  RAB31 former primer
tatgatggcgatacgggagt

SEQ ID NO: 234 - DNA, 16 bp, Artificial Sequence
  RAB31 reverse primer
cgaaaacgcgaaccga

SEQ ID NO: 235 - DNA, 28 bp, Artificial Sequence
  RAB31 probe sequence 2 5′ 6FAM and 3′ BHQ-1
acgaataacgaccaaaactcaccccgaa

SEQ ID NO: 236 - DNA, 87 bp, Artificial Sequence
  RAB31 amplicon sequence unconverted
catgatggcgatacgggagctcaaagtgtgccttctcggggtgagtcctggccgccacccgccggcggaccccggcccgcgctctcg

SEQ ID NO: 237 - DNA, 22 bp, Artificial Sequence
  FOXL2/C3ORF72 forward primer
ggttttatcgagtttcgtttgc

SEQ ID NO: 238 - DNA, 24 bp, Artificial Sequence
  FOXL2/C3ORF72 reverse primer
aacttaaaaataaactcgcccgta

SEQ ID NO: 239 - DNA, 27 bp, Artificial Sequence
  FOXL2/C3ORF72 5′ 6FAM and 3′ BHQ-1
cgactaaccgccccgctataaaaacga

SEQ ID NO: 240 - DNA, 86 bp, Artificial Sequence
  FOXL2/C3ORF72 amplicon sequence unconverted
ggctccaccgagttccgcttgcgtcaggcgccttcgcccctatagcggggcggccagccgcgcacgggcgagttcatctccaagtc

SEQ ID NO: 241 - DNA, 22 bp, Artificial Sequence
  B3GAT2 forward primer sequence
ggcgttgtagagatttggagtc

SEQ ID NO: 242 - DNA, 18 bp, Artificial Sequence
  B3GAT2 reverse primer
cgcctacaccccttatcg

SEQ ID NO: 243 - DNA, 22 bp, Artificial Sequence
  B3GAT2 probe oligo sequence  5′ 6FAM and 3′ BHQ-1
ctaccgctcctccacgcccaaa

SEQ ID NO: 244 - DNA, 19 bp, Artificial Sequence
  FAM78A forward primer
cgtacgatcgcgcgtatta

SEQ ID NO: 245 - DNA, 19 bp, Artificial Sequence
  FAM78A reverse primer
ccctacaacgacaaccgct

SEQ ID NO: 246 - DNA, 23 bp, Artificial Sequence
  FAM78A probe sequence  5′ 6FAM and 3′ BHQ-1
ccgcccgtccgaaacgatatcaa

SEQ ID NO: 247 - DNA, 101 bp, Artificial Sequence
  FAM78A amplicon sequence unconverted
cgcacgaccgcgcgcaccagcgaataatagccgcccgtgacatctccgctgataccgtcccggacgggcggggtggggggcgagcggctgccgctgcaggg

SEQ ID NO: 248 - DNA, 25 bp, Artificial Sequence
  MYOCD forward primer
ggttcgtcgtaaagagttaagagtc

SEQ ID NO: 249 - DNA, 20 bp, Artificial Sequence
  MYOCD reverse primer
caatcaaaaacgacgaacga

SEQ ID NO: 250 - DNA, 23 bp, Artificial Sequence
  MYOCD probe sequence  5′ 6FAM and 3′ BHQ-1
aaaccgccgaaaccgtctcgaaa

SEQ ID NO: 251 - DNA, 85 bp, Artificial Sequence
  MYOCD amplicon sequence unconverted
ggcccgccgcaaagagttaagagccggttcccgagacggcttcggcggctccgggtccccagaccccgctcgccgctcctgattg

SEQ ID NO: 252 - DNA, 26 bp, Artificial Sequence
  KCNC1 forward primer
tagtttagcggaattttagttcgagt

SEQ ID NO: 253 - DNA, 26 bp, Artificial Sequence
  KCNC1 reverse primer
caaaaacacccgaaatattactcgta

SEQ ID NO: 254 - DNA, 28 bp, Artificial Sequence
  KCNC1 probe sequence 5′ 6FAM and 3′ BHQ-1
taacgccgaacgctactctccgtaaacc

SEQ ID NO: 255 - DNA, 87 bp, Artificial Sequence
  KCNC1 amplicon sequence unconverted
cagcccagcggaaccccagctcgagcccgggctcacggagagcagcgctcggcgttagccgcacgagcaacaccccgggtgcccctg

SEQ ID NO: 256 - DNA, 22 bp, Artificial Sequence
  FSTL1 forward primer
tttcggtttttcgtttatttcg

SEQ ID NO: 257 - DNA, 22 bp, Artificial Sequence
  FSTL1 reverse primer
cttccgcaaatataaaaacgct

SEQ ID NO: 258 - DNA, 29 bp, Artificial Sequence
  FSTL1 probe sequence  5′ 6FAM and 3′ BHQ-1
ctcgcgctaataacgatcgcctaaatccg

SEQ ID NO: 259 - DNA, 90 bp, Artificial Sequence
  FSTL1 amplicon sequence unconverted
cctcggcccctcgcctacctcggcgcggacccaggcgaccgccaccagcgcgagcgcgagcgcgagccagcgtttccacatctgcggaag

SEQ ID NO: 260 - DNA, 24 bp, Artificial Sequence
  FSTL1 forward primer 2
catcgaaatttttagcgttatttc

SEQ ID NO: 261 - DNA, 18 bp, Artificial Sequence
  FSTL1 reverse primer 2
aactcgatccccgaaacc

SEQ ID NO: 262 - DNA, 25 bp, Artificial Sequence
  FSTL1 probe sequence 2 5′ 6FAM and 3′ BHQ-1
cgctaaacgaataaacgcgcgtcct

SEQ ID NO: 263 - DNA, 94 bp, Artificial Sequence
  FSTL1 amplicon sequence unconverted 2
gaccgaaactcccagcgccaccccgggagagcatccccaggacgcgcgcccacccgcccagcgcgcagacccaagaggccccggggaccgagtt

SEQ ID NO: 264 - DNA, 22 bp, Artificial Sequence
  FSTL1 reverse primer 3
cccgaaacctcttaaatctacg

SEQ ID NO: 265 - DNA, 85 bp, Artificial Sequence
  FSTL1 amplicon sequence 3 unconverted
gaccgaaactcccagcgccaccccgggagagcatccccaggacgcgcgcccacccgcccagcgcgcagacccaagaggccccggg

SEQ ID NO: 266 - DNA, 20 bp, Artificial Sequence
  SLC6A4 forward primer
cgtatttgtattcgcgggtt

SEQ ID NO: 267 - DNA, 24 bp, Artificial Sequence
  SLC6A4 reverse primer
aaatttattcgcctcaaaataacg

SEQ ID NO: 268 - DNA, 20 bp, Artificial Sequence
  SLC6A4 probe sequence  5′ 6FAM and 3′ MGBFNQ
actctttaacgacgactatc

SEQ ID NO: 269 - DNA, 100 bp, Artificial Sequence
  SLC6A4 amplicon sequence unconverted
cgtatttgtacccgcgggccctcacatggtctgatctctagatagccgccgccaaagagctcttgaagaatttttgcgtcactttgaggcgaataaactt

SEQ ID NO: 270 - DNA, 18 bp, Artificial Sequence
  FAM78A forward primer 2
gacggcgtagttttgggc

SEQ ID NO: 271 - DNA, 25 bp, Artificial Sequence
  FAM78A reverse primer 2
aacgactattattcgctaatacgcg

SEQ ID NO: 272 - DNA, 27 bp, Artificial Sequence
  FAM78A probe sequence 2 5′ 6FAM and 3′ BHQ-1
cgtacgaccgccactaacgaaacgaac

SEQ ID NO: 273 - DNA, 108 bp, Artificial Sequence
  FAM78A amplicon sequence 2 unconverted
gacggcgcagctctgggcggtctccccggaggcggtggcccccgccccccgcgcccgccccgtcagtggcggccgcacgaccgcgcgcaccagcgaataatagccgcc

SEQ ID NO: 274 - DNA, 21 bp, Artificial Sequence
  MLH1 forward primer
aggaagagcggatagcgattt

SEQ ID NO: 275 - DNA, 21 bp, Artificial Sequence
  MLH1 reverse primer
tcttcgtccctccctaaaacg

SEQ ID NO: 276 - DNA, 31 bp, Artificial Sequence
  MLH1 probe sequence  5′ 6FAM and 3′ BHQ-1
cccgctacctaaaaaaatatacgcttacgcg

SEQ ID NO: 277 - DNA, 84 bp, Artificial Sequence
  MLH1 amplicon sequence unconverted
aggaagagcggacagcgatctctaacgcgcaagcgcatatccttctaggtagcgggcagtagccgcttcagggagggacgaaga

SEQ ID NO: 278 - DNA, 92 bp, Artificial Sequence
  B3GAT2  amplicon sequence unconverted
ggcgctgcagagacctggagccgcggggctcactacctgggcgtggaggagcggcaggttcgcgcaagctagagcgacaaggggtgcaggcg

(19) United States
(12) Patent Application Publication
(10) Pub. No.: US 2013/0065228 A1
(43) Pub. Date: Mar. 14, 2013

(54) GENOME-SCALE ANALYSIS OF ABERRANT DNA METHYLATION IN COLORECTAL CANCER

(71) Applicants: TOSHINORI HINOUE, ALHAMBRA, CA (US); HUI SHEN, SAN GABRIEL, CA (US); DANIEL J. WEISENBERGER, LOS ANGELES, CA (US); PETER W. LAIRD, SOUTH PASADENA, CA (US)

(72) Inventors: TOSHINORI HINOUE, ALHAMBRA, CA (US); HUI SHEN, SAN GABRIEL, CA (US); DANIEL J. WEISENBERGER, LOS ANGELES, CA (US); PETER W. LAIRD, SOUTH PASADENA, CA (US)

(73) Assignee: UNIVERSITY OF SOUTHERN CALIFORNIA, LOS ANGELES, CA (US)

(21) Appl. No.: 13/486,988

(22) Filed: Jun. 1, 2012

Related U.S. Application Data
(60) Provisional application No. 61/492,325, filed on Jun. 1, 2011.
(60) Provisional application No. 61/492,749, filed on Jun. 2, 2011.

(51) Int. Cl. C12Q 1/68

(52) U.S. Cl. 435/6.11

(57) ABSTRACT

Particular aspects provide methods and compositions (e.g., gene marker panels) having substantial utility for at least one of diagnosis, identification and classification of colorectal cancer (CRC) (e.g., tumors) relating to distinctive DNA methylation-based subgroups of CRC including CpG island methylator phenotype (CIMP) groups (e.g., CIMP-H and CIMP-L) and non-CIMP groups. Exemplary marker panels include: B3GAT2, FOXL2, KCNK13, RAB31 and SLIT1 (CIMP marker panel); and FAM78A, FSTL1, KCNC1, MYOCD, and SLC6A4 (CIMP-H marker panel). Further aspects relate to genetic mutations, and other epigenetic markers relating to said CRC subgroups that can be used in combination with the gene marker panels for at least one of diagnosis, identification and classification of colorectal cancer (CRC) (e.g., tumors) relating to distinctive CIMP and non-CIMP groups.

### CROSS-REFERENCE TO RELATED APPLICATIONS

This application claims the benefit of priority to U.S. Provisional Patent Application Ser. Nos. 61/492,749 filed 2 Jun. 2011, and 61/492,325 filed 1 Jun. 2011, both of which are incorporated by reference herein in their entirety.

### STATEMENT REGARDING FEDERALLY-SPONSORED RESEARCH

This invention was made with government support under Contract No. 5R01CA118699 awarded by the National Institutes of Health. The government has certain rights in the invention.

### FIELD OF THE INVENTION

Aspects of the present invention relate generally to colorectal cancer (CRC), and more particularly to methods and compositions (e.g., gene marker panels) for at least one of diagnosis, identification and classification of CRC. Further aspects relate to marker identification based on a comprehensive genome-scale analysis of aberrant DNA methylation and/or gene expression in CRC. Particular aspects relate to identification and/or classification of colorectal tumors, corresponding to distinctive DNA methylation-based subgroups of CRC including CpG island methylator phenotype (CIMP) groups and non-CIMP groups. Further aspects related to correlations of genetic mutation, and other epigenetic markers with said CRC subgroups for at least one of diagnosis, identification and classification of CRC including CIMP groups and non-CIMP groups.

### SEQUENCE LISTING

A Sequence Listing (in .txt format) comprising SEQ ID NOS:1-278 was filed as part of this application, and is incorporated by reference herein in its entirety.

### BACKGROUND

Colorectal cancer (CRC) arises through the accumulation of multiple genetic and epigenetic changes. Somatic mutations in APC, BRAF, KRAS, PIK3CA, TP53 and other genes have been frequently observed in CRC and are considered to be drivers of colorectal tumorigenesis (Wood et al., 2007). In addition, the majority of sporadic CRCs (65-70%) display chromosomal instability (CIN), characterized by aneuploidy, amplifications and deletions of subchromosomal genomic regions and loss of heterozygosity (LOH) (Pino and Chung, 2010).

Two major types of epigenetic modifications closely linked to CRC are DNA methylation and covalent histone modifications (Jones and Baylin, 2007). Aberrant DNA methylation of CpG islands has been reported in the earliest detectable lesions in the colonic mucosa, aberrant crypt foci (ACF) (Chan et al., 2002). Promoter CpG island DNA hypermethylation is associated with transcriptional gene silencing, and can cooperate with other genetic mechanisms to alter key signaling pathways critical to colorectal tumorigenesis (Baylin and Ohm, 2006). A recent large-scale comparison between genes mutated and hypermethylated in CRC revealed significant overlap between these two alterations (Chan et al., 2008). Importantly, DNA hypermethylation appeared to be the preferred mechanism when a gene can be inactivated by either mutation or promoter DNA hypermethylation.

New insights into the mechanisms and the role of CpG island hypermethylation in cancer have emerged from recent studies using integrated analyses of the two types of epigenetic modifications. We and other groups have reported that genes that are targeted by Polycomb group (PcG) proteins in embryonic stem (ES) cells are susceptible to cancer-specific DNA hypermethylation (Ohm et al., 2007; Schlesinger et al., 2007; Widschwendter et al., 2007). PcG target genes are characterized by trimethylation of histone H3 lysine 27 (H3K27me3), are maintained at a low expression state and are poised to be activated during development (Bernstein et al., 2007). More recently, it has been found that genes targeted by H3K27me3 in normal tissues acquire DNA methylation and lose the H3K27me3 mark in cancer (Gal-Yam et al., 2008; Rodriguez et al., 2008). Importantly, epigenetic switching of H3K27me3 and DNA methylation mainly occurs at genes that are not expressed in normal tissues. Furthermore, cancer-specific H3K27me3-mediated gene silencing has also been shown to inactivate tumor suppressor genes independent of DNA hypermethylation in CRC (Jiang et al., 2008; Kondo et al., 2008).

Colorectal tumors with a CpG island methylator phenotype (CIMP) exhibit a high frequency of cancer-specific DNA hypermethylation at a subset of genomic loci and are highly enriched for activating mutation of BRAF (BRAF^{V600E}) (Weisenberger et al., 2006). CRCs with CIN and CIMP have been shown to be inversely correlated (Goel et al., 2007; Cheng et al., 2008) and appear to develop in two separate pathways (Leggett and Whitehall, 2010). DNA hypermethylation of some CIMP-associated gene promoters have been detected in early stages of in colorectal tumorigenesis (Ibrahim et al., 2011). Furthermore, an extensive promoter DNA hypermethylation has been observed in the histologically normal colonic mucosa of patients predisposed to multiple serrated polyps, the proposed precursors of CIMP tumors (Young and Jass, 2006). Notably, some of the distinct genetic and histopathological characteristics associated with CIMP tumors may be directly attributable to CIMP-mediated gene silencing. Applicants have reported that CIMP-associated DNA hypermethylation of MLH1 is the dominant mechanism for the development of sporadic CRC with microsatellite instability (MSI) (Weisenberger et al., 2006). Furthermore, the CIMP-specific inactivation of IGFBP7-mediated senescence and apoptosis pathways may provide a permissive environment for the acquisition of BRAF mutations in CIMP-positive tumors (Hinoue et al., 2009; Suzuki et al., 2010).

Recent studies from several groups indicated that colorectal tumors with KRAS mutations may also be associated with a unique DNA methylation profile. CIMP-low (CIMP-L) tumors were originally shown to exhibit DNA hypermethylation of a reduced number of CIMP-defining loci (Ogino et al., 2006). CIMP-L was significantly associated with KRAS mutations, was observed more commonly in men than women and appeared to be independent of MSI status. Shen and colleagues described the CIMP2 subgroup, which also showed DNA hypermethylation of CIMP-associated loci, but was highly correlated (92%) to KRAS mutations and not associated with MSI (Shen et al., 2007). A recent report from Yagi, et al. reported the intermediate-methylation epigenotype (IME), which was also associated with KRAS mutations (Yagi et al., 2010).

In light of these findings, there is confusion in the art with regards to DNA methylation subtypes in CRC. It is not established whether CIMP-L, CIMP2 or IME represent unique DNA methylation-based subgroups in CRC, as limited numbers of genomic regions were used to derive membership in these studies. Moreover, the types of genes targeted for DNA methylation in each subgroup and the effects of DNA hypermethylation on gene expression in each subtype have not yet been fully explored.

### SUMMARY OF THE INVENTION

In particular aspects, four distinct DNA methylation subgroups were identified and characterized in CRC by performing comprehensive, genome-scale DNA methylation profiling of 125 primary colorectal tumors and 29 adjacent non-tumor colonic mucosa samples using the Illumina Infinium DNA methylation assay.

In certain aspects, Applicants developed diagnostic DNA methylation gene marker panels to identify CIMP (CIMP-H and CIMP-L), as well as to segregate CIMP-H tumors from CIMP-L tumors based on the Infinium DNA methylation data (FIG. 5).

In particular aspects, a CIMP-defining marker panel consisting of B3GAT2, FOXL2, KCNK13, RAB31 and SLIT1 was identified. Using the conditions that DNA methylation of three or more markers qualifies a sample as CIMP, this panel identifies CIMP-H and CIMP-L tumors with 100% sensitivity and 95.6% specificity with 2.4% misclassification using a β-value threshold of ≧0.1.

In particular aspects, a second marker panel of FAM78A, FSTL1, KCNC1, MYOCD, and SLC6A4 specifically identifies CIMP-H tumors with 100% sensitivity and 100% specificity (0% misclassification) using conditions that three or more markers show DNA methylation β-value threshold of ≧0.1.

In certain aspects, a tumor sample is classified as CIMP-H if both marker panels are positive (three or more markers with DNA methylation for each panel).

In further aspects, a tumor sample is classified as CIMP-L if the CIMP-defining marker panel is positive while the CIMP-H specific panel is negative (0-2 genes methylated).

Gene expression data was also obtained for paired tumor and adjacent normal samples in order to assess the biological implications of DNA methylation-mediated gene silencing in CRC.

Preferred Exemplary Embodiments.

Preferred aspects provide methods for at least one of diagnosing, detecting and classifying a colorectal cancer belonging to a distinct colorectal cancer (CRC) subgroup having frequent CpG island hypermethylation (CIMP CRC), comprising: determining, by analyzing a human subject biological sample comprising colorectal cancer (CRC) cell genomic DNA using a suitable assay, a CpG methylation status of at least one CpG dinucleotide from each gene of the gene marker panel of B3GAT2, FOXL2, KCNK13, RAB31 and SLIT1 (CIMP marker panel); wherein CpG hypermethylation, relative to normal control values, of at least three genes of the CIMP marker gene panel is indicative of a frequent CpG island hypermethylation colorectal cancer subgroup (CIMP CRC), and wherein a method of at least one of diagnosing, detecting and/or classifying a colorectal cancer belonging to the distinct colorectal cancer (CRC) subgroup having frequent CpG island hypermethylation (CIMP CRC) is afforded. In certain aspects, the CpG island hypermethylation colorectal cancer (CIMP CRC), comprises both CIMP-H and CIMP-L subgroups of CIMP. In particular embodiments, CIMP-H and CIMP-L tumors are identified with about 100% sensitivity and about 95.6% specificity with about 2.4% misclassification using conditions that three or more markers show DNA methylation β-value threshold of ≧0.1. as defined herein. In certain aspects of the methods disclosed herein, determining a CpG methylation status of at least one CpG dinucleotide from each gene of the gene marker panel of B3GAT2, FOXL2, KCNK13, RAB31 and SLIT1 (CIMP marker panel), comprises determining a CpG methylation status of at least one CpG dinucleotide from each of: at least one of SEQ ID NOS:45, 46 and 278 (B3GAT2 promoter, CpG island and amplicon, respectively); at least one of SEQ ID NOS:40, 41 and 240 (FOXL2 promoter, CpG island and amplicon, respectively); at least one of SEQ ID NOS:25, 26 and 224 (KCNK13 promoter, CpG island and amplicon, respectively); at least one of SEQ ID NOS:35, 36 and 236 (RAB31 promoter, CpG island and amplicon, respectively); and at least one of SEQ ID NOS:30, 31, 228 and 232 (SLIT1 promoter, CpG island and amplicons, respectively), respectively. Additional aspects further comprise determining, by analyzing the human subject biological using a suitable assay, a CpG methylation status of at least one CpG dinucleotide from each gene of an additional gene marker panel of FAM78A, FSTL1, KCNC1, MYOCD, and SLC6A4 (CIMP-H marker panel), wherein a CIMP-L subgroup of CIMP is indicated where the CIMP-defining marker panel is positive (hypermethylation of at least three genes of the CIMP marker gene panel) while the CIMP-H marker panel is negative (hypermethylation of only 0-2 genes of the CIMP-H marker gene panel), and wherein a CIMP-H subgroup of CIMP is indicated where both the CIMP-defining marker panel and the CIMP-H marker panel are positive (hypermethylation of at least three genes of each marker gene panel). In additional aspects, the methods further comprise determination of at least one of KRAS, BRAF and TP53 mutant status. In certain aspects, the BRAF mutation status comprises mutation status at codon 600 in exon 15 (e.g., BRAFV600E), wherein the KRAS mutation status comprises mutation status at codon 12 and/or 13 in exon 2, and wherein the TP53 mutation status comprises mutation status at exons 4 through 8. In certain aspects, a positive mutation status comprises at least one of missense mutations, nonsense mutations, splice-site mutations, frame-shift mutations, and in-frame deletions. Yet additional aspects further comprise determining a MLH1 gene methylation status, wherein MLH1 hypermethylation is strongly associated with CIMP-H CRC. In particular embodiments of the methods disclosed herein, determining a CpG methylation status of at least one CpG dinucleotide from each gene of the gene marker panel of FAM78A, FSTL1, KCNC1, MYOCD, and SLC6A4 (CIMP-H marker panel), comprises determining a CpG methylation status of at least one CpG dinucleotide from each of: at least one of SEQ ID NOS:50, 51 and 247 (FAM78A promoter, CpG island and amplicon, respectively); at least one of SEQ ID NOS:65, 66, 259, 263 and 265 (FSTL1 promoter, CpG island and amplicons, respectively); at least one of SEQ ID NOS:60, 61 and 255 (KCNC1 promoter, CpG island and amplicon, respectively); at least one of SEQ ID NOS:55, 56 and 251 (MYOCD promoter, CpG island and amplicon, respectively); and at least one of SEQ ID NOS:70, 71, and 269 (SLC6A4 promoter, CpG island and amplicons, respectively), respectively. In certain embodiments, determining methylation status comprises treating the genomic DNA, or a fragment thereof, with one or more reagents (e.g., bisulfite, hydrogen sulfite, disulfite, and combinations thereof) to convert cytosine bases that are unmethylated in the 5-position thereof to uracil or to another base that is detectably dissimilar to cytosine in terms of hybridization properties.

Yet further aspects provide methods for at least one of diagnosing, detecting and classifying a colorectal cancer belonging to a distinct colorectal cancer (CRC) subgroup having frequent CpG island hypermethylation (CIMP CRC), comprising: determining, by analyzing a human subject biological sample comprising colorectal cancer (CRC) cell genomic DNA using a suitable assay, a CpG methylation status of at least one CpG dinucleotide from each gene of the gene marker panel of FAM78A, FSTL1, KCNC1, MYOCD, and SLC6A4 (CIMP-H marker panel); wherein CpG hypermethylation, relative to normal control values, of at least three genes of the CIMP-H marker gene panel is indicative of a CIMP-H subgroup of CIMP CRC, and wherein a method of at least one of diagnosing, detecting and classifying a colorectal cancer belonging to the CIMP-H subgroup of CIMP CRC is afforded. In certain aspects, CIMP-H tumors are identified with about 100% sensitivity and about 100% specificity (about 0% misclassification) using conditions that three or more markers show DNA methylation β-value threshold of ≧0.1. as defined herein. Certain aspects, further comprise determination of at least one of KRAS, BRAF and TP53 mutant status. In certain aspects, the BRAF mutation status comprises mutation status at codon 600 in exon 15 (e.g., BRAFV600E), wherein the KRAS mutation status comprises mutation status at codon 12 and/or 13 in exon 2, and wherein the TP53 mutation status comprises mutation status at exons 4 through 8. In particular aspects, a positive mutation comprises at least one of missense mutations, nonsense mutations, splice-site mutations, frame-shift mutations, and in-frame deletions. Certain aspects further comprise determining a MLH1 gene methylation status, wherein MLH1 hypermethylation is strongly associated with CIMP-H CRC. In certain aspects of the methods disclosed herein, determining a CpG methylation status of at least one CpG dinucleotide from each gene of the gene marker panel of FAM78A, FSTL1, KCNC1, MYOCD, and SLC6A4 (CIMP-H marker panel), comprises determining a CpG methylation status of at least one CpG dinucleotide from each of: at least one of SEQ ID NOS:50, 51 and 247 (FAM78A promoter, CpG island and amplicon, respectively); at least one of SEQ ID NOS:65, 66, 259, 263 and 265 (FSTL1 promoter, CpG island and amplicons, respectively); at least one of SEQ ID NOS:60, 61 and 255 (KCNC1 promoter, CpG island and amplicon, respectively); at least one of SEQ ID NOS:55, 56 and 251 (MYOCD promoter, CpG island and amplicon, respectively); and at least one of SEQ ID NOS:70, 71, and 269 (SLC6A4 promoter, CpG island and amplicons, respectively), respectively. In particular embodiments, determining methylation status comprises treating the genomic DNA, or a fragment thereof, with one or more reagents (e.g., bisulfite, hydrogen sulfite, disulfite, and combinations thereof) to convert cytosine bases that are unmethylated in the 5-position thereof to uracil or to another base that is detectably dissimilar to cytosine in terms of hybridization properties.

Yet additional aspects, provide kits for performing the methods, comprising, for each gene of the gene marker panel of B3GAT2, FOXL2, KCNK13, RAB31 and SLIT1, at least two oligonucleotides whose sequences in each case are identical, are complementary, or hybridize under stringent or highly stringent conditions to the respective marker gene; and optionally comprising a bisulfite reagent (e.g., bisulfite, hydrogen sulfite, disulfite, and combinations thereof). In certain aspects of the kits disclosed herein, the respective marker gene sequences comprise at least one sequence from each of: at least one of SEQ ID NOS:45, 46 and 278 (B3GAT2 promoter, CpG island and amplicon, respectively); at least one of SEQ ID NOS:40, 41 and 240 (FOXL2 promoter, CpG island and amplicon, respectively); at least one of SEQ ID NOS:25, 26 and 224 (KCNK13 promoter, CpG island and amplicon, respectively); at least one of SEQ ID NOS:35, 36 and 236 (RAB31 promoter, CpG island and amplicon, respectively); and at least one of SEQ ID NOS:30, 31, 228 and 232 (SLIT1 promoter, CpG island and amplicons, respectively), respectively.

Further aspects provide kits suitable for performing the method comprising, for each gene of the gene marker panel of FAM78A, FSTL1, KCNC1, MYOCD, and SLC6A4, at least two oligonucleotides whose sequences in each case are identical, are complementary, or hybridize under stringent or highly stringent conditions to the respective marker gene; and optionally comprising a bisulfite reagent (e.g., bisulfite, hydrogen sulfite, disulfite, and combinations thereof). In certain aspects of the kits disclosed herein, the respective marker gene sequences comprise at least one sequence from each of: at least one of SEQ ID NOS:50, 51 and 247 (FAM78A promoter, CpG island and amplicon, respectively); at least one of SEQ ID NOS:65, 66, 259, 263 and 265 (FSTL1 promoter, CpG island and amplicons, respectively); at least one of SEQ ID NOS:60, 61 and 255 (KCNC1 promoter, CpG island and amplicon, respectively); at least one of SEQ ID NOS:55, 56 and 251 (MYOCD promoter, CpG island and amplicon, respectively); and at least one of SEQ ID NOS:70, 71, and 269 (SLC6A4 promoter, CpG island and amplicons, respectively), respectively.

The data presented and discussed in this specification have also been deposited in NCBI's Gene Expression Omnibus (GEO) and are accessible through GEO Series accession numbers GSE25062 and GSE25070, incorporated by reference herein. The following links have been created to review these records: http://www.ncbi.nlm.nih.gov/geo/query/acc.cgi?token=xpannsgssikcuvq&acc=GSE25062; and http://www.ncbi.nlm.nih.gov/geo/query/acc.cgi?token=rzgzzwyyqqqgklu&acc=GSE25070.

### BRIEF DESCRIPTION OF THE DRAWINGS

FIG. 1 shows, according to particular exemplary aspects, RPMM-based classification and heatmap representation of 1 25 colorectal tumor samples using Infinium DNA methylation data. DNA methylation profiles of 1,401 probes with most variable DNA methylation values (standard deviation>0.20) in the 125 colorectal tumor sample set are shown. The DNA methylation β-values are represented by using a color scale from dark blue (low DNA methylation) to yellow (high DNA methylation, which is herein reproduced in gray-scale). Four subgroups were derived by RPMM-based clustering and are indicated above the heatmap: lightsky blue, cluster 1 (n=28); lightcoral, cluster 2 (n=29); yellow, cluster 3 (n=37) and dark gray, cluster 4 (n=31), all of which colors are herein reproduced in gray-scale. CIMP-positive tumors as classified by the MethyLight five-marker panel (Weisenberger et al., 2006) are indicated by black bars. Presence of MLH1 DNA methylation, BRAF mutation, KRAS mutation, and TP53 mutations are indicated by orange, blue, red, and purple bars, respectively, herein reproduced in gray-scale. Probes that are located within CpG islands (Takai-Jones) are indicated by the horizontal black bars to the right of the heatmap. The probes are arranged based on the order of unsupervised hierarchal cluster analysis using a correlation distance metric and average linkage method. Pie charts below the heatmap show the proportion of tumor samples harboring BRAF mutations (blue), KRAS mutations (red), and those wild-type for both BRAF and KRAS (yellow-green), herein reproduced in grey-scale within each subgroup.

FIGS. 2A-C show, according to particular exemplary aspects, DNA methylation characteristics associated with CIMP-H, CIMP-L, BRAF- and KRAS-mutant colorectal tumors. (A) Comparison of CIMP-H- and CIMP-L-associated DNA methylation profiles. Each data point represents the log₁₀-transformed FDR-adjusted P-value comparing DNA methylation in CIMP-H (n=28) vs. non-CIMP tumors (n=68) (x-axis) and in CIMP-L (n=29) vs. non-CIMP tumors (n=68) (y-axis) for each Infinium DNA methylation probe. For the probes with higher mean DNA methylation in CIMP-H or CIMP-L tumors compared to non-CIMP tumors, −1 is multiplied to log₁₀(FDR-adjusted P-value), providing positive values. The blue and red points, herein reproduced in gray-scale, highlight probes that are significantly hypermethylated in CIMP-H and CIMP-L tumors compared to non-CIMP tumors, respectively. (B) Heatmap representing Infinium DNA methylation β-values for 575 CpG sites that are significantly hypermethylated in CIMP-H compared with non-CIMP tumors (top) and 22 CpG sites that are significantly hypermethylated in CIMP-L compared with non-CIMP tumors (bottom). The four DNA methylation-based subgroups are indicated above the heatmaps. A color gradient from dark blue to yellow, herein reproduced in gray-scale was used to represent the low and high DNA methylation β-values, respectively. (C) Comparison of BRAF mutant- and KRAS mutant-associated DNA hypermethylation signatures in CRC. The log₁₀-transformed FDR-adjusted P-value for each probe is plotted for tumors harboring KRAS mutations (KRAS-M) (n=34) vs. BRAF/KRAS wild-type (n=74) (y-axis) and those containing BRAF mutations (BRAF-M) (n=17) vs. BRAF/KRAS wild-type (n=74) (x-axis). For the probes with higher mean DNA methylation β-values in BRAF or KRAS mutant tumors compared to wild-type tumors, −1 is multiplied to log₁₀(FDR-adjusted P-value), providing positive values.

FIGS. 3A-D show, according to particular exemplary aspects and herein reproduced in gray-scale, that CIMP-L-associated DNA hypermethylation occurs independent of KRAS mutation status in CRC. CIMP-L and non-CIMP tumors were subdivided by their KRAS and BRAF mutation status (KRAS mutant or BRAF/KRAS wild-type), and mean DNA methylation β-values were compared between each group. Scatter plots comparing mean DNA methylation β-values between (A) KRAS mutant and BRAF/KRAS wild-type tumors within the CIMP-L subgroup, (B) KRAS mutant and BRAF/KRAS wild-type tumors within the non-CIMP subgroup, (C) KRAS mutant, CIMP-L tumors versus KRAS mutant, non-CIMP tumors and (D) BRAF/KRAS wild-type, CIMP-L tumors compared to non-CIMP tumors with the same genotype.

FIG. 4 shows, according to particular exemplary aspects and herein reproduced in gray-scale, ES-cell histone marks associated with genes in the five classification groups described in the text. Shown are heatmap representations of DNA methylation β-values for unique gene promoters that belong to five different categories: 1. CIMP-H specific: CIMP-associated DNA methylation markers specific for CIMP-H subgroup only (n=415 genes), 2. CIMP-H & CIMP-L: CIMP-specific DNA methylation shared between the CIMP-H and CIMP-L subgroups (n=73 genes), 3. Non-CIMP: cancer-specific DNA methylation but outside of the CIMP context (n=547 genes), 4. Constitutive-Low: Constitutively unmethylated genes in both tumor and adjacent normal tissue samples (n=500 genes), 5. Constitutive-High: Constitutively methylated in both tumor and adjacent normal tissue samples (n=500 genes). Genes containing CpG islands defined by Takai and Jones are indicated by horizontal black bars immediately to the right of each heatmap. The bar charts to the right of each heatmap show the proportion of gene promoters with occupancy of histone H3 lysine 4 trimethylation (K4) and/or histone H3 lysine 27 trimethylation (K27) in human ES cells. Probes that do not have these histone mark information (listed in Table 5 as “NA”) were not included in the bar chart calculations. The probes in each category are ordered according to the unsupervised hierarchal clustering using correlation distance metric and average linkage method. The RPMM-based cluster assignments are indicated above the heatmaps.

FIG. 5 shows, according to particular exemplary aspects, diagnostic CIMP-defining gene marker panels based on the Infinium DNA methylation data. The Dichotomous heat map of the Infinium DNA methylation data is shown. Black bars indicate DNA methylation β-value ≧0.1, and white bars indicate DNA methylation β-value<0.1. The panel of five markers shown on the top (CIMP-H & CIMP-L) is used to identify CIMP-H and CIMP-L tumors. The panel of five markers shown on the bottom (CIMP-H specific) is used to specifically identify CIMP-H tumors.

FIGS. 6A-C show, according to particular exemplary aspects, an integrated analysis of gene expression and promoter DNA methylation changes between colorectal tumors and matched normal adjacent tissues. (A) Mean DNA methylation β-value differences between CIMP-H tumors and matched normal colonic tissues (n=6) are plotted on the x-axis and mean log₂-transformed gene expression values differences are plotted on the y-axis for each gene. Red data points, herein reproduced in gray-scale, highlight those genes that are hypermethylated with β-value difference>0.20 and show more than 2-fold decrease in their gene expression levels in CIMP-H tumors. (B) Pie chart showing the gene expression changes of 1,534 hypermethylated genes in CIMP-H tumors compared with adjacent normal tissues. (C) Bar chart showing the number of genes that exhibit DNA hypermethylation and/or gene expression changes in non-CIMP tumors among the 112 genes that are hypermethylated and downregulated in CIMP-H tumors.

FIGS. 7A-D show, according to particular exemplary aspects and herein reproduced in gray-scale, (A) Delta area plot showing the relative change in area under the consensus cumulative distribution function (CDF) curve (Monti et al., 2003). (B) Consensus matrix produced by K-means clustering (K=4). (C) The heatmap representation of 125 colorectal tumor samples using the Infinium DNA methylation data as shown in FIG. 1. Cluster membership of each sample derived from RPMM-based clustering and consensus clustering are indicated as vertical bars with distinct colors above the heatmap (herein shown in gray-scale). (D) Contingency table comparing the cluster membership assignments between the two different clustering methods.

FIGS. 8A-B show, according to particular exemplary aspects, histogram analysis of the number of methylated CIMP-defining MethyLight-based markers in colorectal cancer samples. (A) Histogram analysis of the number of CIMP loci methylated in all 125 colorectal tumor samples. (B) Histogram analysis of the number of CIMP-defining loci methylated in each RPMM-based tumor cluster membership.

FIG. 9 shows, according to particular exemplary aspects, scatter plot analyses comparing DNA methylation profiles of colorectal tumor and adjacent-normal samples, stratified by their RPMM-based cluster membership.

FIGS. 10A-B show, according to particular exemplary aspects, a comparison of DNA methylation profiles between CIMP-H and CIMP-L tumors. (A) The volcano plot shows the −1×log₁₀-transformed FDR-adjusted P value vs. the mean DNA methylation difference between CIMP-H and CIMP-L tumors. FDR-adjusted P=0.001 and |Δβ|=0.2 are used as a cutoff for differential methylation. Two CpG sites that are hypermethylated in CIMP-L tumors compared with CIMP-H tumors are indicated in green, herein reproduced in gray-scale. (B) Heatmap representing Infinium DNA methylation β-values for the two CpG sites (labeled in green in panel A, herein reproduced in gray-scale) that are significantly hypermethylated in CIMP-L compared with CIMP-H tumors. The four DNA methylation-based subgroups are indicated above the heatmap. A color gradient from dark blue to yellow, herein reproduced in gray-scale was used to represent the low and high DNA methylation β-values, respectively.

FIGS. 11A-E show, according to particular exemplary aspects, DNA structural and sequence characteristics associated with five different gene categories based on DNA methylation profiles in colorectal tumors. The five categories include: 1, CIMP-associated DNA methylation markers specific for the CIMP-H subgroup only; 2, CIMP-specific DNA methylation shared between both the CIMP-H and CIMP-L subgroups; 3, non-CIMP cancer-specific DNA methylation; 4, constitutively unmethylated across tumor and adjacent normal tissue samples; 5, constitutively methylated across tumor and adjacent normal tissue samples. Distribution of (A) observed CpG/expected CpG ratio and (B) GC content over 250 bp upstream and 250 bp downstream from the interrogated CpG dinucleotide on the Infinium DNA methylation BeadArray, (C) the Takai and Jones-calculated CpG island length (Takai and Jones, 2002), (D, E) distances of Infinium DNA methylation probes to the nearest (D) ALU and (E) LINE repetitive element. In each box plot, the top and bottom edges are the 25th and 75th quartiles, respectively. The horizontal line within each box identifies the median. The whiskers above and below the box extend to at most 1.5 times the interquartile range (IQR).

FIGS. 12A-D show, according to particular exemplary aspects, validation of the Infinium DNA methylation data and gene expression array data using MethyLight and quantitative RT-PCR (qRT-PCR), respectively. The validations were performed for three genes indicated above each scatter plot (A) Comparison of Infinium DNA methylation β-value (x-axis) and log 2-transformed gene expression value from illumina expression array (y-axis). (B) Validation of Infinium DNA methylation data by MethyLight technology. The x-axis represents Infinium DNA methylation β-value and the y-axis represents PMR value from MethyLight assay. Pearson correlation coefficients between the assays: 0.85 for SFRP1, 0.91 for TMEFF2 and 0.96 for LMOD1. (C) Validation of alumina expression array data by qRT-PCR assay. The x-axis represents log 2-transformed array-based gene expression value and the y-axis represents log 2-transformed relative copy number normalized to HTPR1 using qRT-PCR assay. Pearson correlation coefficients between the gene expression platforms: 0.93 for SFRP1, 0.89 for TMEFF2 and 0.91 for LMOD1. (D) Comparison of MethyLight PMR values (x-axis) and log 2-transformed normalized relative copy number from qRT-PCR assay (y-axis). Black open circle: adjacent normal (n=25), red open circle (herein reproduced in gray-scale): tumors in CIMP-L, Cluster 3 and Cluster 4 (n=19), blue open circle (herein reproduced in gray-scale): CIMP-H tumors (n=6).

### DETAILED DESCRIPTION OF THE INVENTION

### Definitions

In particular aspects, “gene’ refers to the respective genomic DNA sequence, including any promoter and regulatory sequences of the gene (e.g., enhancers and other gene sequences involved in regulating expression of the gene), and in particular embodiments, portions of said gene. In certain embodiment a gene sequence may be an expressed sequence (e.g., expressed RNA, mRNA, cDNA). In particular aspects, the term “gene” shall be taken to include all transcript variants thereof (e.g., the term “B3GAT2” shall include for example its transcripts and any truncated transcript, etc) and all promoter and regulatory elements thereof. Furthermore where SNPs are known within genes the term shall be taken to include all sequence variants thereof.

In particular aspects, “promoter” or “gene promoter” refers to the respective contiguous gene DNA sequence extending from 1.5 kb upstream to 1.5 kb downstream relative to the transcription start site (TSS), or contiguous portions thereof. In particular aspects, “promoter” or “gene promoter” refers to the respective contiguous gene DNA sequence extending from 1.5 kb upstream to 0.5 kb downstream relative to the TSS. In certain aspects, “promoter” or “gene promoter” refers to the respective contiguous gene DNA sequence extending from 1.5 kb upstream to the downstream edge of a CpG island that overlaps with the region from 1.5 kb upstream to 1.5 kb downstream from TSS (and is such cases, my thus extend even further beyond 1.5 kb downstream), and contiguous portions thereof. In particular aspects, with respect to any particular recited gene, any CpG dinucleotide of the particular recited gene that is coordinately methylated with the “promoter” or “gene promoter” of said recited gene, has substantial diagnostic/classification utility as disclosed herein, as one of ordinary skill in the art could readily practice the disclosed invention using any such coordinately methylated CpG dinucleotide sequences.

In particular aspects, a “CpG” island (CGI) refers to the NCBI relaxed definition defined bioinformatically as DNA sequences (200 based window) with a GC base composition greater than 50% and a CpG observed/expected ratio [o/e] of more than 0.6 (Takai & Jones Proc. Natl Acad. Sci. USA 99:3740-3745, 2002; Takai & Jones In Silico Biol. 3:235-240, 2003; see also NCBI MapViewer help document describing relaxed vs strick definition of CpG islands at www.ncbi.nlm.nih.gov/projects/mapview/static/humansearch.html#cpg; all of which are incorporated by reference herein in their entirety). In particular aspects “CpG” island (CGI) refers to the more strick definition (Id).

“Stringent hybridisation conditions,” as defined herein, involve hybridising at 68° C. in 5×SSC/5×Denhardt's solution/1.0% SDS, and washing in O.2×SSC/O.1% SDS at room temperature, or involve the art-recognized equivalent thereof (e.g., conditions in which a hybridisation is carried out at 60° C. in 2.5×SSC buffer, followed by several washing steps at 37° C. in a low buffer concentration, and remains stable). Moderately stringent conditions, as defined herein, involve including washing in 3×SSC at 42° C., or the art-recognized equivalent thereof. The parameters of salt concentration and temperature can be varied to achieve the optimal level of identity between the probe and the target nucleic acid. Guidance regarding such conditions is available in the art, for example, by Sambrook et al. 1989, Molecular Cloning, A Laboratory Manual, Cold Spring Harbor Press, N.Y.; and Ausubel et al. (eds.), 1995, Current Protocols in Molecular Biology, (John Wiley & Sons, N.Y.; incorporated herein by reference) at Unit 2.10.

The term “methylation state” or “methylation status” refers to the presence or absence of 5-methylcytosine (“5-mCyt”) at one or a plurality of CpG dinucleotides within a DNA sequence. Methylation states at one or more particular CpG methylation sites (each having two CpG dinucleotide sequences) within a DNA sequence include “unmethylated,” “fully-methylated” and “hemi-methylated.”

The term “hemi-methylation” or “hemimethylation” refers to the methylation state of a double stranded DNA wherein only one strand thereof is methylated.

The term “hypermethylation” refers to the average methylation state corresponding to an increased presence of 5-mCyt at one or a plurality of CpG dinucleotides within a DNA sequence of a test DNA sample, relative to the amount of 5-mCyt found at corresponding CpG dinucleotides within a normal control DNA sample.

The term “hypomethylation” refers to the average methylation state corresponding to a decreased presence of 5-mCyt at one or a plurality of CpG dinucleotides within a DNA sequence of a test DNA sample, relative to the amount of 5-mCyt found at corresponding CpG dinucleotides within a normal control DNA sample.

The term “bisulfite reagent” refers to a reagent comprising bisulfite, disulfite, hydrogen sulfite or combinations thereof, useful as disclosed herein to distinguish between methylated and unmethylated CpG dinucleotide sequences.

The term “Methylation assay” refers to any assay for determining the methylation state of one or more CpG dinucleotide sequences within a sequence of DNA.

The term “MS.AP-PCR” (Methylation-Sensitive Arbitrarily-Primed Polymerase Chain Reaction) refers to the art-recognized technology that allows for a global scan of the genome using CG-rich primers to focus on the regions most likely to contain CpG dinucleotides, and described by Gonzalgo et al., Cancer Research 57:594-599, 1997.

The term “MethyLight™” refers to the art-recognized fluorescence-based real-time PCR technique described by Eads et al., Cancer Res. 59:2302-2306, 1999.

The term “HeavyMethyl™” assay, in the embodiment thereof implemented herein, refers to an assay, wherein methylation specific blocking probes (also referred to herein as blockers) covering CpG positions between, or covered by the amplification primers enable methylation-specific selective amplification of a nucleic acid sample.

The term “HeavyMethyl™ MethyLight™” assay, in the embodiment thereof implemented herein, refers to a HeavyMethyl™ MethyLight™ assay, which is a variation of the MethyLight™ assay, wherein the MethyLight™ assay is combined with methylation specific blocking probes covering CpG positions between the amplification primers.

The term “Ms-SNuPE” (Methylation-sensitive Single Nucleotide Primer Extension) refers to the art-recognized assay described by Gonzalgo & Jones, Nucleic Acids Res. 25:2529-2531, 1997.

The term “MSP” (Methylation-specific PCR) refers to the art-recognized methylation assay described by Herman et al. Proc. Natl. Acad. Sci. USA 93:9821-9826, 1996, and by U.S. Pat. No. 5,786,146.

The term “COBRA” (Combined Bisulfite Restriction Analysis) refers to the art-recognized methylation assay described by Xiong & Laird, Nucleic Acids Res. 25:2532-2534, 1997.

The term “MCA” (Methylated CpG Island Amplification) refers to the methylation assay described by Toyota et al., Cancer Res. 59:2307-12, 1999, and in WO 00/26401A1.

### Colorectal Cancer (CRC):

Colorectal cancer (CRC) is a heterogeneous disease in which unique subtypes are characterized by distinct genetic and epigenetic alterations. Comprehensive genome-scale DNA methylation profiling of 125 colorectal tumors and 29 adjacent normal tissues was performed, and four DNA methylation-based subgroups of CRC were identified using model-based cluster analyses. Each subtype shows characteristic genetic and clinical features, indicating that they represent biologically distinct subgroups.

In particular aspects, a CIMP-high (CIMP-H) subgroup, which exhibits an exceptionally high frequency of cancer-specific DNA hypermethylation, is strongly associated with MLH1 DNA hypermethylation and the BRAFV600E mutation.

In additional aspects, a CIMP-low (CIMP-L) subgroup is enriched for KRAS mutations and characterized by DNA hypermethylation of a subset of CIMP-H associated markers rather than a unique group of CpG islands.

In further aspects, non-CIMP tumors are separated into two distinct clusters. One non-CIMP subgroup is distinguished by a significantly higher frequency of TP53 mutations and frequent occurrence in the distal colon, while the tumors that belong to the fourth group exhibit a low frequency of both cancer-specific DNA hypermethylation and gene mutations, and are significantly enriched for rectal tumors.

In yet further aspects, 112 genes were identified that were downregulated more than 2-fold in CIMP-H tumors together with promoter DNA hypermethylation. These represent approximately 7% of genes that acquired promoter DNA methylation in CIMP-H tumors. Intriguingly, 48/112 genes were also transcriptionally silent in non-CIMP subgroups, but this was not attributable to promoter DNA hypermethylation.

In particular aspects, therefore, four distinct DNA methylation subgroups of CRC were identified, and provide novel insight regarding the role of CIMP-specific DNA hypermethylation in gene silencing.

CRC can be classified based on various molecular features. Identification and characterization of these subtypes has been not only essential to better understand the disease (Jass, 2007), but also valuable in selection of optimal drug treatments, prediction of patient survival, and discovery of risk factors linked to a particular subtype (Walther et al., 2009; Limsui et al., 2010). The Illumina Infinium DNA methylation assay was used herein to investigate DNA methylation-based subgroups in CRC. This BeadArray platform interrogates the gene promoter DNA methylation of all 14,495 consensus coding DNA sequence (CCDS) genes in multiple samples simultaneously and is therefore suitable for a study requiring large-scale promoter DNA methylation profiling of a large number of samples (Bibikova, 2009). Using this platform, four DNA methylation subgroups of CRC were identified herein, based on model-based unsupervised cluster analyses. Importantly, the genetic and clinical correlations observed with each subtype indicate that they represent biologically distinct subgroups.

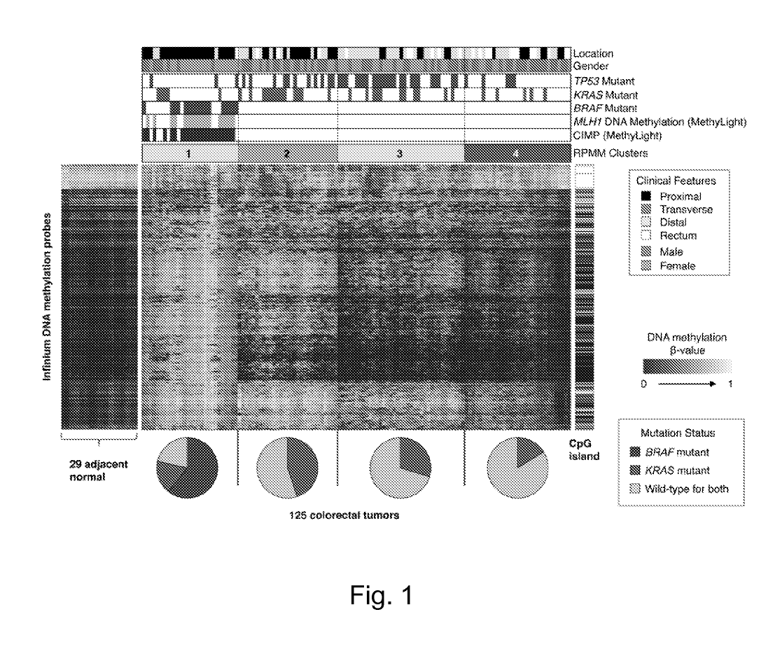

One subgroup, designated here as CIMP-H, contained all of the CIMP-positive tumors characterized by the MethyLight five-marker panel (i.e., CACNA1G, IGF2, NEUROG1, RUNX3, SOCS1)) previously developed in Applicants' laboratory (Weisenberger et al., 2006) (see also FIG. 1 herein). Other features associated with the CIMP-H subgroup we described here are in agreement with those observed in the CIMP1 subtype (Shen et al., 2007) and the high-methylation epigenotype (HME) (Yagi et al., 2010) described previously.

Six CIMP-H tumors were identified herein, based on the Infinium DNA methylation data, that did not meet the criteria for CIMP using the MethyLight five-gene panel. The MethyLight-based marker panel was developed based on the screening of 195 MethyLight markers (Weisenberger et al., 2006). In the current study, Applicants measured DNA methylation at a much larger number of loci using the Illumina Infinium DNA methylation platform (27,578 CpG sites located at 14,495 gene promoters). According to particular aspects, the additional loci present on the array more accurately identified CIMP tumors, compared to the conventional MethyLight-based five-marker panel. This increased accuracy is likely a reflection of both the inclusion of additional markers which are more tightly associated with CIMP, and the mere fact that a larger number of informative loci will usually outperform a small panel of informative loci. The limited MethyLight panel was designed to be particularly compatible with cost-effective processing of large numbers of formalin-fixed, paraffin-embedded (FFPE) samples, and evertheless, the five-marker CIMP panel has been found to be very useful in large-scale studies of FFPE samples. However, any small panel of markers will likely have some misclassification error in identifying a complex molecular profile, regardless of the composition of the panel.

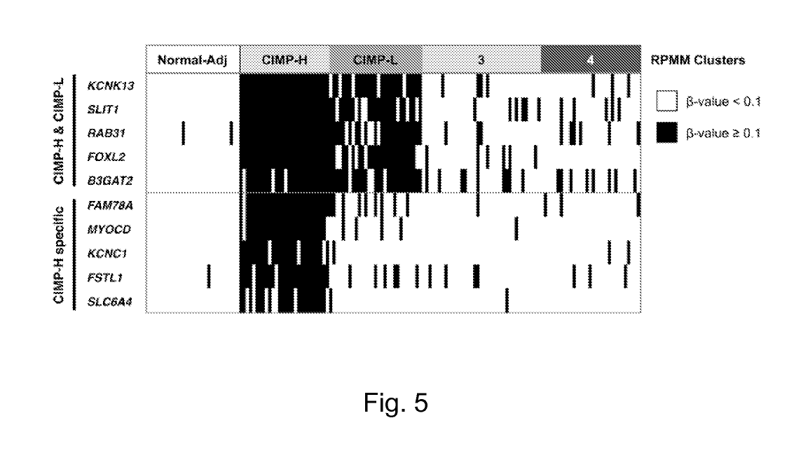

According to particular aspects, the instant results provide new diagnostic DNA methylation marker panels to identify CIMP (CIMP-H and CIMP-L), as well as to segregate CIMP-H tumors from CIMP-L tumors (see EXAMPLE 6, and FIG. 5 herein).

FIG. 5 shows, according to particular exemplary aspects, diagnostic CIMP-defining gene marker panels based on the Infinium DNA methylation data. The Dichotomous heat map of the Infinium DNA methylation data is shown. Black bars indicate DNA methylation β-value ≧0.1, and white bars indicate DNA methylation β-value<0.1. The panel of five markers shown on the top (CIMP-H & CIMP-L) is used to identify CIMP-H and CIMP-L tumors. The panel of five markers shown on the bottom (CIMP-H specific) is used to specifically identify CIMP-H tumors.

Ogino and colleagues proposed the CIMP-low subgroup, which showed DNA hypermethylation of CIMP-defining markers despite at a low frequency and enrichment for KRAS mutations (Ogino et al., 2006). Applicants herein identified the CIMP-L subgroup through a genome-scale approach and provided a comprehensive DNA methylation profile of these tumors. Importantly, the CIMP-L-associated DNA methylation appears to occur only at a subset of CIMP-H-associated sites, as Applicants did not find evidence for strong CIMP-L-specific DNA methylation at a unique set of CpG sites. Moreover, Applicants found that although KRAS mutations are enriched in CIMP-L tumors, this subtype may not be driven by KRAS mutations, since DNA hypermethylation profiles in KRAS wild-type and mutant tumors within CIMP-L tumors were highly correlated across the CpG sites we examined. The independence of KRAS mutations from CIMP-L status suggests that a more complex molecular signature exists in driving CIMP-L DNA methylation profiles. Recently, Applicants and others have hypothesized that BRAF mutations might be favorably selected in the specific environment that CIMP creates (Hinoue et al., 2009; Suzuki et al., 2010). Similar mechanisms may also result in the enrichment of KRAS mutations in the CIMP-L subgroup.

Shen and colleagues (Shen et al., 2007) reported the CIMP2 subset, along with CIMP1 (CIMP-H) and non-CIMP subsets of CRC, using a 28-gene panel. They found a very strong association of CIMP2 with KRAS mutations (92%), together with DNA hypermethylation of several CIMP-H-associated loci. The CIMP2 subgroup may be similar to the CIMP-L subgroup we identified in our study. However, the present Applicants only detected a KRAS mutation frequency of approximately 50% in CIMP-L tumors. The differences in KRAS mutation frequencies between Applicants' CIMP-L and CIMP2 of Shen et al. likely arise from differences in the CRC patient collections and in the genomic features and technologies used to analyze DNA methylation subgroups of CRC in both studies.

Applicants did not find a statistically significant association of MGMT DNA hypermethylation and CIMP-L status. However, Ogino and colleagues reported statistical significance in their recent report (Ogino et al., 2007). The differences between the instant results and those of Ogino and colleagues may arise from several sources. First, Ogino and colleagues used a different criterion for classifying CIMP-L tumors. Specifically, they classified a tumor sample as CIMP-L if one or two markers from the MethyLight-based CIMP panel showed DNA methylation. By contrast, Applicants' CIMP-L classification was based on Infinium DNA methylation data, a more robust resource of CIMP-L gene markers. Additionally, possible disparities in the CRC sample collections between the studies, such as ethnic population differences, may contribute to CIMP-L classification differences. Finally, there are differences in sample sizes between both studies, which may also contribute to statistical evaluation of CIMP in both collections of CRC tumors.

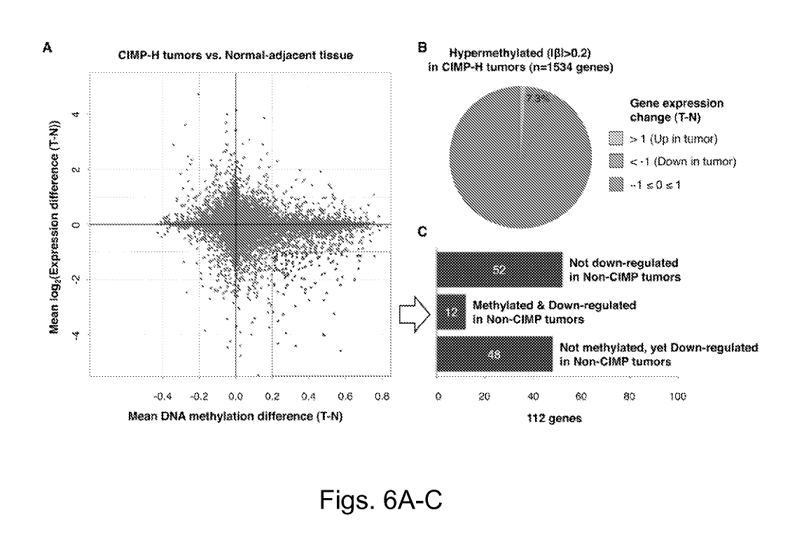

In particular aspects, Applicants also obtained gene expression profiles in pairs of CIMP-H and non-CIMP tumor-normal adjacent tissues to gain insight into the role of CIMP-specific DNA hypermethylation in colorectal tumorigenesis. Aberrant DNA methylation of promoter CpG islands has been established as an important mechanism that inactivates tumor suppressor genes in cancer (Jones and Baylin, 2007). However, many cancer-specific CpG island hypermethylation events are also found in promoter regions of genes that are not normally expressed, and these may represent “passenger” events that do not have functional consequences (Widschwendter et al., 2007; Gal-Yam et al., 2008). In additional aspects, therefore, Applicants examined effects of CIMP-associated DNA hypermethylation on gene expression, and determined found that only 7.3% of the CIMP-H-specific DNA methylation markers showed a strong inverse relationship with their gene expression levels (see EXAMPLE 7, and FIGS. 6A-C herein). Similar observations have been made in the glioma-CpG island methylator phenotype (G-CIMP) (Noushmehr et al., 2010). Although a larger sample size is required for better estimates, the present Applicants' observations might reinforce the hypothesis that CIMP represents a broad epigenetic control defect that accompanies a large number of “passenger” DNA hypermethylation events (Weisenberger et al., 2006).

In particular aspects, 112 genes were identified herein that showed both promoter DNA hypermethylation and reduction in gene expression in CIMP-H tumors (see EXAMPLE 7, and FIGS. 6A-C herein). Importantly, 12 of these genes were found to also show DNA hypermethylation with concomitant reduction in gene expression level in non-CIMP tumors, indicating that aberrant DNA methylation and transcriptional silencing of these genes may be important in the development of CRC, irrespective of molecular subtype. Intriguingly, these include SFRP1 and SFRP2, which function as negative regulators of Wnt signaling. DNA hypermethylation of SFRP genes has been observed in the majority of aberrant crypt foci (ACFs) and tumorigenesis (Baylin and Ohm, 2006). DNA hypermethylation and transcriptional silencing of other genes such as TMEFF2 and SLIT3 have also been reported (Young et al., 2001; Dickinson et al., 2004). However, the functional significance of the inactivation of these genes has not been established in CRC.

In yet further aspects, Applicants observed that of the 112 genes that exhibited DNA hypermethylation and reduced gene expression in CIMP-H tumors, 48 were also silenced in non-CIMP tumors, but without substantial increases in DNA methylation. CIMP status in CRC has been found to be inversely correlated with the occurrence of chromosomal instability (CIN), which is characterized by aneuploidy, gain and loss of subchromosomal genomic regions and high frequencies of loss of heterozygosity (LOH) (Goel et al., 2007; Cheng et al., 2008). Recently, Chan and colleagues identified genes that are inactivated by both genetic mechanisms (mutation or deletion) and DNA hypermethylation in breast and colorectal cancer (Chan et al., 2008). They observed that these genetic and epigenetic changes are generally mutually exclusive in a given tumor, and that silencing of these genes was associated with poor clinical outcome (Chan et al., 2008). Together, these genes may act as key tumor suppressor genes in CRC and the gene silencing mechanisms can be determined by the underlying molecular pathways involved in colorectal tumorigenesis.

The molecular mechanisms that account for CIMP have not been identified. It has been proposed that CIMP arises through a distinct pathway originating in a variant of hyperplastic polyps and sessile serrated adenomas due to the similar histological and molecular features shared by the CIMP tumors and these lesions (O'Brien, 2007). Some individuals and families with hyperplastic polyposis syndrome have an increased risk of developing CIMP CRC, indicating the existence of a genetic predisposition that could lead to CIMP (Young et al., 2007). Environmental exposures might also influence the risk of developing CIMP CRC. Cigarette smoking was found to be associated with increased risk of developing CIMP CRC in a recent report (Limsui et al., 2010)

Applicant's present sturdy provides the most comprehensive genome-scale analysis of DNA methylation-based subgroups of CRC to date. In particular aspects, the unique DNA methylation profiles in CRC, together with genomic changes, provide a detailed molecular landscape of colorectal tumors. According to particular aspects, the findings have substantial clinical utility for identification and diagnosis of colorectal cancer, as well as for determining particular treatments for CRC patients.

### Example 1

### Methods

Primary Colorectal Tissue Sample Collection and Processing.

Twenty-five paired colorectal tumor and histologically normal adjacent colonic tissue samples were obtained from colorectal cancer patients who underwent surgical resection at the department of surgery in the Groene Hart Hospital, Gouda, The Netherlands. Tissue samples were stored at −80° C. within one hour after resection. Tissue sections from the surgical resection margin were examined by a pathologist (C. M. van Dijk) by microscopic observation. All patients provided written informed consent for the collection of samples and subsequent analysis. The study was approved by the Institutional Review Board of the Groene Hart Hospital in Gouda and the Leiden University Medical Center and University of Southern California. An additional collection of 100 fresh-frozen colorectal tumor samples and four matched histologically normal-adjacent colonic mucosa tissue samples were obtained from the Ontario Tumor Bank Network (The Ontario Institute for Cancer Research, Ontario, Canada). The tissue collection and analyses were approved by the University of Southern California Institutional Review Board. Genomic DNA and total RNA were extracted simultaneously from the same tissue sample using the TRIZOL®Reagent (Invitrogen, Burlington, ON) according to the manufacturer's protocol.

Mutation Analysis.

BRAF (NM_{—}004333.4; GI:187608632) mutations at codon 600 in exon 15 and KRAS (NG_{—}007524.1; GI:17686616) mutations at codons 12 and 13 in exon 2 were identified using the pyrosequencing assay. Specifically, a 224 bp fragment of the BRAF gene containing exon 15 was amplified from genomic DNA using the following primers: 5′ TCA TAA TGC TTG CTC TGA TAG GA 3′ (SEQ ID NO:1) and 5′Biotin-GGC CAA AAA TTT AAT CAG TGG A 3′(SEQ ID NO:2), and genotyped with the sequencing primer 5′ CCA CTC CAT CGA GAT T 3′ (SEQ ID NO:3). Similarly, a 214 bp fragment of the KRAS gene containing exon 2 was amplified from each genomic DNA sample using the following primers: 5′Biotin-GTG TGA CAT GTT CTA ATA TAG TCA 3′ (SEQ ID NO:4) and 5′ GAA TGG TCC TGC ACC AGT AA 3′ (SEQ ID NO:5), and genotyped with the sequencing primer 5′ GCA CTC TTG CCT ACG 3′ (SEQ ID NO:6).

Mutations in TP53 exons 4 through 8 were determined by direct sequencing of PCR products. Specifically, TP53 exons 4 through 8 were amplified by PCR using three exon-specific primer sets: Exon 4, 5′-GTT CTG GTA AGG ACA AGG GTT-3′ (forward) (SEQ ID NO:7) and 5′-CCA GGC ATT GAA GTC TCA TG-3′ (reverse) (SEQ ID NO:8) (Tm=49° C.); Exons 5 and 6, 5′-GGT TGC AGG AGG TGC TTA C-3′ (forward) (SEQ ID NO:9) and 5′-CCA CTG ACA ACC ACC CTT AAC-3′ (reverse) (SEQ ID NO:10) (Tm=51° C.); Exons 7 and 8, 5′-CCT GCT TGC CAC AGG TCT C-3′ (forward) (SEQ ID NO:11) and 5′-TGA ATC TGA GGC ATA ACT GCA C-3′ (reverse) (SEQ ID NO:12) (Tm=51° C.). PCR amplification was performed using a touchdown protocol with an initial step of 95° C. for 12 minutes, then 5 cycles of 95° C. for 25 sec, Tm+15° C. for 1 min and 72° C. for 1 min, then 5 cycles of 95° C. for 25 sec, Tm+10° C. for 1 min and 72° C. for 1 min, followed by 5 cycles of 95° C. for 25 sec, Tm+5° C. for 1 min and 72° C. for 1 min, finishing with 35 cycles of 95° C. for 25 sec, Tm° C. for 1 min and 72° C. for 1 min.

Sequencing of the purified PCR products was performed using an ABI PRISM BigDye Terminator Cycle Sequencing Ready Reaction Kit (Applied Biosystems, Foster City, Calif.). Cycle sequencing reactions were performed in a thermal cycler for 25 cycles at 96° C. for 10 sec, annealing at 50° C. for 5 sec, and extension at 60° C. for 4 min. Prior to capillary electrophoresis, unincorporated dye terminators were removed from the extension product using a DyeEx 96 Plate (Qiagen, Valencia, Calif.) according to the manufacturer's instructions. The purified extension products were denatured at 90° C. for 2 min and placed on ice for 1 min. Sequencing was performed on an ABI PRISM 3730×1 DNA Analyzer (Applied Biosystems). The sequencing output files (.ab1) were processed using the Phred/Phrap software package developed at the University of Washington (Nickerson et al., 1997; Ewing and Green, 1998; Ewing et al., 1998; Gordon et al., 1998). Sequence Alignments for each exon read were viewed in the Consed Viewer Software and sequence variations were annotated and recorded.

Samples containing missense mutations, nonsense mutations, splice-site mutations, frame-shift mutations, and in-frame deletions were considered positive for a mutation.

DNA Methylation Assays.

For MethyLight-based assays, genomic DNAs were treated with sodium bisulfite using the Zymo EZ DNA Methylation Kit (Zymo Research, Orange, Calif.) and subsequently analyzed by MethyLight as previously described (Campan et al., 2009; incorporated herein by reference it its entirety). The primer and probe sequences for the MethyLight reactions for the five-gene CIMP marker panel and MLH1 were reported previously (Weisenberger et al., 2006; incorporated herein by reference in its entirety). The results of the MethyLight assays were scored as PMR (Percent of Methylated Reference) values as previously defined, with a PMR of ≧10 was used as a threshold for positive DNA methylation in each sample (Weisenberger et al., 2006; Campan et al., 2009). A sample was scored as CIMP-positive if ≧3 of the five CIMP-defining markers gave PMR values≧10.

The Illumina Infinium HumanMethylation27 DNA methylation assay technology has been described previously (Bibikova, 2009; incorporated herein by reference in its entirety). Briefly, genomic DNA was bisulfite converted using the EZ-96 DNA Methylation Kit (Zymo Research) according to the manufacturer's instructions. The amount of bisulfite converted DNA and completeness of bisulfite conversion was assessed using a panel of MethyLight-based quality control (QC) reactions as previously described (Campan et al., 2009). All of the samples in this study passed Applicants' QC tests and entered into the Infinium DNA methylation assay pipeline. The Infinium DNA methylation assay was performed at the USC Epigenome Center according to the manufacturer's specifications (Illumina, San Diego, Calif.). The Illumina Infinium DNA methylation assay examines DNA methylation status of 27,578 CpG sites located at promoter regions of 14,495 protein-coding genes and 110 microRNAs. A measure of the level of DNA methylation at each CpG site is scored as beta (β) values ranging from 0 to 1, with values close to 0 indicating low levels of DNA methylation and close to 1 high levels of DNA methylation (Bibikova, 2009). The detection P values measure the difference of the signal intensities at the interrogated CpG site compared to those from a set of 16 negative control probes embedded in the assay. All data points with a detection P value >0.05 were identified as not statistically significantly different from background measurements, and therefore not trustworthy measures of DNA methylation. These data points were replaced by “NA” values as previously described (Noushmehr et al., 2010). More specifically, for the Illumina Infinium DNA methylation data analysis, data points were masked as “NA” for probes that might be unreliable (see the Supplemental Methods). All data points with a detection P value >0.05 were identified and replaced by “NA” values. Finally, probes that are designed for sequences on either the X- or Y-chromosome were excluded. DNA methylation data sets which did not contain any “NA”-masked data points were analyzed. DNA methylation βvalues were normalized to eliminate the batch effects. Briefly, the batch means of β-values were brought closer to the overall mean while retaining the original range of DNA methylation data (0 to 1) (Pan et al., manuscript in preparation). Only the tumor samples were used to calculate the batch means and overall mean in estimating the scaling factor for each batch. For the gene expression analysis, unreliable probes (9%), as described by Barbosa-Morais et al., were removed from the subsequent analysis (Barbosa-Morais et al., 2010). Data point were masked as “NA” for probes that contained single-nucleotide polymorphisms (SNPs) (dbSNP NCBI build 130/hg18) within the five base pairs from the interrogated CpG site or that overlap with a repetitive element that covers the targeted CpG dinucleotide. Furthermore, data points were replaced with “NA” for probes that are not uniquely aligned to the human genome (NCBI build 36/hg18) at 20 nucleotides at the 3′ terminus of the probe sequence, and those that overlap with regions of insertions and deletions in the human genome. Together, data points for 4,484 probes were masked. The assay probe sequences and detailed information on each interrogated CpG site and the associated genomic characteristics on the HumanMethylation27 BeadChip can be obtained at www.illumina.com, and these data are incorporated herein by reference in their entirety. All Infinium DNA methylation data are available at the NCBI Gene Expression Omnibus (http://www.ncbi.nlm.nih.gov/geo/) under accession number GSE25062, and these data are incorporated herein by reference in their entirety.

Validation of Infinium DNA Methylation Data by MethyLight Assay.

Genomic DNA from 25 pairs of colorectal tumor and their adjacent normal samples were treated with sodium bisulfite using the Zymo EZ96 DNA Methylation Kit (Zymo Research) and subsequently analyzed by MethyLight as previously described (Campan et al., 2009). Primers and probes used for validation are as follows and are listed as 5′ to 3′: SFRP1, forward primer: 5′ GAA TTC GTT CGC GAG GGA 3′ (SEQ ID NO:13), reverse primer: 5′ AAA CGA ACC GCA CTC GTT ACC 3′ (SEQ ID NO:14), probe: 6FAM-CCG TCA CCG ACG CGA AAA CCA AT-BHQ-1 (SEQ ID NO:15); TMEFF2, forward primer: 5′ GTT AAA TTC GCG TAT GAT TTC GAG A 3′ (SEQ ID NO:16), reverse primer: 5′ TTC CCG CGT CTC CGA C 3′ (SEQ ID NO:17), probe: 6FAM-AAC GAA CGA CCC TCT CGC TCC GAA-BHQ-1 (SEQ ID NO:18); LMOD1, forward primer: 5′ TTT TAA AGA TAA GGG GTT ACG TAA TGA G 3′ (SEQ ID NO:19), reverse primer: 5′ CCG AAC TAA CGA ATT CAC CGA C 3′ (SEQ ID NO:20), probe: 6FAM-TCG TCC CTA CTT ATC TAA CTC TCC GTA-MGBNFQ (SEQ ID NO:21). The results of the MethyLight assays were scored as PMR (Percent of Methylated Reference) values as previously defined (Weisenberger et al., 2006; Campan et al., 2009).

Gene Expression Assay.

Gene expression assay was performed on 25 pairs of colorectal tumor and non-tumor adjacent tissue samples using the Illumina Ref-8 whole-genome expression BeadChip (HumanRef-8 v3.0, 24,526 transcripts) (Illumina). Scanned image and bead-level data processing were performed using the BeadStudio 3.0.1 software (Illumina). The summarized data for each bead type were then processed using the lumi package in Bioconductor (Du et al., 2008). The data were log₂transformed and normalized using Robust Spline Normalization (RSN) as implemented in the lumi package. Specifically, total RNA from 26 pairs of colorectal tumor and non-tumor adjacent tissue samples was isolated using the TRIZOL® Reagent (Invitrogen, Burlington, ON) according to the manufacturer's protocol. The concentrations of RNA samples were measured using the NanoDrop 8000 (Thermo Fisher Scientific, Waltham, Mass.). The quality of the RNA samples was assessed using the Experion RNA StdSens analysis kit (Bio-Rad, Hercules, Calif.). Expression analysis was performed using the Illumina Ref-8-whole-genome expression BeadChip (HumanRef-8 v3.0, 24,526 transcripts) (Illumina, San Diego, Calif.). Briefly, RNA samples were processed using the Illumina TotalPrep RNA Amplification Kit (Illumina). Total RNA (500 ng) from each sample was subject to reverse transcription with an oligo(dT) primer bearing a T7 promoter. The cDNA then underwent second strand synthesis and purification. Biotinylated cRNA was then generated from the double-stranded cDNA template through in vitro transcription with T7 RNA polymerase. The biotinylated cRNA (750 ng) from each patient was then hybridized to the BeadChips. The hybridized chips were stained and scanned using the Illumina HD BeadArray scanner (Illumina). Scanned image and bead-level data processing were performed using the BeadStudio 3.0.1 software (Illumina). The summarized probe profile data and processed expression data are available at the NCBI Gene Expression Omnibus (http://www.ncbi.nlm.nih.gov/geo/) under accession number GSE25070, and these data are incorporated herein by reference in their entirety.

Validation of the Illumina Gene Expression Array Data by Quantitative RT-PCR Assay.

Total RNA sample from 25 pairs of colorectal tumor and non-tumor adjacent tissue samples were treated with DNase using DNA-free™ kit (Applied Biosystems) to remove contaminating DNA. Reverse transcription reaction was performed using iScript Reverse Transcription Supermix for RT-PCR (Bio-Rad). Quantitative RT-PCR assays were performed with primers and probes obtained from Applied Biosystems (SFRPJ: Hs00610060_m1_M; TMEFF2: Hs00249367_m1_M; LMOD1: Hs00201704_m1_M). The raw expression values were normalized to those of HPRT1 (Hs99999909_m1_M).

Unsupervised Clustering.

Recursively partitioned mixture model (RPMM) was used for the identification of colorectal tumor subgroups based on the Illumina Infinium DNA methylation data. RPMM is a model-based unsupervised clustering approach developed for beta-distributed DNA methylation measurements that lie between 0 and 1 and implemented as RPMM Bioconductor package (Houseman et al., 2008). Probes were identified that do not contain any “NA”-masked data points and then RPMM clustering was performed on 2,758 probes (ten percent of original probes) that showed the most variable DNA methylation levels across the colorectal tumor panel. A fanny algorithm (a fuzzy clustering algorithm) was used for initialization and level-weighted version of Bayesian information criterion (BIC) as a split criterion for an existing cluster as implemented in the R-based RPMM package. The logit (logistic) transformation was applied to DNA methylation β-values and each probe was median-centered across the tumor samples. Consensus clustering was then performed using the same 2,728 Infinium DNA methylation probes that were used for RPMM-based clustering. The optimal number of clusters was assessed based on 1,000 re-sampling iterations (seed value: 1022) of K-means clustering for K=2,3,4,5,6 with Pearson correlation as the distance metric as implemented in the R/Bioconductor ConsensusClusterPlus package.

Statistical Analysis and Visualization.

Statistical analysis and data visualization were carried out using the R/Biocoductor software packages (http://www.bioconductor.org). The Wilcoxon Rank Sum test and the Wilcoxon Signed Rank test were used to evaluate the difference in DNA methylation β-value for each probe between two independent groups and between tumor and matched adjacent normal tissues, respectively. False-discovery rate (FDR) adjusted P values for multiple comparisons were calculated using Benjamini and Hochberg approach. The Illumina Infinium DNA methylation βvalues were represented graphically using a heatmap, generated by the R/Bioconductor packages βplots and Heatplus. Ordering of the samples within a RPMM class in the heatmaps was obtained by using the function “seriate” in the seriation package.

Classification and Selection of Cancer-Specific DNA Methylation Markers.

Gene promoters that exhibited cancer-specific DNA methylation were categorized into three groups. Four hundred fifteen (415) unique gene promoters were selected that showed significant CIMP-H-specific DNA hypermethylation (FDR-adjusted P<0.0001 for CIMP-H vs. non-CIMP tumors and P>0.05 for CIMP-L vs. non-CIMP tumors), and seventy three (73) gene promoters were selected that showed DNA hypermethylation in both CIMP-H and CIMP-L tumors (FDR-adjusted P<0.0001 for CIMP-H vs. non-CIMP and CIMP-L vs. non-CIMP). For the third category, five hundred forty seven (547) genes were identified that acquired cancer-specific DNA hypermethylation irrespective of CIMP status (FDR-adjusted P<0.00001 for 29 paired tumor vs. adjacent non-tumor tissue). The genes are listed in Table 4. (Supplemental Table 4 for a list of genes).

Identification of Diagnostic CIMP-Associated DNA Methylation Gene Marker Panels.

The top 20 Infinium DNA methylation probes that are significantly hypermethylated in CIMP (CIMP-H and CIMP-L) compared with non-CIMP tumors based on the Wilcoxon rank-sum test were first selected. Using the conditions that DNA methylation β-value≧0.1 of three or more markers qualifies a sample as CIMP, a five-probe panel was determined that best classify CIMP (CIMP-H and CIMP-L) by calculating sensitivity and specificity, and overall misclassification rate for each random combination of the top 20 probes. For the CIMP-H-specific marker panel, top 20 probes were first selected that are significantly hypermethylated in CIMP-H compared with CIMP-L tumors. A five-marker panel was then chosen that showed the best sensitivity and specificity, and overall misclassification rate to classify CIMP-H using the conditions that three or more markers show DNA methylation β-value threshold of ≧0.1.

Integrated Analyses of the Illumina Infinium DNA Methylation and Gene Expression Data.

One probe was selected for each gene that showed the highest absolute mean β-value difference between tumor and normal-adjacent samples. The DNA methylation was then merged with the gene expression data set using Entrez Gene IDs using the R merge function. Expression data points with a detection P value >0.01, computed by BeadStudio software, were considered as not distinguishable from the negative control measurements, and therefore not expressed. A mean β-value difference (|Δβ|) of 0.20 was used as a threshold for differential DNA methylation. This threshold of |Δβ|=0.20 was determined previously as a stringent estimate of Δβ detection sensitivity across the range of β-values (Bibikova, 2009).

### Example 2

### DNA Methylation-Based Colorectal Cancer Classification was Established; Four Distinct Tumor Subgroups were Identified

Comprehensive genome-scale DNA methylation profiling of 125 colorectal tumor samples and 29 histologically normal-adjacent colonic tissue samples was performed using the Illumina Infinium DNA methylation assay, which assesses the DNA methylation status of 27,578 CpG sites located at the promoter regions of 14,495 protein-coding genes (Bibikova, 2009) (see working Example 1 above for more details). The mutation status of the BRAF, KRAS, and TP53 genes was also identified in the tumor samples. CRC subtypes were first determined based on DNA methylation profiles in the collection of 125 tumor samples. Probes that might be unreliable (see the Supplemental Methods section) and probes that are designed for sequences on either the X- or Y-chromosome were excluded. The top ten percent of probes with the highest DNA methylation variability based on standard deviation of the DNA methylation β-value across the entire colorectal tumor panel (2,758 probes) was selected, and then unsupervised clustering was performed using a recursively partitioned mixture model (RPMM). RPMM is a model-based unsupervised clustering method specifically developed for beta-distributed DNA methylation data such as obtained on the Infinium DNA methylation assay platform (Houseman et al., 2008). We identified four distinct tumor subgroups were identified by this approach, and designated as clusters 1, 2, 3 and 4 (FIG. 1). FIG. 1 shows, according to particular exemplary aspects, RPMM-based classification and heatmap representation of 125 colorectal tumor samples using Infinium DNA methylation data. DNA methylation profiles of 1,401 probes with most variable DNA methylation values (standard deviation>0.20) in the 125 colorectal tumor sample set are shown. The DNA methylation β-values are represented by using in a color scale from dark blue (low DNA methylation) to yellow (high DNA methylation), herein reproduced in gray-scale. Four subgroups were derived by RPMM-based clustering and are indicated above the heatmap: lightsky blue, cluster 1 (n=28); lightcoral, cluster 2 (n=29); yellow, cluster 3 (n=37) and dark gray, cluster 4 (n=31), herein reproduced in gray-scale. CIMP-positive tumors as classified by the MethyLight five-marker panel (Weisenberger et al., 2006) are indicated by black bars. Presence of MLH1 DNA methylation, BRAF mutation, KRAS mutation, and TP53 mutations are indicated by orange, blue, red, and purple bars, respectively, herein reproduced in gray-scale. Probes that are located within CpG islands (Takai-Jones) are indicated by the horizontal black bars to the right of the heatmap. The probes are arranged based on the order of unsupervised hierarchal cluster analysis using a correlation distance metric and average linkage method. Pie charts below the heatmap show the proportion of tumor samples harboring BRAF mutations (blue), KRAS mutations (red), and those wild-type for both BRAF and KRAS (yellow-green) within each subgroup, herein reproduced in gray-scale.

Genetic and clinical features of each cluster are summarized in Table 1 below.

TABLE 1
Genetic and clinical features found in each
of the four DNA methylation-based subtypes

| | | | | Cluster 1 | | Cluster 2 | | | | | |
|---|---|---|---|---|---|---|---|---|---|---|---|
| | | Overall | | (CIMP-H) | | (CIMP-L) | | Cluster 3 | | Cluster 4 | |
| Variable | | n | % | n | % | n | % | n | % | n | % |
| Total | | 125 | 100 | 28 | 22 | 29 | 23 | 37 | 30 | 31 | 25 |
| Gender | Female | 65 | 52 | 20 | 71 | 12 | 41 | 22 | 59 | 11 | 35 |
| | Male | 60 | 48 | 8 | 29 | 17 | 59 | 15 | 41 | 20 | 65 |
| Subsite | Proximal | 54 | 43 | 24 | 86 | 15 | 52 | 7 | 19 | 8 | 26 |
| | Transverse | 7 | 6 | 1 | 4 | 1 | 3 | 2 | 5 | 3 | 10 |
| | Distal | 49 | 39 | 3 | 11 | 11 | 38 | 24 | 65 | 11 | 36 |
| | Rectum | 15 | 12 | 0 | 0 | 2 | 7 | 4 | 11 | 9 | 29 |
| Stage | 1 or 2 | 50 | 50 | 9 | 41 | 16 | 66 | 12 | 41 | 13 | 52 |
| | 3 or 4 | 50 | 50 | 13 | 59 | 8 | 34 | 17 | 59 | 12 | 48 |
| | No info | 25 | | | | | | | | | |
| BRAF | Mutant | 17 | 14 | 17 | 61 | 0 | 0 | 0 | 0 | 0 | 0 |
| mutation | Wild-type | 108 | 86 | 11 | 39 | 29 | 100 | 37 | 100 | 31 | 100 |
| KRAS | Mutant | 34 | 27 | 5 | 18 | 13 | 45 | 11 | 30 | 5 | 16 |
| mutation | Wild-type | 91 | 73 | 23 | 82 | 16 | 55 | 26 | 70 | 26 | 84 |
| TP53 | Mutant | 43 | 34 | 3 | 11 | 11 | 38 | 24 | 65 | 5 | 16 |
| mutation | Wild-type | 82 | 66 | 25 | 89 | 18 | 62 | 13 | 35 | 26 | 84 |
| Age | Median | 68 | | 71 | | 70 | | 65 | | 69 | |
| | Range | 33-90 | | 51-90 | | 33-87 | | 44-88 | | 34-87 | |
| | No info | 25 | | | | | | | | | |

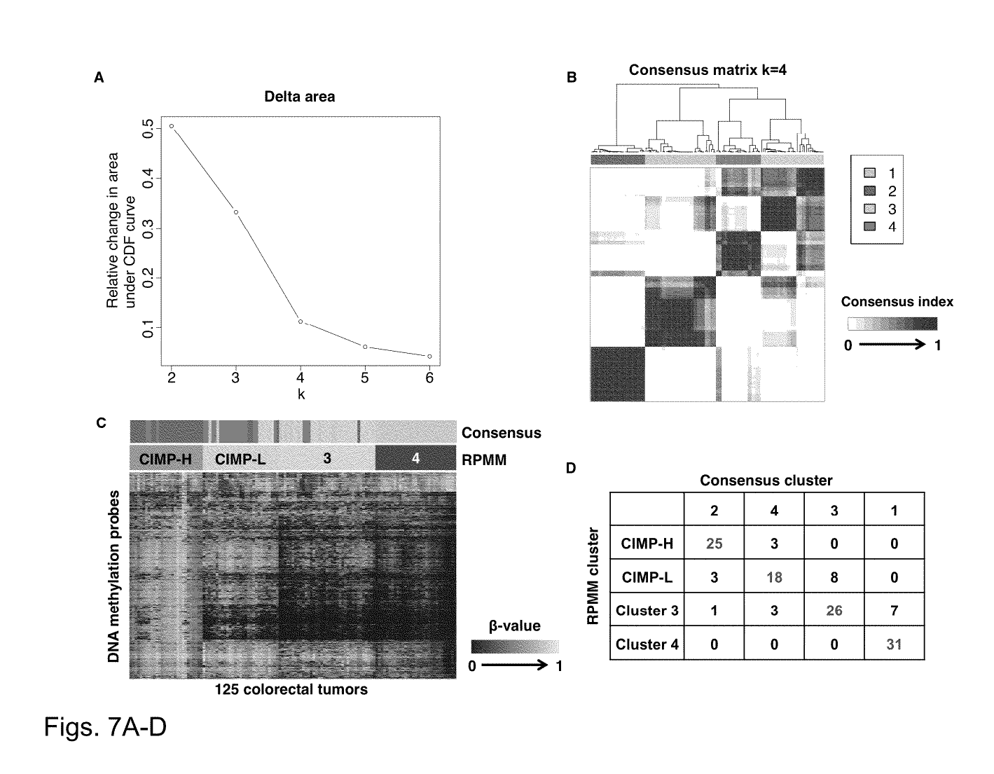

For comparison, resampling-based unsupervised consensus clustering (Monti et al., 2003) of the DNA methylation data set was also performed, and four DNA methylation based clusters were also identified using this method. The DNA methylation consensus cluster assignments for each sample were compared to their RPMM-based cluster assignments and substantial overlap was found with 80% (100/125) of the tumors showing agreement in cluster membership calls between these two different clustering methods (FIGS. 7A-D). FIGS. 7A-D show, according to particular exemplary aspects, (A) Delta area plot showing the relative change in area under the consensus cumulative distribution function (CDF) curve (Monti et al., 2003). (B) Consensus matrix produced by K-means clustering (K=4). (C) The heatmap representation of 125 colorectal tumor samples using the Infinium DNA methylation data as shown in FIG. 1. Cluster membership of each sample derived from RPMM-based clustering and consensus clustering are indicated as vertical bars with distinct colors above the heatmap (herein reproduced in gray-scale). (D) Contingency table comparing the cluster membership assignments between the two different clustering methods.

Subsequent analyses were based on cluster membership derived from RPMM-based unsupervised clustering method, which is particularly well-suited for beta-distributed DNA measurements, and has successfully identified DNA methylation profiles that are clinically relevant in normal and tumor samples from diverse tissues types (e.g., Christensen et al., 2009a; Christensen et al., 2009b; Marsit et al., 2009; Christensen et al., 2010; Christensen et al., 2011; Marsit et al., 2011).

The cluster 1 subgroup is enriched for CIMP-positive colorectal tumors, as determined by the CIMP-specific MethyLight five-marker panel developed previously in Applicants' laboratory (CACNA1G, IGF2, NEUROG1, RUNX3, SOCS1) (Weisenberger et al., 2006), as well as MLH1 DNA hypermethylation using MethyLight technology (see FIG. 1 herein). All of the tumors with BRAF mutation belong to this subgroup, and nearly half of the tumors in this subgroup that do not harbor BRAF mutations carry mutant KRAS (FIG. 1). The cluster 1 subgroup is also characterized by a low frequency of TP53 mutations (11%). Clinically, the majority of these tumors were found in female patients (71%) and have a proximal location in the colon (86%), both of which characteristics have been previously found to be associated with CIMP-positive CRC defined by the MethyLight fivemarker panel (Weisenberger et al., 2006).

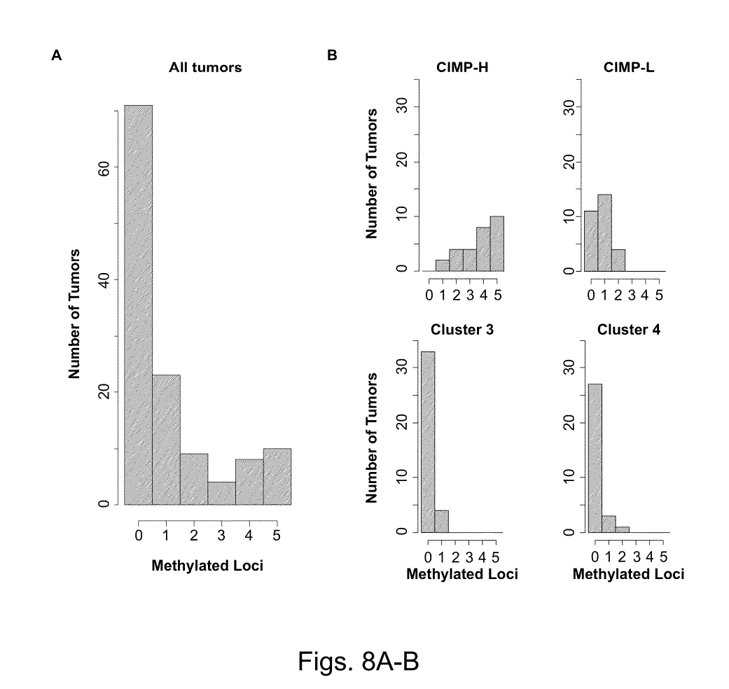

Previous studies with a limited number of DNA methylation markers from several groups indicated the existence of additional DNA methylation-based subtypes in CRC which are associated with KRAS mutations. These subgroups have been variously described as CIMP-low (Ogino et al., 2006), CIMP2 (Shen et al., 2007), and Intermediate-methylation epigenotype (IME) (Yagi et al., 2010). It is not clear whether these classifications represent the same tumor subgroup or different subgroups within CRC. We found that although KRAS mutant tumors are represented across the four classes, they are more common in the cluster 2 subgroup compared to the other clusters (FIG. 1 and Table 1). Interestingly, the proportion of the tumors that show DNA methylation at one or two loci of the MethyLight-based five-marker panel is substantially higher in the cluster 2 subgroup (62%) than in the cluster 3 (11%) or cluster 4 tumors (13%) (FIGS. 8A-B). FIGS. 8A-B show, according to particular exemplary aspects, histogram analysis of the number of methylated CIMP-defining MethyLight-based markers in colorectal cancer samples. (A) Histogram analysis of the number of CIMP (e.g., CIMP-defining) loci methylated in all 125 colorectal tumor samples. (B) Histogram analysis of the number of CIMP-defining loci methylated in each RPMM-based tumor cluster membership.

These genetic and epigenetic characteristics observed in the cluster 2 subgroup are consistent with the CIMP-low subtype described previously (Ogino et al., 2006). Therefore, in this study, we refer to the tumors that belong to the cluster 1 subgroup as CIMP-high (CIMP-H) and the cluster 2 subgroup tumors as CIMP-low (CIMP-L).

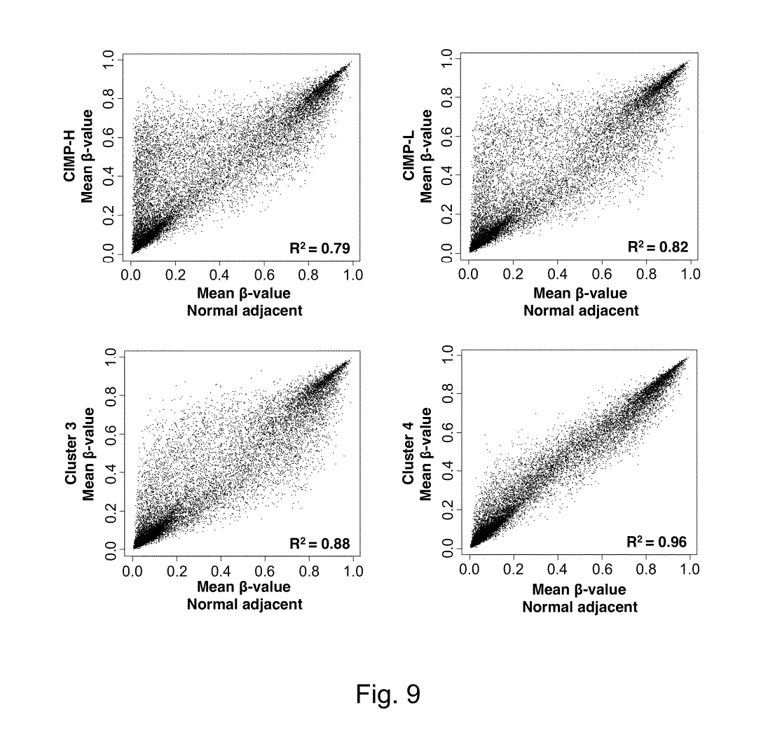

Applicants' RPMM-based clustering analysis identified two other CRC subtypes, designated as clusters 3 and 4, in addition to the CIMP-H and CIMP-L subgroups (FIG. 1 and Table 1). The difference between these two subgroups is not apparent based on DNA hypermethylation at the CIMP-defining five-gene loci (FIG. 8), indicating that DNA methylation signatures unrelated to CIMP might discriminate between these two CRC subsets. The frequency and level of cancer-specific DNA hypermethylation in the tumors in cluster 4 subgroup appear to be the lowest among the four subclasses (FIG. 9). FIG. 9 shows, according to particular exemplary aspects, scatter plot analyses comparing DNA methylation profiles of colorectal tumor and adjacent-normal samples, stratified by their RPMM-based cluster membership.

Importantly, the tumors included in cluster 3 are distinguished by a significantly higher frequency of TP53 mutations (65%) [P=6.5×10−5 (vs. cluster 4), Fisher's exact test] and their location in the distal colon (65%) [P=0.028 (vs. cluster 4), Fisher's exact test]. In contrast, the tumors that belong to cluster 4 exhibit a lower frequency of both KRAS (16%) and TP53 (16%) mutations, and their occurrence shows significant enrichment in the rectum compared to all the other groups (P=2.1×10−3, Fisher's exact test). Cluster 4 tumors also show borderline statistical significance to be more commonly found in males compared to the cluster 3 tumors (P=0.056, Fisher's exact test), providing additional lines of evidence that cluster 3 and 4 tumors are distinct.

A panel of 119 gene promoters was also identified that are constitutively methylated in normal samples, but show variable levels of DNA methylation in tumors (FIG. 1, and see Table 2 for the list of genes). It has long been established that the human genome is comprised primarily of sequences of low CpG density which are usually highly methylated in normal somatic tissues, and which undergo loss of DNA methylation in cancer (Feinberg and Vogelstein, 1983; Gama-Sosa et al., 1983; Miranda and Jones, 2007). Applicants found that indeed the majority of these probes are targeted to low-CpG density regions. The variable loss of DNA methylation among Applicants' tumor clusters is consistent with earlier reports that the degree of global DNA hypomethylation can vary considerably among colorectal tumors (Estecio et al., 2007). A gene set enrichment analysis (GSEA) was performed herein on these 119 genes using The Database for Annotation, Visualization and Integrated Discovery tool (DAVID). Applicants found enrichment of genes involved in secretion (3.1-fold enrichment, P=1.9×10₋₆), signaling (2.2-fold enrichment, P=6.8×10₋₆), signal peptide (2.2-fold enrichment, P=2.5×10₋₅), disulfide bond (2.3-fold enrichment, P=1.8×10₋₅) and extracellular regions (2.3-fold enrichment, P=6.8×10₋₄).

TABLE 2
Genes that are constitutively methylated in normal samples,
but show variable levels of DNA hypomethylation in tumors

| | | | | GC Content | Observed CpG/ | Mean | |
|---|---|---|---|---|---|---|---|
| | | | | over 250 bp | expected CpG | beta- | Standard |
| | | | | upstream | ratio over 250 bp | value | Deviation |
| | | | Chromo- | and 250 bp | upstream and 250 | adjacent | Adjacent |
| Illumin_ID | Symbol | Gene_ID | some | downstream | bp downstream | normal | normal |
| cg24240626 | REG3A | 5068 | 2 | 0.52 | 0.09 | 0.70 | 0.08 |
| cg19728382 | STC2 | 8614 | 5 | 0.53 | 0.61 | 0.46 | 0.16 |
| cg22718139 | HMGCS2 | 3158 | 1 | 0.51 | 0.28 | 0.50 | 0.15 |
| cg26153642 | HTR3E | 285242 | 3 | 0.53 | 0.35 | 0.48 | 0.13 |
| cg26777475 | PCOLCE | 5118 | 7 | 0.64 | 0.3 | 0.64 | 0.07 |
| cg23640701 | ACVRL1 | 94 | 12 | 0.64 | 0.61 | 0.43 | 0.08 |
| cg15914863 | CYP2W1 | 54905 | 7 | 0.69 | 0.29 | 0.47 | 0.09 |
| cg19890739 | GINS2 | 51659 | 16 | 0.57 | 0.44 | 0.41 | 0.09 |
| cg26970800 | GIF | 2694 | 11 | 0.47 | 0.18 | 0.78 | 0.10 |
| cg17741572 | CFB | 629 | 6 | 0.58 | 0.29 | 0.82 | 0.08 |
| cg19524009 | NEK3 | 4752 | 13 | 0.4 | 0.46 | 0.51 | 0.14 |
| cg17044311 | ABCC2 | 1244 | 10 | 0.35 | 0.27 | 0.81 | 0.07 |
| cg21820890 | PLA2G12B | 84647 | 10 | 0.53 | 0.28 | 0.92 | 0.09 |
| cg26628847 | PIP | 5304 | 7 | 0.52 | 0.09 | 0.64 | 0.07 |
| cg22241124 | CNGA3 | 1261 | 2 | 0.49 | 0.27 | 0.77 | 0.05 |
| cg22268164 | TRHR | 7201 | 8 | 0.46 | 0.24 | 0.77 | 0.05 |
| cg12188416 | TP63 | 8626 | 3 | 0.43 | 0.31 | 0.56 | 0.09 |
| cg15320474 | UBD | 10537 | 6 | 0.43 | 0.32 | 0.75 | 0.08 |
| cg01053621 | APOA2 | 336 | 1 | 0.47 | 0.18 | 0.74 | 0.07 |
| cg01430430 | SRRM3 | 222183 | 7 | 0.56 | 0.41 | 0.54 | 0.12 |
| cg10968815 | BPIL1 | 80341 | 20 | 0.59 | 0.14 | 0.75 | 0.04 |
| cg13320683 | RHOBTB1 | 9886 | 10 | 0.5 | 0.55 | 0.72 | 0.06 |
| cg12958813 | ATP6V1G3 | 127124 | 1 | 0.42 | 0.09 | 0.81 | 0.07 |
| cg03483654 | DAK | 26007 | 11 | 0.54 | 0.3 | 0.96 | 0.10 |
| cg06277277 | NR1I3 | 9970 | 1 | 0.49 | 0.17 | 0.66 | 0.09 |
| cg11871280 | SLC16A7 | 9194 | 12 | 0.38 | 0.17 | 0.81 | 0.07 |
| cg05187322 | CARD14 | 79092 | 17 | 0.54 | 0.56 | 0.63 | 0.16 |
| cg04968426 | PPP1R14D | 54866 | 15 | 0.58 | 0.19 | 0.50 | 0.16 |
| cg10321723 | PDZK1 | 5174 | 1 | 0.51 | 0.18 | 0.57 | 0.14 |
| cg11518240 | FKBP4 | 2288 | 12 | 0.5 | 0.86 | 0.88 | 0.06 |
| cg12582008 | OLFM4 | 10562 | 13 | 0.47 | 0.33 | 0.63 | 0.13 |
| cg06806711 | MS4A1 | 931 | 11 | 0.45 | 0.12 | 0.69 | 0.07 |
| cg07703337 | ZNF610 | 162963 | 19 | 0.48 | 0.21 | 0.84 | 0.06 |
| cg10037068 | WIPF1 | 7456 | 2 | 0.5 | 0.09 | 0.85 | 0.03 |
| cg11003133 | AIM2 | 9447 | 1 | 0.48 | 0.21 | 0.64 | 0.13 |
| cg24765446 | WFDC6 | 140870 | 20 | 0.52 | 0.12 | 0.80 | 0.05 |
| cg10379687 | SPINLW1 | 57119 | 20 | 0.51 | 0.12 | 0.67 | 0.05 |
| cg14662172 | CPB2 | 1361 | 13 | 0.42 | 0.18 | 0.82 | 0.03 |
| cg27609819 | PLCL1 | 5334 | 2 | 0.44 | 0.12 | 0.83 | 0.05 |
| cg18678121 | SEC61A2 | 55176 | 10 | 0.51 | 0.88 | 0.75 | 0.12 |
| cg25683185 | ACRBP | 84519 | 12 | 0.62 | 0.58 | 0.61 | 0.10 |
| cg14141399 | HAS1 | 3036 | 19 | 0.61 | 0.37 | 0.69 | 0.12 |
| cg27592318 | HEMGN | 55363 | 9 | 0.41 | 0.16 | 0.89 | 0.03 |
| cg17829936 | TAAR5 | 9038 | 6 | 0.5 | 0.26 | 0.72 | 0.10 |
| cg21660392 | ABCA8 | 10351 | 17 | 0.37 | 0.12 | 0.70 | 0.05 |
| cg14258236 | OR5V1 | 81696 | 6 | 0.44 | 0.17 | 0.86 | 0.04 |
| cg22983092 | KRT25 | 147183 | 17 | 0.49 | 0.23 | 0.91 | 0.03 |
| cg13675849 | TRPV5 | 56302 | 7 | 0.53 | 0.31 | 0.89 | 0.03 |
| cg21122774 | SARDH | 1757 | 9 | 0.6 | 0.23 | 0.72 | 0.10 |
| cg19241311 | DEFB123 | 245936 | 20 | 0.55 | 0.19 | 0.68 | 0.05 |
| cg26390526 | FLG | 2312 | 1 | 0.41 | 0.27 | 0.86 | 0.05 |
| cg18982568 | KRT77 | 374454 | 12 | 0.53 | 0.18 | 0.75 | 0.06 |
| cg25995212 | SCN7A | 6332 | 2 | 0.44 | 0.12 | 0.84 | 0.05 |
| cg23984130 | IGKV7-3 | 28905 | 2 | 0.48 | 0.11 | 0.83 | 0.04 |
| cg14826683 | SPRR2D | 6703 | 1 | 0.45 | 0.12 | 0.75 | 0.06 |
| cg20312687 | DEFB118 | 117285 | 20 | 0.5 | 0.1 | 0.79 | 0.04 |
| cg18152517 | STRA8 | 346673 | 7 | 0.5 | 0.03 | 0.84 | 0.03 |
| cg17423978 | SIRPD | 128646 | 20 | 0.46 | 0.35 | 0.81 | 0.05 |
| cg20556988 | CCL1 | 6346 | 17 | 0.55 | 0.11 | 0.83 | 0.03 |
| cg01910481 | PLUNC | 51297 | 20 | 0.46 | 0.07 | 0.88 | 0.05 |
| cg06531741 | HTR3B | 9177 | 11 | 0.44 | 0.17 | 0.81 | 0.08 |
| cg12718562 | TBC1D21 | 161514 | 15 | 0.49 | 0.17 | 0.83 | 0.04 |
| cg11009736 | MARCO | 8685 | 2 | 0.52 | 0.21 | 0.62 | 0.06 |
| cg00079056 | SPINK4 | 27290 | 9 | 0.46 | 0.43 | 0.84 | 0.04 |
| cg06275635 | PGLYRP3 | 114771 | 1 | 0.44 | 0.09 | 0.76 | 0.04 |
| cg08332212 | MLN | 4295 | 6 | 0.47 | 0.3 | 0.78 | 0.05 |
| cg10539808 | KCTD1 | 284252 | 18 | 0.5 | 0.22 | 0.72 | 0.05 |
| cg10784090 | CLDN18 | 51208 | 3 | 0.52 | 0.39 | 0.87 | 0.04 |
| cg01796228 | LIFR | 3977 | 5 | 0.5 | 0.58 | 0.75 | 0.05 |
| cg09440243 | PTPRD | 5789 | 9 | 0.41 | 0.39 | 0.80 | 0.04 |
| cg10054857 | C18orf20 | 221241 | 18 | 0.44 | 0.25 | 0.76 | 0.06 |
| cg03109316 | ZNF80 | 7634 | 3 | 0.56 | 0.44 | 0.90 | 0.02 |
| cg08947964 | GJA10 | 84694 | 6 | 0.36 | 0.26 | 0.83 | 0.06 |
| cg05241571 | KRTDAP | 388533 | 19 | 0.57 | 0.2 | 0.91 | 0.02 |
| cg03167883 | FLJ46365 | 401459 | 8 | 0.51 | 0.19 | 0.69 | 0.08 |
| cg07950803 | CD1A | 909 | 1 | 0.37 | 0.13 | 0.81 | 0.05 |
| cg00463848 | KRT2 | 3849 | 12 | 0.49 | 0.13 | 0.84 | 0.05 |
| cg09458237 | HSPA12B | 116835 | 20 | 0.52 | 0.18 | 0.72 | 0.06 |
| cg06501070 | LPAR3 | 23566 | 1 | 0.42 | 0.23 | 0.71 | 0.07 |
| cg01497576 | SLC24A5 | 283652 | 15 | 0.55 | 0.19 | 0.76 | 0.04 |
| cg12682367 | FLJ46358 | 400110 | 13 | 0.56 | 0.2 | 0.71 | 0.06 |
| cg03731898 | CPO | 130749 | 2 | 0.43 | 0.13 | 0.80 | 0.04 |
| cg08786003 | FCRL3 | 115352 | 1 | 0.41 | 0.24 | 0.68 | 0.10 |
| cg12878228 | PRSS1 | 5644 | 7 | 0.51 | 0.19 | 0.76 | 0.04 |
| cg01446692 | CER1 | 9350 | 9 | 0.38 | 0.22 | 0.70 | 0.06 |
| cg02786019 | TRPV6 | 55503 | 7 | 0.52 | 0.27 | 0.62 | 0.08 |
| cg10057218 | GSDMB | 55876 | 17 | 0.5 | 0.2 | 0.80 | 0.06 |
| cg04457794 | CTSE | 1510 | 1 | 0.57 | 0.27 | 0.60 | 0.12 |
| cg05109049 | EVI2B | 2124 | 17 | 0.32 | 0.24 | 0.48 | 0.14 |
| cg11783497 | IL1RN | 3557 | 2 | 0.54 | 0.19 | 0.68 | 0.13 |
| cg19099213 | SPP2 | 6694 | 2 | 0.39 | 0.11 | 0.84 | 0.06 |
| cg23067535 | FAM83A | 84985 | 8 | 0.65 | 0.44 | 0.62 | 0.14 |
| cg22442090 | GIMAP5 | 55340 | 7 | 0.52 | 0.21 | 0.72 | 0.04 |
| cg26718420 | C12orf59 | 120939 | 12 | 0.41 | 0.24 | 0.80 | 0.07 |
| cg17030820 | MSMB | 4477 | 10 | 0.49 | 0.3 | 0.84 | 0.08 |
| cg17827767 | LRIT1 | 26103 | 10 | 0.63 | 0.23 | 0.82 | 0.05 |
| cg18959422 | MYBPH | 4608 | 1 | 0.6 | 0.22 | 0.61 | 0.06 |
| cg20227165 | PRDM11 | 56981 | 11 | 0.57 | 0.15 | 0.69 | 0.05 |
| cg17778867 | KRTAP10-8 | 386681 | 21 | 0.6 | 0.14 | 0.70 | 0.11 |
| cg15075718 | MFRP | 83552 | 11 | 0.59 | 0.16 | 0.75 | 0.06 |
| cg21044104 | LYZL4 | 131375 | 3 | 0.46 | 0.16 | 0.67 | 0.08 |
| cg20383064 | BFSP2 | 8419 | 3 | 0.49 | 0.1 | 0.86 | 0.05 |
| cg24490338 | TPM3 | 7170 | 1 | 0.48 | 0.07 | 0.83 | 0.04 |
| cg17761453 | LOR | 4014 | 1 | 0.47 | 0.07 | 0.79 | 0.07 |
| cg18848394 | KRT38 | 8687 | 17 | 0.42 | 0.18 | 0.72 | 0.06 |
| cg02034222 | DQX1 | 165545 | 2 | 0.49 | 0.24 | 0.56 | 0.14 |
| cg14401837 | NPSR1 | 387129 | 7 | 0.45 | 0.27 | 0.52 | 0.14 |
| cg18738906 | SCNN1A | 6337 | 12 | 0.62 | 0.49 | 0.61 | 0.15 |
| cg24607535 | CDH26 | 60437 | 20 | 0.44 | 0.21 | 0.63 | 0.11 |
| cg00808492 | REG4 | 83998 | 1 | 0.4 | 0.36 | 0.50 | 0.14 |
| cg21682902 | HAL | 3034 | 12 | 0.4 | 0.2 | 0.75 | 0.13 |
| cg14898779 | STK31 | 56164 | 7 | 0.51 | 0.84 | 0.76 | 0.10 |
| cg22213042 | CPA2 | 1358 | 7 | 0.41 | 0.09 | 0.43 | 0.15 |
| cg14934821 | GPSM1 | 26086 | 9 | 0.65 | 0.44 | 0.70 | 0.17 |
| cg15021292 | PIK3R1 | 5295 | 5 | 0.49 | 0.29 | 0.50 | 0.14 |
| cg21906716 | TP73 | 7161 | 1 | 0.55 | 0.59 | 0.68 | 0.12 |
| cg17003970 | CHFR | 55743 | 12 | 0.56 | 0.73 | 0.78 | 0.21 |
| cg04117029 | UROS | 7390 | 10 | 0.41 | 0.24 | 0.61 | 0.14 |
| cg20916523 | VHL | 7428 | 3 | 0.52 | 0.43 | 0.71 | 0.12 |

### Example 3

### The CIMP-H and CIMP-L Subgroups were Characterized

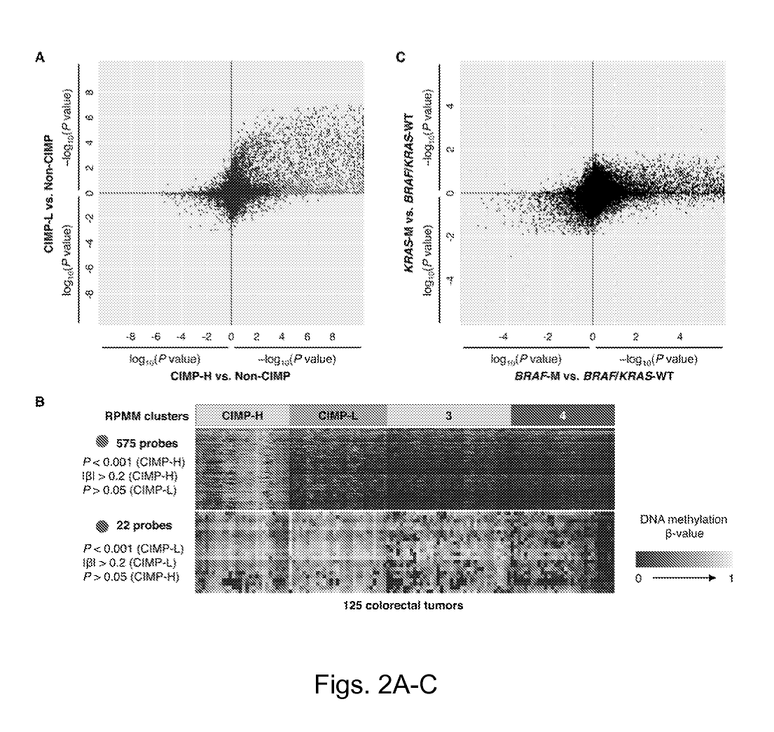

DNA methylation markers associated with CIMP-H and CIMP-L subgroups were investigated. To accomplish this, the DNA methylation β-values for each probe was compared between CIMP-H and non-CIMP tumors (cluster 3 and 4 combined) as well as the β-values between CIMP-L and non-CIMP tumors using the Wilcoxon rank-sum test. Applicants identified 1,618 CpG sites that showed significant DNA hypermethylation in CIMP-H versus non-CIMP tumors (FDR-adjusted P<0.0001) (FIG. 2A). In contrast, 435 CpG sites were found that are significantly hypermethylated in CIMP-L tumors compared with non-CIMP tumors (FDRadjusted P<0.0001) (FIG. 2A). Substantial overlap was observed between the CIMP-H- and CIMP-L-associated markers, as these appear to exhibit a higher frequency of promoter DNA hypermethylation in both tumor subgroups compared with non-CIMP tumors (FIG. 2A). Interestingly, 20% of CIMP-H-associated CpG sites (318 CpGs) were also found to be methylated in CIMP-L tumors (FDR-adjusted P<0.0001 vs. non-CIMP; see list of genes in Table 3).

Specifically, FIGS. 2A-C show, according to particular exemplary aspects, DNA methylation characteristics associated with CIMP-H, CIMP-L, BRAF- and KRAS-mutant colorectal tumors. (A) Comparison of CIMP-H- and CIMP-L-associated DNA methylation profiles. Each data point represents the log₁₀-transformed FDR-adjusted P-value comparing DNA methylation in CIMP-H (n=28) vs. non-CIMP tumors (n=68) (x-axis) and in CIMP-L (n=29) vs. non-CIMP tumors (n=68) (y-axis) for each Infinium DNA methylation probe. For the probes with higher mean DNA methylation in CIMP-H or CIMP-L tumors compared to non-CIMP tumors, −1 is multiplied to log₁₀(FDR-adjusted P-value), providing positive values. The blue and red points (herein reproduced in gray-scale) highlight probes that are significantly hypermethylated in CIMP-H and CIMP-L tumors compared to non-CIMP tumors, respectively. (B) Heatmap representing Infinium DNA methylation β-values for 575 CpG sites that are significantly hypermethylated in CIMP-H compared with non-CIMP tumors (top) and 22 CpG sites that are significantly hypermethylated in CIMP-L compared with non-CIMP tumors (bottom). The four DNA methylation-based subgroups are indicated above the heatmaps. A color gradient from dark blue to yellow (herein reproduced in gray-scale) was used to represent the low and high DNA methylation β-values, respectively. (C) Comparison of BRAF mutant- and KRAS mutant-associated DNA hypermethylation signatures in CRC. The log₁₀-transformed FDR-adjusted P-value for each probe is plotted for tumors harboring KRAS mutations (KRAS-M) (n=34) vs. BRAF/KRAS wild-type (n=74) (y-axis) and those containing BRAF mutations (BRAF-M) (n=17) vs. BRAF/KRAS wild-type (n=74) (x-axis). For the probes with higher mean DNA methylation β-values in BRAF or KRAS mutant tumors compared to wild-type tumors, −1 is multiplied to log₁₀(FDR-adjusted P-value), providing positive values.

TABLE 3
List of probes that are significantly more methylated in both
CIMP-H and CIMP-L tumors compared with non-CIMP tumors.

| | | | CIMP-H tumors | | | CIMP-L tumors | | |
|---|---|---|---|---|---|---|---|---|
| | | HUGO | Mean | P value | FDR- | Mean | P value | FDR- |
| Infinium | ENTREZ | Gene | beta- | (vs. Non- | adjusted | beta- | (vs. Non- | adjusted |
| Probe ID | Gene ID | Symbol | value | CIMP) | P value | value | CIMP) | P value |
| cg00107187 | 388021 | TMEM179 | 0.65 | 1.87E−10 | 8.17E−09 | 0.63 | 4.24E−09 | 1.23E−06 |
| cg00243313 | 50805 | IRX4 | 0.60 | 5.23E−07 | 9.07E−06 | 0.66 | 3.13E−10 | 2.69E−07 |
| cg00273068 | 90187 | EMILIN3 | 0.57 | 8.49E−08 | 1.79E−06 | 0.58 | 7.13E−09 | 1.70E−06 |
| cg00318573 | 1137 | CHRNA4 | 0.66 | 1.07E−09 | 3.63E−08 | 0.64 | 6.80E−09 | 1.66E−06 |
| cg00472814 | 9510 | ADAMTS1 | 0.66 | 5.67E−08 | 1.25E−06 | 0.65 | 1.50E−07 | 1.48E−05 |
| cg00512279 | 6571 | SLC18A2 | 0.55 | 2.77E−06 | 4.03E−05 | 0.60 | 5.29E−08 | 6.88E−06 |
| cg00557354 | 8874 | ARHGEF7 | 0.78 | 3.03E−14 | 3.54E−11 | 0.49 | 1.97E−08 | 3.48E−06 |
| cg00565688 | 7161 | TP73 | 0.54 | 3.60E−08 | 8.30E−07 | 0.53 | 5.29E−08 | 6.88E−06 |
| cg00625653 | 7476 | WNT7A | 0.70 | 1.07E−12 | 1.41E−10 | 0.54 | 1.86E−07 | 1.71E−05 |
| cg00654814 | 146664 | MGAT5B | 0.72 | 1.69E−09 | 5.42E−08 | 0.74 | 6.64E−11 | 1.83E−07 |
| cg00685836 | 8499 | PPFIA2 | 0.48 | 3.72E−09 | 1.08E−07 | 0.41 | 1.83E−06 | 9.35E−05 |
| cg00687686 | 65009 | NDRG4 | 0.71 | 3.80E−12 | 3.34E−10 | 0.66 | 3.13E−10 | 2.69E−07 |
| cg00746981 | 3068 | HDGF | 0.43 | 6.34E−12 | 5.01E−10 | 0.36 | 1.32E−07 | 1.35E−05 |
| cg00756058 | 22873 | DZIP1 | 0.66 | 1.87E−10 | 8.17E−09 | 0.54 | 7.94E−07 | 5.03E−05 |
| cg00826384 | 5803 | PTPRZ1 | 0.52 | 4.66E−11 | 2.62E−09 | 0.41 | 2.21E−07 | 1.96E−05 |
| cg00902195 | 341359 | SYT10 | 0.60 | 1.01E−07 | 2.10E−06 | 0.59 | 3.36E−07 | 2.63E−05 |
| cg00995327 | 9435 | CHST2 | 0.77 | 1.21E−07 | 2.45E−06 | 0.74 | 9.33E−07 | 5.67E−05 |
| cg01173186 | 140767 | NRSN1 | 0.61 | 3.24E−06 | 4.67E−05 | 0.61 | 1.83E−06 | 9.35E−05 |
| cg01192900 | 54766 | BTG4 | 0.76 | 2.61E−08 | 6.22E−07 | 0.75 | 2.36E−08 | 3.92E−06 |
| cg01291404 | 1280 | COL2A1 | 0.51 | 4.67E−13 | 7.99E−11 | 0.36 | 1.14E−08 | 2.42E−06 |
| cg01313514 | 89780 | WNT3A | 0.55 | 3.80E−12 | 3.34E−10 | 0.50 | 1.69E−10 | 2.16E−07 |
| cg01322134 | 89780 | WNT3A | 0.72 | 5.06E−12 | 4.19E−10 | 0.65 | 1.27E−09 | 7.05E−07 |
| cg01468621 | 9024 | BRSK2 | 0.57 | 2.18E−10 | 9.37E−09 | 0.46 | 1.14E−06 | 6.58E−05 |
| cg01519742 | 152789 | JAKMIP1 | 0.70 | 1.51E−10 | 6.84E−09 | 0.63 | 4.66E−09 | 1.28E−06 |
| cg01555431 | 9590 | AKAP12 | 0.74 | 1.35E−06 | 2.13E−05 | 0.74 | 1.14E−06 | 6.58E−05 |
| cg01593190 | 9509 | ADAMTS2 | 0.53 | 1.42E−08 | 3.63E−07 | 0.50 | 2.26E−08 | 3.77E−06 |
| cg01643580 | 3777 | KCNK3 | 0.63 | 4.52E−08 | 1.02E−06 | 0.62 | 2.06E−08 | 3.58E−06 |
| cg01656955 | 84618 | NT5C1A | 0.56 | 8.88E−11 | 4.38E−09 | 0.43 | 1.83E−06 | 9.35E−05 |
| cg01697732 | 54757 | FAM20A | 0.85 | 3.27E−13 | 6.76E−11 | 0.55 | 1.14E−06 | 6.58E−05 |
| cg01699584 | 386617 | KCTD8 | 0.44 | 5.48E−11 | 2.97E−09 | 0.27 | 7.32E−07 | 4.72E−05 |
| cg01775414 | 112885 | PHF21B | 0.71 | 2.64E−09 | 7.99E−08 | 0.66 | 2.72E−07 | 2.28E−05 |
| cg01946574 | 5797 | PTPRM | 0.69 | 5.67E−08 | 1.25E−06 | 0.71 | 8.20E−09 | 1.89E−06 |
| cg02136132 | 56659 | KCNK13 | 0.55 | 2.36E−14 | 3.54E−11 | 0.27 | 1.40E−09 | 7.40E−07 |
| cg02361557 | 22854 | NTNG1 | 0.56 | 9.95E−12 | 7.15E−10 | 0.40 | 1.78E−07 | 1.67E−05 |
| cg02407785 | 5101 | PCDH9 | 0.32 | 7.17E−10 | 2.59E−08 | 0.26 | 1.34E−06 | 7.49E−05 |
| cg02503850 | 140766 | ADAMTS14 | 0.57 | 5.01E−07 | 8.73E−06 | 0.58 | 3.65E−07 | 2.82E−05 |
| cg02508567 | 83439 | TCF7L1 | 0.66 | 2.15E−13 | 5.69E−11 | 0.41 | 3.09E−07 | 2.49E−05 |
| cg02860342 | 10021 | HCN4 | 0.61 | 1.86E−06 | 2.84E−05 | 0.61 | 1.45E−06 | 7.85E−05 |
| cg02899772 | 54550 | NECAB2 | 0.56 | 2.91E−09 | 8.67E−08 | 0.52 | 3.88E−08 | 5.65E−06 |
| cg02932167 | 9427 | ECEL1 | 0.77 | 5.16E−06 | 7.04E−05 | 0.79 | 2.26E−08 | 3.77E−06 |
| cg02982690 | 27319 | BHLHE22 | 0.48 | 2.55E−12 | 2.46E−10 | 0.39 | 2.39E−09 | 9.25E−07 |
| cg03038003 | 79656 | BEND5 | 0.62 | 2.51E−09 | 7.67E−08 | 0.56 | 1.71E−07 | 1.64E−05 |
| cg03168582 | 1761 | DMRT1 | 0.71 | 5.23E−07 | 9.07E−06 | 0.73 | 1.32E−07 | 1.35E−05 |
| cg03285457 | 10660 | LBX1 | 0.52 | 5.36E−06 | 7.29E−05 | 0.53 | 1.50E−06 | 8.08E−05 |
| cg03414321 | 3055 | HCK | 0.40 | 1.51E−10 | 6.84E−09 | 0.29 | 8.58E−08 | 9.86E−06 |
| cg03455458 | 79805 | VASH2 | 0.35 | 9.74E−09 | 2.54E−07 | 0.38 | 3.68E−09 | 1.11E−06 |
| cg03732545 | 6900 | CNTN2 | 0.55 | 1.38E−09 | 4.57E−08 | 0.54 | 1.78E−07 | 1.67E−05 |
| cg03734874 | 388021 | TMEM179 | 0.73 | 2.73E−08 | 6.44E−07 | 0.73 | 4.04E−09 | 1.20E−06 |
| cg03777459 | 140628 | GATA5 | 0.59 | 1.56E−08 | 3.94E−07 | 0.62 | 1.97E−09 | 8.97E−07 |
| cg03848675 | 2295 | FOXF2 | 0.30 | 3.00E−08 | 7.01E−07 | 0.29 | 3.80E−07 | 2.87E−05 |
| cg04080057 | 59285 | CACNG6 | 0.65 | 9.29E−09 | 2.44E−07 | 0.63 | 3.71E−08 | 5.48E−06 |
| cg04101379 | 22873 | DZIP1 | 0.60 | 6.04E−09 | 1.66E−07 | 0.55 | 8.61E−07 | 5.34E−05 |
| cg04251363 | 10402 | ST3GAL6 | 0.41 | 2.23E−07 | 4.21E−06 | 0.32 | 1.39E−06 | 7.66E−05 |
| cg04270799 | 3798 | KIF5A | 0.62 | 1.07E−08 | 2.79E−07 | 0.59 | 4.43E−08 | 6.05E−06 |
| cg04274487 | 11031 | RAB31 | 0.59 | 2.22E−14 | 3.54E−11 | 0.29 | 1.64E−08 | 3.07E−06 |
| cg04330449 | 4762 | NEUROG1 | 0.76 | 3.37E−06 | 4.81E−05 | 0.77 | 2.72E−07 | 2.28E−05 |
| cg04369341 | 84969 | TOX2 | 0.59 | 4.52E−10 | 1.76E−08 | 0.50 | 1.27E−07 | 1.32E−05 |
| cg04391111 | 7161 | TP73 | 0.56 | 2.77E−06 | 4.03E−05 | 0.62 | 3.34E−09 | 1.04E−06 |
| cg04418492 | 9420 | CYP7B1 | 0.55 | 1.44E−07 | 2.86E−06 | 0.52 | 2.03E−07 | 1.83E−05 |
| cg04549333 | 60529 | ALX4 | 0.61 | 8.42E−11 | 4.18E−09 | 0.51 | 1.38E−07 | 1.40E−05 |
| cg04603031 | 1136 | CHRNA3 | 0.68 | 1.36E−08 | 3.48E−07 | 0.71 | 7.36E−10 | 4.70E−07 |
| cg04713521 | 51450 | PRRX2 | 0.65 | 3.15E−10 | 1.27E−08 | 0.62 | 7.73E−10 | 4.80E−07 |
| cg04765277 | 399717 | FLJ45983 | 0.64 | 3.73E−07 | 6.69E−06 | 0.62 | 9.71E−07 | 5.84E−05 |
| cg04897683 | 4762 | NEUROG1 | 0.72 | 3.44E−08 | 7.96E−07 | 0.71 | 2.47E−08 | 4.01E−06 |
| cg04981492 | 85360 | SYDE1 | 0.60 | 1.72E−07 | 3.34E−06 | 0.57 | 3.65E−07 | 2.82E−05 |
| cg04988423 | 60529 | ALX4 | 0.65 | 5.22E−09 | 1.45E−07 | 0.65 | 1.37E−08 | 2.77E−06 |
| cg05028467 | 6620 | SNCB | 0.66 | 3.91E−13 | 7.25E−11 | 0.52 | 1.18E−10 | 1.83E−07 |
| cg05056120 | 1879 | EBF1 | 0.52 | 9.71E−08 | 2.01E−06 | 0.51 | 3.09E−07 | 2.49E−05 |
| cg05421688 | 148753 | FAM163A | 0.49 | 7.51E−12 | 5.66E−10 | 0.35 | 3.96E−07 | 2.96E−05 |
| cg05436658 | 5579 | PRKCB | 0.60 | 3.77E−08 | 8.61E−07 | 0.59 | 2.84E−07 | 2.35E−05 |
| cg05774801 | 6423 | SFRP2 | 0.60 | 1.94E−06 | 2.93E−05 | 0.61 | 7.32E−07 | 4.72E−05 |
| cg05882522 | 30845 | EHD3 | 0.54 | 2.86E−08 | 6.73E−07 | 0.48 | 1.63E−06 | 8.57E−05 |
| cg05899618 | 151449 | GDF7 | 0.64 | 1.80E−12 | 1.92E−10 | 0.55 | 2.69E−10 | 2.65E−07 |
| cg05942574 | 8913 | CACNA1G | 0.43 | 2.27E−09 | 7.02E−08 | 0.39 | 3.80E−07 | 2.87E−05 |
| cg06110728 | 4753 | NELL2 | 0.48 | 8.86E−09 | 2.34E−07 | 0.45 | 1.05E−06 | 6.22E−05 |
| cg06243556 | 65982 | ZSCAN18 | 0.67 | 4.59E−06 | 6.33E−05 | 0.70 | 2.61E−07 | 2.21E−05 |
| cg06268694 | 9620 | CELSR1 | 0.79 | 1.60E−12 | 1.79E−10 | 0.65 | 1.19E−06 | 6.81E−05 |
| cg06321883 | 1310 | COL19A1 | 0.62 | 3.43E−14 | 3.54E−11 | 0.40 | 1.56E−06 | 8.34E−05 |
| cg06339657 | 8622 | PDE8B | 0.61 | 1.88E−08 | 4.65E−07 | 0.57 | 7.63E−07 | 4.84E−05 |
| cg06357925 | 5800 | PTPRO | 0.66 | 1.59E−10 | 7.16E−09 | 0.61 | 1.72E−08 | 3.14E−06 |
| cg06557358 | 124842 | TMEM132E | 0.60 | 1.77E−09 | 5.67E−08 | 0.61 | 1.47E−09 | 7.41E−07 |
| cg06668300 | 4118 | MAL | 0.59 | 2.43E−07 | 4.56E−06 | 0.65 | 3.83E−10 | 2.87E−07 |
| cg06894812 | 4163 | MCC | 0.54 | 1.55E−11 | 1.02E−09 | 0.37 | 1.45E−06 | 7.85E−05 |
| cg06905514 | 816 | CAMK2B | 0.72 | 7.51E−12 | 5.66E−10 | 0.63 | 2.47E−08 | 4.01E−06 |
| cg07015629 | 2066 | ERBB4 | 0.67 | 6.49E−08 | 1.40E−06 | 0.66 | 2.96E−07 | 2.41E−05 |
| cg07017374 | 2322 | FLT3 | 0.81 | 1.43E−12 | 1.73E−10 | 0.70 | 1.72E−08 | 3.14E−06 |
| cg07075930 | 5797 | PTPRM | 0.56 | 3.87E−10 | 1.54E−08 | 0.45 | 2.40E−07 | 2.09E−05 |
| cg07109287 | 9355 | LHX2 | 0.85 | 3.22E−14 | 3.54E−11 | 0.52 | 1.94E−07 | 1.76E−05 |
| cg07143898 | 6585 | SLIT1 | 0.60 | 9.19E−14 | 3.63E−11 | 0.36 | 2.06E−09 | 8.97E−07 |
| cg07236943 | 23089 | PEG10 | 0.29 | 2.07E−08 | 5.04E−07 | 0.25 | 8.96E−07 | 5.53E−05 |
| cg07295678 | 10570 | DPYSL4 | 0.69 | 1.38E−07 | 2.75E−06 | 0.71 | 1.31E−08 | 2.70E−06 |
| cg07570142 | 26002 | MOXD1 | 0.73 | 7.93E−10 | 2.80E−08 | 0.68 | 1.11E−07 | 1.18E−05 |
| cg07651242 | 107 | ADCY1 | 0.73 | 4.10E−09 | 1.17E−07 | 0.68 | 4.30E−07 | 3.10E−05 |
| cg07696033 | 60529 | ALX4 | 0.41 | 2.43E−10 | 1.02E−08 | 0.41 | 2.89E−09 | 9.97E−07 |
| cg07703401 | 3049 | HBQ1 | 0.72 | 1.16E−07 | 2.36E−06 | 0.69 | 1.39E−06 | 7.66E−05 |
| cg07710481 | 26050 | SLITRK5 | 0.41 | 5.36E−06 | 7.29E−05 | 0.47 | 2.59E−08 | 4.13E−06 |
| cg07935568 | 2862 | MLNR | 0.66 | 4.67E−13 | 7.99E−11 | 0.48 | 3.96E−07 | 2.96E−05 |
| cg08045570 | 2295 | FOXF2 | 0.65 | 1.12E−08 | 2.91E−07 | 0.69 | 2.43E−10 | 2.65E−07 |
| cg08132931 | 119 | ADD2 | 0.62 | 2.14E−07 | 4.05E−06 | 0.65 | 1.47E−09 | 7.41E−07 |
| cg08190044 | 57198 | ATP8B2 | 0.75 | 2.73E−08 | 6.44E−07 | 0.75 | 7.83E−09 | 1.83E−06 |
| cg08209133 | 201780 | SLC10A4 | 0.54 | 2.57E−13 | 6.07E−11 | 0.40 | 4.24E−08 | 5.86E−06 |
| cg08244522 | 7056 | THBD | 0.46 | 1.88E−07 | 3.60E−06 | 0.46 | 4.06E−08 | 5.75E−06 |
| cg08315770 | 89822 | KCNK17 | 0.70 | 9.77E−14 | 3.66E−11 | 0.50 | 4.87E−07 | 3.42E−05 |
| cg08555612 | 60675 | PROK2 | 0.65 | 1.35E−12 | 1.65E−10 | 0.37 | 7.32E−07 | 4.72E−05 |
| cg08575537 | 2056 | EPO | 0.79 | 3.43E−07 | 6.22E−06 | 0.82 | 6.19E−09 | 1.58E−06 |
| cg08859916 | 5728 | PTEN | 0.55 | 5.29E−14 | 3.54E−11 | 0.32 | 9.78E−08 | 1.10E−05 |
| cg08876932 | 401 | PHOX2A | 0.55 | 6.74E−06 | 8.92E−05 | 0.61 | 3.04E−09 | 9.98E−07 |
| cg08896945 | 797 | CALCB | 0.57 | 1.91E−12 | 1.99E−10 | 0.43 | 1.76E−06 | 9.10E−05 |
| cg09053680 | 8433 | UTF1 | 0.75 | 1.94E−06 | 2.93E−05 | 0.75 | 5.98E−07 | 4.09E−05 |
| cg09147222 | 131034 | CPNE4 | 0.49 | 7.27E−06 | 9.55E−05 | 0.56 | 4.45E−09 | 1.25E−06 |
| cg09191327 | 59335 | PRDM12 | 0.61 | 2.77E−09 | 8.31E−08 | 0.58 | 1.19E−08 | 2.51E−06 |
| cg09231514 | 125988 | C19orf70 | 0.37 | 3.37E−09 | 9.94E−08 | 0.30 | 2.61E−07 | 2.21E−05 |
| cg09313439 | 1000 | CDH2 | 0.55 | 1.79E−06 | 2.74E−05 | 0.57 | 4.43E−08 | 6.05E−06 |
| cg09416313 | 4145 | MATK | 0.63 | 2.91E−09 | 8.67E−08 | 0.62 | 6.49E−09 | 1.62E−06 |
| cg09437522 | 2778 | GNAS | 0.60 | 4.10E−09 | 1.17E−07 | 0.56 | 7.63E−07 | 4.84E−05 |
| cg09440289 | 5800 | PTPRO | 0.58 | 3.75E−11 | 2.17E−09 | 0.46 | 7.63E−07 | 4.84E−05 |
| cg09495977 | 94031 | HTRA3 | 0.53 | 5.06E−12 | 4.19E−10 | 0.49 | 7.00E−11 | 1.83E−07 |
| cg09622447 | 875 | CBS | 0.68 | 7.96E−13 | 1.09E−10 | 0.43 | 1.71E−07 | 1.64E−05 |
| cg09628601 | 4861 | NPAS1 | 0.65 | 5.84E−10 | 2.19E−08 | 0.65 | 1.54E−09 | 7.60E−07 |
| cg09660171 | 4010 | LMX1B | 0.53 | 1.36E−10 | 6.27E−09 | 0.47 | 1.57E−07 | 1.54E−05 |
| cg09750385 | 2982 | GUCY1A3 | 0.29 | 4.59E−06 | 6.33E−05 | 0.31 | 4.30E−07 | 3.10E−05 |
| cg09874752 | 6425 | SFRP5 | 0.58 | 1.18E−11 | 8.13E−10 | 0.45 | 1.14E−08 | 2.42E−06 |
| cg09945801 | 7486 | WRN | 0.67 | 7.43E−08 | 1.59E−06 | 0.68 | 2.17E−09 | 8.97E−07 |
| cg09949775 | 1311 | COMP | 0.66 | 6.79E−11 | 3.48E−09 | 0.55 | 2.30E−07 | 2.02E−05 |
| cg09979256 | 84870 | RSPO3 | 0.58 | 6.04E−09 | 1.66E−07 | 0.58 | 9.89E−09 | 2.19E−06 |
| cg10158080 | 6660 | SOX5 | 0.64 | 9.72E−10 | 3.35E−08 | 0.68 | 2.55E−11 | 1.30E−07 |
| cg10247252 | 8811 | GALR2 | 0.43 | 7.17E−11 | 3.65E−09 | 0.40 | 2.61E−07 | 2.21E−05 |
| cg10486998 | 2587 | GALR1 | 0.65 | 8.61E−07 | 1.43E−05 | 0.63 | 1.90E−06 | 9.61E−05 |
| cg10605520 | 11255 | HRH3 | 0.52 | 1.04E−10 | 5.02E−09 | 0.29 | 1.90E−06 | 9.61E−05 |
| cg10646402 | 5800 | PTPRO | 0.69 | 4.08E−10 | 1.61E−08 | 0.63 | 7.53E−08 | 8.88E−06 |
| cg10647513 | 3039 | HBA1 | 0.50 | 3.89E−07 | 6.95E−06 | 0.50 | 2.16E−08 | 3.66E−06 |
| cg10920957 | 57338 | JPH3 | 0.57 | 5.48E−11 | 2.97E−09 | 0.44 | 5.07E−07 | 3.54E−05 |
| cg11189837 | 9510 | ADAMTS1 | 0.61 | 5.01E−10 | 1.92E−08 | 0.54 | 3.36E−07 | 2.63E−05 |
| cg11248413 | 4762 | NEUROG1 | 0.75 | 1.20E−12 | 1.55E−10 | 0.54 | 1.07E−07 | 1.16E−05 |
| cg11260848 | 60529 | ALX4 | 0.60 | 6.79E−11 | 3.48E−09 | 0.52 | 6.75E−07 | 4.46E−05 |
| cg11319389 | 84969 | TOX2 | 0.60 | 6.47E−10 | 2.37E−08 | 0.56 | 7.53E−08 | 8.88E−06 |
| cg11399100 | 25789 | TMEM59L | 0.59 | 6.29E−13 | 9.54E−11 | 0.40 | 9.44E−09 | 2.11E−06 |
| cg11428724 | 5081 | PAX7 | 0.83 | 6.34E−12 | 5.01E−10 | 0.82 | 1.33E−11 | 1.14E−07 |
| cg11438428 | 256297 | PTF1A | 0.79 | 4.97E−09 | 1.40E−07 | 0.76 | 3.18E−09 | 1.02E−06 |
| cg11668923 | 8038 | ADAM12 | 0.70 | 4.67E−13 | 7.99E−11 | 0.59 | 3.47E−10 | 2.69E−07 |
| cg11670211 | 50507 | NOX4 | 0.50 | 7.10E−08 | 1.52E−06 | 0.42 | 1.34E−06 | 7.49E−05 |
| cg11747771 | 2731 | GLDC | 0.71 | 4.76E−10 | 1.84E−08 | 0.64 | 8.96E−08 | 1.02E−05 |
| cg11768886 | 55351 | STK32B | 0.34 | 3.94E−06 | 5.51E−05 | 0.37 | 1.76E−06 | 9.10E−05 |
| cg11847808 | 2046 | EPHA8 | 0.58 | 7.95E−12 | 5.95E−10 | 0.53 | 4.46E−10 | 3.23E−07 |
| cg11935147 | 9659 | PDE4DIP | 0.70 | 6.36E−14 | 3.54E−11 | 0.58 | 8.60E−09 | 1.96E−06 |
| cg11939071 | 1840 | DTX1 | 0.65 | 6.24E−06 | 8.32E−05 | 0.70 | 4.24E−09 | 1.23E−06 |
| cg11981631 | 6833 | ABCC8 | 0.64 | 1.25E−13 | 4.16E−11 | 0.44 | 1.14E−08 | 2.42E−06 |
| cg12005098 | 387700 | SLC16A12 | 0.71 | 8.26E−07 | 1.38E−05 | 0.71 | 4.67E−07 | 3.33E−05 |
| cg12374431 | 25806 | VAX2 | 0.53 | 1.13E−09 | 3.81E−08 | 0.43 | 5.53E−08 | 7.15E−06 |
| cg12539975 | 59335 | PRDM12 | 0.50 | 3.28E−08 | 7.64E−07 | 0.52 | 2.63E−09 | 9.81E−07 |
| cg12699371 | 2587 | GALR1 | 0.56 | 7.93E−10 | 2.80E−08 | 0.49 | 4.30E−07 | 3.10E−05 |
| cg12768605 | 284348 | LYPD5 | 0.65 | 5.36E−06 | 7.29E−05 | 0.66 | 1.34E−06 | 7.49E−05 |
| cg12874092 | 7431 | VIM | 0.60 | 6.65E−09 | 1.81E−07 | 0.63 | 8.54E−10 | 5.01E−07 |
| cg12995941 | 3645 | INSRR | 0.56 | 2.43E−10 | 1.02E−08 | 0.44 | 1.76E−06 | 9.10E−05 |
| cg13031432 | 65009 | NDRG4 | 0.66 | 1.24E−11 | 8.49E−10 | 0.61 | 1.09E−09 | 6.25E−07 |
| cg13168683 | 152789 | JAKMIP1 | 0.60 | 9.95E−12 | 7.15E−10 | 0.51 | 2.16E−08 | 3.66E−06 |
| cg13216057 | 27122 | DKK3 | 0.51 | 7.67E−09 | 2.06E−07 | 0.42 | 2.96E−07 | 2.41E−05 |
| cg13274713 | 6909 | TBX2 | 0.65 | 5.92E−13 | 9.53E−11 | 0.43 | 5.98E−07 | 4.09E−05 |
| cg13297865 | 6785 | ELOVL4 | 0.67 | 1.91E−12 | 1.99E−10 | 0.55 | 1.78E−07 | 1.67E−05 |
| cg13346411 | 887 | CCKBR | 0.64 | 1.60E−12 | 1.79E−10 | 0.54 | 4.45E−09 | 1.25E−06 |
| cg13351583 | 53358 | SHC3 | 0.52 | 2.46E−06 | 3.61E−05 | 0.53 | 1.63E−06 | 8.57E−05 |
| cg13378388 | 7424 | VEGFC | 0.62 | 1.10E−06 | 1.78E−05 | 0.64 | 3.36E−07 | 2.63E−05 |
| cg13436799 | 4036 | LRP2 | 0.54 | 6.29E−13 | 9.54E−11 | 0.31 | 1.83E−06 | 9.35E−05 |
| cg13488201 | 8038 | ADAM12 | 0.77 | 1.56E−08 | 3.94E−07 | 0.77 | 1.88E−08 | 3.38E−06 |
| cg13562542 | 2850 | GPR27 | 0.68 | 6.10E−11 | 3.25E−09 | 0.64 | 5.91E−09 | 1.55E−06 |
| cg13686115 | 84457 | PHYHIPL | 0.37 | 1.72E−08 | 4.29E−07 | 0.36 | 4.67E−07 | 3.33E−05 |
| cg13749822 | 64399 | HHIP | 0.66 | 1.07E−09 | 3.63E−08 | 0.56 | 5.07E−07 | 3.54E−05 |
| cg13756879 | 3481 | IGF2 | 0.66 | 1.90E−13 | 5.10E−11 | 0.61 | 1.57E−11 | 1.14E−07 |
| cg13878010 | 111 | ADCY5 | 0.68 | 9.89E−11 | 4.79E−09 | 0.59 | 6.90E−08 | 8.36E−06 |
| cg14046986 | 92241 | RCSD1 | 0.43 | 7.50E−13 | 1.06E−10 | 0.22 | 1.07E−07 | 1.16E−05 |
| cg14049461 | 2895 | GRID2 | 0.38 | 2.43E−10 | 1.02E−08 | 0.31 | 1.14E−06 | 6.58E−05 |
| cg14135551 | 23500 | DAAM2 | 0.38 | 2.36E−06 | 3.49E−05 | 0.41 | 5.29E−08 | 6.88E−06 |
| cg14144305 | 60529 | ALX4 | 0.53 | 1.16E−07 | 2.36E−06 | 0.53 | 3.39E−08 | 5.15E−06 |
| cg14242042 | 6660 | SOX5 | 0.62 | 7.17E−10 | 2.59E−08 | 0.64 | 1.12E−10 | 1.83E−07 |
| cg14312526 | 668 | FOXL2 | 0.61 | 4.67E−14 | 3.54E−11 | 0.40 | 7.03E−07 | 4.60E−05 |
| cg14662379 | 547 | KIF1A | 0.66 | 1.25E−13 | 4.16E−11 | 0.49 | 3.50E−09 | 1.07E−06 |
| cg14823162 | 5454 | POU3F2 | 0.46 | 2.55E−12 | 2.46E−10 | 0.31 | 1.11E−07 | 1.18E−05 |
| cg14958635 | 4762 | NEUROG1 | 0.66 | 1.06E−06 | 1.72E−05 | 0.70 | 1.50E−08 | 2.98E−06 |
| cg15014549 | 55244 | SLC47A1 | 0.60 | 1.59E−13 | 4.48E−11 | 0.35 | 4.87E−07 | 3.42E−05 |
| cg15057581 | 140885 | SIRPA | 0.50 | 8.13E−14 | 3.54E−11 | 0.40 | 2.06E−08 | 3.58E−06 |
| cg15107670 | 7490 | WT1 | 0.64 | 4.30E−09 | 1.22E−07 | 0.64 | 2.50E−09 | 9.54E−07 |
| cg15205507 | 55422 | ZNF331 | 0.56 | 1.51E−10 | 6.84E−09 | 0.58 | 2.69E−10 | 2.65E−07 |
| cg15461516 | 8534 | CHST1 | 0.65 | 5.22E−09 | 1.45E−07 | 0.63 | 7.13E−09 | 1.70E−06 |
| cg15565872 | 5806 | PTX3 | 0.50 | 4.13E−08 | 9.36E−07 | 0.45 | 1.71E−07 | 1.64E−05 |
| cg15640375 | 79948 | LPPR3 | 0.66 | 3.39E−12 | 3.11E−10 | 0.60 | 3.00E−11 | 1.30E−07 |
| cg15749748 | 140628 | GATA5 | 0.61 | 2.65E−07 | 4.93E−06 | 0.63 | 4.06E−08 | 5.75E−06 |
| cg15753757 | 26053 | AUTS2 | 0.59 | 1.32E−13 | 4.17E−11 | 0.35 | 1.11E−07 | 1.18E−05 |
| cg15817236 | 60529 | ALX4 | 0.62 | 8.05E−09 | 2.15E−07 | 0.63 | 8.13E−10 | 4.90E−07 |
| cg16041660 | 144165 | PRICKLE1 | 0.74 | 1.64E−11 | 1.07E−09 | 0.64 | 1.32E−07 | 1.35E−05 |
| cg16042149 | 4744 | NEFH | 0.65 | 2.07E−08 | 5.04E−07 | 0.62 | 2.21E−07 | 1.96E−05 |
| cg16248277 | 2253 | FGF8 | 0.64 | 1.36E−10 | 6.27E−09 | 0.61 | 1.97E−08 | 3.48E−06 |
| cg16257091 | 627 | BDNF | 0.50 | 4.73E−08 | 1.06E−06 | 0.47 | 1.45E−06 | 7.85E−05 |
| cg16584573 | 2253 | FGF8 | 0.73 | 1.27E−12 | 1.60E−10 | 0.63 | 3.47E−10 | 2.69E−07 |
| cg16604516 | 2199 | FBLN2 | 0.74 | 5.06E−12 | 4.19E−10 | 0.63 | 3.80E−07 | 2.87E−05 |
| cg16708281 | 200350 | FOXD4L1 | 0.56 | 2.33E−07 | 4.38E−06 | 0.55 | 1.50E−07 | 1.48E−05 |
| cg16852892 | 4325 | MMP16 | 0.11 | 3.57E−07 | 6.47E−06 | 0.12 | 1.94E−07 | 1.76E−05 |
| cg16884569 | 9770 | RASSF2 | 0.50 | 1.01E−07 | 2.10E−06 | 0.48 | 7.03E−07 | 4.60E−05 |
| cg16907566 | 7373 | COL14A1 | 0.61 | 1.96E−09 | 6.17E−08 | 0.57 | 1.01E−06 | 6.04E−05 |
| cg16969623 | 55422 | ZNF331 | 0.57 | 5.01E−10 | 1.92E−08 | 0.55 | 2.17E−09 | 8.97E−07 |
| cg17018527 | 53346 | TM6SF1 | 0.35 | 8.49E−08 | 1.79E−06 | 0.34 | 2.72E−07 | 2.28E−05 |
| cg17108819 | 925 | CD8A | 0.73 | 4.42E−07 | 7.78E−06 | 0.75 | 5.07E−08 | 6.75E−06 |
| cg17133183 | 1381 | CRABP1 | 0.61 | 3.88E−14 | 3.54E−11 | 0.22 | 1.34E−06 | 7.49E−05 |
| cg17188046 | 6862 | T | 0.68 | 6.49E−08 | 1.40E−06 | 0.64 | 1.90E−06 | 9.61E−05 |
| cg17194182 | 2056 | EPO | 0.60 | 2.56E−10 | 1.06E−08 | 0.52 | 1.05E−06 | 6.22E−05 |
| cg17503456 | 668 | FOXL2 | 0.68 | 2.36E−14 | 3.54E−11 | 0.39 | 2.71E−08 | 4.29E−06 |
| cg17775235 | 4884 | NPTX1 | 0.67 | 9.77E−14 | 3.66E−11 | 0.50 | 1.53E−10 | 2.07E−07 |
| cg17834752 | 51305 | KCNK9 | 0.62 | 1.22E−10 | 5.69E−09 | 0.57 | 3.65E−07 | 2.82E−05 |
| cg17880199 | 4629 | MYH11 | 0.61 | 3.91E−13 | 7.25E−11 | 0.46 | 5.07E−08 | 6.75E−06 |
| cg18396533 | 143241 | DYDC1 | 0.70 | 9.71E−08 | 2.01E−06 | 0.68 | 3.36E−07 | 2.63E−05 |
| cg18403396 | 135152 | B3GAT2 | 0.53 | 7.57E−11 | 3.80E−09 | 0.34 | 9.10E−11 | 1.83E−07 |
| cg18581445 | 56961 | SHD | 0.52 | 5.48E−11 | 2.97E−09 | 0.39 | 3.80E−07 | 2.87E−05 |
| cg18602314 | 9945 | GFPT2 | 0.43 | 2.30E−10 | 9.82E−09 | 0.38 | 3.10E−08 | 4.81E−06 |
| cg18938204 | 90187 | EMILIN3 | 0.66 | 1.07E−08 | 2.79E−07 | 0.70 | 8.63E−11 | 1.83E−07 |
| cg18943599 | 6752 | SSTR2 | 0.24 | 8.26E−07 | 1.38E−05 | 0.22 | 3.39E−08 | 5.15E−06 |
| cg18952560 | 140885 | SIRPA | 0.62 | 3.27E−13 | 6.76E−11 | 0.48 | 6.49E−09 | 1.62E−06 |
| cg19063972 | 11166 | SOX21 | 0.61 | 6.15E−10 | 2.27E−08 | 0.57 | 1.78E−07 | 1.67E−05 |
| cg19141563 | 22843 | PPM1E | 0.56 | 8.88E−11 | 4.38E−09 | 0.47 | 2.40E−07 | 2.09E−05 |
| cg19332710 | 140730 | RIMS4 | 0.78 | 6.29E−13 | 9.54E−11 | 0.57 | 4.49E−07 | 3.22E−05 |
| cg19355190 | 1959 | EGR2 | 0.63 | 3.39E−12 | 3.11E−10 | 0.50 | 4.87E−07 | 3.42E−05 |
| cg19358442 | 60529 | ALX4 | 0.60 | 3.73E−07 | 6.69E−06 | 0.61 | 1.57E−08 | 3.01E−06 |
| cg19358493 | 2018 | EMX2 | 0.41 | 4.30E−09 | 1.22E−07 | 0.44 | 4.06E−08 | 5.75E−06 |
| cg19439399 | 6785 | ELOVL4 | 0.53 | 1.53E−09 | 4.99E−08 | 0.50 | 1.39E−06 | 7.66E−05 |
| cg19461621 | 81035 | COLEC12 | 0.68 | 1.24E−06 | 1.99E−05 | 0.68 | 6.23E−07 | 4.24E−05 |
| cg19674669 | 112937 | GLB1L3 | 0.74 | 2.16E−09 | 6.72E−08 | 0.69 | 1.07E−07 | 1.16E−05 |
| cg19697981 | 7101 | NR2E1 | 0.58 | 3.73E−07 | 6.69E−06 | 0.62 | 5.13E−09 | 1.37E−06 |
| cg19850348 | 26108 | PYGO1 | 0.68 | 1.80E−08 | 4.47E−07 | 0.66 | 6.90E−08 | 8.36E−06 |
| cg19917856 | 342897 | NCCRP1 | 0.69 | 9.74E−09 | 2.54E−07 | 0.66 | 3.80E−07 | 2.87E−05 |
| cg19918758 | 10451 | VAV3 | 0.44 | 6.24E−06 | 8.32E−05 | 0.46 | 1.11E−07 | 1.18E−05 |
| cg20025656 | 58 | ACTA1 | 0.68 | 3.60E−08 | 8.30E−07 | 0.68 | 2.16E−08 | 3.66E−06 |
| cg20161179 | 4487 | MSX1 | 0.39 | 2.54E−07 | 4.74E−06 | 0.40 | 4.24E−08 | 5.86E−06 |
| cg20209009 | 30009 | TBX21 | 0.61 | 2.06E−09 | 6.47E−08 | 0.64 | 2.43E−10 | 2.65E−07 |
| cg20256494 | 164633 | CABP7 | 0.74 | 3.91E−13 | 7.25E−11 | 0.59 | 7.00E−10 | 4.61E−07 |
| cg20291049 | 5455 | POU3F3 | 0.77 | 1.96E−07 | 3.75E−06 | 0.78 | 7.53E−08 | 8.88E−06 |
| cg20339230 | 8128 | ST8SIA2 | 0.77 | 1.69E−09 | 5.42E−08 | 0.80 | 4.59E−11 | 1.66E−07 |
| cg20357628 | 116154 | PHACTR3 | 0.75 | 6.44E−11 | 3.36E−09 | 0.70 | 2.59E−08 | 4.13E−06 |
| cg20530314 | 185 | AGTR1 | 0.71 | 7.93E−07 | 1.33E−05 | 0.78 | 5.13E−09 | 1.37E−06 |
| cg20624391 | 11149 | BVES | 0.60 | 2.43E−07 | 4.56E−06 | 0.59 | 2.84E−07 | 2.35E−05 |
| cg20674577 | 116154 | PHACTR3 | 0.57 | 1.10E−06 | 1.78E−05 | 0.60 | 2.27E−09 | 8.97E−07 |
| cg20699736 | 25806 | VAX2 | 0.38 | 1.18E−08 | 3.04E−07 | 0.34 | 3.39E−08 | 5.15E−06 |
| cg20792294 | 51214 | IGF2AS | 0.71 | 6.10E−11 | 3.25E−09 | 0.63 | 9.36E−08 | 1.06E−05 |
| cg20804555 | 145258 | GSC | 0.60 | 9.37E−11 | 4.57E−09 | 0.62 | 6.91E−12 | 1.14E−07 |
| cg21017752 | 5507 | PPP1R3C | 0.55 | 3.68E−10 | 1.46E−08 | 0.52 | 2.83E−08 | 4.42E−06 |
| cg21269934 | 339983 | NAT8L | 0.65 | 1.22E−10 | 5.69E−09 | 0.62 | 2.89E−09 | 9.97E−07 |
| cg21321735 | 547 | KIF1A | 0.55 | 1.74E−11 | 1.12E−09 | 0.30 | 2.76E−09 | 9.81E−07 |
| cg21435336 | 126549 | ANKLE1 | 0.62 | 1.35E−12 | 1.65E−10 | 0.52 | 6.80E−09 | 1.66E−06 |
| cg21513553 | 1292 | COL6A2 | 0.72 | 3.05E−09 | 9.07E−08 | 0.74 | 2.69E−10 | 2.65E−07 |
| cg21547708 | 6752 | SSTR2 | 0.60 | 3.79E−06 | 5.33E−05 | 0.63 | 5.78E−08 | 7.39E−06 |
| cg21553524 | 130733 | TMEM178 | 0.53 | 6.97E−09 | 1.88E−07 | 0.47 | 1.16E−07 | 1.23E−05 |
| cg21604803 | 126129 | CPT1C | 0.54 | 6.79E−08 | 1.46E−06 | 0.50 | 1.76E−06 | 9.10E−05 |
| cg21652958 | 7058 | THBS2 | 0.45 | 6.21E−08 | 1.35E−06 | 0.43 | 4.13E−07 | 3.05E−05 |
| cg21937886 | 9945 | GFPT2 | 0.83 | 1.39E−11 | 9.32E−10 | 0.67 | 4.64E−08 | 6.29E−06 |
| cg21942082 | 134526 | ACOT12 | 0.45 | 4.29E−10 | 1.69E−08 | 0.39 | 1.98E−06 | 9.88E−05 |
| cg22007439 | 63951 | DMRTA1 | 0.44 | 8.35E−10 | 2.91E−08 | 0.37 | 1.07E−07 | 1.16E−05 |
| cg22036988 | 92369 | SPSB4 | 0.69 | 5.55E−10 | 2.10E−08 | 0.64 | 1.38E−07 | 1.40E−05 |
| cg22063989 | 284654 | RSPO1 | 0.52 | 7.67E−09 | 2.06E−07 | 0.55 | 5.19E−10 | 3.63E−07 |
| cg22123464 | 6543 | SLC8A2 | 0.62 | 4.10E−09 | 1.17E−07 | 0.54 | 5.29E−07 | 3.68E−05 |
| cg22197787 | 3756 | KCNH1 | 0.25 | 1.64E−11 | 1.07E−09 | 0.19 | 4.30E−07 | 3.10E−05 |
| cg22336401 | 377841 | ENTPD8 | 0.10 | 8.42E−11 | 4.18E−09 | 0.08 | 3.80E−07 | 2.87E−05 |
| cg22594309 | 127833 | SYT2 | 0.64 | 7.50E−13 | 1.06E−10 | 0.51 | 6.90E−08 | 8.36E−06 |
| cg22679003 | 1000 | CDH2 | 0.59 | 1.80E−07 | 3.47E−06 | 0.63 | 6.19E−09 | 1.58E−06 |
| cg22777952 | 27023 | FOXB1 | 0.47 | 3.18E−11 | 1.90E−09 | 0.35 | 3.50E−07 | 2.73E−05 |
| cg22815110 | 27022 | FOXD3 | 0.70 | 6.65E−09 | 1.81E−07 | 0.66 | 1.16E−07 | 1.23E−05 |
| cg22967284 | 6585 | SLIT1 | 0.24 | 9.74E−09 | 2.54E−07 | 0.16 | 9.71E−07 | 5.84E−05 |
| cg22975913 | 7490 | WT1 | 0.49 | 3.77E−08 | 8.61E−07 | 0.54 | 2.27E−09 | 8.97E−07 |
| cg22994720 | 25884 | CHRDL2 | 0.46 | 1.94E−06 | 2.93E−05 | 0.46 | 6.04E−08 | 7.58E−06 |
| cg23029193 | 133584 | EGFLAM | 0.50 | 2.46E−06 | 3.61E−05 | 0.53 | 3.71E−08 | 5.48E−06 |
| cg23040064 | 57338 | JPH3 | 0.55 | 5.06E−12 | 4.19E−10 | 0.42 | 4.24E−08 | 5.86E−06 |
| cg23089840 | 81543 | LRRC3 | 0.59 | 1.10E−06 | 1.78E−05 | 0.64 | 2.30E−07 | 2.02E−05 |
| cg23166362 | 5293 | PIK3CD | 0.63 | 4.18E−11 | 2.38E−09 | 0.37 | 7.63E−07 | 4.84E−05 |
| cg23196831 | 7373 | COL14A1 | 0.61 | 1.64E−08 | 4.10E−07 | 0.63 | 3.04E−09 | 9.98E−07 |
| cg23219720 | 219578 | ZNF804B | 0.49 | 1.94E−06 | 2.93E−05 | 0.52 | 5.78E−08 | 7.39E−06 |
| cg23273897 | 4311 | MME | 0.47 | 4.52E−08 | 1.02E−06 | 0.50 | 2.17E−09 | 8.97E−07 |
| cg23473904 | 1292 | COL6A2 | 0.56 | 7.07E−13 | 1.02E−10 | 0.42 | 5.29E−08 | 6.88E−06 |
| cg23582408 | 1917 | EEF1A2 | 0.50 | 1.88E−08 | 4.65E−07 | 0.50 | 1.31E−08 | 2.70E−06 |
| cg24053587 | 5800 | PTPRO | 0.38 | 7.57E−11 | 3.80E−09 | 0.28 | 1.50E−06 | 8.08E−05 |
| cg24068372 | 349136 | WDR86 | 0.81 | 4.24E−07 | 7.49E−06 | 0.80 | 8.27E−07 | 5.19E−05 |
| cg24396745 | 10021 | HCN4 | 0.67 | 2.02E−12 | 2.08E−10 | 0.54 | 6.23E−07 | 4.24E−05 |
| cg24662718 | 10451 | VAV3 | 0.73 | 2.33E−07 | 4.38E−06 | 0.73 | 1.71E−07 | 1.64E−05 |
| cg24723331 | 6489 | ST8SIA1 | 0.44 | 7.00E−06 | 9.23E−05 | 0.49 | 1.63E−06 | 8.57E−05 |
| cg24834740 | 26051 | PPP1R16B | 0.65 | 7.93E−07 | 1.33E−05 | 0.69 | 3.71E−08 | 5.48E−06 |
| cg24879335 | 7018 | TF | 0.57 | 2.43E−10 | 1.02E−08 | 0.51 | 8.27E−07 | 5.19E−05 |
| cg24924779 | 3755 | KCNG1 | 0.74 | 1.35E−06 | 2.13E−05 | 0.76 | 6.90E−08 | 8.36E−06 |
| cg25014318 | 2740 | GLP1R | 0.71 | 7.54E−10 | 2.70E−08 | 0.65 | 2.21E−07 | 1.96E−05 |
| cg25070637 | 6383 | SDC2 | 0.51 | 2.89E−07 | 5.31E−06 | 0.47 | 8.96E−07 | 5.53E−05 |
| cg25094569 | 7490 | WT1 | 0.60 | 6.49E−08 | 1.40E−06 | 0.59 | 6.04E−08 | 7.58E−06 |
| cg25167643 | 5803 | PTPRZ1 | 0.75 | 3.90E−09 | 1.13E−07 | 0.67 | 1.39E−06 | 7.66E−05 |
| cg25228126 | 2535 | FZD2 | 0.55 | 5.78E−06 | 7.80E−05 | 0.56 | 7.32E−07 | 4.72E−05 |
| cg25302419 | 1501 | CTNND2 | 0.65 | 2.70E−11 | 1.64E−09 | 0.61 | 2.76E−09 | 9.81E−07 |
| cg25332298 | 1995 | ELAVL3 | 0.54 | 1.70E−12 | 1.87E−10 | 0.47 | 2.17E−09 | 8.97E−07 |
| cg25363445 | 60529 | ALX4 | 0.54 | 1.72E−08 | 4.29E−07 | 0.53 | 1.21E−07 | 1.27E−05 |
| cg25431974 | 9427 | ECEL1 | 0.86 | 2.89E−07 | 5.31E−06 | 0.89 | 1.18E−10 | 1.83E−07 |
| cg25434223 | 1995 | ELAVL3 | 0.56 | 9.35E−07 | 1.53E−05 | 0.57 | 2.50E−07 | 2.16E−05 |
| cg25465406 | 3000 | GUCY2D | 0.60 | 8.26E−07 | 1.38E−05 | 0.59 | 9.71E−07 | 5.84E−05 |
| cg25834568 | 84870 | RSPO3 | 0.25 | 6.79E−11 | 3.48E−09 | 0.22 | 1.57E−08 | 3.01E−06 |
| cg25875213 | 163115 | ZNF781 | 0.59 | 6.44E−11 | 3.36E−09 | 0.49 | 6.75E−07 | 4.46E−05 |
| cg25905812 | 1761 | DMRT1 | 0.58 | 6.49E−08 | 1.40E−06 | 0.59 | 6.04E−08 | 7.58E−06 |
| cg25942450 | 30012 | TLX3 | 0.75 | 6.01E−06 | 8.07E−05 | 0.78 | 1.71E−07 | 1.64E−05 |
| cg25971347 | 2294 | FOXF1 | 0.59 | 3.44E−08 | 7.96E−07 | 0.63 | 1.53E−10 | 2.07E−07 |
| cg25999867 | 112937 | GLB1L3 | 0.67 | 2.91E−09 | 8.67E−08 | 0.60 | 9.71E−07 | 5.84E−05 |
| cg26164310 | 9890 | LPPR4 | 0.64 | 1.94E−06 | 2.93E−05 | 0.63 | 7.63E−07 | 4.84E−05 |
| cg26195812 | 56896 | DPYSL5 | 0.78 | 5.98E−14 | 3.54E−11 | 0.60 | 4.66E−09 | 1.28E−06 |
| cg26232187 | 4916 | NTRK3 | 0.27 | 1.58E−07 | 3.10E−06 | 0.30 | 6.66E−10 | 4.52E−07 |
| cg26365854 | 60529 | ALX4 | 0.64 | 2.77E−07 | 5.10E−06 | 0.67 | 3.18E−09 | 1.02E−06 |
| cg26466094 | 26289 | AK5 | 0.51 | 1.65E−07 | 3.21E−06 | 0.52 | 7.87E−08 | 9.18E−06 |
| cg26525091 | 8174 | MADCAM1 | 0.61 | 2.07E−08 | 5.04E−07 | 0.60 | 2.06E−09 | 8.97E−07 |
| cg26557658 | 163933 | FAM43B | 0.54 | 1.51E−07 | 2.98E−06 | 0.56 | 7.83E−09 | 1.83E−06 |
| cg26607785 | 30009 | TBX21 | 0.56 | 2.05E−11 | 1.29E−09 | 0.42 | 1.28E−06 | 7.31E−05 |
| cg26702254 | 3751 | KCND2 | 0.46 | 7.43E−08 | 1.59E−06 | 0.43 | 7.32E−07 | 4.72E−05 |
| cg26705553 | 64386 | MMP25 | 0.47 | 1.80E−07 | 3.47E−06 | 0.47 | 3.22E−07 | 2.58E−05 |
| cg26747293 | 133584 | EGFLAM | 0.68 | 3.72E−09 | 1.08E−07 | 0.69 | 1.70E−09 | 8.20E−07 |
| cg26756083 | 4325 | MMP16 | 0.57 | 3.77E−08 | 8.61E−07 | 0.56 | 7.87E−08 | 9.18E−06 |
| cg27138584 | 56660 | KCNK12 | 0.41 | 5.84E−10 | 2.19E−08 | 0.37 | 1.80E−08 | 3.26E−06 |
| cg27196745 | 5800 | PTPRO | 0.73 | 2.43E−10 | 1.02E−08 | 0.70 | 2.76E−09 | 9.81E−07 |
| cg27286999 | 10439 | OLFM1 | 0.39 | 3.77E−08 | 8.61E−07 | 0.43 | 1.18E−10 | 1.83E−07 |
| cg27319898 | 219578 | ZNF804B | 0.63 | 4.42E−07 | 7.78E−06 | 0.64 | 8.22E−08 | 9.49E−06 |
| cg27320127 | 56660 | KCNK12 | 0.52 | 3.01E−11 | 1.81E−09 | 0.46 | 4.24E−08 | 5.86E−06 |
| cg27351358 | 627 | BDNF | 0.49 | 6.74E−06 | 8.92E−05 | 0.51 | 6.90E−08 | 8.36E−06 |
| cg27376271 | 147381 | CBLN2 | 0.29 | 2.39E−09 | 7.31E−08 | 0.22 | 1.07E−07 | 1.16E−05 |

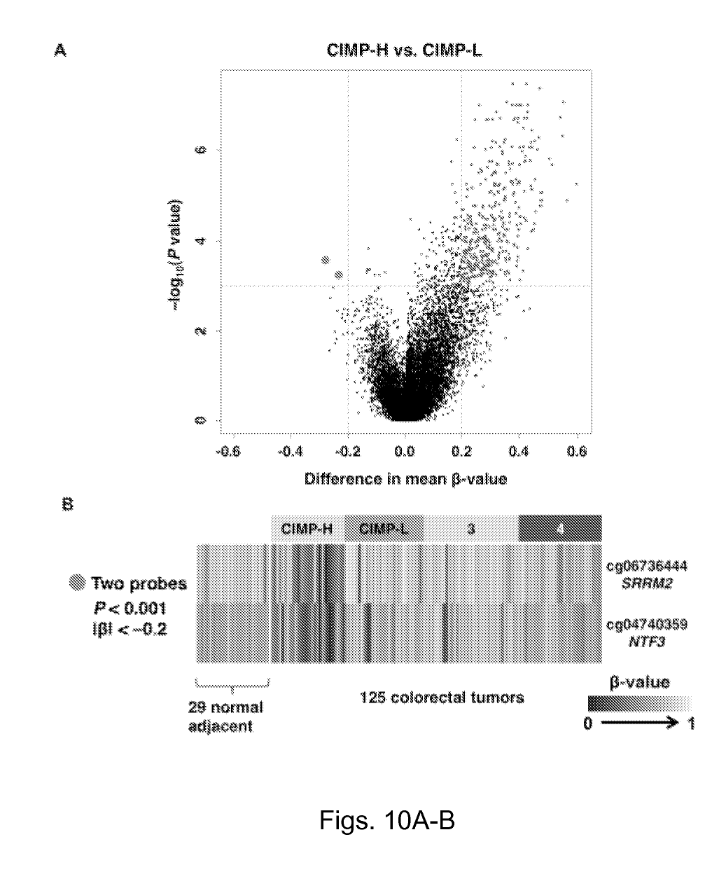

In order to determine whether there are DNA methylation markers specifically associated with CIMP-L subgroup, 22 CpG sites were examined that showed significant DNA hypermethylation in CIMP-L tumors, but not in CIMP-H tumors, as compared to non-CIMP tumors [FDR-adjusted P<0.001 (CIMP-L vs. non-CIMP) and P>0.05 (CIMP-H vs. non-CIMP)] (FIG. 2A). Although these markers exhibited statistically significant DNA methylation differences, they did not show strong CIMP-L-specificity when visualized and compared with individual tumor samples using a heatmap (FIG. 2B). The DNA methylation levels of each CpG locus was also directly compared between CIMP-H tumor and CIMP-L tumors (FIG. 10A). Two CpG loci in the promoter regions of SRRM2 and NTF3 were identified that are significantly hypermethylated in CIMP-L tumors compared with CIMP-H tumors (P<0.001 and mean β-value difference>0.2). Interestingly however, these two gene loci exhibit CIMP-H-specific DNA hypomethylation, as these are methylated in normal-adjacent tissues, as well as in tumors that belong to the cluster 3 and cluster 4 subgroups (FIG. 10B).

FIGS. 10A-B show, according to particular exemplary aspects, a comparison of DNA methylation profiles between CIMP-H and CIMP-L tumors. (A) The volcano plot shows the −1×log₁₀-transformed FDR-adjusted P value vs. the mean DNA methylation difference between CIMP-H and CIMP-L tumors. FDR-adjusted P=0.001 and |Δβ|=0.2 are used as a cutoff for differential methylation. Two CpG sites that are hypermethylated in CIMP-L tumors compared with CIMP-H tumors are indicated in green. (B) Heatmap representing Infinium DNA methylation β-values for the two CpG sites (labeled in green in panel A, herein reproduced in gray-scale, herein reproduced in gray-scale, that are significantly hypermethylated in CIMP-L compared with CIMP-H tumors. The four DNA methylation-based subgroups are indicated above the heatmap. A color gradient from dark blue to yellow (herein reproduced in gray-scale) was used to represent the low and high DNA methylation β-values, respectively.

Specifically, we also did not find a significant increase in MGMT DNA hypermethylation in CIMP-L tumors compared with non-CIMP tumors (P>0.05), as reported previously (Ogino et al., 2007). Clinically, Ogino and colleagues observed a significant association between CIMP-L and male sex (Ogino et al., 2006). Present Applicants also found that CIMP-L tumors are slightly more common in men (59%) than women (41%), although the association did not achieve statistical significance (P>0.05, Fisher's exact test).

### Example 4

### DNA Methylation Associated with KRAS-Mutant Tumors was Analyzed

Significant enrichment of KRAS mutations in the CIMP-L may suggest that KRAS mutations either induce DNA hypermethylation of a group of CpG loci or they might synergize with a specific DNA methylation profile associated with CIMP-L tumors. Interestingly, Shen et al. proposed a CIMP2 subtype of CRC, found to be tightly linked with KRAS mutations (92% of cases), using a limited number of DNA methylation markers (Shen et al., 2007).

In this Example, Applicants investigated whether KRAS mutations themselves are associated with DNA hypermethylation of specific sets of genes in CRC. We stratified tumors into three groups by their BRAF and KRAS mutation status: 1) BRAF mutant (n=17), 2) KRAS mutant (n=34), and 3) wild-type for both BRAF and KRAS (n=74), and then compared DNA methylation profiles between each group. A large number of CpG sites (715, FDR-adjusted P<0.0001) were identified that are significantly hypermethylated in tumors with BRAF mutation, all of which belong to the CIMP-H subgroup, as compared with tumors with wild-type for BRAF and KRAS (FIG. 2C). In contrast, only one CpG locus located in the promoter of JPH3 showed DNA hypermethylation in the KRAS-mutant tumors compared to the BRAF/KRAS wild-type tumors at the 0.01 significance level (FIG. 2C). Using a less stringent significance threshold (FDR-adjusted P<0.05), 157 CpGs were identified that showed more frequent DNA methylation in KRAS-mutant tumors (FIG. 2C). However, the mean β-value differences for the majority of these probes between tumors with KRAS mutation and those with BRAF/KRAS wild-type were found to be small (0.08±0.09, mean |Δβ|± s.d.). Among the 157 probes, the 22 CpG sites that showed substantial mean β-value difference ((|Δβ|>0.20) between KRAS-mutant tumors and BRAF/KRAS wild-type tumors were further examined. Importantly, we found that all of these CpG sites exhibit CIMP-L-specific DNA hypermethylation with much higher significance levels (Wilcoxon rank-sum test between CIMP-L and Non-CIMP tumors) (see Table 4 below). These observations indicate that the significant association between DNA methylation at these loci and KRAS mutation is mainly due to CIMP-L-based DNA hypermethylation.

TABLE 4
CpG sites associated with KRAS mutant tumors based on P value <0.05 (Wilcoxon
rank-sum test) and mean DNA methylation β-value difference >0.20 between KRAS mutant and
BRAF/KRAS wild-type tumors.

| | | -log10 (FDR adjusted P value) | | | |
|---|---|---|---|---|---|
| | | KRAS-M (34) | | Difference in | |
| | | vs. | | mean DNA methylation value | |
| | Gene | BRAF/KRAS- | CIMP-L (29) vs. | KRAS-M (34) vs. | CIMP-L (29) vs. |
| Probe ID | Symbol | WT (74) | Non-CIMP (68) | BRAF/KRAS-WT (74) | Non-CIMP (68) |
| cg23040064 | JPH3 | 2.94 | 5.23 | 0.23 | 0.26 |
| cg09053680 | UTF1 | 1.89 | 4.39 | 0.29 | 0.34 |
| cg07028533 | CNTNAP2 | 1.82 | 3.20 | 0.21 | 0.23 |
| cg25302419 | CTNND2 | 1.81 | 6.01 | 0.24 | 0.37 |
| cg16969623 | ZNF331 | 1.81 | 6.05 | 0.23 | 0.34 |
| cg13756879 | IGF2 | 1.76 | 6.94 | 0.21 | 0.37 |
| cg16041660 | PRICKLE1 | 1.75 | 4.87 | 0.27 | 0.34 |
| cg13168683 | JAKMIP1 | 1.66 | 5.44 | 0.22 | 0.34 |
| cg04713521 | PRRX2 | 1.65 | 6.32 | 0.20 | 0.34 |
| cg01322134 | WNT3A | 1.65 | 6.15 | 0.23 | 0.40 |
| cg01519742 | JAKMIP1 | 1.64 | 5.89 | 0.28 | 0.42 |
| cg26747293 | EGFLAM | 1.51 | 6.09 | 0.22 | 0.39 |
| cg26195812 | DPYSL5 | 1.51 | 5.89 | 0.22 | 0.36 |
| cg03168582 | DMRT1 | 1.51 | 4.87 | 0.22 | 0.33 |
| cg27420236 | RPRM | 1.50 | 2.95 | 0.22 | 0.24 |
| cg00687686 | NDRG4 | 1.46 | 6.57 | 0.21 | 0.39 |
| cg13031432 | NDRG4 | 1.45 | 6.20 | 0.21 | 0.41 |
| cg12874092 | VIM | 1.44 | 6.30 | 0.21 | 0.39 |
| cg01049530 | BMP3 | 1.44 | 2.11 | 0.24 | 0.28 |
| cg01557297 | SLC22A17 | 1.37 | 3.78 | 0.22 | 0.28 |
| cg02748539 | SLC9A3 | 1.35 | 5.35 | 0.21 | 0.37 |
| cg25157874 | QKI | 1.33 | 2.44 | 0.20 | 0.25 |

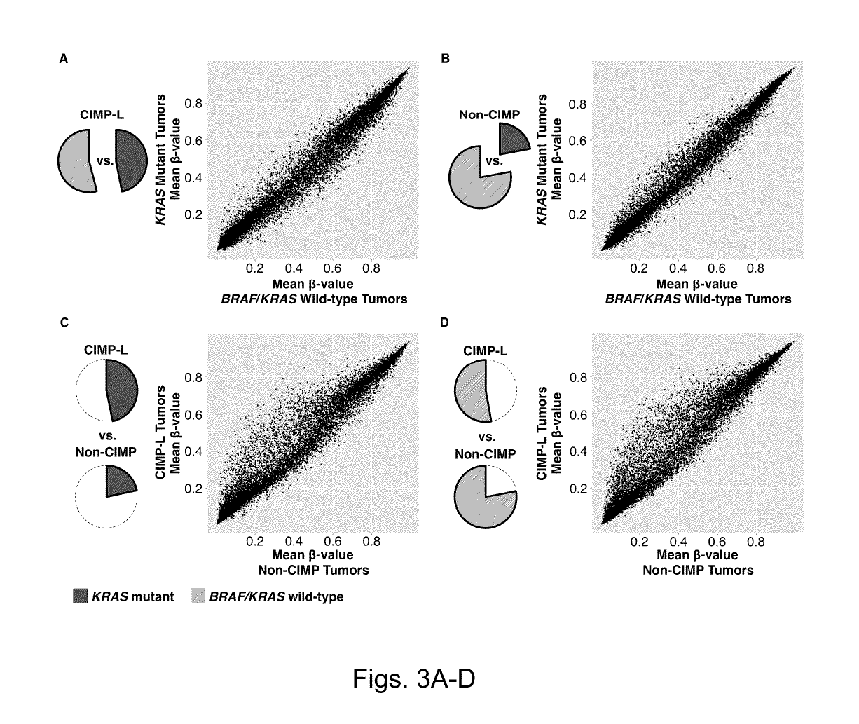

To further examine the DNA methylation profiles in KRAS mutant tumors and BRAF/KRAS wild-type tumors, CIMP-L and non-CIMP tumors were subdivided by their KRAS mutation status and the mean DNA methylation β-values were compared among these groups. Mean DNA methylation β-values for KRAS mutant tumors and those BRAF/KRAS wild-type tumors were observed to be well correlated within both the CIMP-L and non-CIMP subgroups (FIGS. 3A and 3B). Moreover, the CIMP-L subgroup exhibits higher mean DNA methylation in a number of CpG sites irrespective of KRAS mutation status (FIGS. 3C and 3D). These observations highlight the involvement of more complex molecular mechanisms in driving these DNA methylation clusters.

Specifically, FIGS. 3A-D show, according to particular exemplary aspects, that CIMP-L-associated DNA hypermethylation occurs independent of KRAS mutation status in CRC. CIMP-L and non-CIMP tumors were subdivided by their KRAS and BRAF mutation status (KRAS mutant or BRAF/KRAS wild-type), and mean DNA methylation β-values were compared between each group. Scatter plots comparing mean DNA methylation β-values between (A) KRAS mutant and BRAF/KRAS wild-type tumors within the CIMP-L subgroup, (B) KRAS mutant and BRAF/KRAS wild-type tumors within the non-CIMP subgroup, (C) KRAS mutant, CIMP-L tumors versus KRAS mutant, non-CIMP tumors and (D) BRAF/KRAS wild-type, CIMP-L tumors compared to non-CIMP tumors with the same genotype.

### Example 5

### Sequence Characteristics of CIMP-Associated Gene Promoters were Analyzed

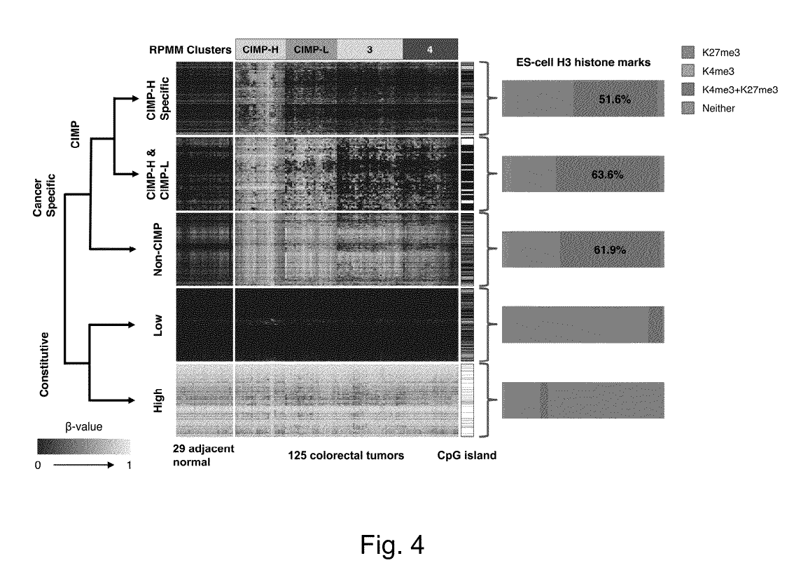

In this working example, gene promoters that acquired cancer-specific DNA methylation were classified into three categories based on their DNA methylation level profiles across colorectal tumor subtypes (see Methods of Example 1 herein, and Table 5 below): 1) CIMP-associated DNA methylation markers specific for the CIMP-H subgroup only, 2) CIMP-specific DNA methylation shared between both the CIMP-H and CIMP-L subgroups, and 3) non-CIMP cancer-specific DNA methylation. For comparison, 500 gene promoters were included in two additional groups that did not exhibit cancer-specific DNA methylation profiles, and were either constitutively methylated or unmethylated across tumor and adjacent-normal tissue samples (FIG. 4).

Applicants explored whether the distinction between these groups of promoters can be attributable to simple structural and sequence characteristics. The majority of genes in all three groups that exhibited cancer-specific DNA methylation as well as the genes that were constitutively unmethylated in normal and tumor tissues are located within CpG islands defined by Takai and Jones (Takai and Jones, 2002) (see FIG. 4 herein).

FIG. 4 shows, according to particular exemplary aspects, ES-cell histone marks associated with genes in the five classification groups described in the text. Shown are heatmap representations of DNA methylation β-values for unique gene promoters that belong to five different categories: 1. CIMP-H specific: CIMP-associated DNA methylation markers specific for CIMP-H subgroup only (n=415 genes), 2. CIMP-H & CIMP-L: CIMP-specific DNA methylation shared between the CIMP-H and CIMP-L subgroups (n=73 genes), 3. Non-CIMP: cancer-specific DNA methylation but outside of the CIMP context (n=547 genes), 4. Constitutive-Low: Constitutively unmethylated genes in both tumor and adjacent normal tissue samples (n=500 genes), 5. Constitutive-High: Constitutively methylated in both tumor and adjacent normal tissue samples (n=500 genes). Genes containing CpG islands defined by Takai and Jones are indicated by horizontal black bars immediately to the right of each heatmap. The bar charts to the right of each heatmap show the proportion of gene promoters with occupancy of histone H3 lysine 4 trimethylation (K4) and/or histone H3 lysine 27 trimethylation (K27) in human ES cells. Probes that do not have these histone mark information (listed in Table 5 as “NA”) were not included in the bar chart calculations. The probes in each category are ordered according to the unsupervised hierarchal clustering using correlation distance metric and average linkage method. The RPMM-based cluster assignments are indicated above the heatmaps.

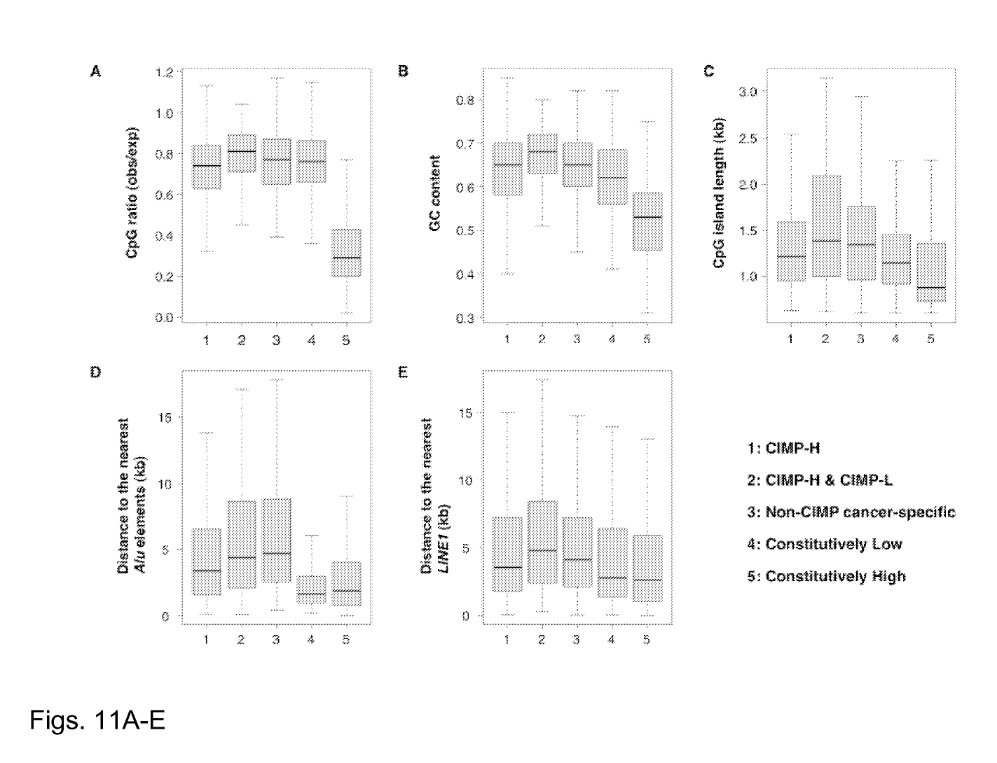

Present Applicants did not observe significant differences in the overall distribution with respect to the CpG observed-to-expected ratio, G:C content, and CpG island length among these four groups of DNA sequences (FIG. 11A-C). Therefore, these DNA sequence characteristics do not discriminate among CIMP-associated, non-CIMP-associated, and constitutively unmethylated sequences.

FIGS. 11A-E show, according to particular exemplary aspects, DNA structural and sequence characteristics associated with five different gene categories based on DNA methylation profiles in colorectal tumors. The five categories include: 1, CIMP-associated DNA methylation markers specific for the CIMP-H subgroup only; 2, CIMP-specific DNA methylation shared between both the CIMP-H and CIMP-L subgroups; 3, non-CIMP cancer-specific DNA methylation; 4, constitutively unmethylated across tumor and adjacent normal tissue samples; 5, constitutively methylated across tumor and adjacent normal tissue samples. Distribution of (A) observed CpG/expected CpG ratio and (B) GC content over 250 bp upstream and 250 bp downstream from the interrogated CpG dinucleotide on the Infinium DNA methylation BeadArray, (C) the Takai and Jones-calculated CpG island length (Takai and Jones, 2002), (D, E) distances of Infinium DNA methylation probes to the nearest (D) ALU and (E) LINE repetitive element. In each box plot, the top and bottom edges are the 25th and 75th quartiles, respectively. The horizontal line within each box identifies the median. The whiskers above and below the box extend to at most 1.5 times the interquartile range (IQR).

Applicants also considered that specific sequence motifs or repeat sequences surrounding CpG islands may have a role in differential DNA hypermethylation specifically in CIMP tumors. There was no enrichment or depletion of any di- or tetranucleotide sequences and known transcription factor binding sites in the CIMP-associated CpG islands (data not shown). Recently, Estecio and colleagues reported that retrotransposons are more frequently associated with CpG islands that are resistant to DNA hypermethylation than those that are susceptible to DNA hypermethylation (Estecio et al., 2010). Consistent with their observations, we found that the distances of Infinium DNA methylation probes to the nearest ALU repetitive element were significantly different between cancer-specifically methylated DNA promoter sequences (median distance: 4,300 bp) and those that do not exhibit cancer-specific DNA methylation changes (median distance: 1,730 bp) (P<2.2×10₋₁₆, Wilcoxon rank-sum test) (FIG. 11D). Similarly, cancer-specifically methylated DNA promoter sequences showed a greater median distance to LINE repetitive elements compared with those that do not show cancer-specific DNA methylation changes (3,880 bp vs. 2,710 bp; P=1.9×10₋₁₃, Wilcoxon rank-sum test). Interestingly, differences in the proximity to ALU repeat sequences between CIMP-H-associated and non-CIMP-associated promoters were observed to be statistically significant with median distances of 3,410 bp and 4,730 bp respectively (P=1.8×10₋₆, Wilcoxon rank-sum test; FIG. 11D). However, no such significant differences for LINE repetitive element between CIMP-H-associated and non-CIMP-associated promoters (P=0.18) were observed.

The trimethylation status of histone H3 lysine 4 (H3K4me3) and histone H3 lysine 27 (H3K27me3) were next identified in human ES cells for genes in the five classification groups described above using a previously published dataset (Ku et al., 2008). The genes that are constitutively unmethylated across tumor and adjacent-normal tissue samples were found to be highly enriched for H3K4me3, whereas those that are constitutively methylated are enriched for chromatin states with neither marks in ES cells (FIG. 4). As has previously been reported, the fraction of genes that coincide with ES-cell bivalent domains is substantially higher for the genes that undergo cancer-specific DNA methylation than those that are constitutively methylated or unmethylated across tumor and adjacent-normal tissue samples. Applicants found that more than 50% of colorectal cancer-specific DNA hypermethylation occurs at ES-cell bivalent domains. However, the proportion of the ES-cell bivalent domains among CIMP-associated and non-CIMP-associated genes is similar, suggesting that the features associated with these targets are not specific for CIMP-positive tumors nor CIMP genes, but general features of colorectal cancer (FIG. 4).

TABLE 5
Gene promoter classification among colorectal samples.

| | | | | CIMP-H vs. | | CIMP-L vs. | | |
|---|---|---|---|---|---|---|---|---|
| | | | CIMP-H | nonCIMP | CIMP-L | nonCIMP | | |
| | Gene | | mean | FDR-adjusted | mean | FDR-adjusted | ES cell histone H3 | Gene |
| Probe ID | Symbol | Gene ID | beta-value | P value | beta-value | P value | status | category |
| cg02873524 | PAPPA | 5069 | 0.54 | 3.85E−11 | 0.21 | 0.056 | K4me3 + K27me3 | CIMP-H |
| cg03447931 | BMP6 | 654 | 0.58 | 3.85E−11 | 0.16 | 0.235 | K4me3 + K27me3 | CIMP-H |
| cg06638966 | COL19A1 | 1310 | 0.52 | 3.85E−11 | 0.22 | 0.396 | K4me3 + K27me3 | CIMP-H |
| cg06954481 | GBX2 | 2637 | 0.79 | 3.85E−11 | 0.34 | 0.098 | K4me3 + K27me3 | CIMP-H |
| cg16778809 | ADAM23 | 8745 | 0.75 | 3.85E−11 | 0.34 | 0.074 | K4me3 + K27me3 | CIMP-H |
| cg17497271 | GPR176 | 11245 | 0.59 | 3.85E−11 | 0.18 | 0.336 | K4me3 | CIMP-H |
| cg19283196 | SLC10A4 | 201780 | 0.53 | 3.85E−11 | 0.27 | 0.099 | K4me3 + K27me3 | CIMP-H |
| cg21665000 | MYOCD | 93649 | 0.47 | 3.85E−11 | 0.09 | 0.790 | K4me3 | CIMP-H |
| cg24317255 | RGS17 | 26575 | 0.53 | 3.85E−11 | 0.18 | 0.242 | K4me3 + K27me3 | CIMP-H |
| cg24686358 | COL9A2 | 1298 | 0.57 | 3.85E−11 | 0.19 | 0.807 | K4me3 + K27me3 | CIMP-H |
| cg26359204 | NKX6-1 | 4825 | 0.38 | 3.85E−11 | 0.08 | 0.630 | K4me3 + K27me3 | CIMP-H |
| cg27049761 | B3GNT4 | 79369 | 0.63 | 3.85E−11 | 0.29 | 0.067 | K4me3 + K27me3 | CIMP-H |
| cg19219437 | PCOLCE2 | 26577 | 0.54 | 4.35E−11 | 0.12 | 0.636 | K4me3 | CIMP-H |
| cg06638433 | IGF2BP1 | 10642 | 0.56 | 4.37E−11 | 0.17 | 0.924 | K4me3 | CIMP-H |
| cg15613048 | KIF17 | 57576 | 0.63 | 4.37E−11 | 0.25 | 0.110 | K4me3 | CIMP-H |
| cg04528819 | KLF14 | 136259 | 0.54 | 4.65E−11 | 0.22 | 0.064 | K4me3 + K27me3 | CIMP-H |
| cg14223995 | UCP1 | 7350 | 0.64 | 4.93E−11 | 0.17 | 0.636 | K4me3 + K27me3 | CIMP-H |
| cg22619563 | EPHA3 | 2042 | 0.28 | 4.93E−11 | 0.12 | 0.053 | K4me3 + K27me3 | CIMP-H |
| cg23391006 | OXTR | 5021 | 0.35 | 4.93E−11 | 0.06 | 0.448 | K4me3 + K27me3 | CIMP-H |
| cg06379754 | CACNA2D1 | 781 | 0.52 | 5.09E−11 | 0.15 | 0.693 | NA | CIMP-H |
| cg18618334 | CXCL12 | 6387 | 0.58 | 5.66E−11 | 0.31 | 0.353 | NA | CIMP-H |
| cg06836772 | PRKAA2 | 5563 | 0.56 | 6.41E−11 | 0.22 | 0.106 | NA | CIMP-H |
| cg15980408 | TMEM22 | 80723 | 0.58 | 6.54E−11 | 0.13 | 0.515 | K4me3 | CIMP-H |
| cg22469841 | FSTL1 | 11167 | 0.52 | 6.67E−11 | 0.08 | 0.253 | K4me3 | CIMP-H |
| cg06055013 | ATRNL1 | 26033 | 0.52 | 6.91E−11 | 0.18 | 0.087 | K4me3 + K27me3 | CIMP-H |
| cg18815943 | FOXE3 | 2301 | 0.62 | 6.91E−11 | 0.16 | 0.600 | K27me3 | CIMP-H |
| cg09339301 | QKI | 9444 | 0.70 | 7.26E−11 | 0.30 | 0.229 | K4me3 | CIMP-H |
| cg09881855 | SNAI2 | 6591 | 0.49 | 7.37E−11 | 0.18 | 0.607 | NA | CIMP-H |
| cg07965823 | ISM2 | 145501 | 0.71 | 8.02E−11 | 0.23 | 0.292 | K4me3 + K27me3 | CIMP-H |
| cg25735280 | PDZD2 | 23037 | 0.36 | 8.02E−11 | 0.05 | 0.649 | NA | CIMP-H |
| cg01425670 | NEGR1 | 257194 | 0.37 | 8.42E−11 | 0.08 | 0.166 | NA | CIMP-H |
| cg24493940 | MMP17 | 4326 | 0.41 | 8.77E−11 | 0.17 | 0.226 | K4me3 + K27me3 | CIMP-H |
| cg04993257 | PLAC2 | 257000 | 0.60 | 8.96E−11 | 0.36 | 0.107 | NA | CIMP-H |
| cg02867079 | HHIPL1 | 84439 | 0.45 | 1.07E−10 | 0.18 | 0.053 | K4me3 | CIMP-H |
| cg07336230 | KIF6 | 221458 | 0.46 | 1.07E−10 | 0.16 | 0.066 | K4me3 | CIMP-H |
| cg07850604 | INSM2 | 84684 | 0.50 | 1.07E−10 | 0.19 | 0.118 | K4me3 + K27me3 | CIMP-H |
| cg25917510 | HOXC8 | 3224 | 0.60 | 1.07E−10 | 0.21 | 0.347 | K27me3 | CIMP-H |
| cg17398595 | SH3GL2 | 6456 | 0.63 | 1.11E−10 | 0.23 | 0.879 | K4me3 + K27me3 | CIMP-H |
| cg25301180 | ERC2 | 26059 | 0.47 | 1.15E−10 | 0.17 | 0.381 | K4me3 | CIMP-H |
| cg12373771 | CECR6 | 27439 | 0.66 | 1.18E−10 | 0.26 | 0.298 | None | CIMP-H |
| cg06763078 | KCNC1 | 3746 | 0.59 | 1.27E−10 | 0.04 | 0.500 | NA | CIMP-H |
| cg25097436 | RTN1 | 6252 | 0.57 | 1.27E−10 | 0.18 | 0.216 | K4me3 + K27me3 | CIMP-H |
| cg08997253 | GRIN3A | 116443 | 0.46 | 1.41E−10 | 0.14 | 0.220 | K4me3 + K27me3 | CIMP-H |
| cg09754413 | RNF182 | 221687 | 0.49 | 1.78E−10 | 0.17 | 0.482 | K4me3 + K27me3 | CIMP-H |
| cg08179907 | RAB39 | 54734 | 0.54 | 1.83E−10 | 0.12 | 0.473 | K4me3 | CIMP-H |
| cg23698058 | PRKACB | 5567 | 0.58 | 1.83E−10 | 0.16 | 0.121 | K4me3 | CIMP-H |
| cg26491213 | SIX3 | 6496 | 0.30 | 1.83E−10 | 0.08 | 0.876 | K4me3 + K27me3 | CIMP-H |
| cg15447479 | SMO | 6608 | 0.64 | 1.96E−10 | 0.28 | 0.066 | K4me3 | CIMP-H |
| cg20123891 | NXPH3 | 11248 | 0.48 | 1.96E−10 | 0.27 | 0.118 | K4me3 + K27me3 | CIMP-H |
| cg25462291 | HEYL | 26508 | 0.60 | 1.96E−10 | 0.26 | 0.242 | K4me3 + K27me3 | CIMP-H |
| cg10613381 | UPB1 | 51733 | 0.77 | 2.01E−10 | 0.59 | 0.073 | NA | CIMP-H |
| cg05881135 | SYNM | 23336 | 0.52 | 2.15E−10 | 0.09 | 0.576 | K4me3 + K27me3 | CIMP-H |
| cg06222851 | OGDHL | 55753 | 0.65 | 2.15E−10 | 0.11 | 0.884 | K4me3 | CIMP-H |
| cg10692870 | FN1 | 2335 | 0.39 | 2.15E−10 | 0.13 | 0.461 | K4me3 | CIMP-H |
| cg22886089 | SCG3 | 29106 | 0.48 | 2.15E−10 | 0.09 | 0.765 | K4me3 | CIMP-H |
| cg21296230 | GREM1 | 26585 | 0.54 | 2.24E−10 | 0.27 | 0.271 | K4me3 + K27me3 | CIMP-H |
| cg23214267 | EYA2 | 2139 | 0.30 | 2.34E−10 | 0.10 | 0.376 | K4me3 + K27me3 | CIMP-H |
| cg06866657 | LHX6 | 26468 | 0.37 | 2.57E−10 | 0.16 | 0.180 | K4me3 + K27me3 | CIMP-H |
| cg16632715 | HOXD11 | 3237 | 0.44 | 2.66E−10 | 0.20 | 0.461 | K27me3 | CIMP-H |
| cg21229859 | MYEF2 | 50804 | 0.42 | 2.78E−10 | 0.12 | 0.192 | K4me3 | CIMP-H |
| cg05098471 | MEIS1 | 4211 | 0.48 | 3.04E−10 | 0.12 | 0.113 | K4me3 + K27me3 | CIMP-H |
| cg10720654 | PTENP1 | 11191 | 0.57 | 3.35E−10 | 0.40 | 0.192 | NA | CIMP-H |
| cg26096837 | FGF19 | 9965 | 0.53 | 3.35E−10 | 0.35 | 0.145 | NA | CIMP-H |
| cg02780295 | PCDHGC3 | 5098 | 0.61 | 3.48E−10 | 0.34 | 0.051 | K4me3 + K27me3 | CIMP-H |
| cg16944093 | LIMS2 | 55679 | 0.61 | 3.79E−10 | 0.45 | 0.137 | K4me3 + K27me3 | CIMP-H |
| cg25835225 | ZNF350 | 59348 | 0.43 | 3.79E−10 | 0.19 | 0.070 | K4me3 | CIMP-H |
| cg12220493 | NKX2-1 | 7080 | 0.47 | 3.95E−10 | 0.13 | 0.098 | NA | CIMP-H |
| cg17460095 | FERMT2 | 10979 | 0.38 | 3.95E−10 | 0.06 | 0.559 | K4me3 | CIMP-H |
| cg27426707 | CACNA1G | 8913 | 0.42 | 3.95E−10 | 0.19 | 0.187 | K4me3 + K27me3 | CIMP-H |
| cg05647859 | LIN7A | 8825 | 0.69 | 4.62E−10 | 0.37 | 0.054 | K4me3 | CIMP-H |
| cg02548238 | LOX | 4015 | 0.51 | 4.78E−10 | 0.06 | 0.969 | K4me3 + K27me3 | CIMP-H |
| cg09872233 | ALOX15 | 246 | 0.54 | 4.99E−10 | 0.19 | 0.118 | NA | CIMP-H |
| cg24975564 | PDE3A | 5139 | 0.42 | 6.03E−10 | 0.14 | 0.118 | K4me3 | CIMP-H |
| cg05016953 | SLC6A4 | 6532 | 0.43 | 6.20E−10 | 0.03 | 0.900 | K27me3 | CIMP-H |
| cg17252960 | ID4 | 3400 | 0.60 | 6.20E−10 | 0.27 | 0.496 | K4me3 | CIMP-H |
| cg21965997 | CALY | 50632 | 0.52 | 6.20E−10 | 0.22 | 0.117 | K27me3 | CIMP-H |
| cg23423382 | ZNF287 | 57336 | 0.48 | 6.20E−10 | 0.20 | 0.123 | K4me3 + K27me3 | CIMP-H |
| cg18695917 | FSTL5 | 56884 | 0.44 | 6.82E−10 | 0.20 | 0.180 | K4me3 | CIMP-H |
| cg07684796 | DKK1 | 22943 | 0.53 | 7.50E−10 | 0.24 | 0.437 | K4me3 + K27me3 | CIMP-H |
| cg09068492 | CALCA | 796 | 0.51 | 8.22E−10 | 0.23 | 0.916 | K4me3 + K27me3 | CIMP-H |
| cg09156233 | BMPR1B | 658 | 0.54 | 8.22E−10 | 0.15 | 0.245 | K4me3 + K27me3 | CIMP-H |
| cg25361106 | TLX2 | 3196 | 0.33 | 8.22E−10 | 0.11 | 0.539 | NA | CIMP-H |
| cg18956481 | CYP24A1 | 1591 | 0.70 | 9.02E−10 | 0.35 | 0.580 | K4me3 + K27me3 | CIMP-H |
| cg23828595 | PRKG1 | 5592 | 0.43 | 9.02E−10 | 0.09 | 0.333 | NA | CIMP-H |
| cg06994747 | ADAMTS10 | 81794 | 0.41 | 9.35E−10 | 0.11 | 0.505 | K4me3 | CIMP-H |
| cg07558455 | KANK4 | 163782 | 0.55 | 9.35E−10 | 0.14 | 0.338 | K4me3 + K27me3 | CIMP-H |
| cg12717594 | RECK | 8434 | 0.49 | 9.35E−10 | 0.13 | 0.226 | K4me3 | CIMP-H |
| cg20673481 | KCNS3 | 3790 | 0.48 | 9.35E−10 | 0.20 | 0.245 | K4me3 | CIMP-H |
| cg01530101 | KCNQ1DN | 55539 | 0.72 | 9.77E−10 | 0.45 | 0.074 | NA | CIMP-H |
| cg02515725 | PDLIM3 | 27295 | 0.54 | 9.77E−10 | 0.16 | 0.477 | K4me3 | CIMP-H |
| cg22334000 | C4orf22 | 255119 | 0.44 | 9.77E−10 | 0.09 | 0.751 | K4me3 + K27me3 | CIMP-H |
| cg14436761 | RAMP2 | 10266 | 0.63 | 1.08E−09 | 0.32 | 0.180 | K4me3 | CIMP-H |
| cg09722397 | GRIN2C | 2905 | 0.44 | 1.29E−09 | 0.08 | 0.616 | K4me3 + K27me3 | CIMP-H |
| cg01836044 | PCDH20 | 64881 | 0.41 | 1.49E−09 | 0.20 | 0.208 | K4me3 + K27me3 | CIMP-H |
| cg11832722 | DSC3 | 1825 | 0.61 | 1.65E−09 | 0.18 | 0.907 | K4me3 + K27me3 | CIMP-H |
| cg19803671 | UBE2E2 | 7325 | 0.30 | 1.65E−09 | 0.06 | 0.983 | K4me3 | CIMP-H |
| cg08918749 | LPL | 4023 | 0.60 | 1.72E−09 | 0.37 | 0.271 | K4me3 + K27me3 | CIMP-H |
| cg14133708 | EMILIN2 | 84034 | 0.51 | 1.89E−09 | 0.20 | 0.110 | K4me3 + K27me3 | CIMP-H |
| cg07623294 | ELAVL2 | 1993 | 0.25 | 1.99E−09 | 0.12 | 0.088 | K4me3 + K27me3 | CIMP-H |
| cg01899253 | FLT1 | 2321 | 0.50 | 2.09E−09 | 0.27 | 0.724 | NA | CIMP-H |
| cg24371225 | MGC42105 | 167359 | 0.47 | 2.30E−09 | 0.17 | 0.088 | K4me3 + K27me3 | CIMP-H |
| cg26090652 | C1QTNF5 | 114902 | 0.50 | 2.30E−09 | 0.18 | 0.636 | K4me3 + K27me3 | CIMP-H |
| cg12515638 | SFRP4 | 6424 | 0.49 | 2.41E−09 | 0.23 | 0.146 | K4me3 + K27me3 | CIMP-H |
| cg24003542 | MCC | 4163 | 0.38 | 2.52E−09 | 0.10 | 0.083 | K4me3 + K27me3 | CIMP-H |
| cg24265806 | FAM126A | 84668 | 0.43 | 2.52E−09 | 0.06 | 0.751 | K4me3 | CIMP-H |
| cg00308133 | GAMT | 2593 | 0.46 | 2.64E−09 | 0.07 | 0.643 | K4me3 | CIMP-H |
| cg07212894 | SLC38A3 | 10991 | 0.44 | 2.64E−09 | 0.10 | 0.161 | K4me3 + K27me3 | CIMP-H |
| cg15757271 | WNT5A | 7474 | 0.30 | 2.74E−09 | 0.03 | 0.657 | K4me3 + K27me3 | CIMP-H |
| cg24417499 | HPCA | 3208 | 0.50 | 2.74E−09 | 0.28 | 0.630 | K4me3 | CIMP-H |
| cg18438777 | NPY5R | 4889 | 0.65 | 2.89E−09 | 0.23 | 0.969 | K4me3 + K27me3 | CIMP-H |
| cg09536738 | EFHD1 | 80303 | 0.44 | 3.01E−09 | 0.26 | 0.470 | K4me3 + K27me3 | CIMP-H |
| cg23559331 | KCNH4 | 23415 | 0.56 | 3.01E−09 | 0.18 | 0.190 | K4me3 | CIMP-H |
| cg24273512 | POPDC3 | 64208 | 0.43 | 3.16E−09 | 0.23 | 0.064 | K4me3 | CIMP-H |
| cg25484904 | CWH43 | 80157 | 0.64 | 3.16E−09 | 0.32 | 0.876 | K4me3 + K27me3 | CIMP-H |
| cg15075170 | TMEFF1 | 8577 | 0.65 | 3.27E−09 | 0.05 | 0.559 | K4me3 | CIMP-H |
| cg19037167 | TLR2 | 7097 | 0.45 | 3.27E−09 | 0.08 | 0.776 | K4me3 + K27me3 | CIMP-H |
| cg21801378 | BRUNOL6 | 60677 | 0.49 | 3.27E−09 | 0.09 | 0.884 | K4me3 + K27me3 | CIMP-H |
| cg23699324 | CTNNA2 | 1496 | 0.48 | 3.42E−09 | 0.34 | 0.074 | K4me3 | CIMP-H |
| cg07102705 | HTR4 | 3360 | 0.56 | 3.76E−09 | 0.21 | 0.381 | K4me3 + K27me3 | CIMP-H |
| cg09068528 | ACADL | 33 | 0.57 | 3.76E−09 | 0.23 | 0.157 | K4me3 + K27me3 | CIMP-H |
| cg25680829 | LHX4 | 89884 | 0.36 | 3.76E−09 | 0.14 | 0.657 | NA | CIMP-H |
| cg00282347 | CHD5 | 26038 | 0.46 | 3.87E−09 | 0.19 | 0.408 | NA | CIMP-H |
| cg20051033 | CPNE9 | 151835 | 0.35 | 3.87E−09 | 0.06 | 0.846 | K4me3 | CIMP-H |
| cg24014661 | TCTE1 | 202500 | 0.31 | 3.87E−09 | 0.03 | 0.856 | K4me3 | CIMP-H |
| cg04324308 | COL4A3 | 1285 | 0.53 | 4.03E−09 | 0.06 | 0.884 | K4me3 + K27me3 | CIMP-H |
| cg16539629 | C14orf132 | 56967 | 0.63 | 4.03E−09 | 0.32 | 0.051 | NA | CIMP-H |
| cg07674153 | TSHR | 7253 | 0.28 | 4.84E−09 | 0.12 | 0.320 | K4me3 + K27me3 | CIMP-H |
| cg02886284 | CPE | 1363 | 0.53 | 5.28E−09 | 0.13 | 0.268 | K4me3 + K27me3 | CIMP-H |
| cg06150468 | BATF3 | 55509 | 0.36 | 5.28E−09 | 0.07 | 0.113 | NA | CIMP-H |
| cg08186362 | HRH3 | 11255 | 0.59 | 5.28E−09 | 0.14 | 0.646 | K4me3 + K27me3 | CIMP-H |
| cg16063112 | C10orf107 | 219621 | 0.47 | 5.28E−09 | 0.19 | 0.920 | K4me3 | CIMP-H |
| cg11932564 | TNFRSF13C | 115650 | 0.39 | 5.52E−09 | 0.11 | 0.197 | K4me3 + K27me3 | CIMP-H |
| cg26057752 | PGAM2 | 5224 | 0.62 | 5.79E−09 | 0.35 | 0.943 | K4me3 | CIMP-H |
| cg04473302 | SLC26A4 | 5172 | 0.23 | 6.04E−09 | 0.05 | 0.767 | K4me3 + K27me3 | CIMP-H |
| cg08785534 | GAL | 51083 | 0.36 | 6.04E−09 | 0.15 | 0.415 | K4me3 | CIMP-H |
| cg04001333 | FLVCR2 | 55640 | 0.63 | 6.27E−09 | 0.32 | 0.827 | K4me3 | CIMP-H |
| cg12422450 | CHGA | 1113 | 0.70 | 6.27E−09 | 0.42 | 0.063 | K4me3 | CIMP-H |
| cg19378133 | A2BP1 | 54715 | 0.55 | 6.56E−09 | 0.31 | 0.373 | K4me3 + K27me3 | CIMP-H |
| cg25010118 | DSEL | 92126 | 0.61 | 7.24E−09 | 0.16 | 0.884 | K4me3 | CIMP-H |
| cg25179291 | FNBP1 | 23048 | 0.45 | 7.58E−09 | 0.05 | 0.748 | K4me3 | CIMP-H |
| cg25211525 | C6orf145 | 221749 | 0.65 | 7.58E−09 | 0.08 | 0.860 | K4me3 | CIMP-H |
| cg24794531 | TRPC1 | 7220 | 0.48 | 7.95E−09 | 0.19 | 0.418 | K4me3 | CIMP-H |
| cg06722216 | NOL4 | 8715 | 0.46 | 8.36E−09 | 0.12 | 0.704 | K4me3 + K27me3 | CIMP-H |
| cg04623955 | DIO3 | 1735 | 0.50 | 8.78E−09 | 0.22 | 0.920 | NA | CIMP-H |
| cg13619915 | SLITRK3 | 22865 | 0.47 | 9.17E−09 | 0.11 | 0.171 | K4me3 + K27me3 | CIMP-H |
| cg07634191 | SCARA5 | 286133 | 0.46 | 9.52E−09 | 0.16 | 0.717 | None | CIMP-H |
| cg14831838 | CDK5R2 | 8941 | 0.46 | 9.52E−09 | 0.09 | 0.633 | K4me3 + K27me3 | CIMP-H |
| cg09017174 | SLC1A2 | 6506 | 0.46 | 1.00E−08 | 0.31 | 0.118 | K4me3 + K27me3 | CIMP-H |
| cg01226811 | KCNJ8 | 3764 | 0.42 | 1.04E−08 | 0.14 | 0.567 | K4me3 + K27me3 | CIMP-H |
| cg09892203 | CACNG4 | 27092 | 0.26 | 1.04E−08 | 0.06 | 0.576 | K4me3 + K27me3 | CIMP-H |
| cg15105703 | DUOXA1 | 90527 | 0.44 | 1.09E−08 | 0.10 | 0.892 | K4me3 + K27me3 | CIMP-H |
| cg07744166 | WASF3 | 10810 | 0.47 | 1.19E−08 | 0.31 | 0.068 | K4me3 + K27me3 | CIMP-H |
| cg16158681 | MT3 | 4504 | 0.56 | 1.24E−08 | 0.39 | 0.051 | K4me3 + K27me3 | CIMP-H |
| cg16793061 | EYA1 | 2138 | 0.36 | 1.24E−08 | 0.09 | 0.336 | K4me3 | CIMP-H |
| cg03751813 | ZNF585B | 92285 | 0.43 | 1.29E−08 | 0.13 | 0.787 | None | CIMP-H |
| cg21948783 | WNT1 | 7471 | 0.54 | 1.35E−08 | 0.20 | 0.660 | K4me3 + K27me3 | CIMP-H |
| cg20557202 | SLC5A5 | 6528 | 0.31 | 1.41E−08 | 0.15 | 0.085 | K27me3 | CIMP-H |
| cg10182321 | STK32B | 55351 | 0.42 | 1.48E−08 | 0.22 | 0.050 | NA | CIMP-H |
| cg12770741 | NXN | 64359 | 0.71 | 1.48E−08 | 0.49 | 0.986 | NA | CIMP-H |
| cg20937139 | PDGFC | 56034 | 0.63 | 1.48E−08 | 0.20 | 0.668 | K4me3 | CIMP-H |
| cg08126211 | KAAG1 | 353219 | 0.41 | 1.55E−08 | 0.16 | 0.989 | K4me3 | CIMP-H |
| cg08030082 | POMC | 5443 | 0.73 | 1.62E−08 | 0.61 | 0.051 | K4me3 + K27me3 | CIMP-H |
| cg25920792 | HTRA1 | 5654 | 0.47 | 1.70E−08 | 0.15 | 0.422 | K4me3 + K27me3 | CIMP-H |
| cg16358826 | GABRA4 | 2557 | 0.38 | 1.87E−08 | 0.24 | 0.071 | NA | CIMP-H |
| cg26465611 | MEGF10 | 84466 | 0.64 | 1.87E−08 | 0.48 | 0.079 | K4me3 | CIMP-H |
| cg12858460 | EOMES | 8320 | 0.50 | 2.05E−08 | 0.24 | 0.356 | K4me3 + K27me3 | CIMP-H |
| cg06637774 | P2RY6 | 5031 | 0.62 | 2.15E−08 | 0.25 | 0.903 | K27me3 | CIMP-H |
| cg18119529 | ZNF114 | 163071 | 0.27 | 2.26E−08 | 0.16 | 0.289 | K4me3 | CIMP-H |
| cg09649610 | GNG4 | 2786 | 0.66 | 2.34E−08 | 0.26 | 0.396 | K4me3 | CIMP-H |
| cg14155416 | L3MBTL4 | 91133 | 0.54 | 2.34E−08 | 0.30 | 0.388 | K4me3 + K27me3 | CIMP-H |
| cg17054360 | MTERF | 7978 | 0.42 | 2.34E−08 | 0.28 | 0.067 | K4me3 | CIMP-H |
| cg05860890 | KCNV1 | 27012 | 0.45 | 2.46E−08 | 0.14 | 0.457 | K4me3 + K27me3 | CIMP-H |
| cg23495733 | CPNE8 | 144402 | 0.48 | 2.57E−08 | 0.22 | 0.096 | K4me3 | CIMP-H |
| cg22377389 | GJB6 | 10804 | 0.58 | 2.68E−08 | 0.25 | 0.963 | NA | CIMP-H |
| cg04747322 | SNCAIP | 9627 | 0.52 | 2.80E−08 | 0.32 | 0.101 | NA | CIMP-H |
| cg09038885 | ADRA1B | 147 | 0.28 | 2.80E−08 | 0.05 | 0.985 | NA | CIMP-H |
| cg23472215 | GSTM3 | 2947 | 0.58 | 2.80E−08 | 0.17 | 0.751 | K4me3 + K27me3 | CIMP-H |
| cg04624659 | SPAG17 | 200162 | 0.47 | 3.18E−08 | 0.26 | 0.310 | K4me3 | CIMP-H |
| cg00247489 | CR2 | 1380 | 0.30 | 3.31E−08 | 0.07 | 0.265 | K4me3 + K27me3 | CIMP-H |
| cg05674944 | SLC30A2 | 7780 | 0.52 | 3.31E−08 | 0.11 | 0.721 | K4me3 + K27me3 | CIMP-H |
| cg22578204 | TIMP3 | 7078 | 0.47 | 3.31E−08 | 0.21 | 0.708 | K4me3 | CIMP-H |
| cg18275051 | CYB5R1 | 51706 | 0.50 | 3.45E−08 | 0.23 | 0.250 | NA | CIMP-H |
| cg01144286 | C20orf103 | 24141 | 0.45 | 3.62E−08 | 0.27 | 0.056 | K4me3 + K27me3 | CIMP-H |
| cg00892798 | NGFR | 4804 | 0.47 | 3.79E−08 | 0.19 | 0.235 | K4me3 + K27me3 | CIMP-H |
| cg00910067 | SLC7A10 | 56301 | 0.37 | 3.97E−08 | 0.11 | 0.714 | NA | CIMP-H |
| cg14917512 | GNA11 | 2767 | 0.52 | 3.97E−08 | 0.21 | 0.885 | K4me3 | CIMP-H |
| cg07540118 | ADAM19 | 8728 | 0.42 | 4.12E−08 | 0.14 | 0.668 | K4me3 + K27me3 | CIMP-H |
| cg27152280 | NRG2 | 9542 | 0.31 | 4.12E−08 | 0.06 | 0.657 | K4me3 + K27me3 | CIMP-H |
| cg14939652 | NPAS3 | 64067 | 0.35 | 5.21E−08 | 0.12 | 0.544 | NA | CIMP-H |
| cg25416372 | S1PR5 | 53637 | 0.34 | 5.21E−08 | 0.10 | 0.250 | K4me3 + K27me3 | CIMP-H |
| cg27094076 | ZPBP | 11055 | 0.32 | 5.71E−08 | 0.16 | 0.231 | K4me3 | CIMP-H |
| cg09134003 | NEUROG2 | 63973 | 0.50 | 5.95E−08 | 0.04 | 0.737 | K4me3 + K27me3 | CIMP-H |
| cg04683240 | HLX | 3142 | 0.53 | 6.20E−08 | 0.11 | 0.950 | K4me3 + K27me3 | CIMP-H |
| cg16003238 | IGDCC3 | 9543 | 0.68 | 6.20E−08 | 0.49 | 0.743 | K4me3 + K27me3 | CIMP-H |
| cg16731240 | ZNF577 | 84765 | 0.66 | 6.20E−08 | 0.38 | 0.623 | K4me3 | CIMP-H |
| cg07621046 | C10orf82 | 143379 | 0.66 | 6.48E−08 | 0.37 | 0.980 | K4me3 | CIMP-H |
| cg12379775 | NCRNA00176 | 284739 | 0.48 | 6.78E−08 | 0.15 | 0.832 | K4me3 | CIMP-H |
| cg14894144 | LAMA3 | 3909 | 0.70 | 6.78E−08 | 0.42 | 0.952 | K4me3 + K27me3 | CIMP-H |
| cg04686412 | PSD2 | 84249 | 0.46 | 7.04E−08 | 0.27 | 0.096 | K4me3 + K27me3 | CIMP-H |
| cg12839593 | SIX1 | 6495 | 0.53 | 7.04E−08 | 0.17 | 0.866 | K4me3 + K27me3 | CIMP-H |
| cg20647888 | CCDC3 | 83643 | 0.59 | 7.04E−08 | 0.26 | 0.773 | K4me3 + K27me3 | CIMP-H |
| cg21246783 | CLGN | 1047 | 0.37 | 7.04E−08 | 0.10 | 0.539 | K4me3 + K27me3 | CIMP-H |
| cg00059225 | GLRA1 | 2741 | 0.59 | 7.37E−08 | 0.45 | 0.399 | K4me3 | CIMP-H |
| cg07038400 | PPP2R3A | 5523 | 0.48 | 7.73E−08 | 0.25 | 0.660 | K4me3 | CIMP-H |
| cg00884221 | IGFBP7 | 3490 | 0.35 | 8.03E−08 | 0.11 | 0.971 | K4me3 + K27me3 | CIMP-H |
| cg02995295 | FAM124A | 220108 | 0.43 | 8.03E−08 | 0.14 | 0.619 | K4me3 | CIMP-H |
| cg24824266 | SV2B | 9899 | 0.41 | 8.03E−08 | 0.20 | 0.133 | NA | CIMP-H |
| cg22614355 | HTR6 | 3362 | 0.28 | 8.40E−08 | 0.06 | 0.906 | NA | CIMP-H |
| cg09082287 | DNAJC6 | 9829 | 0.51 | 8.74E−08 | 0.31 | 0.066 | K4me3 | CIMP-H |
| cg16111791 | CACNB4 | 785 | 0.40 | 8.74E−08 | 0.10 | 0.461 | K4me3 + K27me3 | CIMP-H |
| cg25511429 | NRN1 | 51299 | 0.45 | 8.74E−08 | 0.31 | 0.247 | K4me3 + K27me3 | CIMP-H |
| cg10604168 | RASL12 | 51285 | 0.54 | 9.58E−08 | 0.19 | 0.830 | K4me3 | CIMP-H |
| cg12053284 | SYT4 | 6860 | 0.40 | 9.58E−08 | 0.13 | 0.550 | K4me3 | CIMP-H |
| cg13904771 | C10orf47 | 254427 | 0.31 | 9.58E−08 | 0.03 | 0.584 | K4me3 | CIMP-H |
| cg15457899 | SCN3B | 55800 | 0.30 | 9.58E−08 | 0.13 | 0.960 | K4me3 + K27me3 | CIMP-H |
| cg25725843 | ST6GAL2 | 84620 | 0.50 | 9.98E−08 | 0.39 | 0.085 | NA | CIMP-H |
| cg27603796 | CTTNBP2 | 83992 | 0.42 | 9.98E−08 | 0.13 | 0.934 | K4me3 | CIMP-H |
| cg04499325 | EPDR1 | 54749 | 0.56 | 1.09E−07 | 0.20 | 0.910 | K4me3 + K27me3 | CIMP-H |
| cg19187185 | RRAGD | 58528 | 0.35 | 1.19E−07 | 0.12 | 0.868 | NA | CIMP-H |
| cg07393322 | A4GALT | 53947 | 0.38 | 1.24E−07 | 0.14 | 0.852 | K4me3 | CIMP-H |
| cg25870420 | ITGA9 | 3680 | 0.55 | 1.24E−07 | 0.16 | 0.607 | K4me3 + K27me3 | CIMP-H |
| cg17729667 | NINL | 22981 | 0.53 | 1.29E−07 | 0.36 | 0.087 | K4me3 | CIMP-H |
| cg03213216 | FLJ40125 | 147699 | 0.28 | 1.35E−07 | 0.09 | 0.226 | K4me3 | CIMP-H |
| cg23614979 | NAV2 | 89797 | 0.36 | 1.41E−07 | 0.11 | 0.078 | K4me3 + K27me3 | CIMP-H |
| cg04515001 | DCDC2 | 51473 | 0.44 | 1.47E−07 | 0.13 | 0.559 | K4me3 | CIMP-H |
| cg09893305 | HAPLN1 | 1404 | 0.62 | 1.47E−07 | 0.40 | 0.945 | K4me3 + K27me3 | CIMP-H |
| cg14443380 | SEMA7A | 8482 | 0.38 | 1.61E−07 | 0.07 | 0.085 | K4me3 | CIMP-H |
| cg08223748 | MEF2C | 4208 | 0.31 | 1.67E−07 | 0.03 | 0.285 | K4me3 + K27me3 | CIMP-H |
| cg00830029 | NRIP2 | 83714 | 0.67 | 1.75E−07 | 0.38 | 0.877 | K4me3 | CIMP-H |
| cg07849904 | MN1 | 4330 | 0.33 | 1.75E−07 | 0.10 | 0.518 | K4me3 + K27me3 | CIMP-H |
| cg01301664 | FST | 10468 | 0.34 | 1.83E−07 | 0.11 | 0.840 | NA | CIMP-H |
| cg09350274 | GFRA3 | 2676 | 0.56 | 1.91E−07 | 0.32 | 0.482 | K4me3 + K27me3 | CIMP-H |
| cg27327588 | ZNF345 | 25850 | 0.42 | 1.91E−07 | 0.16 | 0.441 | K4me3 | CIMP-H |
| cg13614181 | C13orf15 | 28984 | 0.36 | 2.00E−07 | 0.22 | 0.079 | K4me3 + K27me3 | CIMP-H |
| cg08221207 | FEZ1 | 9638 | 0.26 | 2.26E−07 | 0.08 | 0.292 | K4me3 + K27me3 | CIMP-H |
| cg22882178 | PITX3 | 5309 | 0.38 | 2.26E−07 | 0.14 | 0.250 | K4me3 + K27me3 | CIMP-H |
| cg03310469 | SIX2 | 10736 | 0.42 | 2.91E−07 | 0.08 | 0.863 | K4me3 + K27me3 | CIMP-H |
| cg04628008 | ZBTB16 | 7704 | 0.31 | 2.91E−07 | 0.06 | 0.616 | K4me3 + K27me3 | CIMP-H |
| cg09010998 | ZEB1 | 6935 | 0.40 | 2.91E−07 | 0.15 | 0.888 | K4me3 | CIMP-H |
| cg20449692 | CLDN11 | 5010 | 0.70 | 3.04E−07 | 0.48 | 0.247 | K4me3 | CIMP-H |
| cg01868782 | HEY2 | 23493 | 0.35 | 3.82E−07 | 0.11 | 0.918 | K4me3 + K27me3 | CIMP-H |
| cg22634689 | TMEM121 | 80757 | 0.36 | 3.82E−07 | 0.16 | 0.060 | K4me3 + K27me3 | CIMP-H |
| cg04113075 | RAB32 | 10981 | 0.35 | 4.00E−07 | 0.13 | 0.969 | K4me3 | CIMP-H |
| cg21815667 | HOXD8 | 3234 | 0.41 | 4.00E−07 | 0.24 | 0.402 | NA | CIMP-H |
| cg27154163 | KIT | 3815 | 0.32 | 4.55E−07 | 0.07 | 0.163 | K4me3 | CIMP-H |
| cg09137696 | MT1A | 4489 | 0.40 | 4.76E−07 | 0.11 | 0.839 | K4me3 + K27me3 | CIMP-H |
| cg02564523 | ORAI2 | 80228 | 0.51 | 4.96E−07 | 0.20 | 0.773 | K4me3 | CIMP-H |
| cg06981182 | RNLS | 55328 | 0.54 | 4.96E−07 | 0.30 | 0.405 | K4me3 | CIMP-H |
| cg12973651 | CNFN | 84518 | 0.40 | 5.18E−07 | 0.16 | 0.852 | None | CIMP-H |
| cg20550118 | CRABP1 | 1381 | 0.40 | 5.39E−07 | 0.21 | 0.989 | K4me3 | CIMP-H |
| cg13849691 | ACSL5 | 51703 | 0.85 | 5.62E−07 | 0.63 | 0.972 | NA | CIMP-H |
| cg22736354 | NHLRC1 | 378884 | 0.58 | 5.62E−07 | 0.34 | 0.947 | K4me3 | CIMP-H |
| cg00720137 | DYNLRB2 | 83657 | 0.51 | 5.87E−07 | 0.15 | 0.253 | K4me3 | CIMP-H |
| cg11191210 | VGLL2 | 245806 | 0.53 | 5.87E−07 | 0.36 | 0.141 | NA | CIMP-H |
| cg06493994 | SCGN | 10590 | 0.32 | 6.09E−07 | 0.15 | 0.765 | K4me3 + K27me3 | CIMP-H |
| cg20544605 | SORBS2 | 8470 | 0.72 | 6.09E−07 | 0.48 | 0.926 | None | CIMP-H |
| cg27239157 | MCF2L2 | 23101 | 0.37 | 6.09E−07 | 0.19 | 0.093 | K4me3 + K27me3 | CIMP-H |
| cg06374075 | ADAM11 | 4185 | 0.38 | 6.35E−07 | 0.17 | 0.866 | K4me3 + K27me3 | CIMP-H |
| cg21602520 | BCL2 | 596 | 0.49 | 6.35E−07 | 0.21 | 0.983 | K4me3 + K27me3 | CIMP-H |
| cg00893636 | EPM2AIP1 | 9852 | 0.25 | 6.61E−07 | 0.02 | 0.866 | K4me3 | CIMP-H |
| cg17561435 | BMPER | 168667 | 0.52 | 6.61E−07 | 0.13 | 0.940 | K4me3 + K27me3 | CIMP-H |
| cg10154926 | HAP1 | 9001 | 0.40 | 6.90E−07 | 0.11 | 0.894 | K4me3 + K27me3 | CIMP-H |
| cg18429742 | ZDHHC11 | 79844 | 0.76 | 7.21E−07 | 0.61 | 0.261 | NA | CIMP-H |
| cg16114640 | THSD1 | 55901 | 0.34 | 7.51E−07 | 0.10 | 0.518 | K4me3 + K27me3 | CIMP-H |
| cg20430063 | MSRB3 | 253827 | 0.47 | 7.51E−07 | 0.29 | 0.084 | K4me3 | CIMP-H |
| cg01757745 | C10orf93 | 255352 | 0.60 | 7.79E−07 | 0.47 | 0.078 | K4me3 | CIMP-H |
| cg05523047 | VLDLR | 7436 | 0.31 | 7.79E−07 | 0.03 | 0.452 | K4me3 | CIMP-H |
| cg09374949 | ISYNA1 | 51477 | 0.40 | 7.79E−07 | 0.11 | 0.980 | K4me3 | CIMP-H |
| cg19237879 | SCUBE2 | 57758 | 0.55 | 7.79E−07 | 0.31 | 0.751 | K4me3 + K27me3 | CIMP-H |
| cg09721427 | HHEX | 3087 | 0.55 | 8.84E−07 | 0.28 | 0.807 | NA | CIMP-H |
| cg01606998 | PANX2 | 56666 | 0.27 | 9.68E−07 | 0.06 | 0.129 | NA | CIMP-H |
| cg09381003 | SHKBP1 | 92799 | 0.44 | 1.01E−06 | 0.26 | 0.202 | K4me3 | CIMP-H |
| cg21835643 | RBPJL | 11317 | 0.60 | 1.01E−06 | 0.42 | 0.360 | K4me3 | CIMP-H |
| cg08568512 | FHOD3 | 80206 | 0.45 | 1.05E−06 | 0.04 | 0.392 | K4me3 + K27me3 | CIMP-H |
| cg25550573 | C4orf31 | 79625 | 0.29 | 1.10E−06 | 0.13 | 0.976 | NA | CIMP-H |
| cg10059959 | PAX5 | 5079 | 0.34 | 1.19E−06 | 0.13 | 0.869 | NA | CIMP-H |
| cg25397076 | RBP7 | 116362 | 0.27 | 1.19E−06 | 0.05 | 0.799 | K4me3 + K27me3 | CIMP-H |
| cg06353318 | OTOP2 | 92736 | 0.51 | 1.24E−06 | 0.34 | 0.051 | K4me3 + K27me3 | CIMP-H |
| cg19267846 | PHOSPHO1 | 162466 | 0.23 | 1.24E−06 | 0.05 | 0.592 | K4me3 + K27me3 | CIMP-H |
| cg16652259 | DLX1 | 1745 | 0.33 | 1.29E−06 | 0.16 | 0.173 | NA | CIMP-H |
| cg08820801 | FBXO17 | 115290 | 0.70 | 1.35E−06 | 0.55 | 0.255 | K4me3 | CIMP-H |
| cg01356829 | IL12RB2 | 3595 | 0.67 | 1.40E−06 | 0.55 | 0.150 | K4me3 + K27me3 | CIMP-H |
| cg09331011 | GNAL | 2774 | 0.37 | 1.46E−06 | 0.04 | 0.976 | K4me3 + K27me3 | CIMP-H |
| cg17853587 | NDST3 | 9348 | 0.31 | 1.72E−06 | 0.08 | 0.937 | NA | CIMP-H |
| cg07861564 | HSPB8 | 26353 | 0.37 | 1.80E−06 | 0.17 | 0.666 | K4me3 | CIMP-H |
| cg21621248 | LRRTM1 | 347730 | 0.37 | 1.95E−06 | 0.24 | 0.338 | K4me3 + K27me3 | CIMP-H |
| cg20770175 | COL3A1 | 1281 | 0.59 | 2.03E−06 | 0.41 | 0.998 | None | CIMP-H |
| cg02279071 | MLH1 | 4292 | 0.38 | 2.11E−06 | 0.13 | 0.807 | K4me3 | CIMP-H |
| cg12758687 | DRD2 | 1813 | 0.46 | 2.20E−06 | 0.35 | 0.141 | K4me3 + K27me3 | CIMP-H |
| cg24680602 | ZNF232 | 7775 | 0.37 | 2.29E−06 | 0.18 | 0.157 | NA | CIMP-H |
| cg02320454 | GPR150 | 285601 | 0.54 | 2.37E−06 | 0.15 | 0.982 | K4me3 + K27me3 | CIMP-H |
| cg04600618 | RSPH9 | 221421 | 0.57 | 2.37E−06 | 0.40 | 0.298 | K4me3 | CIMP-H |
| cg04862249 | ZFP3 | 124961 | 0.27 | 2.37E−06 | 0.09 | 0.399 | K4me3 | CIMP-H |
| cg04230060 | SUSD1 | 64420 | 0.64 | 2.47E−06 | 0.36 | 0.811 | K4me3 | CIMP-H |
| cg10978355 | CKMT2 | 1160 | 0.57 | 2.58E−06 | 0.25 | 0.426 | K4me3 | CIMP-H |
| cg05840031 | PAX6 | 5080 | 0.34 | 2.79E−06 | 0.13 | 0.990 | K4me3 + K27me3 | CIMP-H |
| cg01805540 | CACNB2 | 783 | 0.30 | 2.90E−06 | 0.11 | 0.261 | K4me3 | CIMP-H |
| cg08998501 | ZNF615 | 284370 | 0.25 | 2.90E−06 | 0.07 | 0.827 | K4me3 | CIMP-H |
| cg15147516 | MIXL1 | 83881 | 0.34 | 2.90E−06 | 0.04 | 0.958 | K4me3 + K27me3 | CIMP-H |
| cg23727043 | ADAMTS7 | 11173 | 0.33 | 3.01E−06 | 0.16 | 0.653 | K4me3 + K27me3 | CIMP-H |
| cg26709950 | RRAD | 6236 | 0.38 | 3.01E−06 | 0.09 | 0.580 | K4me3 + K27me3 | CIMP-H |
| cg25552492 | LGI3 | 203190 | 0.46 | 3.13E−06 | 0.23 | 0.783 | K4me3 + K27me3 | CIMP-H |
| cg15915418 | TLE1 | 7088 | 0.51 | 3.24E−06 | 0.36 | 0.496 | K4me3 | CIMP-H |
| cg23352579 | RDX | 5962 | 0.33 | 3.37E−06 | 0.03 | 0.522 | K4me3 | CIMP-H |
| cg19524009 | NEK3 | 4752 | 0.68 | 3.51E−06 | 0.39 | 0.773 | NA | CIMP-H |
| cg27560922 | ARHGDIG | 398 | 0.39 | 3.51E−06 | 0.19 | 0.245 | K4me3 | CIMP-H |
| cg26620157 | PAX9 | 5083 | 0.56 | 3.66E−06 | 0.36 | 0.329 | NA | CIMP-H |
| cg02441647 | COL8A1 | 1295 | 0.36 | 3.80E−06 | 0.22 | 0.402 | K4me3 + K27me3 | CIMP-H |
| cg23263923 | CD70 | 970 | 0.30 | 3.94E−06 | 0.18 | 0.088 | K4me3 + K27me3 | CIMP-H |
| cg10240853 | MOSPD3 | 64598 | 0.38 | 4.09E−06 | 0.24 | 0.240 | K4me3 | CIMP-H |
| cg01405761 | CLVS1 | 157807 | 0.29 | 4.26E−06 | 0.10 | 0.985 | K4me3 | CIMP-H |
| cg01352108 | KCNK4 | 50801 | 0.67 | 4.43E−06 | 0.49 | 0.105 | K4me3 + K27me3 | CIMP-H |
| cg16428251 | SOX14 | 8403 | 0.53 | 4.61E−06 | 0.38 | 0.278 | K4me3 + K27me3 | CIMP-H |
| cg18342279 | ZAR1 | 326340 | 0.40 | 4.61E−06 | 0.08 | 0.746 | K4me3 + K27me3 | CIMP-H |
| cg25583174 | FGF2 | 2247 | 0.57 | 4.78E−06 | 0.32 | 0.150 | K4me3 | CIMP-H |
| cg02735486 | ANK2 | 287 | 0.58 | 4.98E−06 | 0.40 | 0.205 | None | CIMP-H |
| cg04797323 | SOCS2 | 8835 | 0.56 | 5.17E−06 | 0.15 | 0.711 | NA | CIMP-H |
| cg10193817 | CADM1 | 23705 | 0.44 | 5.17E−06 | 0.28 | 0.060 | K4me3 + K27me3 | CIMP-H |
| cg08145625 | TRAM1L1 | 133022 | 0.28 | 5.38E−06 | 0.13 | 0.220 | K4me3 | CIMP-H |
| cg00293409 | LRRC33 | 375387 | 0.41 | 5.83E−06 | 0.07 | 0.907 | K4me3 | CIMP-H |
| cg08228917 | LHFP | 10186 | 0.38 | 5.83E−06 | 0.10 | 0.388 | K4me3 + K27me3 | CIMP-H |
| cg09835543 | DIRAS2 | 54769 | 0.35 | 5.83E−06 | 0.12 | 0.842 | K4me3 + K27me3 | CIMP-H |
| cg25250358 | PLOD2 | 5352 | 0.39 | 6.06E−06 | 0.15 | 0.663 | K4me3 | CIMP-H |
| cg02717046 | GPR133 | 283383 | 0.34 | 6.27E−06 | 0.22 | 0.054 | None | CIMP-H |
| cg12902039 | OCA2 | 4948 | 0.53 | 6.27E−06 | 0.36 | 0.385 | K4me3 + K27me3 | CIMP-H |
| cg26928603 | KIRREL | 55243 | 0.28 | 6.27E−06 | 0.21 | 0.133 | K4me3 | CIMP-H |
| cg07197823 | INHBB | 3625 | 0.44 | 7.35E−06 | 0.18 | 0.992 | NA | CIMP-H |
| cg09851465 | C1orf87 | 127795 | 0.42 | 7.35E−06 | 0.17 | 0.657 | K4me3 + K27me3 | CIMP-H |
| cg20973210 | C19orf35 | 374872 | 0.44 | 7.35E−06 | 0.32 | 0.080 | NA | CIMP-H |
| cg16933388 | BSN | 8927 | 0.35 | 7.65E−06 | 0.24 | 0.099 | K4me3 | CIMP-H |
| cg03072378 | DLX4 | 1748 | 0.24 | 7.95E−06 | 0.05 | 0.846 | K4me3 + K27me3 | CIMP-H |
| cg08101264 | ACOT8 | 10005 | 0.28 | 7.95E−06 | 0.10 | 0.835 | K4me3 | CIMP-H |
| cg13271951 | FAM57B | 83723 | 0.56 | 7.95E−06 | 0.43 | 0.118 | K4me3 | CIMP-H |
| cg09636671 | LMOD1 | 25802 | 0.30 | 8.23E−06 | 0.06 | 0.913 | K4me3 + K27me3 | CIMP-H |
| cg10065825 | CDH11 | 1009 | 0.56 | 8.23E−06 | 0.36 | 0.530 | NA | CIMP-H |
| cg11530960 | DMRT2 | 10655 | 0.48 | 8.23E−06 | 0.28 | 0.229 | K4me3 + K27me3 | CIMP-H |
| cg12563178 | PLXDC2 | 84898 | 0.31 | 8.53E−06 | 0.10 | 0.482 | K4me3 | CIMP-H |
| cg04726446 | C2orf39 | 92749 | 0.41 | 8.83E−06 | 0.20 | 0.627 | K4me3 | CIMP-H |
| cg17918501 | FNDC4 | 64838 | 0.35 | 8.83E−06 | 0.20 | 0.381 | K4me3 | CIMP-H |
| cg22774472 | COL5A2 | 1290 | 0.38 | 9.18E−06 | 0.23 | 0.619 | K4me3 | CIMP-H |
| cg25519930 | CACNA1C | 775 | 0.37 | 9.56E−06 | 0.11 | 0.945 | NA | CIMP-H |
| cg09630404 | STAR | 6770 | 0.38 | 9.91E−06 | 0.24 | 0.083 | K4me3 | CIMP-H |
| cg21820890 | PLA2G12B | 84647 | 0.91 | 9.91E−06 | 0.74 | 0.657 | None | CIMP-H |
| cg23922708 | PARD6G | 84552 | 0.35 | 9.91E−06 | 0.10 | 0.350 | K4me3 | CIMP-H |
| cg05520656 | ZNF681 | 148213 | 0.46 | 1.07E−05 | 0.27 | 0.796 | K4me3 | CIMP-H |
| cg02244695 | TMEM176A | 55365 | 0.32 | 1.16E−05 | 0.12 | 0.920 | None | CIMP-H |
| cg26124016 | RARB | 5915 | 0.48 | 1.20E−05 | 0.27 | 0.418 | K4me3 | CIMP-H |
| cg26385286 | GCNT2 | 2651 | 0.65 | 1.20E−05 | 0.44 | 0.956 | NA | CIMP-H |
| cg17191178 | SHOX2 | 6474 | 0.41 | 1.25E−05 | 0.11 | 0.876 | K4me3 + K27me3 | CIMP-H |
| cg19836283 | ITGA11 | 22801 | 0.33 | 1.30E−05 | 0.16 | 0.689 | K4me3 + K27me3 | CIMP-H |
| cg06048156 | ITGB3 | 3690 | 0.43 | 1.35E−05 | 0.10 | 0.754 | K4me3 + K27me3 | CIMP-H |
| cg21926138 | C1orf88 | 128344 | 0.35 | 1.35E−05 | 0.14 | 0.910 | K4me3 | CIMP-H |
| cg26401870 | NPM2 | 10361 | 0.49 | 1.35E−05 | 0.27 | 0.949 | K4me3 + K27me3 | CIMP-H |
| cg02885771 | LTV1 | 84946 | 0.75 | 1.40E−05 | 0.53 | 0.976 | NA | CIMP-H |
| cg22734480 | ABHD8 | 79575 | 0.25 | 1.40E−05 | 0.08 | 0.814 | K4me3 | CIMP-H |
| cg03127334 | ERG | 2078 | 0.44 | 1.56E−05 | 0.27 | 0.677 | K4me3 + K27me3 | CIMP-H |
| cg09214254 | SMOC2 | 64094 | 0.40 | 1.56E−05 | 0.28 | 0.240 | NA | CIMP-H |
| cg10279685 | NPHP1 | 4867 | 0.30 | 1.56E−05 | 0.13 | 0.275 | K4me3 | CIMP-H |
| cg17210604 | HIC1 | 3090 | 0.26 | 1.75E−05 | 0.06 | 0.640 | NA | CIMP-H |
| cg08478189 | KLF7 | 8609 | 0.39 | 1.87E−05 | 0.14 | 0.949 | K4me3 | CIMP-H |
| cg02286642 | ZNF254 | 9534 | 0.52 | 1.94E−05 | 0.40 | 0.229 | NA | CIMP-H |
| cg20880234 | ZMYM2 | 7750 | 0.28 | 1.94E−05 | 0.09 | 0.869 | NA | CIMP-H |
| cg25568243 | DEM1 | 64789 | 0.36 | 1.94E−05 | 0.09 | 0.612 | K4me3 | CIMP-H |
| cg21870884 | GPR25 | 2848 | 0.77 | 2.02E−05 | 0.60 | 0.717 | None | CIMP-H |
| cg05607127 | KCNG3 | 170850 | 0.27 | 2.10E−05 | 0.05 | 0.970 | K4me3 + K27me3 | CIMP-H |
| cg27457941 | RBP1 | 5947 | 0.46 | 2.17E−05 | 0.39 | 0.056 | K4me3 + K27me3 | CIMP-H |
| cg05670408 | MAN1C1 | 57134 | 0.36 | 2.25E−05 | 0.07 | 0.653 | K4me3 + K27me3 | CIMP-H |
| cg15242570 | CTSL1 | 1514 | 0.45 | 2.34E−05 | 0.20 | 0.767 | K4me3 | CIMP-H |
| cg08005849 | HGF | 3082 | 0.46 | 2.52E−05 | 0.28 | 0.811 | None | CIMP-H |
| cg11476211 | PRKCE | 5581 | 0.25 | 2.52E−05 | 0.03 | 0.885 | K4me3 + K27me3 | CIMP-H |
| cg23065097 | FKBP1B | 2281 | 0.60 | 2.52E−05 | 0.42 | 0.779 | NA | CIMP-H |
| cg24662961 | IRX3 | 79191 | 0.37 | 2.52E−05 | 0.14 | 0.603 | K4me3 + K27me3 | CIMP-H |
| cg01966465 | RUNDC3B | 154661 | 0.30 | 2.60E−05 | 0.02 | 0.773 | K4me3 + K27me3 | CIMP-H |
| cg03588357 | GPR68 | 8111 | 0.37 | 2.60E−05 | 0.16 | 0.485 | K4me3 | CIMP-H |
| cg09472203 | AP3B2 | 8120 | 0.38 | 2.69E−05 | 0.07 | 0.934 | K4me3 | CIMP-H |
| cg00514407 | SERPINE2 | 5270 | 0.28 | 2.78E−05 | 0.07 | 0.884 | NA | CIMP-H |
| cg02699167 | FBXL2 | 25827 | 0.38 | 2.78E−05 | 0.14 | 0.289 | K4me3 | CIMP-H |
| cg05702737 | WNT10A | 80326 | 0.36 | 2.78E−05 | 0.21 | 0.120 | K4me3 + K27me3 | CIMP-H |
| cg08631151 | RPRML | 388394 | 0.31 | 2.78E−05 | 0.21 | 0.141 | K4me3 + K27me3 | CIMP-H |
| cg08359956 | TMEM176B | 28959 | 0.39 | 2.88E−05 | 0.23 | 0.310 | None | CIMP-H |
| cg00888479 | SLC24A3 | 57419 | 0.41 | 2.98E−05 | 0.24 | 0.220 | K4me3 + K27me3 | CIMP-H |
| cg11269533 | FEV | 54738 | 0.32 | 2.98E−05 | 0.09 | 0.231 | K4me3 + K27me3 | CIMP-H |
| cg02280309 | PKLR | 5313 | 0.70 | 3.21E−05 | 0.49 | 0.989 | None | CIMP-H |
| cg10453365 | RHCG | 51458 | 0.34 | 3.44E−05 | 0.08 | 0.985 | K4me3 + K27me3 | CIMP-H |
| cg02525756 | RAB42 | 115273 | 0.47 | 3.57E−05 | 0.24 | 0.879 | K4me3 + K27me3 | CIMP-H |
| cg21612046 | ZNF550 | 162972 | 0.35 | 3.57E−05 | 0.22 | 0.479 | NA | CIMP-H |
| cg27063986 | NDST4 | 64579 | 0.67 | 3.57E−05 | 0.47 | 0.981 | None | CIMP-H |
| cg27554782 | CHRNB4 | 1143 | 0.48 | 3.69E−05 | 0.28 | 0.942 | K4me3 | CIMP-H |
| cg18702197 | HOXD3 | 3232 | 0.49 | 3.96E−05 | 0.32 | 0.732 | NA | CIMP-H |
| cg19791277 | KHDRBS3 | 10656 | 0.29 | 3.96E−05 | 0.04 | 0.640 | K4me3 + K27me3 | CIMP-H |
| cg00024396 | ELOVL5 | 60481 | 0.30 | 4.08E−05 | 0.15 | 0.496 | K4me3 | CIMP-H |
| cg02282237 | PRKCH | 5583 | 0.28 | 4.08E−05 | 0.12 | 0.636 | K4me3 + K27me3 | CIMP-H |
| cg07699362 | GPX3 | 2878 | 0.32 | 4.08E−05 | 0.11 | 0.956 | K4me3 | CIMP-H |
| cg18125479 | PYGL | 5836 | 0.29 | 4.08E−05 | 0.09 | 0.055 | K4me3 | CIMP-H |
| cg07359545 | GP1BB | 2812 | 0.58 | 4.23E−05 | 0.34 | 0.728 | K4me3 + K27me3 | CIMP-H |
| cg10983208 | SPOCK2 | 9806 | 0.48 | 5.07E−05 | 0.36 | 0.242 | K4me3 + K27me3 | CIMP-H |
| cg16253412 | TPST1 | 8460 | 0.29 | 5.46E−05 | 0.11 | 0.700 | K4me3 | CIMP-H |
| cg01200060 | SCRT2 | 85508 | 0.31 | 5.66E−05 | 0.17 | 0.173 | K4me3 + K27me3 | CIMP-H |
| cg11115702 | SPNS1 | 83985 | 0.32 | 5.83E−05 | 0.18 | 0.757 | K4me3 | CIMP-H |
| cg22156632 | WNT6 | 7475 | 0.52 | 5.83E−05 | 0.36 | 0.754 | K4me3 + K27me3 | CIMP-H |
| cg15705469 | ZNF71 | 58491 | 0.29 | 6.25E−05 | 0.11 | 0.765 | K4me3 | CIMP-H |
| cg02104644 | SYT7 | 9066 | 0.35 | 6.44E−05 | 0.12 | 0.717 | K4me3 + K27me3 | CIMP-H |
| cg19439331 | TET1 | 80312 | 0.25 | 6.44E−05 | 0.07 | 0.711 | K4me3 | CIMP-H |
| cg21180599 | TLE6 | 79816 | 0.47 | 6.44E−05 | 0.31 | 0.757 | NA | CIMP-H |
| cg22646528 | DTNA | 1837 | 0.37 | 6.44E−05 | 0.09 | 0.130 | K4me3 | CIMP-H |
| cg05847778 | BBS5 | 129880 | 0.27 | 6.66E−05 | 0.09 | 0.563 | K4me3 + K27me3 | CIMP-H |
| cg12955583 | KNDC1 | 85442 | 0.28 | 6.66E−05 | 0.10 | 0.062 | K4me3 | CIMP-H |
| cg06630737 | C1orf187 | 374946 | 0.39 | 6.88E−05 | 0.24 | 0.159 | K4me3 | CIMP-H |
| cg04001842 | DUOXA2 | 405753 | 0.32 | 7.11E−05 | 0.06 | 0.987 | K4me3 + K27me3 | CIMP-H |
| cg21794225 | PRKD1 | 5587 | 0.54 | 7.11E−05 | 0.44 | 0.145 | NA | CIMP-H |
| cg06493386 | TRPA1 | 8989 | 0.56 | 7.38E−05 | 0.39 | 0.874 | K4me3 + K27me3 | CIMP-H |
| cg02927346 | RASL10B | 91608 | 0.55 | 7.91E−05 | 0.41 | 0.302 | K4me3 | CIMP-H |
| cg11572744 | DPYSL3 | 1809 | 0.38 | 7.91E−05 | 0.15 | 0.717 | K4me3 | CIMP-H |
| cg12294121 | GABRB1 | 2560 | 0.33 | 8.20E−05 | 0.14 | 0.942 | K4me3 + K27me3 | CIMP-H |
| cg05705366 | SNX18 | 112574 | 0.29 | 9.42E−05 | 0.07 | 0.975 | K4me3 | CIMP-H |

### Example 6

### Diagnostic CIMP-Associated DNA Methylation Gene Marker Panels were Identified

In this working example, Applicants developed diagnostic DNA methylation gene marker panels to identify CIMP (CIMP-H and CIMP-L), as well as to segregate CIMP-H tumors from CIMP-L tumors based on the Infinium DNA methylation data (FIG. 5).

In particular aspects, a CIMP-defining marker panel consisting of B3GAT2, FOXL2, KCNK13, RAB31 and SLIT1 was identified. Using the conditions that DNA methylation of three or more markers qualifies a sample as CIMP, this panel identifies CIMP-H and CIMP-L tumors with 100% sensitivity and 95.6% specificity with 2.4% misclassification using a β-value threshold of ≧0.1.

In particular aspects, a second marker panel of FAM78A, FSTL1, KCNC1, MYOCD, and SLC6A4 specifically identifies CIMP-H tumors with 100% sensitivity and 100% specificity (0% misclassification) using conditions that three or more markers show DNA methylation β-value threshold of ≧0.1.

In certain aspects, a tumor sample is classified as CIMP-H if both marker panels are positive (three or more markers with DNA methylation for each panel).

In further aspects, a tumor sample is classified as CIMP-L if the CIMP-defining marker panel is positive while the CIMP-H specific panel is negative (0-2 genes methylated).

Table 7 lists the gene and CpG island locations and sequences for the 10 marker genes comprising these two marker panels (i.e., B3GAT2, FOXL2, KCNK13, RAB31 and SLIT1; and FAM78A, FSTL1, KCNC1, MYOCD, and SLC6A4).

Table 11 lists the primer, probe and unconverted amplicon sequences for the MethyLight reactions for the 10 marker genes comprising these two marker panels (i.e., B3GAT2, FOXL2, KCNK13, RAB31 and SLIT1; and FAM78A, FSTL1, KCNC1, MYOCD, and SLC6A4), and for the MLH1 gene.

In yet further aspects, identification and/or classification of CIMP-H and CIMP-L subgroups is provided by a panel comprising at least one of the additional markers listed in Table 8. According to particular aspects,

In yet further aspects, identification and/or classification of CIMP-H subgroups is provided by a panel comprising at least one of the additional markers listed in Table 9.

In additional aspects the MethyLight five-marker panel (i.e., CACNA1G, IGF2, NEUROG1, RUNX3, SOCS1), or markers thereof, previously developed in Applicants' laboratory (Weisenberger et al., Nat Genet 38: 787-793, 2006; see also published U.S. patent application Ser. No. 11/913,535, DNA METHYLATION MARKERS ASSOCIATED WITH THE CPG ISLAND METHYLATOR PHENOTYPE (CIMP) IN HUMAN COLORECTAL CANCER, published as US-2009-0053706-A1 to Laird; all incorporated by reference herein in their entirety; and see Table 10) are used in combination with the panels disclosed herein to provide for identification and/or classification of CRC.

TABLE 7
Gene and CpG island locations and sequences for the 10 marker genes comprising two
preferred marker panels for identification and/or classification of CRC.

| | | | | | | Unmethy- |
|---|---|---|---|---|---|---|
| | | | | | | lated |
| | Entrez | Illumina | | | | AlleleA |
| HUGO | Gene | Probe | Chromo- | Genome | Source | Probe |
| Symbol | ID | ID | some | Build | Sequence | Sequence |
| KCNK13 | 56659 | cg02136132 | chr 14 | Human | GTAG | ATAAA |
| | | | | Feb. | GTGC | TACCT |
| | | | | 2009 | CTCC | CCCCA |
| | | | | (GRCh37/ | CCAG | AATAA |
| | | | | hg19) | GTAG | ATCAA |
| | | | | | ATCG | CAATA |
| | | | | | ACGA | ATACC |
| | | | | | TGGT | TCCTA |
| | | | | | GCCT | ATTAT |
| | | | | | CCTA | AATCA |
| | | | | | GTTG | (SEQ ID |
| | | | | | TGGT | NO: 23) |
| | | | | | CG | |
| | | | | | (SEQ | |
| | | | | | ID | |
| | | | | | NO: 22) | |
| SLIT1 | 6585 | cg07143898 | chr10 | Human | CGGT | AAATA |
| | | | | Feb. | GGAC | TATTC |
| | | | | 2009 | TGCC | TTAAA |
| | | | | (GRCh37/ | ACGG | AATAA |
| | | | | hg19) | CACG | CCTAC |
| | | | | | GGGC | AACCC |
| | | | | | TGCA | CATAC |
| | | | | | GGCC | CATAA |
| | | | | | ATTC | CAATC |
| | | | | | CCAA | CACCA |
| | | | | | GAAT | (SEQ ID |
| | | | | | ATAC | NO: 28) |
| | | | | | CT | |
| | | | | | (SEQ | |
| | | | | | ID | |
| | | | | | NO: 27) | |
| RAB31 | 11031 | cg04274487 | chr18 | Human | CGGC | ATAAC |
| | | | | Feb. | CAGG | AATAC |
| | | | | 2009 | ACTC | AAAAA |
| | | | | (GRCh37/ | ACCC | CTCAA |
| | | | | hg19) | CGAG | AATAT |
| | | | | | AAGG | ACCTT |
| | | | | | CACA | CTCAA |
| | | | | | CTTT | AATAA |
| | | | | | GAGC | ATCCT |
| | | | | | TCCC | AACCA |
| | | | | | GTAT | (SEQ ID |
| | | | | | CGCC | NO: 33) |
| | | | | | AT | |
| | | | | | (SEQ | |
| | | | | | ID | |
| | | | | | NO: 32) | |
| FOXL2 | 668 | cg17503456 | chr3 | Human | CGGG | CAAAC |
| | | | | Feb. | CGAG | TATAC |
| | | | | 2009 | TTCA | AAAAC |
| | | | | (GRCh37/ | TCTC | ATTTA |
| | | | | hg19) | CAAG | CAAAA |
| | | | | | TCAC | AATAA |
| | | | | | TTTTT | CTTAA |
| | | | | | GTAA | AAATA |
| | | | | | ACGC | AACTC |
| | | | | | CCCG | ACCCA |
| | | | | | CACA | (SEQ ID |
| | | | | | GCCT | NO: 38 |
| | | | | | G | |
| | | | | | (SEQ | |
| | | | | | ID | |
| | | | | | NO: 37) | |
| B3GAT2 | 135152 | cg18403396 | chr6 | Human | GATG | AATAA |
| | | | | Feb. | GGTG | ATACA |
| | | | | 2009 | CGCT | CTATC |
| | | | | (GRCh37/ | GTCC | CATAA |
| | | | | hg19) | ATGG | AACCA |
| | | | | | GGCC | AAAAC |
| | | | | | GAGG | ACTAC |
| | | | | | GCGC | AAAAA |
| | | | | | TGCA | CCTAA |
| | | | | | GAGA | AACCA |
| | | | | | CCTG | (SEQ ID |
| | | | | | GAGC | NO: 43) |
| | | | | | CG | |
| | | | | | (SEQ | |
| | | | | | ID | |
| | | | | | NO: 42) | |
| FAM78A | 286336 | cg12998491 | chr9 | Human | GGAC | AAACA |
| | | | | Feb. | GGTA | ATATC |
| | | | | 2009 | TCAG | AACAA |
| | | | | (GRCh37/ | CGGA | AAATA |
| | | | | hg19) | GATG | TCACA |
| | | | | | TCAC | AACAA |
| | | | | | GGGC | CTATT |
| | | | | | GGCT | ATTCA |
| | | | | | ATTA | CTAAT |
| | | | | | TTCG | ACACA |
| | | | | | CTGG | (SEQ ID |
| | | | | | TGCG | NO: 48 |
| | | | | | CG | |
| | | | | | (SEQ | |
| | | | | | ID | |
| | | | | | NO: 47) | |
| MYOCD | 93649 | cg21665000 | chr17 | Human | CGCC | TTATA |
| | | | | Feb. | TGTC | AAAAT |
| | | | | 2009 | AGTA | CCCAA |
| | | | | (GRCh37/ | GTAA | CTTTA |
| | | | | hg19) | AGGG | CCATC |
| | | | | | TATC | TAATA |
| | | | | | AGAT | CCCTT |
| | | | | | GGCA | TACTA |
| | | | | | AAGT | CTAAC |
| | | | | | TGGG | AAACA |
| | | | | | ACCT | (SEQ ID |
| | | | | | TCAT | NO: 53) |
| | | | | | AA | |
| | | | | | (SEQ | |
| | | | | | ID | |
| | | | | | NO: 52) | |
| KCNC1 | 3746 | cg06763078 | chr11 | Human | CTGG | CTAAA |
| | | | | Feb. | AGGA | AAAAA |
| | | | | 2009 | GATG | TAACA |
| | | | | (GRCh37/ | GCGG | AACCC |
| | | | | hg19) | GCCC | CCTAA |
| | | | | | CCTG | ACAAA |
| | | | | | GGCA | AACAC |
| | | | | | GGGG | CCAAA |
| | | | | | CACC | ATATT |
| | | | | | CGGG | ACTCA |
| | | | | | GTGT | (SEQ ID |
| | | | | | TGCT | NO: 58) |
| | | | | | CG | |
| | | | | | (SEQ | |
| | | | | | ID | |
| | | | | | NO: 57 | |
| FSTL1 | 11167 | cg22469841 | chr3 | Human | TCCC | TCCCA |
| | | | | Feb. | GCTT | CTTAC |
| | | | | 2009 | ACGG | AACCC |
| | | | | (GRCh37/ | CCCG | AAACT |
| | | | | hg19) | AACT | ACTTT |
| | | | | | ACTT | TCCTA |
| | | | | | TTCC | CTTTA |
| | | | | | TGCT | AAAAT |
| | | | | | TTAA | TTAAA |
| | | | | | AGAT | TTTCA |
| | | | | | TTAA | (SEQ ID |
| | | | | | GTTT | NO: 63 |
| | | | | | CG | |
| | | | | | (SEQ | |
| | | | | | ID | |
| | | | | | NO: 62) | |
| SLC6A4 | 6532 | cg05016953 | chr17 | Human | CGCA | ATCTA |
| | | | | Feb. | AAAA | ATCTC |
| | | | | 2009 | TTCTT | TAAAT |
| | | | | (GRCh37/ | CAAG | AACCA |
| | | | | hg19) | AGCT | CCACC |
| | | | | | CTTT | AAAAA |
| | | | | | GGCG | ACTCT |
| | | | | | GCGG | TAAAA |
| | | | | | CTAT | AATTT |
| | | | | | CTAG | TTACA |
| | | | | | AGAT | (SEQ ID |
| | | | | | CAGA | NO: 68) |
| | | | | | C (SEQ | |
| | | | | | ID | |
| | | | | | NO: 67 | |
| | | | | UCSC | | Genomic |
| | | | | CpG | | promoter |
| | | | | island | UCSC | sequence; |
| | | | | Start and | CpG | and |
| | Methylated | Accession; | | End; | island | Genomic |
| | AlleleB | and | Promoter | (CpG | Number | CpG |
| HUGO | Probe | Version | sequence | island | of | island |
| Symbol | Sequence | (GI) | position | length) | CpGs | sequence |
| KCNK13 | ATAAAT | NM_022054.2; | chr14: | 89596449 | 240 | (SEQ |
| | ACCTCC | GI: 16306554; | 90526608- | to | | ID |
| | CCAAAT | NC_000014.8 | 90529608 | 89598704 | | NO: 25); |
| | AAATCG | (90,528,108 | | (2255) | | and |
| | ACGATA | to | | | | (SEQ |
| | ATACCT | 90,652,195); | | | | ID |
| | CCTAAT | GI: 224589805 | | | | NO: 26) |
| | TATAAT | | | | | |
| | CG (SEQ | | | | | |
| | ID | | | | | |
| | NO: 24) | | | | | |
| SLIT1 | AAATAT | NM_003061.2; | chr10: | chr10: | 108 | SEQ |
| | ATTCTT | GI: 188528674 | 98944183- | 98945063- | | ID |
| | AAAAAT | | 98947183 | 98946239 | | NO: 30); |
| | AACCTA | | | (1177) | | and |
| | CAACCC | | | | | (SEQ |
| | CGTACC | | | | | ID |
| | GTAACA | | | | | NO: 31) |
| | ATCCAC | | | | | |
| | CG (SEQ | | | | | |
| | ID | | | | | |
| | NO: 29) | | | | | |
| RAB31 | ATAACG | NM_006868.3; | chr18: | chr18: | 151 | SEQ |
| | ATACGA | GI: 170295841 | 9706728- | 9707753- | | ID |
| | AAACTC | | 9709728 | 9709311 | | NO: 35); |
| | AAAATA | | | (1559) | | and |
| | TACCTT | | | | | (SEQ |
| | CTCGAA | | | | | ID |
| | ATAAAT | | | | | NO: 36) |
| | CCTAAC | | | | | |
| | CG (SEQ | | | | | |
| | ID | | | | | |
| | NO: 34) | | | | | |
| FOXL2 | CAAACT | NM_023067.3 | chr3: | chr3: | 230 | SEQ |
| | ATACGA | GI: 239735513 | 138664482- | 138663719- | | ID |
| | AACGTT | | 138667482 | 138666346 | | NO: 40); |
| | TACAAA | | | (2628) | | and |
| | AAATAA | | | | | (SEQ |
| | CTTAAA | | | | | ID |
| | AATAAA | | | | | NO: 41) |
| | CTCGCC | | | | | |
| | CG (SEQ | | | | | |
| | ID NO: 39 | | | | | |
| B3GAT2 | AATAAA | NM_080742.2; | chr6: | chr6: | 181 | SEQ |
| | TACGCT | GI: 109637792 | 71665288- | 71665361- | | ID |
| | ATCCAT | | 71668288 | 71667132 | | NO: 45); |
| | AAAACC | | | (1772) | | and |
| | GAAAA | | | | | (SEQ |
| | CGCTAC | | | | | ID |
| | AAAAA | | | | | NO: 46) |
| | CCTAAA | | | | | |
| | ACCG | | | | | |
| | (SEQ ID | | | | | |
| | NO: 44 | | | | | |
| FAM78A | AAACG | NM_033387.3; | chr9: | chr9: | 161 | SEQ |
| | ATATCA | GI: 118766331 | 134150406- | 134151854- | | ID |
| | ACGAA | | 134153406 | 134153015 | | NO: 50); |
| | AATATC | | | (1162) | | and |
| | ACGAAC | | | | | (SEQ |
| | GACTAT | | | | | ID |
| | TATTCG | | | | | NO: 51 |
| | CTAATA | | | | | |
| | CGCG | | | | | |
| | (SEQ ID | | | | | |
| | NO: 49 | | | | | |
| MYOCD | TTATAA | NM_153604.2; | chr17: | chr17: | 70 | SEQ |
| | AAATCC | GI: 226423887 | 12567707- | 12568668- | | ID |
| | CAACTT | | 12570707 | 12569335 | | NO: 55); |
| | TACCAT | | | (668) | | and |
| | CTAATA | | | | | (SEQ |
| | CCCTTT | | | | | ID |
| | ACTACT | | | | | NO: 56) |
| | AACAA | | | | | |
| | ACG | | | | | |
| | (SEQ ID | | | | | |
| | NO: 54 | | | | | |
| KCNC1 | CTAAAA | NM_004976.4; | chr11: | chr11: | 226 | SEQ |
| | AAAATA | GI: 163792199 | 17755995- | 17756057- | | ID |
| | ACGAAC | | 17758995 | 17758286 | | NO: 60); |
| | CCCCTA | | | 2230) | | and |
| | AACAA | | | | | (SEQ |
| | AAACAC | | | | | ID |
| | CCGAAA | | | | | NO: 61) |
| | TATTAC | | | | | |
| | TCG | | | | | |
| | (SEQ ID | | | | | |
| | NO: 59) | | | | | |
| FSTL1 | TCCCGC | NM_007085.4; | chr3: | chr3: | 127 | SEQ |
| | TTACGA | GI: 197304788 | 120168418- | 120169203- | | ID |
| | CCCGAA | | 120171418 | 120170519 | | NO: 65); |
| | CTACTT | | | (1317) | | and |
| | TTCCTA | | | | | (SEQ |
| | CTTTAA | | | | | ID |
| | AAATTT | | | | | NO: 66) |
| | AAATTT | | | | | |
| | CG (SEQ | | | | | |
| | ID | | | | | |
| | NO: 64) | | | | | |
| SLC6A4 | ATCTAA | NM_001045.4; | chr17: | chr17: | 81 | SEQ |
| | TCTCTA | GI: 225007595 | 28561454- | 28562388- | | ID |
| | AATAAC | | 28564454 | 28563186 | | NO: 70); |
| | CGCCGC | | | (799) | | and |
| | CAAAA | | | | | (SEQ |
| | AACTCT | | | | | ID |
| | TAAAAA | | | | | NO: 71) |
| | ATTTTT | | | | | |
| | ACG | | | | | |
| | (SEQ ID | | | | | |
| | NO: 69) | | | | | |

TABLE 8
Gene and CpG island locations and sequences for additional markers comprising
preferred marker panels for identification and/or classification of of CIMP-H and CIMP-L CRC
subgroups.

| | | | | | Source | Unmethy- |
|---|---|---|---|---|---|---|
| | | | | | Sequence: | lated- |
| | Entrez | | | | Position | AlleleA |
| HUGO | Gene | Illumina | Chromo- | Genome | and | Probe |
| Symbol | ID | Probe ID | some | Build | sequence | Sequence |
| NPTX1 | 4884 | cg17775235 | | Human | chr17: | TTAAAC |
| | | | | Feb. | 78450807- | CAAAAT |
| | | | | 2009 | 78450856 | CATTTA |
| | | | | (GRCh37/ | CGAC | AAACCA |
| | | | | hg19) | CTGG | AACTAA |
| | | | | | GTCC | ATACCC |
| | | | | | TTGG | AAAAA |
| | | | | | GCAC | CCCAAA |
| | | | | | CCAG | TCA |
| | | | | | CCCG | (SEQ ID |
| | | | | | GCTC | NO: 73) |
| | | | | | CAAA | |
| | | | | | CGAC | |
| | | | | | CCCG | |
| | | | | | GCCC | |
| | | | | | AA | |
| | | | | | (SEQ | |
| | | | | | ID | |
| | | | | | NO: 72) | |
| SNCB | 6620 | cg05028467 | | Human | chr5: | TATATA |
| | | | | Feb. | 176057097- | AATATA |
| | | | | 2009 | 176057146 | CTCCAA |
| | | | | (GRCh37/ | CGTC | TATTTA |
| | | | | hg19) | CCCA | CAACTA |
| | | | | | CAGC | CCCAAA |
| | | | | | CGCC | CAACTA |
| | | | | | CGGG | TAAAAA |
| | | | | | CAGC | CA |
| | | | | | TGCA | (SEQ ID |
| | | | | | AACA | NO: 78) |
| | | | | | CCGG | |
| | | | | | AGCA | |
| | | | | | TACT | |
| | | | | | CACA | |
| | | | | | TA | |
| | | | | | (SEQ ID | |
| | | | | | NO: 77) | |
| ARHGEF7 | 8874 | cg00557354 | | Human | chr13: | CAAAAT |
| | | | | Feb. | 111767899- | AATTTT |
| | | | | 2009 | 111767948 | TTTAAA |
| | | | | (GRCh37/ | CGTG | CAACTC |
| | | | | hg19) | GCTC | CAACAC |
| | | | | | ATCA | CCCCAA |
| | | | | | CTCT | AATAAT |
| | | | | | GGGG | AAACCA |
| | | | | | GTGC | CA |
| | | | | | TGGA | (SEQ ID |
| | | | | | GTCG | NO: 83) |
| | | | | | CCCA | |
| | | | | | AAAA | |
| | | | | | AACC | |
| | | | | | ATCT | |
| | | | | | CG | |
| | | | | | (SEQ | |
| | | | | | ID | |
| | | | | | NO: 82) | |
| ABCC8 | 6833 | cg11981631 | | Human | chr11: | AAACA |
| | | | | Feb. | 17497919- | AAACTT |
| | | | | 2009 | 17497968 | AATAAT |
| | | | | (GRCh37/ | AAGC | CCCATA |
| | | | | hg19) | AAAA | AATCAC |
| | | | | | CTTG | AAACAT |
| | | | | | GTGA | CCACTA |
| | | | | | TCCC | TATTTA |
| | | | | | ATGG | ACA |
| | | | | | GTCA | (SEQ ID |
| | | | | | CAAA | NO: 87) |
| | | | | | CGTC | |
| | | | | | CGCT | |
| | | | | | GTGT | |
| | | | | | TTGG | |
| | | | | | CG | |
| | | | | | (SEQ | |
| | | | | | ID | |
| | | | | | NO: 86) | |
| SIRPA | 140885 | cg18952560 | | Human | chr20: | TTTACA |
| | | | | Feb. | 1875040- | CAAACT |
| | | | | 2009 | 1875089 | TATTTT |
| | | | | (GRCh37/ | TTTG | TCTAAA |
| | | | | hg19) | CGCA | ATCAAC |
| | | | | | AACT | ACTACA |
| | | | | | TGTT | AACTAA |
| | | | | | TTTCT | CTACAT |
| | | | | | AAGG | CA |
| | | | | | TCAG | (SEQ ID |
| | | | | | CGCT | NO: 92) |
| | | | | | GCGA | |
| | | | | | GCTG | |
| | | | | | GCTA | |
| | | | | | CATC | |
| | | | | | G | |
| | | | | | (SEQ | |
| | | | | | ID | |
| | | | | | NO: 91) | |
| BHLHE22 | 27319 | cg02982690 | | Human | chr8: | AAAAA |
| | | | | Feb. | 65492846- | AAACTC |
| | | | | 2009 | 65492895 | ACCTAT |
| | | | | (GRCh37/ | CGTT | TAAAAC |
| | | | | hg19) | AACC | AACACT |
| | | | | | TGAT | TTCTAC |
| | | | | | TGGG | CCAATC |
| | | | | | TAGA | AAATTA |
| | | | | | AAGC | ACA |
| | | | | | GCTG | (SEQ ID |
| | | | | | TCCC | NO: 97) |
| | | | | | AACA | |
| | | | | | GGCG | |
| | | | | | AGTC | |
| | | | | | TTCTT | |
| | | | | | C | |
| | | | | | (SEQ | |
| | | | | | ID | |
| | | | | | NO: 96) | |
| COL2A1 | 1280 | cg01291404 | | Human | chr12: | CCTAAA |
| | | | | Feb. | 48397824- | ACAAA |
| | | | | 2009 | 48397873 | ATCCTT |
| | | | | (GRCh37/ | CGCA | AATTAA |
| | | | | hg19) | GAAG | CAAAAC |
| | | | | | TTCA | TCTTCT |
| | | | | | CCAA | TAATAA |
| | | | | | GAAG | ACTTCT |
| | | | | | AGTT | ACA |
| | | | | | CTGC | (SEQ ID |
| | | | | | CAAT | NO: 102) |
| | | | | | CAAG | |
| | | | | | GACT | |
| | | | | | CTGT | |
| | | | | | CCCA | |
| | | | | | GG | |
| | | | | | (SEQ | |
| | | | | | ID | |
| | | | | | NO: 101) | |
| PTEN | 5728 | cg08859916 | | Human | chr10: | AATAAA |
| | | | | Feb. | 89624102- | AATAAC |
| | | | | 2009 | 89624151 | TCTAAA |
| | | | | (GRCh37/ | GATG | CTTAAC |
| | | | | hg19) | GAAA | AATAAC |
| | | | | | TGGC | TAATAC |
| | | | | | TCTG | CCCTCA |
| | | | | | GACT | CTCTAC |
| | | | | | TGGC | CA |
| | | | | | GGTA | (SEQ ID |
| | | | | | GCTG | NO: 107) |
| | | | | | ATGC | |
| | | | | | CCCT | |
| | | | | | CGCT | |
| | | | | | CTGC | |
| | | | | | CG | |
| | | | | | (SEQ | |
| | | | | | ID | |
| | | | | | NO: 106) | |
| AUTS2 | 26053 | cg15753757 | | Human | chr7: | AATATA |
| | | | | Feb. | 69064347- | AAACTC |
| | | | | 2009 | 69064396 | CCCACA |
| | | | | (GRCh37/ | AGTG | ACACCA |
| | | | | hg19) | TGGG | AAAATC |
| | | | | | GCTC | AAAAAT |
| | | | | | CCCA | ACCTAA |
| | | | | | CAGC | AAACA |
| | | | | | ACCG | ACA |
| | | | | | AGGG | (SEQ ID |
| | | | | | TCGG | NO: 112) |
| | | | | | AGAT | |
| | | | | | GCCT | |
| | | | | | GGGA | |
| | | | | | GCAG | |
| | | | | | CG | |
| | | | | | (SEQ | |
| | | | | | ID | |
| | | | | | NO: 111) | |
| KIF1A | 547 | cg21321735 | | Human | chr2: | CTTACA |
| | | | | Feb. | 241760116- | CCAAAA |
| | | | | 2009 | 241760 | ATACTA |
| | | | | (GRCh37/ | 165 | ACTCCA |
| | | | | hg19) | CTTG | AATATA |
| | | | | | CGCC | ACAAAT |
| | | | | | AGGG | ACACAA |
| | | | | | ATGC | TAAAAT |
| | | | | | TGGC | CA |
| | | | | | TCCG | (SEQ ID |
| | | | | | GGTG | NO: 117) |
| | | | | | TAAC | |
| | | | | | AGGT | |
| | | | | | GCGC | |
| | | | | | GGTG | |
| | | | | | AAAT | |
| | | | | | CG | |
| | | | | | (SEQ | |
| | | | | | ID | |
| | | | | | NO: 116) | |
| COL6A2 | 1292 | cg23473904 | | Human | chr21: | AACCTA |
| | | | | Feb. | 47517690- | ACTAAA |
| | | | | 2009 | 47517739 | ACTATA |
| | | | | (GRCh37/ | GACC | TCCACC |
| | | | | hg19) | TGGC | TCCAAA |
| | | | | | TGGG | ACCCTA |
| | | | | | GCTG | AATATT |
| | | | | | TGTC | AAAAA |
| | | | | | CGCC | CCA |
| | | | | | TCCG | (SEQ ID |
| | | | | | GGGC | NO: 122) |
| | | | | | CCTG | |
| | | | | | GGTG | |
| | | | | | TTGG | |
| | | | | | GGAC | |
| | | | | | CG | |
| | | | | | (SEQ | |
| | | | | | ID | |
| | | | | | NO: 121) | |
| SFRP5 | 6425 | cg09874752 | | Human | chr10: | AACACA |
| | | | | Feb. | 99531309- | AAAACC |
| | | | | 2009 | 99531358 | TAACCA |
| | | | | (GRCh37/ | CGGC | AAATAA |
| | | | | hg19) | AGCC | AACAAC |
| | | | | | AGCT | AAACA |
| | | | | | GCTC | AACAAC |
| | | | | | GCCT | TAACTA |
| | | | | | GCTG | CCA |
| | | | | | CTTC | (SEQ ID |
| | | | | | ACTT | NO: 127) |
| | | | | | CGGC | |
| | | | | | CAGG | |
| | | | | | CTCT | |
| | | | | | CGTG | |
| | | | | | CT | |
| | | | | | (SEQ | |
| | | | | | ID | |
| | | | | | NO: 126) | |
| FOXL2 | 668 | cg14312526 | | Human | chr3: | AACTAA |
| | | | | Feb. | 138665243- | AATTAT |
| | | | | 2009 | 138665292 | AACAA |
| | | | | (GRCh37/ | CGAG | ATACTA |
| | | | | hg19) | AAGA | TTTTAC |
| | | | | | ATAA | CAACCC |
| | | | | | GAAG | TTCTTA |
| | | | | | GGCT | TTCTTC |
| | | | | | GGCA | TCA |
| | | | | | AAAT | (SEQ ID |
| | | | | | AGCA | NO: 132) |
| | | | | | TCCG | |
| | | | | | CCAC | |
| | | | | | AACC | |
| | | | | | TCAG | |
| | | | | | CC | |
| | | | | | (SEQ | |
| | | | | | ID | |
| | | | | | NO: 131) | |
| RCSD1 | 92241 | cg14046986 | | Human | chr1: | CCCACT |
| | | | | Feb. | 167599323- | CTTAAA |
| | | | | 2009 | 167599372 | TCCTAC |
| | | | | (GRCh37/ | CGCA | CCTAAA |
| | | | | hg19) | AATC | AAAAA |
| | | | | | TAAA | CAATAT |
| | | | | | CAGA | CTATTT |
| | | | | | TACT | AAATTT |
| | | | | | GTCC | ACA |
| | | | | | CTCC | (SEQ ID |
| | | | | | CAGG | NO: 137) |
| | | | | | GCAG | |
| | | | | | GACT | |
| | | | | | CAAG | |
| | | | | | AGCG | |
| | | | | | GG | |
| | | | | | (SEQ | |
| | | | | | ID | |
| | | | | | NO: 136) | |
| SLC47A1 | 55244 | cg15014549 | | Human | chr17: | TCAAAC |
| | | | | Feb. | 19436955- | TTTACC |
| | | | | 2009 | 19437004 | TTCCAA |
| | | | | (GRCh37/ | CGGG | ATACAA |
| | | | | hg19) | TCTT | AAATTC |
| | | | | | GCAA | CTTAAT |
| | | | | | AGAC | CTTTAC |
| | | | | | CAAG | AAAACC |
| | | | | | GAAC | CA |
| | | | | | TCCT | (SEQ ID |
| | | | | | GCAC | NO: 142) |
| | | | | | TTGG | |
| | | | | | AAGG | |
| | | | | | CAAA | |
| | | | | | GTTT | |
| | | | | | GA | |
| | | | | | (SEQ | |
| | | | | | ID | |
| | | | | | NO: 141) | |
| | | | | UCSC | | Genomic |
| | | | | CpG | | promoter |
| | | | | island | UCSC | sequence; |
| | | | | Start and | CpG | and |
| | Methylated | Accession; | | End; | island | Genomic |
| | AlleleB | and | Promoter | (CpG | Number | CpG |
| HUGO | Probe | Version | sequence | island | of | island |
| Symbol | Sequence | (GI) | position | length) | CpGs | sequence |
| NPTX1 | TTAAAC | NM_002522.3 | chr17: | chr17: | | (SEQ |
| | CGAAAT | GI: 219842351 | 78448904- | 78449508- | | ID |
| | CGTTTA | | 78451904 | 78452783 | | NO: 75); |
| | AAACCG | | | | | and |
| | AACTAA | | | | | (SEQ |
| | ATACCC | | | | | ID |
| | AAAAA | | | | | NO: 76) |
| | CCCAAA | | | | | |
| | TCG | | | | | |
| | (SEQ ID | | | | | |
| | NO: 74) | | | | | |
| SNCB | TATATA | NM_003085.3 | chr5: | chr5: | | (SEQ |
| | AATATA | GI: 48255901 | 176056057- | 176056521- | | ID |
| | CTCCGA | | 176059057 | 176057494 | | NO: 80); |
| | TATTTA | | | | | and |
| | CAACTA | | | | | (SEQ |
| | CCCGAA | | | | | ID |
| | CGACTA | | | | | NO: 81) |
| | TAAAAA | | | | | |
| | CG | | | | | |
| | (SEQ ID | | | | | |
| | NO: 79) | | | | | |
| ARHGEF7 | CGAAAT | NM_001113511.1 | chr13: | | | (SEQ |
| | AATTTT | GI: 166064033 | 111766124- | | | ID |
| | TTTAAA | | 111769124 | | | NO: 85); |
| | CGACTC | | | | | |
| | CAACAC | | | | | |
| | CCCCAA | | | | | |
| | AATAAT | | | | | |
| | AAACCA | | | | | |
| | CG | | | | | |
| | (SEQ ID | | | | | |
| | NO: 84) | | | | | |
| ABCC8 | AAACA | NM_000352.3 | chr11: | chr11: | | (SEQ |
| | AAACTT | GI: 118582254 | 17496949- | 17497464- | | ID |
| | AATAAT | | 17499949 | 17498626 | | NO: 89); |
| | CCCATA | | | | | and |
| | AATCAC | | | | | (SEQ |
| | AAACGT | | | | | ID |
| | CCGCTA | | | | | NO: 90) |
| | TATTTA | | | | | |
| | ACG | | | | | |
| | (SEQ ID | | | | | |
| | NO: 88) | | | | | |
| SIRPA | TTTACG | NM_080792.2 | chr20: | chr20: | | (SEQ |
| | CAAACT | GI: 91105786 | 1873925- | 1874934- | | ID |
| | TATTTT | | 1876925 | 1875718 | | NO: 94); |
| | TCTAAA | | | | | and |
| | ATCAAC | | | | | (SEQ |
| | GCTACG | | | | | ID |
| | AACTAA | | | | | NO: 95) |
| | CTACAT | | | | | |
| | CG | | | | | |
| | (SEQ ID | | | | | |
| | NO: 93) | | | | | |
| BHLHE22 | AAAAA | NM_152414.4 | chr8: | chr8: | | (SEQ |
| | AAACTC | GI: 319803059 | 65491314- | 65492936- | | ID |
| | GCCTAT | | 65494314 | 65494452 | | NO: 99); |
| | TAAAAC | | | | | and |
| | AACGCT | | | | | (SEQ |
| | TTCTAC | | | | | ID |
| | CCAATC | | | | | NO: 100) |
| | AAATTA | | | | | |
| | ACG | | | | | |
| | (SEQ ID | | | | | |
| | NO: 98) | | | | | |
| COL2A1 | CCTAAA | NM_001844.4 | chr12: | chr12: | | (SEQ |
| | ACAAA | GI: 111118975 | 48396785- | 48397890- | | ID |
| | ATCCTT | | 48399785 | 48398731 | | NO: 104); |
| | AATTAA | | | | | and |
| | CAAAAC | | | | | (SEQ |
| | TCTTCT | | | | | ID |
| | TAATAA | | | | | NO: 105) |
| | ACTTCT | | | | | |
| | ACG | | | | | |
| | (SEQ ID | | | | | |
| | NO: 103) | | | | | |
| PTEN | AATAAA | NM_000314.4 | chr10: | chr10: | | (SEQ |
| | AATAAC | GI: 110224474 | 89621695- | 89621773- | | ID |
| | TCTAAA | | 89624695 | 89624128 | | NO: 109); |
| | CTTAAC | | | | | and |
| | GATAAC | | | | | (SEQ |
| | TAATAC | | | | | ID |
| | CCCTCG | | | | | NO: 110) |
| | CTCTAC | | | | | |
| | CG | | | | | |
| | (SEQ ID | | | | | |
| | NO: 108) | | | | | |
| AUTS2 | AATATA | NM_015570.2 | chr7: | chr7: | | (SEQ |
| | AAACTC | GI: 187829443 | 69062406- | 69062375- | | ID |
| | CCCACA | | 69065406 | 69065037 | | NO: 114); |
| | ACACCG | | | | | and |
| | AAAATC | | | | | (SEQ |
| | GAAAAT | | | | | ID |
| | ACCTAA | | | | | NO: 115) |
| | AAACA | | | | | |
| | ACG | | | | | |
| | (SEQ ID | | | | | |
| | NO: 113) | | | | | |
| KIF1A | CTTACG | NM_004321.4 | chr2: | chr2: | | (SEQ |
| | CCAAAA | GI: 41327743 | 241758124- | 241758142- | | ID |
| | ATACTA | | 241761124 | 241760783 | | NO: 119); |
| | ACTCCG | | | | | and |
| | AATATA | | | | | (SEQ |
| | ACAAAT | | | | | ID |
| | ACGCGA | | | | | NO: 120) |
| | TAAAAT | | | | | |
| | CG | | | | | |
| | (SEQ ID | | | | | |
| | NO: 118) | | | | | |
| COL6A2 | GACCTA | NM_001849.3 | chr21: | chr21: | | (SEQ |
| | ACTAAA | GI: 115527061 | 47516533- | 47517652- | | ID |
| | ACTATA | | 47519533 | 47518999 | | NO: 124); |
| | TCCGCC | | | | | and |
| | TCCGAA | | | | | (SEQ |
| | ACCCTA | | | | | ID |
| | AATATT | | | | | NO: 125) |
| | AAAAA | | | | | |
| | CCG | | | | | |
| | (SEQ ID | | | | | |
| | NO: 123) | | | | | |
| SFRP5 | AACACG | NM_003015.3 | chr10: | chr10: | | (SEQ |
| | AAAACC | GI: 188528608 | 99530256- | 99531026- | | ID |
| | TAACCG | | 99533256 | 99531968 | | NO: 129); |
| | AAATAA | | | | | and |
| | AACAAC | | | | | (SEQ |
| | AAACG | | | | | ID |
| | AACAAC | | | | | NO: 130) |
| | TAACTA | | | | | |
| | CCG | | | | | |
| | (SEQ ID | | | | | |
| | NO: 128) | | | | | |
| FOXL2 | AACTAA | NM_023067.3 | chr3: | chr3: | | (SEQ |
| | AATTAT | GI: 239735513 | 138664482- | 138663719- | | ID |
| | AACGA | | 138667482 | 138666346 | | NO: 134); |
| | ATACTA | | | | | and |
| | TTTTAC | | | | | (SEQ |
| | CAACCC | | | | | ID |
| | TTCTTA | | | | | NO: 135) |
| | TTCTTC | | | | | |
| | TCG | | | | | |
| | (SEQ ID | | | | | |
| | NO: 133) | | | | | |
| RCSD1 | CCCGCT | NM_052862.3 | chr1: | chr1: | | (SEQ |
| | CTTAAA | GI: 217035153 | 167597974- | 167599465- | | ID |
| | TCCTAC | | 167600974 | 167599839 | | NO: 139); |
| | CCTAAA | | | | | and |
| | AAAAA | | | | | (SEQ |
| | CAATAT | | | | | ID |
| | CTATTT | | | | | NO: 140) |
| | AAATTT | | | | | |
| | ACG | | | | | |
| | (SEQ ID | | | | | |
| | NO: 138) | | | | | |
| SLC47A1 | TCAAAC | NM_018242.2 | chr17: | chr17: | | (SEQ |
| | TTTACC | GI: 22907059 | 19435667- | 19436789- | | ID |
| | TTCCAA | | 19438667 | 19437692 | | NO: 144); |
| | ATACAA | | | | | and |
| | AAATTC | | | | | (SEQ |
| | CTTAAT | | | | | ID |
| | CTTTAC | | | | | NO: 145) |
| | AAAACC | | | | | |
| | CG | | | | | |
| | (SEQ ID | | | | | |
| | NO: 143) | | | | | |

TABLE 9
Gene and CpG island locations and sequences for additional markers comprising
preferred marker panels for identification and/or classification of of CIMP-H CRC subgroups.

| | | | | | | Unmethy- |
|---|---|---|---|---|---|---|
| | | | | | Source | lated |
| | | | | | Sequence | Allele |
| | Entrez | Illumina | | | Position | A |
| HUGO | Gene | Probe | Chromo- | Genome | and | Probe |
| Symbol | ID | ID | some | Build | sequence | Sequence |
| HTR7 | 3363 | cg26332534 | | Human | chr10: | AAAA |
| | | | | Feb. | 92618063- | TACC |
| | | | | 2009 | 92618112 | CCCA |
| | | | | (GRCh37/ | AAAA | CTAA |
| | | | | hg19) | TGCC | ACTA |
| | | | | | CCCA | TAAC |
| | | | | | CTGA | TAAC |
| | | | | | ACTG | TAAT |
| | | | | | TGGC | ATAC |
| | | | | | TGAC | AAAA |
| | | | | | TAGT | ACTA |
| | | | | | GTGC | AAAT |
| | | | | | GGAG | CA |
| | | | | | GCTG | (SEQ |
| | | | | | GGGT | ID |
| | | | | | CG | NO: 147) |
| | | | | | (SEQ | |
| | | | | | ID | |
| | | | | | NO: 6 | |
| ACSL6 | 23305 | cg19986872 | | Human | chr5: | CTCA |
| | | | | Feb. | 131347725- | CAAC |
| | | | | 2009 | 131347774 | CTAA |
| | | | | (GRCh37/ | CTCG | ATTT |
| | | | | hg19) | CAGC | TATA |
| | | | | | CTGG | ACTA |
| | | | | | GTTT | AACA |
| | | | | | TATG | AACT |
| | | | | | GCTG | CAAA |
| | | | | | GGCA | TAAC |
| | | | | | GGCT | AACC |
| | | | | | CGAA | AAAT |
| | | | | | TGGC | CA |
| | | | | | AGCC | (SEQ |
| | | | | | GGGT | ID |
| | | | | | CG | NO: 152) |
| | | | | | (SEQ | |
| | | | | | ID | |
| | | | | | NO: 151) | |
| SCG3 | 29106 | cg22886089 | | Human | chr15: | CTCC |
| | | | | Feb. | 51973543- | TTTA |
| | | | | 2009 | 51973592 | TTCC |
| | | | | (GRCh37/ | CTCC | ATTC |
| | | | | hg19) | TTTG | CCAA |
| | | | | | TTCC | AAAA |
| | | | | | ATTC | TTAA |
| | | | | | CCGG | AATA |
| | | | | | GGGA | ACAT |
| | | | | | TTGG | TAAA |
| | | | | | AGTA | ATCA |
| | | | | | GCGT | CCAA |
| | | | | | TGGA | CA |
| | | | | | GTCA | (SEQ |
| | | | | | CCGA | ID |
| | | | | | CG | NO: 157) |
| | | | | | (SEQ | |
| | | | | | ID | |
| | | | | | NO: 156) | |
| LOX | 4015 | cg01824804 | | Human | chr5: | AAAC |
| | | | | Feb. | 121413478- | AAAT |
| | | | | 2009 | 121413527 | ATTC |
| | | | | (GRCh37/ | GGGC | AACT |
| | | | | hg19) | AGGT | TACT |
| | | | | | GTTC | AAAC |
| | | | | | AGCT | CTAA |
| | | | | | TGCT | ACTC |
| | | | | | GAGC | ACAA |
| | | | | | CTGG | TACC |
| | | | | | GCTC | AACC |
| | | | | | ACAG | TCAA |
| | | | | | TACC | CA |
| | | | | | AGCC | (SEQ |
| | | | | | TCAG | ID |
| | | | | | CG | NO: 162) |
| | | | | | (SEQ | |
| | | | | | ID | |
| | | | | | NO: 161) | |
| OGDHL | 55753 | cg06222851 | | Human | chr10: | ACAA |
| | | | | Feb. | 50970310- | CAAA |
| | | | | 2009 | 50970359 | ATCC |
| | | | | (GRCh37/ | GCAG | AAAA |
| | | | | hg19) | CGAG | ACTA |
| | | | | | GTCC | CAAA |
| | | | | | GGAG | TCAA |
| | | | | | GCTG | AAAA |
| | | | | | CAGG | CTAC |
| | | | | | TCAG | ACAA |
| | | | | | GGGG | AAAA |
| | | | | | CTGC | AATA |
| | | | | | GCGG | CA |
| | | | | | AAGG | (SEQ |
| | | | | | GGTG | ID |
| | | | | | CG | NO: 167) |
| | | | | | (SEQ | |
| | | | | | ID | |
| | | | | | NO: 166) | |
| SYNM | 23336 | cg05881135 | | Human | chr15: | AAAA |
| | | | | Feb. | 99644925- | AACC |
| | | | | 2009 | 99644974 | ACCA |
| | | | | (GRCh37/ | GAAA | ATCT |
| | | | | hg19) | AGCC | CCAA |
| | | | | | ACCA | AACC |
| | | | | | ATCT | TCAA |
| | | | | | CCAG | ATAA |
| | | | | | GGCC | AAAA |
| | | | | | TCGG | TCAC |
| | | | | | GTAA | TCCA |
| | | | | | GAAG | AAAT |
| | | | | | TCGC | CA |
| | | | | | TCCA | (SEQ |
| | | | | | AAAT | ID |
| | | | | | CG | NO: 172) |
| | | | | | (SEQ | |
| | | | | | ID | |
| | | | | | NO: 171) | |
| PCOLCE2 | 26577 | cg19219437 | | Human | chr3: | CTAC |
| | | | | Feb. | 142607667- | TACC |
| | | | | 2009 | 142607716 | ACAA |
| | | | | (GRCh37/ | CTGC | AAAC |
| | | | | hg19) | TGCC | TAAA |
| | | | | | GCGA | TAAC |
| | | | | | GAGC | AACA |
| | | | | | TGGG | ACCA |
| | | | | | TGGC | ACAA |
| | | | | | GGCA | CAAA |
| | | | | | GCCA | CAAA |
| | | | | | GCAG | ATAA |
| | | | | | CAGG | CA |
| | | | | | CAGA | (SEQ |
| | | | | | GTGG | ID |
| | | | | | CG | NO: 177) |
| | | | | | (SEQ | |
| | | | | | ID | |
| | | | | | NO: 176) | |
| No | 23037 | cg25735280 | | Human | chr5: | CTTT |
| over- | | | | Feb. | 31638903- | ACTA |
| lapping | | | | 2009 | 31638952 | TATA |
| gene | | | | (GRCh37/ | CTTT | AAAT |
| | | | | hg19) | GCTG | ACTA |
| | | | | | TGTA | TACA |
| | | | | | AAGT | AAAA |
| | | | | | GCTA | CAAC |
| | | | | | TGCA | TTCA |
| | | | | | GAGG | CATC |
| | | | | | CAAC | CCCT |
| | | | | | TTCA | AACT |
| | | | | | CGTC | CA |
| | | | | | CCCT | (SEQ |
| | | | | | AGCT | ID |
| | | | | | CG | NO: 182) |
| | | | | | (SEQ | |
| | | | | | ID | |
| | | | | | NO: 181) | |
| UBE2E2 | 7325 | cg19803671 | | Human | chr3: | ATTA |
| | | | | Feb. | 23245374- | CTAC |
| | | | | 2009 | 23245423 | ACAT |
| | | | | (GRCh37/ | ATTG | AATA |
| | | | | hg19) | CTGC | TATC |
| | | | | | ACAT | TCCT |
| | | | | | GGTG | AACA |
| | | | | | TGTC | AATA |
| | | | | | TCCT | CCAA |
| | | | | | GGCG | ATTT |
| | | | | | AGTG | AACT |
| | | | | | CCGG | CCTT |
| | | | | | GTTT | CA |
| | | | | | GGCT | (SEQ |
| | | | | | CCTT | ID |
| | | | | | CG | NO: 187) |
| | | | | | (SEQ | |
| | | | | | ID | |
| | | | | | NO: 186) | |
| OXTR | 5021 | cg23391006 | | Human | chr3: | TTTT |
| | | | | Feb. | 8811279- | AAAC |
| | | | | 2009 | 8811328 | CACT |
| | | | | (GRCh37/ | TTTT | ACAA |
| | | | | hg19) | AAAC | AATA |
| | | | | | CACT | AACC |
| | | | | | GCAA | CATT |
| | | | | | AATA | TATT |
| | | | | | AACC | AAAA |
| | | | | | CATT | CTCT |
| | | | | | TGTT | AAAA |
| | | | | | AAGG | CCAA |
| | | | | | CTCT | CA |
| | | | | | GGGA | (SEQ |
| | | | | | CCAA | ID |
| | | | | | CG | NO: 192) |
| | | | | | (SEQ | |
| | | | | | ID | |
| | | | | | NO: 191) | |
| CELF6 | 60677 | cg21801378 | | Human | chr15: | TAAT |
| | | | | Feb. | 72612077- | AACA |
| | | | | 2009 | 72612126 | TCAT |
| | | | | (GRCh37/ | CGGG | AATC |
| | | | | hg 19) | CTAA | CTTC |
| | | | | | ACCC | ATAA |
| | | | | | CGGT | ATAC |
| | | | | | CCCG | AACA |
| | | | | | CCGT | AAAC |
| | | | | | ACCC | CAAA |
| | | | | | ATGA | ATTT |
| | | | | | AGGA | AACC |
| | | | | | CCAC | CA |
| | | | | | GACG | (SEQ |
| | | | | | CCAT | ID |
| | | | | | CA | NO: 197) |
| | | | | | (SEQ | |
| | | | | | ID | |
| | | | | | NO: 196) | |
| RAB39 | 54734 | cg08179907 | | Human | chr11: | TAAA |
| | | | | Feb. | 107798919- | ACCT |
| | | | | 2009 | 107798968 | TACT |
| | | | | (GRCh37/ | TGAG | TCAT |
| | | | | hg19) | ACCT | TTTC |
| | | | | | TGCT | CTTA |
| | | | | | TCAT | ATTA |
| | | | | | TTTC | TTAC |
| | | | | | CTTG | TACT |
| | | | | | GTTG | ATCA |
| | | | | | TTGC | CATC |
| | | | | | TGCT | TTCC |
| | | | | | GTCA | CA |
| | | | | | CGTC | (SEQ |
| | | | | | TTCC | ID |
| | | | | | CG | NO: 202) |
| | | | | | (SEQ | |
| | | | | | ID | |
| | | | | | NO: 201) | |
| LOX | 4015 | cg02548238 | | Human | chr5: | CTAA |
| | | | | Feb. | 121413002- | AAAC |
| | | | | 2009 | 121413051 | CAAA |
| | | | | (GRCh37/ | CGGT | TACA |
| | | | | hg19) | AAGT | CAAA |
| | | | | | ACCC | TACT |
| | | | | | CCAA | TCCA |
| | | | | | GTCC | ACAA |
| | | | | | GCTG | ACTT |
| | | | | | GAAG | AAAA |
| | | | | | CACC | ATAC |
| | | | | | CGTG | TTAC |
| | | | | | CACC | CA |
| | | | | | TGGT | (SEQ |
| | | | | | CCCC | ID |
| | | | | | AG | NO: 207) |
| | | | | | (SEQ | |
| | | | | | ID | |
| | | | | | NO: 206) | |
| RUNX3 | 864 | cg06377278 | | Human | chr1: | AAAA |
| | | | | Feb. | 25256321- | CAAC |
| | | | | 2009 | 25,256370 | AACT |
| | | | | (GRCh37/ | CGGC | AATA |
| | | | | hg19) | CGCT | CTTA |
| | | | | | GTTA | AATC |
| | | | | | TGCG | TACA |
| | | | | | TATT | AAAA |
| | | | | | CCCG | TACA |
| | | | | | TAGA | CATA |
| | | | | | CCCA | ACAA |
| | | | | | AGCA | CAAC |
| | | | | | CCAG | CA |
| | | | | | CCGC | (SEQ |
| | | | | | CGCT | ID |
| | | | | | TC | NO: 212) |
| | | | | | (SEQ | |
| | | | | | ID | |
| | | | | | NO: 211) | |
| COL4A3 | 1285 | cg04324308 | | Human | chr2: | AAAA |
| | | | | Feb. | 228028741- | CTAA |
| | | | | 2009 | 228028790 | AAAT |
| | | | | (GRCh37/ | CGCC | ACAC |
| | | | | hg19) | AGGA | ATCC |
| | | | | | GCTG | CATA |
| | | | | | CCGC | AAAT |
| | | | | | CTTG | AACA |
| | | | | | CCAC | AAAC |
| | | | | | CCCA | AACA |
| | | | | | CGGG | ACTC |
| | | | | | ACGC | CTAA |
| | | | | | GCAC | CA |
| | | | | | CTCC | (SEQ |
| | | | | | AGCC | ID |
| | | | | | CC | NO: 217) |
| | | | | | (SEQ | |
| | | | | | ID | |
| | | | | | NO: 216) | |
| | | | | UCSC | | Genomic |
| | | | | CpG | | promoter |
| | | | | island | UCSC | sequence; |
| | Methy- | | | Start and | CpG | and |
| | lated | Accession; | | End; | island | Genomic |
| | AlleleB | and | Promoter | (CpG | Number | CpG |
| HUGO | Probe | Version | sequence | island | of | island |
| Symbol | Sequence | (GI) | position | length) | CpGs | sequence |
| HTR7 | AAAATA | NM_019859.3 | chr10: | chr10: | | (SEQ |
| | CCCCCA | GI: 197276639 | 92616171- | 92616821- | | ID |
| | CTAAAC | | 92619171 | 92618034 | | NO: 149); |
| | TATAAC | | | | | and |
| | TAACTA | | | | | (SEQ |
| | ATATAC | | | | | ID |
| | GAAAA | | | | | NO: 150) |
| | CTAAAA | | | | | |
| | TCG | | | | | |
| | (SEQ ID | | | | | |
| | NO: 148) | | | | | |
| ACSL6 | CTCGCA | NM_015256.3 | chr5: | chr5: | | (SEQ |
| | ACCTAA | GI: 327412318 | 131345855- | 131346893- | | ID |
| | ATTTTA | | 131348855 | 131347776 | | NO: 154); |
| | TAACTA | | | | | and' |
| | AACAA | | | | | (SEQ ID |
| | ACTCGA | | | | | NO: 155) |
| | ATAACA | | | | | |
| | ACCGAA | | | | | |
| | TCG | | | | | |
| | (SEQ ID | | | | | |
| | NO: 153) | | | | | |
| SCG3 | CTCCTT | NM_013243.3 | chr15: | chr15: | | (SEQ |
| | TATTCC | GI: 259089431 | 51972050- | 51973534- | | ID |
| | ATTCCC | | 51975050 | 51973838 | | NO: 159); |
| | GAAAA | | | | | and |
| | ATTAAA | | | | | (SEQ |
| | ATAACG | | | | | ID |
| | TTAAAA | | | | | NO: 160) |
| | TCACCG | | | | | |
| | ACG | | | | | |
| | (SEQ ID | | | | | |
| | NO: 158) | | | | | |
| LOX | GAACA | NM_002317.5 | chr5: | chr5: | | (SEQ |
| | AATATT | GI: 296010938 | 121412555- | 121412501- | | ID |
| | CAACTT | | 121415555 | 121414077 | | NO: 164); |
| | ACTAAA | | | | | and |
| | CCTAAA | | | | | (SEQ |
| | CTCACA | | | | | ID |
| | ATACCA | | | | | NO: 165) |
| | ACCTCA | | | | | |
| | ACG | | | | | |
| | (SEQ ID | | | | | |
| | NO: 163) | | | | | |
| OGDHL | ACAACG | NM_018245.2 | chr10: | chr10: | | (SEQ |
| | AAATCC | GI: 221316660 | 50968925- | 50969659- | | ID |
| | GAAAA | | 50971925 | 50970605 | | NO: 169); |
| | CTACAA | | | | | and |
| | ATCAAA | | | | | (SEQ |
| | AAACTA | | | | | ID |
| | CGCGAA | | | | | NO: 170) |
| | AAAAAT | | | | | |
| | ACG | | | | | |
| | (SEQ ID | | | | | |
| | NO: 168) | | | | | |
| SYNM | AAAAA | NM_145728.2 | chr15: | chr15: | | (SEQ |
| | ACCACC | GI: 112382236 | 99643786- | 99645031- | | ID |
| | AATCTC | | 99646786 | 99646444 | | NO: 174); |
| | CAAAAC | | | | | and |
| | CTCGAA | | | | | (SEQ |
| | TAAAAA | | | | | ID |
| | ATCGCT | | | | | NO: 175) |
| | CCAAAA | | | | | |
| | TCG | | | | | |
| | (SEQ ID | | | | | |
| | NO: 173) | | | | | |
| PCOLCE2 | CTACTA | NM_013363.3 | chr3: | chr3: | | (SEQ |
| | CCGCGA | GI: 296317252 | 142606545- | 142607196- | | ID |
| | AAACTA | | 142609545 | 142608229 | | NO: 179); |
| | AATAAC | | | | | and |
| | GACAAC | | | | | (SEQ |
| | CAACAA | | | | | ID |
| | CAAACA | | | | | NO: 180) |
| | AAATAA | | | | | |
| | CG | | | | | |
| | (SEQ ID | | | | | |
| | NO: 178) | | | | | |
| No | CTTTAC | NM_178140.2 | chr5: | chr5: | | (SEQ |
| over- | TATATA | GI: 87196342 | 31637451- | 31639054- | | ID |
| lapping | AAATAC | | 31640451 | 31640104 | | NO: 184); |
| gene | TATACA | | | | | and |
| | AAAAC | | | | | (SEQ |
| | AACTTC | | | | | ID |
| | ACGTCC | | | | | NO: 185) |
| | CCTAAC | | | | | |
| | TCG | | | | | |
| | (SEQ ID | | | | | |
| | NO: 183) | | | | | |
| UBE2E2 | ATTACT | NM_152653.3 | chr3: | chr3: | | (SEQ |
| | ACACAT | GI: 195976814 | 23243284- | 23244051- | | ID |
| | AATATA | | 23246284 | 23245071 | | NO: 189); |
| | TCTCCT | | | | | and |
| | AACGA | | | | | (SEQ |
| | ATACCG | | | | | ID |
| | AATTTA | | | | | NO: 190) |
| | ACTCCT | | | | | |
| | TCG | | | | | |
| | (SEQ ID | | | | | |
| | NO: 188) | | | | | |
| OXTR | TTTTAA | NM_000916.3 | chr3: | chr3: | | (SEQ |
| | ACCACT | GI: 32307151 | 8809800- | 8808962- | | ID |
| | ACAAA | | 8812800 | 8811280 | | NO: 194); |
| | ATAAAC | | | | | and |
| | CCATTT | | | | | (SEQ |
| | ATTAAA | | | | | ID |
| | ACTCTA | | | | | NO: 195) |
| | AAACCA | | | | | |
| | ACG | | | | | |
| | (SEQ ID | | | | | |
| | NO: 193) | | | | | |
| CELF6 | TAATAA | NM_052840.4 | chr15: | chr15: | | (SEQ |
| | CGTCGT | GI: 219878492 | 72611025- | 72611947- | | ID |
| | AATCCT | | 72614025 | 72612802 | | NO: 199); |
| | TCATAA | | | | | and |
| | ATACGA | | | | | (SEQ |
| | CGAAAC | | | | | ID |
| | CGAAAT | | | | | NO: 200) |
| | TTAACC | | | | | |
| | CG | | | | | |
| | (SEQ ID | | | | | |
| | NO: 198) | | | | | |
| RAB39 | TAAAAC | NM_017516.1 | chr11: | chr11: | | (SEQ |
| | CTTACT | GI: 39930370 | 107797777- | 107798959- | | ID |
| | TCATTT | | 107800777 | 107799980 | | NO: 204); |
| | TCCTTA | | | | | and |
| | ATTATT | | | | | (SEQ |
| | ACTACT | | | | | ID |
| | ATCACG | | | | | NO: 205) |
| | TCTTCC | | | | | |
| | CG | | | | | |
| | (SEQ ID | | | | | |
| | NO: 203) | | | | | |
| LOX | CTAAAA | NM_002317.5 | chr5: | chr5: | | (SEQ |
| | ACCAAA | GI: 296010938 | 121412555- | 121412501- | | ID |
| | TACACG | | 121415555 | 121414077 | | NO: 209); |
| | AATACT | | | | | and |
| | TCCAAC | | | | | (SEQ |
| | GAACTT | | | | | ID |
| | AAAAAT | | | | | NO: 210) |
| | ACTTAC | | | | | |
| | CG | | | | | |
| | (SEQ ID | | | | | |
| | NO: 208) | | | | | |
| RUNX3 | AAAAC | NM_004350.2 | chr1: | chr1: | | (SEQ |
| | GACGAC | GI: 110735400 | 25255270- | 25255528- | | ID |
| | TAATAC | | 25258270 | 25259005 | | NO: 214); |
| | TTAAAT | | | | | and |
| | CTACGA | | | | | (SEQ |
| | AAATAC | | | | | ID |
| | GCATAA | | | | | NO: 215) |
| | CAACGA | | | | | |
| | CCG | | | | | |
| | (SEQ ID | | | | | |
| | NO: 213) | | | | | |
| COL4A3 | AAAACT | NM_000091.4 | chr2: | chr2: | | (SEQ |
| | AAAAAT | GI: 297632355 | 228027781- | 228028680- | | ID |
| | ACGCGT | | 228030781 | 228029733 | | NO: 219); |
| | CCCGTA | | | | | and |
| | AAATAA | | | | | (SEQ |
| | CAAAAC | | | | | ID |
| | GACAAC | | | | | NO: 220) |
| | TCCTAA | | | | | |
| | CG | | | | | |
| | (SEQ ID | | | | | |
| | NO: 218) | | | | | |

TABLE 10
Table 6 of published US-2009-0053706-A1 to Laird.

| | | | | MethyLight | MethyLight | | |
|---|---|---|---|---|---|---|---|
| HUGO | | | GenBank | Amplicon | Amplicon | CpG Island | CpG Island |
| Gene | Reaction | Reaction | Accession | Start (GenBank | End (GenBank | Start (GenBank | End (GenBank |
| Nomenclature | Number | ID | Number | Numbering) | Numbering) | Numbering) | Numbering) |
| BCL2 | HB-140 | BCL2-M1 | AY220759 | 1221 | 1304 | 746 | 1876 |
| BDNF | HB-258 | BDNF-M2 | AC103796 | 3794 | 3866 | 3351 | 4751 |
| CACNA1G | HB-158 | CACNA1G-M1 | AC021491 | 48345 | 48411 | 47327 | 49295 |
| CALCA | HB-166 | CALCA-M1 | X15943 | 1706 | 1806 | 1614 | 2359 |
| CRABP1 | HB-197 | CRABP1-M1 | AC011270 | 122223 | 122142 | 122717 | 120620 |
| DLEC1 | HB-225 | DLEC1-M1 | AP006309 | 19959 | 20088 | 19425 | 20529 |
| GATA3 | HB-327 | GATA3-M1 | AL390294 | 51880 | 51959 | 50613 | 54089 |
| HOXA1 | HB-268 | HOXA1-M2 | AC004079 | 78220 | 78138 | 79793 | 77693 |
| IGF2 | HB-319 | IGF2-M2 | AC132217 | 108633 | 108720 | 106219 | 110017 |
| KL | HB-175 | KL-M1 | AB009667 | 2062 | 2189 | 1239 | 3185 |
| NEUROG1 | HB-261 | NEUROG1-M1 | AC005738 | 75429 | 75342 | 76036 | 73946 |
| NR3C1 | HB-067 | NR3C1-M1 | AY436590 | 1786 | 1860 | 32 | 3034 |
| RUNX3 | HB-181 | RUNX3-M1 | AL023096 | 64762 | 64646 | 67973 | 63661 |
| SOCS1 | HB-042 | SOCS1-M1 | AC009121 | 108803 | 108888 | 107037 | 109517 |

TABLE 11
Primer, probe and unconverted amplicon sequences for the MethyLight reactions for
the 10 marker genes comprising these two marker panels (i.e., B3GAT2, FOXL2, KCNK13,
RAB31 and SLIT1; and FAM78A, FSTL1, KCNC1, MYOCD, and SLC6A4), and for the MLH1
gene.

| | | | | Forward | Reverse | Probe | Amplicon |
|---|---|---|---|---|---|---|---|
| Reaction | HUGO Gene | Reaction | Infinium | Primer | Primer | Oligo | sequence |
| No. | Nomenclature | ID | Target cg | Sequence | Sequence | Sequence | unconverted |
| HB-973 | KCNK13 | H- | CIMP | TTTATT | GACGAT | 6FAM- | TCCATCCCTAA |
| | | KCNK13- | Infinium | TTTAAG | AATACC | TCGCGC | GCCCCGGCAG |
| | | M1B | target | TTTCGG | TCCTAA | TAAACC | CCGATTCGGAG |
| | | | cg02136132 | TAGTCG | TTATAA | TATAAC | ACTCGGGAGG |
| | | | | AT | TCGTAA | CTCCCG | CCACAGGCTCA |
| | | | | (SEQ ID | (SEQ ID | AATC- | GCGCGACACC |
| | | | | NO: 221) | NO: 222) | BHQ1 | ACGACCACAA |
| | | | | | | (SEQ ID | CTAGGAGGCA |
| | | | | | | NO: 223) | CCATCGTC |
| | | | | | | | (SEQ ID NO: 224) |
| HB-974 | SLIT1 | H- | CIMP | AGGATT | CGAACG | 6FAM- | AGGACCCCCA |
| | | SLIT1- | Infinium | TTTATT | AAAATA | CCGTCT | CCCGGGAGTC |
| | | M1B | target | CGGGAG | ATCAAC | AACTCG | AGCGCCATGGT |
| | | | cg07143898 | TTAGC | GACTAC | CGAACG | GCCCTCACAGC |
| | | | (not | (SEQ ID | (SEQ ID | AAACGC | GTCCCGCTCGC |
| | | | overlapping) | NO: 225) | NO: 226) | TATAAA | GAGCCAGACG |
| | | | | | | -BHQ1 | GCAGCAGCCG |
| | | | | | | (SEQ ID | CTGACCATCCC |
| | | | | | | NO: 227) | CGTCCG |
| | | | | | | | (SEQ ID NO: 228) |
| HB-975 | SLIT1 | H- | CIMP | AATGGT | ACGCCT | 6FAM- | AATGGCCTGCA |
| | | SLIT1- | Infinium | TTGTAG | AAATAC | CCCTCT | GCCCCGTGCCG |
| | | M2B | target | TTTCGT | CTCGAC | ACACCT | TGGCAGTCCAC |
| | | | cg07143898 | GTCG | GT | ACACCG | CGTGGTTCCGG |
| | | | | (SEQ ID | (SEQ ID | AAACCA | TGCAGGTGCA |
| | | | | NO: 229) | NO: 230) | CGA- | GAGGGCGGGG |
| | | | | | | BHQ1 | CACGCCGAGG |
| | | | | | | (SEQ ID | CACCCAGGCG |
| | | | | | | NO: 231) | C |
| | | | | | | | (SEQ ID NO: 232) |
| HB-976 | RAB31 | H- | CIMP | TATGAT | CGAAAA | 6FAM- | CATGATGGCG |
| | | RAB31- | Infinium | GGCGAT | CGCGAA | ACGAAT | ATACGGGAGC |
| | | M1B | target | ACGGGA | CCGA | AACGAC | TCAAAGTGTGC |
| | | | cg04274487 | GT | (SEQ ID | CAAAAC | CTTCTCGGGGT |
| | | | | (SEQ ID | NO: 234) | TCACCC | GAGTCCTGGCC |
| | | | | NO: 233) | | CGAA- | GCCACCCGCCG |
| | | | | | | BHQ1 | GCGGACCCCG |
| | | | | | | (SEQ ID | GCCCGCGCTCT |
| | | | | | | NO: 235) | CG |
| | | | | | | | (SEQ ID NO: 236) |
| HB-977 | FOXL2/ | H- | CIMP | GGTTTT | AACTTA | 6FAM- | GGCTCCACCGA |
| | C3ORF72 | C3ORF72- | Infinium | ATCGAG | AAAATA | CGACTA | GTTCCGCTTGC |
| | | M1B | target | TTTCGT | AACTCG | ACCGCC | GTCAGGCGCCT |
| | | | cg17503456 | TTGC | CCCGTA | CCGCTA | TCGCCCCTATA |
| | | | | (SEQ ID | (SEQ ID | TAAAAA | GCGGGGCGGC |
| | | | | NO: 237) | NO: 238) | CGA- | CAGCCGCGCA |
| | | | | | | BHQ1 | CGGGCGAGTTC |
| | | | | | | (SEQ ID | ATCTCCAAGTC |
| | | | | | | NO: 239) | (SEQ ID NO: 240) |
| HB-978 | B3GAT2 | H- | CIMP | GGCGTT | CGCCTA | 6FAM- | GGCGCTGCAG |
| | | B3GAt2- | Infinium | GTAGAG | CACCCC | CTACCG | AGACCTGGAG |
| | | M1B | target | ATTTGG | TTATCG | CTCCTC | CCGCGGGGCTC |
| | | | cg18403396 | AGTC | (SEQ ID | CACGCC | ACTACCTGGGC |
| | | | | (SEQ ID | NO: 242) | CAAA- | GTGGAGGAGC |
| | | | | NO: 241) | | BHQ1 | GGCAGGTTCGC |
| | | | | | | (SEQ ID | GCAAGCTAGA |
| | | | | | | NO: 243) | GCGACAAGGG |
| | | | | | | | GTGCAGGCG |
| | | | | | | | (SEQ ID NO: 278) |
| HB-979 | FAM78A | H- | CIMP | CGTACG | CCCTAC | 6FAM- | CGCACGACCG |
| | | FAM78A- | Infinium | ATCGCG | AACGAC | CCGCCC | CGCGCACCAG |
| | | M2B | target | CGTATT | AACCGC | GTCCGA | CGAATAATAG |
| | | | cg12998491 | A | T | AACGAT | CCGCCCGTGAC |
| | | | | (SEQ ID | (SEQ ID | ATCAA- | ATCTCCGCTGA |
| | | | | NO: 244) | NO: 245) | BHQ1 | TACCGTCCCGG |
| | | | | | | (SEQ ID | ACGGGCGGGG |
| | | | | | | NO: 246) | TGGGGGGCGA |
| | | | | | | | GCGGCTGCCGC |
| | | | | | | | TGCAGGG |
| | | | | | | | (SEQ ID NO: 247) |
| HB-980 | MYOCD | H- | CIMP | GGTTCG | CAATCA | 6FAM- | GGCCCGCCGC |
| | | MYOCD- | Infinium | TCGTAA | AAAACG | AAACCG | AAAGAGTTAA |
| | | M1B | target | AGAGTT | ACGAAC | CCGAAA | GAGCCGGTTCC |
| | | | cg21665000 | AAGAGT | GA | CCGTCT | CGAGACGGCTT |
| | | | | C | (SEQ ID | CGAAA- | CGGCGGCTCCG |
| | | | | (SEQ ID | NO: 249) | BHQ1 | GGTCCCCAGAC |
| | | | | NO: 248) | | (SEQ ID | CCCGCTCGCCG |
| | | | | | | NO: 250) | CTCCTGATTG |
| | | | | | | | (SEQ ID NO: 251) |
| HB-981 | KCNC1 | H- | CIMP | TAGTTT | CAAAAA | 6FAM- | CAGCCCAGCG |
| | | KCNC1- | Infinium | AGCGGA | CACCCG | TAACGC | GAACCCCAGCT |
| | | M1B | target | ATTTTA | AAATAT | CGAACG | CGAGCCCGGG |
| | | | cg06763078 | GTTCGA | TACTCG | CTACTC | CTCACGGAGA |
| | | | | GT | TA | TCCGTA | GCAGCGCTCG |
| | | | | (SEQ ID | (SEQ ID | AACC- | GCGTTAGCCGC |
| | | | | NO: 252) | NO: 253) | BHQ1 | ACGAGCAACA |
| | | | | | | (SEQ ID | CCCCGGGTGCC |
| | | | | | | NO: 254) | CCTG |
| | | | | | | | (SEQ ID NO: 255) |
| HB-982 | FSTL1 | H- | CIMP | TTTCGG | CTTCCG | 6FAM- | CCTCGGCCCCT |
| | | FSTL1- | Infinium | TTTTTC | CAAATA | CTCGCG | CGCCTACCTCG |
| | | M1B | target | GTTTAT | TAAAAA | CTAATA | GCGCGGACCC |
| | | | cg22469841 | TTCG | CGCT | ACGATC | AGGCGACCGC |
| | | | (not | (SEQ ID | (SEQ ID | GCCTAA | CACCAGCGCG |
| | | | overlapping) | NO: 256) | NO: 257) | ATCCG- | AGCGCGAGCG |
| | | | | | | BHQ1 | CGAGCCAGCG |
| | | | | | | (SEQ ID | TTTCCACATCT |
| | | | | | | NO: 258) | GCGGAAG |
| | | | | | | | (SEQ ID NO: 259) |
| HB-983 | FSTL1 | H- | CIMP | CATCGA | AACTCG | 6FAM- | GACCGAAACT |
| | | FSTL1- | Infinium | AATTTT | ATCCCC | CGCTAA | CCCAGCGCCAC |
| | | M2B | target | TAGCGT | GAAACC | ACGAAT | CCCGGGAGAG |
| | | | cg22469841 | TATTTC | (SEQ ID | AAACGC | CATCCCCAGGA |
| | | | (not | (SEQ ID | NO: 261) | GCGTCC | CGCGCGCCCAC |
| | | | overlapping) | NO: 260) | | T-BHQ1 | CCGCCCAGCGC |
| | | | | | | (SEQ ID | GCAGACCCAA |
| | | | | | | NO: 262) | GAGGCCCCGG |
| | | | | | | | GGACCGAGTT |
| | | | | | | | (SEQ ID NO: 263) |
| HB-984 | FSTL1 | H- | CIMP | CATCGA | CCCGAA | 6FAM- | GACCGAAACT |
| | | FSTL1- | Infinium | AATTTT | ACCTCT | CGCTAA | CCCAGCGCCAC |
| | | M3B | target | TAGCGT | TAAATC | ACGAAT | CCCGGGAGAG |
| | | | cg22469841 | TATTTC | TACG | AAACGC | CATCCCCAGGA |
| | | | (not | (SEQ ID | (SEQ ID | GCGTCC | CGCGCGCCCAC |
| | | | overlapping) | NO: 260) | NO: 264) | T-BHQ1 | CCGCCCAGCGC |
| | | | | | | (SEQ ID | GCAGACCCAA |
| | | | | | | NO: 262) | GAGGCCCCGG |
| | | | | | | | G |
| | | | | | | | (SEQ ID NO: 265) |
| HB-985 | SLC6A4 | H- | CIMP | CGTATT | AAATTT | 6FAM- | CGTATTTGTAC |
| | | SLC6A4- | Infinium | TGTATT | ATTCGC | ACTCTT | CCGCGGGCCCT |
| | | M1M | target | CGCGGG | CTCAAA | TAACGA | CACATGGTCTG |
| | | | cg05016953 | TT | ATAACG | CGACTA | ATCTCTAGATA |
| | | | | (SEQ ID | (SEQ ID | TC- | GCCGCCGCCA |
| | | | | NO: 266) | NO: 267) | MGBFNQ | AAGAGCTCTTG |
| | | | | | | (SEQ ID | AAGAATTTTTG |
| | | | | | | NO: 268) | CGTCACTTTGA |
| | | | | | | | GGCGAATAAA |
| | | | | | | | CTT |
| | | | | | | | (SEQ ID NO: 269) |
| HB-633 | FAM78A | H- | Infinium | GACGGC | AACGAC | 6FAM- | GACGGCGCAG |
| | | FAM78 | targeted | GTAGTT | TATTAT | CGTACG | CTCTGGGCGGT |
| | | A-M1B | cg12998491 | TTGGGC | TCGCTA | ACCGCC | CTCCCCGGAGG |
| | | | | (SEQ ID | ATACGC | ACTAAC | CGGTGGCCCCC |
| | | | | NO: 270) | G | GAAACG | GCCCCCCGCGC |
| | | | | | (SEQ ID | AAC- | CCGCCCCGTCA |
| | | | | | NO: 271) | BHQ-1 | GTGGCGGCCG |
| | | | | | | (SEQ ID | CACGACCGCG |
| | | | | | | NO: 272) | CGCACCAGCG |
| | | | | | | | AATAATAGCC |
| | | | | | | | GCC |
| | | | | | | | (SEQ ID NO: 273) |
| HB-150 | MLH1 | H- | | AGGAA | TCTTCG | 6FAM- | AGGAAGAGCG |
| | | MLH1- | | GAGCGG | TCCCTC | CCCGCT | GACAGCGATCT |
| | | M2B | | ATAGCG | CCTAAA | ACCTAA | CTAACGCGCA |
| | | | | ATTT | ACG | AAAAAT | AGCGCATATCC |
| | | | | (SEQ ID | (SEQ ID | ATACGC | TTCTAGGTAGC |
| | | | | NO: 274) | NO: 275) | TTACGC | GGGCAGTAGC |
| | | | | | | G-BHQ-1 | CGCTTCAGGGA |
| | | | | | | (SEQ ID | GGGACGAAGA |
| | | | | | | NO: 276) | (SEQ ID NO: 277) |

### Example 7

### Effects of DNA Hypermethylation on Gene Expression were Characterized

Promoter CpG island DNA hypermethylation can lead to transcriptional silencing of the associated gene. However, the majority of cancer-specific CpG island hypermethylation may occur in gene promoters that are not normally expressed, and therefore may not be involved in tumor initiation or progression (Widschwendter et al., 2007; Gal-Yam et al., 2008).

In this working example, Applicants examined the extent to which cancer-specific DNA hypermethylation affects gene expression in colorectal tumors, by performing an integrated analysis of promoter DNA methylation and gene expression data from six CIMP-H normal adjacent-tumor pairs and 13 pairs of non-CIMP tumors and adjacent-normal tissues. Applicants found that 7.3% of genes that showed DNA hypermethylation (|Δβ|>0.20) in CIMP-H tumors also showed more than a 2-fold reduction in gene expression (FIGS. 6A and 6B). Applicants identified 464 genes that are downregulated more than 2-fold in CIMP-H tumors compared with adjacent normal tissue (FIG. 6A).

FIGS. 6A-C show, according to particular exemplary aspects, an integrated analysis of gene expression and promoter DNA methylation changes between colorectal tumors and matched normal adjacent tissues. (A) Mean DNA methylation β-value differences between CIMP-H tumors and matched normal colonic tissues (n=6) are plotted on the x-axis and mean log₂-transformed gene expression values differences are plotted on the y-axis for each gene. Red data points highlight those genes that are hypermethylated with β-value difference >0.20 and show more than 2-fold decrease in their gene expression levels in CIMP-H tumors. (B) Pie chart showing the gene expression changes of 1,534 hypermethylated genes in CIMP-H tumors compared with adjacent normal tissues. (C) Bar chart showing the number of genes that exhibit DNA hypermethylation and/or gene expression changes in non-CIMP tumors among the 112 genes that are hypermethylated and downregulated in CIMP-H tumors.

Applicants found that 112 genes (24%) that are downregulated in CIMP-H are directly associated with promoter DNA hypermethylation (Table 6 below).

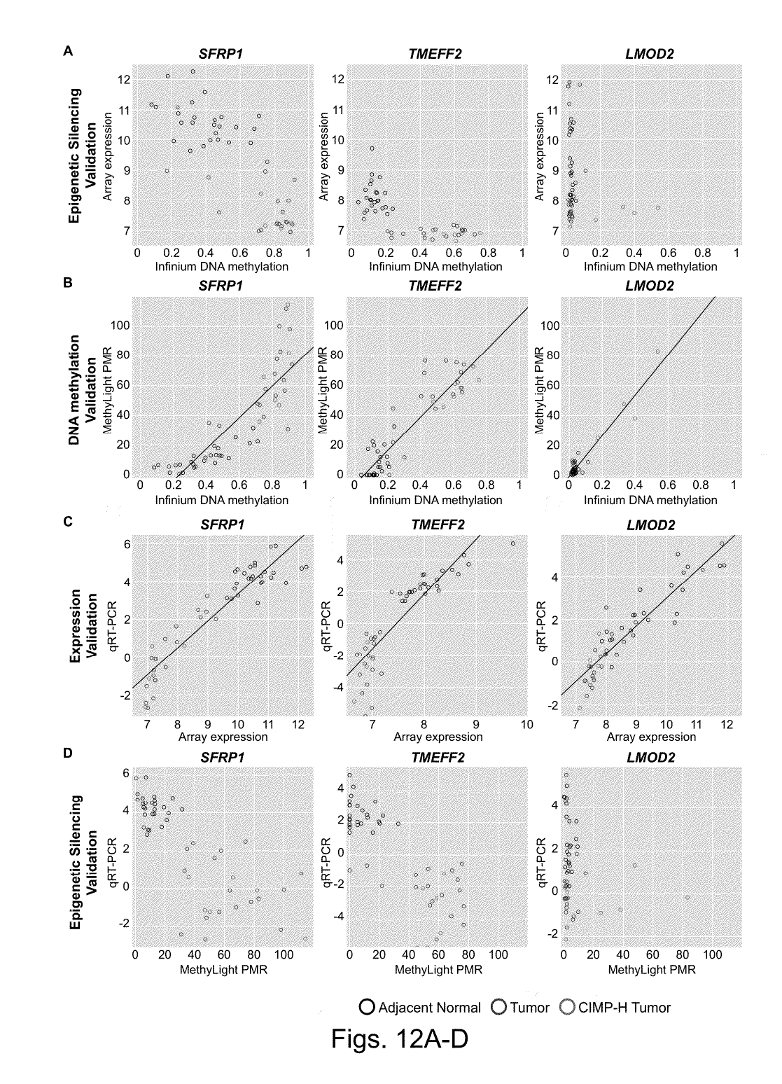

Furthermore, 12 genes were identified that are both downregulated and cancer-specifically hypermethylated in both CIMP-H and non-CIMP tumors (FIG. 6C and Table 6 below). DNA hypermethylation and transcriptional silencing of these genes may play a critical role in the development of CRC, irrespective of molecular subgroups. These include SFRP1 and SFRP2, which function as negative regulators of Wnt signaling and have been proposed as epigenetic gatekeeper genes in colorectal tumorigenesis (Baylin and Ohm, 2006). Applicants validated the DNA methylation and gene expression findings for SFRP1 and TMEFF2 using MethyLight and quantitative RT-PCR (qRT-PCR) technologies, respectively (FIG. 12).

FIGS. 12A-D show, according to particular exemplary aspects, validation of the Infinium DNA methylation data and gene expression array data using MethyLight and quantitative RT-PCR (qRT-PCR), respectively. The validations were performed for three genes indicated above each scatter plot (A) Comparison of Infinium DNA methylation β-value (x-axis) and log 2-transformed gene expression value from Illumina expression array (y-axis). (B) Validation of Infinium DNA methylation data by MethyLight technology. The x-axis represents Infinium DNA methylation β-value and the y-axis represents PMR value from MethyLight assay. Pearson correlation coefficients between the assays: 0.85 for SFRP1, 0.91 for TMEFF2 and 0.96 for LMOD1. (C) Validation of Illumina expression array data by qRT-PCR assay. The x-axis represents log 2-transformed array-based gene expression value and the y-axis represents log 2-transformed relative copy number normalized to HTPR1 using qRT-PCR assay. Pearson correlation coefficients between the gene expression platforms: 0.93 for SFRP1, 0.89 for TMEFF2 and 0.91 for LMOD1. (D) Comparison of MethyLight PMR values (x-axis) and log 2-transformed normalized relative copy number from qRT-PCR assay (y-axis). Black open circle: adjacent normal (n=25), red open circle (herein reproduced in gray-scale): tumors in CIMP-L, Cluster 3 and Cluster 4 (n=19), blue open circle (herein reproduced in gray-scale): CIMP-H tumors (n=6).

Intriguingly, 48/112 genes were also identified that are downregulated in both CIMP-H and non-CIMP tumors compared with the matched adjacent normal colon. However, substantial increases in promoter DNA methylation for these genes were observed only in CIMP-H tumors. This finding was confirmed for the LMOD1 gene using MethyLight and qRT-PCR technologies (FIG. 12). LMOD1 has been found to be somatically mutated in human cancer and cancer cell lines (http://www.sanger.ac.uk/genetics/CGP/cosmic/). However, DNA hypermethylation of this gene has not yet been reported. These findings indicate that genetic or other epigenetic mechanismd such as chromatin modifications might be involved in silencing of these genes in non-CIMP tumors.

TABLE 6
Genes that are hypermethylated with β-value difference > 0.2 and show more than a 2-
fold decrease in their gene expression levels in CIMP-H tumors compared with normal adjacent tissue.

| | | | | | | | | | | Expres- | Expres- |
|---|---|---|---|---|---|---|---|---|---|---|---|
| | | | | | | | | | | sion | sion |
| | | | | | | | | | | CIMP- | Non- |
| | | | | DNA methylation | | | DNA methylation | | | H | CIMP |
| | | | | CIMP-H | | | Non-CIMP | | | Fold | Fold |
| | Expression | | | Mean | Mean | Mean | Mean | Mean | Mean | change | change |
| Expression | probe | | Gene | Tu- | Nor- | Differ- | Tu- | Nor- | Differ- | Normal/ | Normal/ |
| Probe ID | nuID | Refseq | Symbol | mor | mal | ence | mor | mal | ence | Tumor | Tumor |
| ILMN_1814327 | 9QSiOIvUj73tS.UCpI | NM_032049 | AGTR1 | 0.77 | 0.10 | 0.67 | 0.45 | 0.10 | 0.35 | 2.2 | 2.0 |
| | | NM_031850 | | | | | | | | | |
| | | NM_004835 | | | | | | | | | |
| | | NM_009585 | | | | | | | | | |
| | | NM_000685 | | | | | | | | | |
| ILMN_1671478 | EA4JWlRFYRSV559cHw | NM_001823 | CKB | 0.36 | 0.05 | 0.31 | 0.24 | 0.04 | 0.20 | 4.0 | 3.5 |
| ILMN_1789400 | cmZ53ab3e1Hq7k_PwE | NM_004474 | FOXD2 | 0.67 | 0.29 | 0.38 | 0.52 | 0.27 | 0.25 | 5.1 | 2.2 |
| ILMN_2153916 | E7X7lCxiNz76C3_szk | NM_021979 | HSPA2 | 0.63 | 0.27 | 0.36 | 0.43 | 0.19 | 0.23 | 2.3 | 2.3 |
| ILMN_2327860 | 6dFQSN.UitTroIYwV4 | NM_022440 | MAL | 0.63 | 0.05 | 0.58 | 0.30 | 0.05 | 0.25 | 3.7 | 3.3 |
| | | NM_022439 | | | | | | | | | |
| | | NM_002371 | | | | | | | | | |
| | | NM_022438 | | | | | | | | | |
| ILMN_2149164 | rR_nfrF7q8k0L3VSrI | NM_003012 | SFRP1 | 0.82 | 0.49 | 0.33 | 0.68 | 0.36 | 0.32 | 7.8 | 6.3 |
| ILMN_1722898 | xpUB_6tEBXviMCenvM | NM_003013 | SFRP2 | 0.65 | 0.09 | 0.56 | 0.46 | 0.11 | 0.36 | 4.0 | 3.0 |
| ILMN_2126038 | cHUr4Ht6eCQqKt6RlU | NM_007029 | STMN2 | 0.68 | 0.40 | 0.28 | 0.56 | 0.34 | 0.22 | 3.4 | 2.5 |
| ILMN_2109197 | xntytbWu7SP9dI8juU | NM_012307 | EPB41L3 | 0.68 | 0.46 | 0.22 | 0.54 | 0.32 | 0.23 | 3.0 | 2.3 |
| ILMN_1730645 | 0cCok79EunocWIn6HE | NM_016192 | TMEFF2 | 0.60 | 0.16 | 0.44 | 0.43 | 0.12 | 0.31 | 2.1 | 2.4 |
| ILMN_1689088 | TuROh4NEyPddbSwW5g | NM_130386 | COLEC12 | 0.67 | 0.22 | 0.45 | 0.38 | 0.18 | 0.20 | 2.3 | 2.0 |
| ILMN_1679391 | 3EKQ5xffz1V.gcX_C0 | NM_153267 | MAMDC2 | 0.73 | 0.19 | 0.54 | 0.32 | 0.12 | 0.20 | 9.4 | 6.2 |
| ILMN_1759330 | oFYu8lcp17Hubkfinc | NM_004321 | KIF1A | 0.50 | 0.05 | 0.45 | 0.04 | 0.03 | 0.01 | 2.3 | 2.0 |
| ILMN_1695157 | TSXd75dJ3UL1KfWtfI | NM_000717 | CA4 | 0.69 | 0.11 | 0.58 | 0.11 | 0.08 | 0.04 | 23.8 | 20.4 |
| ILMN_1761789 | cp5d53UIVAhXqBdd60 | NM_005182 | CA7 | 0.43 | 0.07 | 0.36 | 0.11 | 0.06 | 0.04 | 4.8 | 4.4 |
| | | NM_001014435 | | | | | | | | | |
| ILMN_1669410 | BlUQfwE_GPX3eBEpJ8 | NM_001275 | CHGA | 0.69 | 0.21 | 0.48 | 0.19 | 0.14 | 0.05 | 15.6 | 12.1 |
| ILMN_1809456 | K1XhXnXNUSz8OV7S1c | NM_147164 | CNTFR | 0.52 | 0.03 | 0.49 | 0.05 | 0.02 | 0.02 | 3.4 | 2.7 |
| | | NM_001842 | | | | | | | | | |
| ILMN_1701441 | Ef7_Kh281J36yUReQ4 | NM_057159 | LPAR1 | 0.26 | 0.05 | 0.21 | 0.03 | 0.04 | −0.01 | 3.2 | 2.5 |
| | | NM_001401 | | | | | | | | | |
| ILMN_1703205 | Tq7UqVbIiE.COuzhIk | NM_207034 | EDN3 | 0.54 | 0.33 | 0.21 | 0.45 | 0.31 | 0.15 | 4.5 | 3.2 |
| | | NM_207033 | | | | | | | | | |
| | | NM_000114 | | | | | | | | | |
| | | NM_207032 | | | | | | | | | |
| ILMN_2358886 | ripRim2n8hEjTJ3Eos | NM_207034 | EDN3 | 0.54 | 0.33 | 0.21 | 0.45 | 0.31 | 0.15 | 4.6 | 3.1 |
| | | NM_207033 | | | | | | | | | |
| | | NM_000114 | | | | | | | | | |
| | | NM_207032 | | | | | | | | | |
| ILMN_2322996 | NdaDznfsXu6er98tvo | NM_172111 | EYA2 | 0.63 | 0.13 | 0.50 | 0.25 | 0.10 | 0.15 | 5.6 | 2.3 |
| | | NM_172110 | | | | | | | | | |
| | | NM_005244 | | | | | | | | | |
| | | NM_172113 | | | | | | | | | |
| | | NM_172112 | | | | | | | | | |
| ILMN_1715748 | 6SeonnyugB1AwLmntk | NM_001458 | FLNC | 0.49 | 0.01 | 0.47 | 0.02 | 0.01 | 0.01 | 3.5 | 2.0 |
| ILMN_1680390 | HIky7_py6v3hJU6g5U | NM_001491 | GCNT2 | 0.66 | 0.35 | 0.31 | 0.49 | 0.47 | 0.01 | 2.7 | 2.0 |
| | | NM_145655 | | | | | | | | | |
| | | NM_145649 | | | | | | | | | |
| ILMN_1810716 | QUUJV.eJX134cUo0Ug | NM_001496 | GFRA3 | 0.53 | 0.07 | 0.46 | 0.14 | 0.08 | 0.06 | 2.2 | 2.3 |
| ILMN_1775814 | crpRMJTz0cs4cvu3pI | NM_000163 | GHR | 0.39 | 0.07 | 0.32 | 0.05 | 0.06 | −0.01 | 2.9 | 2.5 |
| ILMN_1802397 | lZpbgoRTXknQXQosQU | NM_002067 | GNA11 | 0.50 | 0.15 | 0.35 | 0.16 | 0.13 | 0.03 | 2.6 | 2.1 |
| ILMN_1795257 | u0oleop3olR7H5345c | NM_005309 | GPT | 0.90 | 0.64 | 0.26 | 0.77 | 0.69 | 0.09 | 5.8 | 3.6 |
| ILMN_1726666 | 6faKG5XRV0etR6fiHQ | NM_002084 | GPX3 | 0.32 | 0.05 | 0.26 | 0.09 | 0.05 | 0.04 | 3.4 | 3.7 |
| ILMN_1815203 | cpX09d1OeQ0e16nzrk | NM_005518 | HMGCS2 | 0.82 | 0.49 | 0.33 | 0.60 | 0.53 | 0.08 | 21.0 | 7.3 |
| ILMN_1808258 | c0CeLS6d_YOVTJILVo | NM_001040173 | HTR4 | 0.53 | 0.21 | 0.32 | 0.06 | 0.10 | −0.04 | 3.7 | 2.2 |
| | | NM_001040172 | | | | | | | | | |
| | | NM_000870 | | | | | | | | | |
| ILMN_1660086 | l1V3Xr9RF0hGRL0ULk | NM_001040114 | MYH11 | 0.56 | 0.07 | 0.48 | 0.15 | 0.07 | 0.08 | 16.2 | 3.1 |
| | | NM_001040113 | | | | | | | | | |
| | | NM_022844 | | | | | | | | | |
| | | NM_002474 | | | | | | | | | |
| ILMN_1814221 | Kkl.6s0Dq0AU6heUXU | NM_002522 | NPTX1 | 0.71 | 0.04 | 0.67 | 0.12 | 0.04 | 0.09 | 3.0 | 3.6 |
| ILMN_1685387 | rdxCJV5V3vopEv8gwo | NM_002644 | PIGR | 0.79 | 0.51 | 0.27 | 0.75 | 0.56 | 0.18 | 15.2 | 6.3 |
| ILMN_1769394 | uFlO1F07cIGqFJNTSU | NM_006225 | PLCD1 | 0.63 | 0.34 | 0.29 | 0.45 | 0.31 | 0.14 | 3.6 | 2.4 |
| ILMN_2062714 | rl55P5uN0IXUlILV9Q | NM_000953 | PTGDR | 0.52 | 0.17 | 0.35 | 0.17 | 0.14 | 0.03 | 3.1 | 2.1 |
| ILMN_1740917 | 630Xr1xXbVxe141614 | NM_000336 | SCNN1B | 0.37 | 0.14 | 0.23 | 0.24 | 0.15 | 0.08 | 5.4 | 6.0 |
| ILMN_1689111 | 0SXicSOVOWPfYCU75Q | NM_000609 | CXCL12 | 0.61 | 0.23 | 0.38 | 0.20 | 0.25 | −0.05 | 2.9 | 2.9 |
| | | NM_001033886 | | | | | | | | | |
| | | NM_199168 | | | | | | | | | |
| ILMN_1791447 | BS4MBTTflHFV14JRJI | NM_199168 | CXCL12 | 0.61 | 0.23 | 0.38 | 0.20 | 0.25 | −0.05 | 5.6 | 5.6 |
| ILMN_1812824 | Knnt4FQUhog45XoF4I | NM_001048 | SST | 0.63 | 0.35 | 0.27 | 0.44 | 0.25 | 0.19 | 3.7 | 6.7 |
| ILMN_1728570 | KYi4XgILrhZiWfO6BQ | NM_003206 | TCF21 | 0.47 | 0.19 | 0.28 | 0.42 | 0.28 | 0.14 | 3.0 | 2.1 |
| | | NM_198392 | | | | | | | | | |
| ILMN_1766712 | WXd3tUVZiBH8QYYoj0 | NM_003206 | TCF21 | 0.47 | 0.19 | 0.28 | 0.42 | 0.28 | 0.14 | 3.0 | 2.0 |
| ILMN_1752214 | uSUBX5cKA6CL_5Ineg | NM_001077 | UGT2B17 | 0.58 | 0.35 | 0.23 | 0.54 | 0.37 | 0.17 | 19.7 | 9.5 |
| ILMN_2373670 | N1ALxVEQC8eJ9fO.RE | NM_001077 | UGT2B17 | 0.58 | 0.35 | 0.23 | 0.54 | 0.37 | 0.17 | 11.3 | 5.9 |
| ILMN_2305407 | 3RCViiNWVh6KMiCGxc | NM_001018011 | ZBTB16 | 0.26 | 0.03 | 0.24 | 0.02 | 0.02 | 0.00 | 3.2 | 2.5 |
| | | NM_006006 | | | | | | | | | |
| ILMN_1715991 | cpUoJA1Xl.LOBRzCwU | NM_004657 | SDPR | 0.27 | 0.04 | 0.22 | 0.07 | 0.04 | 0.03 | 2.4 | 2.5 |
| ILMN_1680987 | 3cXFOXvudK7_9NeU1U | NM_004821 | HAND1 | 0.47 | 0.06 | 0.41 | 0.09 | 0.05 | 0.04 | 4.8 | 3.1 |
| ILMN_1776953 | 3CBVEhgxeipOOJilWo | NM_006097 | MYL9 | 0.46 | 0.24 | 0.21 | 0.32 | 0.19 | 0.13 | 2.3 | 2.4 |
| ILMN_1675062 | Tbs1URA5CdFCtV3S1U | NM_181526 | MYL9 | 0.46 | 0.24 | 0.21 | 0.32 | 0.19 | 0.13 | 2.8 | 2.5 |
| | | NM_006097 | | | | | | | | | |
| ILMN_1696657 | iX_6IDu0XVUFY9Hf90 | NM_201630 | LRRN2 | 0.39 | 0.13 | 0.27 | 0.16 | 0.07 | 0.10 | 3.6 | 3.1 |
| | | NM_006338 | | | | | | | | | |
| ILMN_1789648 | odUCHSdV7yOneXtfVQ | NM_006998 | SCGN | 0.49 | 0.23 | 0.26 | 0.20 | 0.18 | 0.02 | 3.3 | 3.6 |
| ILMN_1666536 | 93j6rnXqbnvntL55vc | NM_014312 | VSIG2 | 0.56 | 0.35 | 0.20 | 0.56 | 0.37 | 0.19 | 9.1 | 9.5 |
| ILMN_1680948 | 6PV1FR_XtFUtFwkNHo | NM_012134 | LMOD1 | 0.25 | 0.04 | 0.21 | 0.04 | 0.03 | 0.01 | 2.5 | 2.2 |
| ILMN_1791280 | it1ISdTSgUJApSMl4Q | NM_014365 | HSPB8 | 0.38 | 0.12 | 0.26 | 0.07 | 0.09 | −0.02 | 3.1 | 2.5 |
| ILMN_1746888 | uSKJJzePoBeWHy5rjI | NM_013363 | PCOLCE2 | 0.65 | 0.07 | 0.57 | 0.04 | 0.04 | −0.01 | 4.6 | 5.8 |
| ILMN_1778650 | TEHjkUp74oXlIqboV4 | NM_015873 | VILL | 0.49 | 0.13 | 0.37 | 0.37 | 0.19 | 0.19 | 2.3 | 2.0 |
| ILMN_1671891 | 6WKAX5VTR0k_R8jEg4 | NM_001100818 | PID1 | 0.36 | 0.13 | 0.23 | 0.14 | 0.13 | 0.01 | 3.1 | 2.1 |
| | | NM_017933 | | | | | | | | | |
| ILMN_1657373 | KU6Ur5couq1HjgCK78 | NM_018192 | LEPREL1 | 0.48 | 0.05 | 0.44 | 0.05 | 0.05 | 0.00 | 4.3 | 2.7 |
| ILMN_1703572 | KuD33Ip_7Ecevv8vqU | NM_022843 | PCDH20 | 0.37 | 0.07 | 0.30 | 0.08 | 0.05 | 0.03 | 3.2 | 2.2 |
| ILMN_2180885 | QuER.qBRgjhdhKAeSk | NM_025087 | CWH43 | 0.68 | 0.28 | 0.40 | 0.28 | 0.21 | 0.07 | 6.7 | 5.1 |
| ILMN_1715612 | Bro.cacdXeprTbQTeU | NM_178160 | OTOP2 | 0.44 | 0.07 | 0.38 | 0.10 | 0.07 | 0.03 | 3.9 | 3.4 |
| ILMN_1788942 | ZnR56dKVQXel.wvFyE | NM_001122890 | GGT6 | 0.75 | 0.47 | 0.28 | 0.72 | 0.57 | 0.15 | 4.6 | 2.9 |
| | | NM_153338 | | | | | | | | | |
| ILMN_1721283 | rX0IVB4kVnePVRJf3U | NM_144617 | HSPB6 | 0.60 | 0.32 | 0.27 | 0.43 | 0.26 | 0.17 | 4.9 | 4.2 |
| ILMN_1808157 | lXy6UiHh9Cv89SIdSI | NM_138290 | RUNDC3B | 0.33 | 0.02 | 0.31 | 0.02 | 0.02 | 0.00 | 2.6 | 2.0 |
| ILMN_1789096 | fUheh_BdAlwro6foQ4 | NM_152672 | OSTalpha | 0.73 | 0.48 | 0.26 | 0.61 | 0.46 | 0.15 | 9.5 | 2.9 |
| ILMN_1751062 | xKBJBHXp4QcJQzpXK0 | NM_173833 | SCARA5 | 0.62 | 0.13 | 0.48 | 0.16 | 0.13 | 0.02 | 6.7 | 5.9 |
| ILMN_1788267 | fT36geVWVRIp.zd4nk | NM_017726 | PPP1R14D | 0.77 | 0.51 | 0.27 | 0.53 | 0.56 | −0.03 | 3.8 | 1.9 |
| ILMN_2115434 | oUu_eMzp95937u5dJ0 | NM_006834 | RAB32 | 0.55 | 0.16 | 0.39 | 0.09 | 0.10 | −0.01 | 2.1 | 1.3 |
| ILMN_1689176 | ifgoH5T1_f4UeTgefc | NM_024574 | C4orf31 | 0.29 | 0.06 | 0.23 | 0.04 | 0.04 | 0.00 | 2.1 | 1.4 |
| ILMN_1742544 | ou.dPsAp5Z6ukglCIU | NM_002397 | MEF2C | 0.32 | 0.01 | 0.31 | 0.01 | 0.02 | 0.00 | 2.2 | 1.4 |
| ILMN_1779448 | Hs8SyFnhvVC4SNSpf0 | NM_025202 | EFHD1 | 0.35 | 0.13 | 0.22 | 0.13 | 0.13 | 0.00 | 2.2 | 1.3 |
| ILMN_1731374 | TgCvBIxJ2i_4kdx5CE | NM_001873 | CPE | 0.47 | 0.05 | 0.41 | 0.04 | 0.03 | 0.00 | 2.5 | 1.2 |
| ILMN_2087692 | cVeeL6A9S5JHj4US_U | NM_024843 | CYBRD1 | 0.26 | 0.01 | 0.25 | 0.02 | 0.01 | 0.00 | 2.7 | 1.6 |
| ILMN_2369666 | olf.CgpHACJ7XrNyFQ | NM_001877 | CR2 | 0.30 | 0.02 | 0.27 | 0.03 | 0.02 | 0.01 | 2.2 | 1.3 |
| | | NM_001006658 | | | | | | | | | |
| ILMN_1760493 | Q_7Ylfq6pSiiuOm0ig | NM_017980 | LIMS2 | 0.62 | 0.40 | 0.22 | 0.37 | 0.37 | 0.01 | 2.4 | 1.8 |
| ILMN_1779071 | ZiQmZqtf5b6fcucsNM | NM_005103 | FEZ1 | 0.24 | 0.03 | 0.20 | 0.06 | 0.04 | 0.01 | 2.0 | 1.4 |
| ILMN_2374234 | QU5Gvc5J0jLnBPYsXU | NM_002731 | PRKACB | 0.57 | 0.03 | 0.55 | 0.02 | 0.01 | 0.01 | 2.1 | 1.7 |
| | | NM_207578 | | | | | | | | | |
| | | NM_182948 | | | | | | | | | |
| ILMN_1755850 | cbLX7qElLrSO5JdKIw | NM_021632 | ZNF350 | 0.49 | 0.13 | 0.36 | 0.15 | 0.13 | 0.02 | 2.1 | 1.2 |
| ILMN_1797342 | 96KT.nu4pY2UewK1yI | NM_015033 | FNBP1 | 0.53 | 0.03 | 0.51 | 0.05 | 0.03 | 0.02 | 2.4 | 1.5 |
| ILMN_2067656 | 6d0S7Hcwiu6u3v60o4 | NM_001759 | CCND2 | 0.33 | 0.08 | 0.25 | 0.09 | 0.07 | 0.02 | 2.2 | 1.2 |
| ILMN_1782079 | Kl1d9h8fqLSTeFEtuE | NM_153018 | ZFP3 | 0.23 | 0.02 | 0.21 | 0.06 | 0.03 | 0.03 | 2.3 | 1.4 |
| ILMN_1711928 | rde0g1Ben85FyO1UkU | NM_025149 | ACSF2 | 0.30 | 0.08 | 0.22 | 0.11 | 0.08 | 0.03 | 2.4 | 1.4 |
| ILMN_1680874 | ircfyu5nq3eud9Hvl4 | NM_178012 | TUBB2B | 0.41 | 0.04 | 0.37 | 0.08 | 0.04 | 0.04 | 2.4 | 1.9 |
| ILMN_2173294 | NFA1Ar3pUX7SKfUV50 | NM_018271 | THNSL2 | 0.53 | 0.14 | 0.38 | 0.18 | 0.13 | 0.06 | 2.2 | 1.1 |
| ILMN_2377900 | 0ni05u_DVH0uRe6XdI | NM_005909 | MAP1B | 0.68 | 0.07 | 0.61 | 0.11 | 0.05 | 0.06 | 2.2 | 1.8 |
| ILMN_1789166 | uf_LXalSDbNUCV1Tpc | NM_020209 | SHD | 0.89 | 0.13 | 0.76 | 0.17 | 0.10 | 0.07 | 2.2 | 1.8 |
| ILMN_1722713 | 0W6n1p3e6OkrTuCE0M | NM_001996 | FBLN1 | 0.66 | 0.38 | 0.28 | 0.48 | 0.40 | 0.08 | 2.1 | 1.4 |
| | | NM_006486 | | | | | | | | | |
| | | NM_006485 | | | | | | | | | |
| | | NM_006487 | | | | | | | | | |
| ILMN_1700541 | iOI6N3dlGkW0nX_6UI | NM_001996 | FBLN1 | 0.66 | 0.38 | 0.28 | 0.48 | 0.40 | 0.08 | 2.7 | 1.5 |
| | | XM_001718348 | | | | | | | | | |
| ILMN_1672536 | Z0Uqg6eo.dpHeTTU38 | NM_006486 | FBLN1 | 0.66 | 0.38 | 0.28 | 0.48 | 0.40 | 0.08 | 2.7 | 1.5 |
| ILMN_1806710 | o4V7t1B55FU1YV7dZQ | NM_031475 | ESPN | 0.46 | 0.19 | 0.28 | 0.28 | 0.19 | 0.09 | 2.4 | 1.3 |
| ILMN_2246956 | 3TkXAX.oA3lupXUjJ0 | NM_000633 | BCL2 | 0.53 | 0.06 | 0.47 | 0.19 | 0.09 | 0.10 | 2.0 | 1.8 |
| ILMN_1812031 | ZX5zBVd5967uGKVWO8 | NM_002579 | PALM | 0.52 | 0.23 | 0.29 | 0.31 | 0.21 | 0.10 | 2.5 | 1.6 |
| | | NM_001040134 | | | | | | | | | |
| ILMN_2072178 | cJAK6QVOikgIKGUl4U | NM_024693 | ECHDC3 | 0.46 | 0.13 | 0.33 | 0.21 | 0.10 | 0.10 | 2.0 | 1.0 |
| ILMN_1763433 | rHdQ4h0QnqW6yHTL10 | NM_015163 | TRIM9 | 0.60 | 0.02 | 0.58 | 0.12 | 0.02 | 0.10 | 2.1 | 1.8 |
| ILMN_2350634 | EFju.lS3QOU7liB5VI | NM_001039349 | EFEMP1 | 0.50 | 0.23 | 0.27 | 0.36 | 0.25 | 0.11 | 2.0 | 1.7 |
| | | NM_001039348 | | | | | | | | | |
| | | NM_004105 | | | | | | | | | |
| ILMN_1735877 | 916TUi4S4HwkDxXXFQ | NM_001039349 | EFEMP1 | 0.50 | 0.23 | 0.27 | 0.36 | 0.25 | 0.11 | 2.1 | 1.6 |
| | | NM_001039348 | | | | | | | | | |
| | | NM_004105 | | | | | | | | | |
| ILMN_1739594 | lKF_Xd6De0FS.6nUro | NM_147161 | ACOT11 | 0.55 | 0.24 | 0.31 | 0.39 | 0.27 | 0.13 | 2.2 | 1.7 |
| ILMN_1807493 | 33SlydI45qLnrFQFXc | NM_001077401 | ACVRL1 | 0.80 | 0.54 | 0.25 | 0.64 | 0.51 | 0.13 | 2.0 | 1.8 |
| | | NM_000020 | | | | | | | | | |
| ILMN_1779416 | 96ih0v3USX3Ul7uMQs | NM_020974 | SCUBE2 | 0.57 | 0.16 | 0.41 | 0.28 | 0.14 | 0.14 | 2.6 | 1.6 |
| ILMN_1736670 | ZkINSxfkTILJBU1flw | NM_005398 | PPP1R3C | 0.67 | 0.13 | 0.55 | 0.24 | 0.09 | 0.15 | 3.1 | 1.6 |
| ILMN_1718520 | 310RfkX1cf94PnOHHU | NM_001031709 | RNLS | 0.63 | 0.07 | 0.55 | 0.21 | 0.07 | 0.15 | 2.7 | 1.5 |
| ILMN_1749131 | Hq6ejtcV.NNXRYXFBs | NM_005021 | ENPP3 | 0.76 | 0.40 | 0.36 | 0.55 | 0.41 | 0.15 | 3.8 | 1.5 |
| ILMN_1742025 | Td6.WOoSgLTGpBLOaA | NM_014279 | OLFM1 | 0.43 | 0.10 | 0.33 | 0.23 | 0.07 | 0.16 | 2.3 | 1.5 |
| ILMN_2108735 | BJnZRnS5W.xQwHiYVQ | NM_001958 | EEF1A2 | 0.49 | 0.11 | 0.38 | 0.23 | 0.07 | 0.16 | 2.0 | 1.8 |
| ILMN_1692058 | 6pX04X56kSAEV26LEc | NM_002487 | NDN | 0.84 | 0.61 | 0.23 | 0.76 | 0.59 | 0.16 | 2.2 | 1.7 |
| ILMN_1653828 | 04qkX6fWXucn_U7Teg | NM_018223 | CHFR | 0.70 | 0.03 | 0.67 | 0.22 | 0.05 | 0.17 | 2.2 | 1.1 |
| ILMN_1776363 | NlAMIiljrgH.4hFc6U | NM_020977 | ANK2 | 0.65 | 0.36 | 0.29 | 0.42 | 0.24 | 0.18 | 2.1 | 1.9 |
| | | NM_001148 | | | | | | | | | |
| ILMN_1748323 | EXexMxXwgZVdREeVVI | NM_004887 | CXCL14 | 0.55 | 0.09 | 0.46 | 0.22 | 0.04 | 0.18 | 2.8 | 1.3 |
| ILMN_1676088 | 3oLk6h.V3eR1J3x7FM | NM_198080 | MSRB3 | 0.74 | 0.07 | 0.68 | 0.23 | 0.05 | 0.18 | 2.5 | 1.8 |
| | | NM_001031679 | | | | | | | | | |
| ILMN_2332553 | 3dJTSbUhTXILflQ3RI | NM_198080 | MSRB3 | 0.74 | 0.07 | 0.68 | 0.23 | 0.05 | 0.18 | 2.2 | 1.7 |
| | | NM_001031679 | | | | | | | | | |
| ILMN_1736078 | B3ptFMhFEObopYfpv8 | XM_001715879 * | THBS4 | 0.51 | 0.14 | 0.37 | 0.34 | 0.13 | 0.22 | 3.0 | 1.8 |
| | | XM_001714714 * | | | | | | | | | |
| | | NM_003248 | | | | | | | | | |
| | | XM_001715927 * | | | | | | | | | |
| ILMN_1680973 | NZmj0o_aoZICvCkf8A | NM_001451 | FOXF1 | 0.62 | 0.10 | 0.52 | 0.29 | 0.06 | 0.23 | 2.1 | 1.2 |
| ILMN_1731062 | HVIHafghXkO646gOIc | NM_000905 | NPY | 0.64 | 0.37 | 0.27 | 0.53 | 0.29 | 0.24 | 2.1 | 1.9 |
| ILMN_1765620 | Nk1T5SX57SPQD5.9yI | | VSTM2A | 0.46 | 0.15 | 0.31 | 0.39 | 0.10 | 0.29 | 2.1 | 1.8 |
| ILMN_1772627 | B_mSa7k0im7niCrrvA | NM_001040101 | D4S234E | 0.52 | 0.18 | 0.33 | 0.43 | 0.14 | 0.29 | 2.1 | 1.5 |
| | | NM_014392 | | | | | | | | | |
| ILMN_1741688 | uUguq6Xgld3efis_Vc | NM_198148 | CPXM2 | 0.56 | 0.15 | 0.41 | 0.48 | 0.17 | 0.30 | 2.8 | 1.8 |
| ILMN_1769575 | 0g7h0IdiljGKe495f4 | XM_001726649 | JAM3 | 0.50 | 0.09 | 0.41 | 0.40 | 0.09 | 0.31 | 2.0 | 1.5 |
| | | NM_032801 | | | | | | | | | |
| ILMN_1746359 | QifBTl1PqC3If4lKxI | NM_032918 | RERG | 0.63 | 0.09 | 0.53 | 0.39 | 0.07 | 0.32 | 2.5 | 1.7 |
| ILMN_1673566 | uloEAOK7sgSrVEr_qE | NM_006988 | ADAMTS1 | 0.69 | 0.11 | 0.58 | 0.39 | 0.07 | 0.32 | 2.2 | 1.8 |
| ILMN_1676449 | KeGnXX_6GK_IC64Lmk | NM_004787 | SLIT2 | 0.61 | 0.09 | 0.52 | 0.41 | 0.08 | 0.33 | 2.6 | 1.6 |
| ILMN_1789074 | oon0If5P1yz97_0vdA | NM_005345 | HSPA1A | 0.76 | 0.07 | 0.68 | 0.45 | 0.05 | 0.40 | 2.2 | 1.1 |
| ILMN_2399463 | 0f7fl7OSV27DGuDyLo | NM_001079874 | VAV3 | 0.76 | 0.11 | 0.65 | 0.52 | 0.10 | 0.42 | 4.0 | 1.1 |
| | | NM_006113 | | | | | | | | | |
| ILMN_1657679 | lvk.TqXqr9ckijPolU | NM_001079874 | VAV3 | 0.76 | 0.11 | 0.65 | 0.52 | 0.10 | 0.42 | 4.0 | 1.1 |
| | | NM_006113 | | | | | | | | | |

### CITED REFERENCES; AND THAT ARE INCORPORATED BY REFERENCE HEREIN IN THEIR ENTIRETY

Barbosa-Morais, N. L., M. J. Dunning, S. A. Samarajiwa, J. F. Darot, M. E. Ritchie, A. G. Lynch and S. Tavare. 2010. A re-annotation pipeline for Illumina BeadArrays: improving the interpretation of gene expression data. Nucleic Acids Res 38: e17.
Baylin, S. B. and J. E. Ohm. 2006. Epigenetic gene silencing in cancer—a mechanism for early oncogenic pathway addiction? Nat Rev Cancer 6: 107-116.
Bernstein, B. E., A. Meissner and E. S. Lander. 2007. The mammalian epigenome. Cell 128: 669-681.
Bibikova, M. 2009. Genome-wide DNA methylation profiling using Infinium assay. Epigenomics 1: 177-200.
Campan, M., D. J. Weisenberger, B. Trinh and P. W. Laird. 2009. MethyLight. Methods Mol Biol 507: 325-337. Chan, A. O., R. R. Broaddus, P. S. Houlihan, J. P. Issa, S. R. Hamilton and A. Rashid. 2002. CpG island methylation in aberrant crypt foci of the colorectum. Am J Pathol 160: 1823-1830.
Chan, T. A., S. Glockner, J. M. Yi, W. Chen, L. Van Neste, L. Cope, J. G. Herman, V. Velculescu, K. E. Schuebel, N. Ahuja et al. 2008. Convergence of mutation and epigenetic alterations identifies common genes in cancer that predict for poor prognosis. PLoS Med 5: e114.
Cheng, Y. W., H. Pincas, M. D. Bacolod, G. Schemmann, S. F. Giardina, J. Huang, S. Banal, K. Idrees, S. A. Khan, Z. Zeng et al. 2008. CpG island methylator phenotype associates with low-degree chromosomal abnormalities in colorectal cancer. Clin Cancer Res 14: 6005-6013.
Christensen, B. C., E. A. Houseman, J. J. Godleski, C. J. Marsit, J. L. Longacker, C. R. Roelofs, M. R. Karagas, M. R. Wrensch, R. F. Yeh, H. H. Nelson et al. 2009a. Epigenetic Profiles Distinguish Pleural Mesothelioma from Normal Pleura and Predict Lung Asbestos Burden and Clinical Outcome. Cancer Res 69: 227-234.
Christensen, B. C., E. A. Houseman, C. J. Marsit, S. Zheng, M. R. Wrensch, J. L. Wiemels, H. H. Nelson, M. R. Karagas, J. F. Padbury, R. Bueno et al. 2009b. Aging and environmental exposures alter tissue-specific DNA methylation dependent upon CpG island context. PLoS Genet 5: e1000602.
Christensen, B. C., K. T. Kelsey, S. Zheng, E. A. Houseman, C. J. Marsit, M. R. Wrensch, J. L. Wiemels, H. H. Nelson, M. R. Karagas, L. H. Kushi et al. 2010. Breast cancer DNA methylation profiles are associated with tumor size and alcohol and folate intake. PLoS Genet 6: e1001043.
Christensen, B. C., A. A. Smith, S. Zheng, D. C. Koestler, E. A. Houseman, C. J. Marsit, J. L. Wiemels, H. H. Nelson, M. R. Karagas, M. R. Wrensch et al. 2011. DNA methylation, isocitrate dehydrogenase mutation, and survival in glioma. J Natl Cancer Inst 103: 143-153.
Dickinson, R. E., A. Dallol, I. Bieche, D. Krex, D. Morton, E. R. Maher and F. Latif. 2004. Epigenetic inactivation of SLIT3 and SLIT1 genes in human cancers. Br J Cancer 91: 2071-2078.
Du, P., W. A. Kibbe and S. M. Lin. 2008. lumi: a pipeline for processing Illumina microarray. Bioinformatics 24: 1547-1548.
Estecio, M. R., J. Gallegos, C. Vallot, R. J. Castoro, W. Chung, S. Maegawa, Y. Oki, Y. Kondo, J. Jelinek, L. Shen et al. 2010. Genome architecture marked by retrotransposons modulates predisposition to DNA methylation in cancer. Genome Res 20: 1369-1382.
Estecio, M. R., V. Gharibyan, L. Shen, A. E. Ibrahim, K. Doshi, R. He, J. Jelinek, A. S. Yang, P. S. Yan, T. H. Huang et al. 2007. LINE-1 hypomethylation in cancer is highly variable and inversely correlated with microsatellite instability. PLoS ONE 2: e399.
Ewing, B. and P. Green. 1998. Base-calling of automated sequencer traces usingPhred. II. error probabilities. Genome research 8: 186.
Ewing, B., L. D. Hillier, M. C. Wendl and P. Green. 1998. Base-calling of automated sequencer traces usingPhred. I. Accuracy assessment. Genome research 8: 175.
Feinberg, A. P. and B. Vogelstein. 1983. Hypomethylation distinguishes genes of some human cancers from their normal counterparts. Nature 301: 89-92.
Gal-Yam, E. N., G. Egger, L. Iniguez, H. Holster, S. Einarsson, X. Zhang, J. C. Lin, G. Liang, P. A. Jones and A. Tanay. 2008. Frequent switching of Polycomb repressive marks and DNA hypermethylation in the PC3 prostate cancer cell line. Proc Natl Acad Sci USA 105: 12979-12984.
Gama-Sosa, M. A., V. A. Slagel, R. W. Trewyn, R. Oxenhandler, K. C. Kuo, C. W. Gehrke and M. Ehrlich. 1983. The 5-methylcytosine content of DNA from human tumors. Nucleic Acids Res 11: 6883-6894.
Goel, A., T. Nagasaka, C. N. Arnold, T. Inoue, C. Hamilton, D. Niedzwiecki, C. Compton, R. J. Mayer, R. Goldberg, M. M. Bertagnolli et al. 2007. The CpG island methylator phenotype and chromosomal instability are inversely correlated in sporadic colorectal cancer. Gastroenterology 132: 127-138.
Gordon, D., C. Abajian and P. Green. 1998. Consed: a graphical tool for sequence finishing. Genome research 8: 195.
Hinoue, T., D. J. Weisenberger, F. Pan, M. Campan, M. Kim, J. Young, V. L. Whitehall, B. A. Leggett and P. W. Laird. 2009. Analysis of the Association between CIMP and BRAF in Colorectal Cancer by DNA Methylation Profiling. PLoS One 4: e8357.
Houseman, E. A., B. C. Christensen, R. F. Yeh, C. J. Marsit, M. R. Karagas, M. Wrensch, H. H. Nelson, J. Wiemels, S. Zheng, J. K. Wiencke et al. 2008. Model-based clustering of DNA methylation array data: a recursive-partitioning algorithm for high-dimensional data arising as a mixture of beta distributions. BMC Bioinformatics 9: 365.
Ibrahim, A. E., M. J. Arends, A. L. Silva, A. H. Wyllie, L. Greger, Y. Ito, S. L. Vowler, T. H. Huang, S. Tavare, A. Murrell et al. 2011. Sequential DNA methylation changes are associated with DNMT3B overexpression in colorectal neoplastic progression. Gut 60: 499-508.
Joss, J. R. 2007. Classification of colorectal cancer based on correlation of clinical, morphological and molecular features. Histopathology 50: 113-130.
Jiang, X., J. Tan, J. Li, S. Kivimae, X. Yang, L. Zhuang, P. L. Lee, M. T. Chan, L. W. Stanton, E. T. Liu et al. 2008. DACT3 is an epigenetic regulator of Wnt/beta-catenin signaling in colorectal cancer and is a therapeutic target of histone modifications. Cancer Cell 13: 529-541.
Jones, P. A. and S. B. Baylin. 2007. The epigenomics of cancer. Cell 128: 683-692.
Kondo, Y., L. Shen, A. S. Cheng, S. Ahmed, Y. Boumber, C. Charo, T. Yamochi, T. Urano, K. Furukawa, B. Kwabi-Addo et al. 2008. Gene silencing in cancer by histone H3 lysine 27 trimethylation independent of promoter DNA methylation. Nat Genet 40: 741-750.
Ku, M., R. P. Koche, E. Rheinbay, E. M. Mendenhall, M. Endoh, T. S. Mikkelsen, A. Presser, C. Nusbaum, X. Xie, A. S. Chi et al. 2008. Genomewide Analysis of PRC1 and PRC2 Occupancy Identifies Two Classes of Bivalent Domains. PLoS Genet 4: e1000242.
Leggett, B. and V. Whitehall. 2010. Role of the serrated pathway in colorectal cancer pathogenesis. Gastroenterology 138: 2088-2100.
Limsui, D., R. A. Vierkant, L. S. Tillmans, A. H. Wang, D. J. Weisenberger, P. W. Laird, C. F. Lynch, K. E. Anderson, A. J. French, R. W. Haile et al. 2010. Cigarette smoking and colorectal cancer risk by molecularly defined subtypes. J Natl Cancer Inst 102: 1012-1022.
Marsit, C. J., B. C. Christensen, E. A. Houseman, M. R. Karagas, M. R. Wrensch, R. F. Yeh, H. H. Nelson, J. L. Wiemels, S. Zheng, M. R. Posner et al. 2009. Epigenetic profiling reveals etiologically distinct patterns of DNA methylation in head and neck squamous cell carcinoma. Carcinogenesis 30: 416-422.
Marsit, C. J., D. C. Koestler, B. C. Christensen, M. R. Karagas, E. A. Houseman and K. T. Kelsey. 2011. DNA methylation array analysis identifies profiles of blood-derived DNA methylation associated with bladder cancer. J Clin Oncol 29: 1133-1139.
Miranda, T. B. and P. A. Jones. 2007. DNA methylation: the nuts and bolts of repression. J Cell Physiol 213: 384-390.
Monti, S., P. Tamayo, J. Mesirov, T. Golub. 2003. Consensus Clustering: A Resampling-Based Method for Class Discovery and Visualization of Gene Expression Microarray Data. Machine Learning Journal 52 (1-2): 91-118.
Nickerson, D. A., V. O. To be and S. L. Taylor. 1997. PolyPhred: automating the detection and genotyping of single nucleotide substitutions using fluorescence-based resequencing. Nucleic Acids Res 25: 2745-2751.
Noushmehr, H., D. J. Weisenberger, K. Diefes, H. S. Phillips, K. Pujara, B. P. Berman, F. Pan, C. E. Pelloski, E. P. Sulman, K. P. Bhat et al. 2010. Identification of a CpG island methylator phenotype that defines a distinct subgroup of glioma. Cancer Cell 17: 510-522.
O'Brien, M. J. 2007. Hyperplastic and serrated polyps of the colorectum. Gastroenterol Clin North Am 36: 947-68, viii.
Ogino, S., T. Kawasaki, G. J. Kirkner, M. Loda and C. S. Fuchs. 2006. CpG island methylator phenotype-low (CIMP-low) in colorectal cancer: possible associations with male sex and KRAS mutations. J Mol Diagn 8: 582-588.
Ogino, S., T. Kawasaki, G. J. Kirkner, Y. Suemoto, J. A. Meyerhardt and C. S. Fuchs. 2007. Molecular correlates with MGMT promoter methylation and silencing support CpG island methylator phenotype-low (CIMP-low) in colorectal cancer. Gut 56: 1564-1571.
Ohm, J. E., K. M. McGarvey, X. Yu, L. Cheng, K. E. Schuebel, L. Cope, H. P. Mohammad, W. Chen, V. C. Daniel, W. Yu et al. 2007. A stem cell-like chromatin pattern may predispose tumor suppressor genes to DNA hypermethylation and heritable silencing. Nat Genet 39: 237-242.
Pino, M. S. and D. C. Chung. 2010. The chromosomal instability pathway in colon cancer. Gastroenterology 138: 2059-2072.
Rodriguez, J., M. Munoz, L. Vives, C. G. Frangou, M. Groudine and M. A. Peinado. 2008. Bivalent domains enforce transcriptional memory of DNA methylated genes in cancer cells. Proc Natl Acad Sci USA 105: 19809-19814.
Schlesinger, Y., R. Straussman, I. Keshet, S. Farkash, M. Hecht, J. Zimmerman, E. Eden, Z. Yakhini, E. Ben-Shushan, B. E. Reubinoff et al. 2007. Polycomb-mediated methylation on Lys27 of histone H3 pre-marks genes for de novo methylation in cancer. Nat Genet 39: 232-236.
Shen, L., M. Toyota, Y. Kondo, E. Lin, L. Zhang, Y. Guo, N. S. Hernandez, X. Chen, S. Ahmed, K. Konishi et al. 2007. Integrated genetic and epigenetic analysis identifies three different subclasses of colon cancer. Proc Natl Acad Sci USA 104: 18654-18659.
Suzuki, H., S. Igarashi, M. Nojima, R. Maruyama, E. Yamamoto, M. Kai, H. Akashi, Y. Watanabe, H. Yamamoto, Y. Sasaki et al. 2010. IGFBP7 is a p53-responsive gene specifically silenced in colorectal cancer with CpG island methylator phenotype. Carcinogenesis 31: 342-349.
Takai, D. and P. A. Jones. 2002. Comprehensive analysis of CpG islands in human chromosomes 21 and 22. Proc Natl Acad Sci USA 99: 3740-3745.
Walther, A., E. Johnstone, C. Swanton, R. Midgley, I. Tomlinson and D. Kerr. 2009. Genetic prognostic and predictive markers in colorectal cancer. Nat Rev Cancer 9: 489-499.
Weisenberger, D. J., K. D. Siegmund, M. Campan, J. Young, T. I. Long, M. A. Faasse, G. H. Kang, M. Widschwendter, D. Weener, D. Buchanan et al. 2006. CpG island methylator phenotype underlies sporadic microsatellite instability and is tightly associated with BRAF mutation in colorectal cancer. Nat Genet 38: 787-793.
Widschwendter, M., H. Fiegl, D. Egle, E. Mueller-Holzner, G. Spizzo, C. Marth, D. J. Weisenberger, M. Campan, J. Young, I. Jacobs et al. 2007. Epigenetic stem cell signature in cancer. Nat Genet 39: 157-158.
Wood, L. D., D. W. Parsons, S. Jones, J. Lin, T. Sjoblom, R. J. Leary, D. Shen, S. M. Boca, T. Barber, J. Ptak et al. 2007. The genomic landscapes of human breast and colorectal cancers. Science 318: 1108-1113.
Yagi, K., K. Akagi, H. Hayashi, G. Nagae, S. Tsuji, T. Isagawa, Y. Midorikawa, Y. Nishimura, H. Sakamoto, Y. Seto et al. 2010. Three DNA methylation epigenotypes in human colorectal cancer. Clin Cancer Res 16: 21-33.
Young, J., K. G. Biden, L. A. Simms, P. Huggard, R. Karamatic, H. J. Eyre, G. R. Sutherland, N. Herath, M. Barker, G. J. Anderson et al. 2001. HPP1: a transmembrane protein-encoding gene commonly methylated in colorectal polyps and cancers. Proc Natl Acad Sci USA 98: 265-270.
Young, J. and J. R. Jass. 2006. The case for a genetic predisposition to serrated neoplasia in the colorectum: hypothesis and review of the literature. Cancer Epidemiol Biomarkers Prev 15: 1778-1784.
Young, J., M. Jenkins, S. Parry, B. Young, D. Nancarrow, D. English, G. Giles and J. Jass. 2007. Serrated pathway colorectal cancer in the population: genetic consideration. Gut 56: 1453-1459.

1. A method of at least one of diagnosing, detecting and classifying a colorectal cancer belonging to a distinct colorectal cancer (CRC) subgroup having frequent CpG island hypermethylation (CIMP CRC), comprising:
determining, by analyzing a human subject biological sample comprising colorectal cancer (CRC) cell genomic DNA using a suitable assay, a CpG methylation status of at least one CpG dinucleotide from each gene of the gene marker panel of B3GAT2, FOXL2, KCNK13, RAB31 and SLIT1 (CIMP marker panel); wherein CpG hypermethylation, relative to normal control values, of at least three genes of the CIMP marker gene panel is indicative of a frequent CpG island hypermethylation colorectal cancer subgroup (CIMP CRC), and wherein a method of at least one of diagnosing, detecting and/or classifying a colorectal cancer belonging to the distinct colorectal cancer (CRC) subgroup having frequent CpG island hypermethylation (CIMP CRC) is afforded.

2. The method of claim 1, wherein CpG island hypermethylation colorectal cancer (CIMP CRC), comprises both CIMP-H and CIMP-L subgroups of CIMP.

3. The method of claim 1, wherein CIMP-H and CIMP-L tumors are identified with about 100% sensitivity and about 95.6% specificity with about 2.4% misclassification using conditions that three or more markers show DNA methylation β-value threshold of ≧0.1. as defined herein.

4. The method of claim 1, further comprising:
determining, by analyzing the human subject biological using a suitable assay, a CpG methylation status of at least one CpG dinucleotide from each gene of an additional gene marker panel of FAM78A, FSTL1, KCNC1, MYOCD, and SLC6A4 (CIMP-H marker panel), wherein a CIMP-L subgroup of CIMP is indicated where the CIMP-defining marker panel is positive (hypermethylation of at least three genes of the CIMP marker gene panel) while the CIMP-H marker panel is negative (hypermethylation of only 0-2 genes of the CIMP-H marker gene panel), and wherein a CIMP-H subgroup of CIMP is indicated where both the CIMP-defining marker panel and the CIMP-H marker panel are positive (hypermethylation of at least three genes of each marker gene panel).

5. The method of claim 1, wherein determining a CpG methylation status of at least one CpG dinucleotide from each gene of the gene marker panel of B3GAT2, FOXL2, KCNK13, RAB31 and SLIT1 (CIMP marker panel), comprises determining a CpG methylation status of at least one CpG dinucleotide from each of: at least one of SEQ ID NOS:45, 46 and 278 (B3GAT2 promoter, CpG island and amplicon, respectively); at least one of SEQ ID NOS:40, 41 and 240 (FOXL2 promoter, CpG island and amplicon, respectively); at least one of SEQ ID NOS:25, 26 and 224 (KCNK13 promoter, CpG island and amplicon, respectively); at least one of SEQ ID NOS:35, 36 and 236 (RAB31 promoter, CpG island and amplicon, respectively); and at least one of SEQ ID NOS:30, 31, 228 and 232 (SLIT1 promoter, CpG island and amplicons, respectively), respectively.

6. The method of claim 4, wherein determining a CpG methylation status of at least one CpG dinucleotide from each gene of the gene marker panel of FAM78A, FSTL1, KCNC1, MYOCD, and SLC6A4 (CIMP-H marker panel), comprises determining a CpG methylation status of at least one CpG dinucleotide from each of: at least one of SEQ ID NOS:50, 51 and 247 (FAM78A promoter, CpG island and amplicon, respectively); at least one of SEQ ID NOS:65, 66, 259, 263 and 265 (FSTL1 promoter, CpG island and amplicons, respectively); at least one of SEQ ID NOS:60, 61 and 255 (KCNC1 promoter, CpG island and amplicon, respectively); at least one of SEQ ID NOS:55, 56 and 251 (MYOCD promoter, CpG island and amplicon, respectively); and at least one of SEQ ID NOS:70, 71, and 269 (SLC6A4 promoter, CpG island and amplicon, respectively), respectively.

7. The method of claim 1, further comprising determination of at least one of KRAS, BRAF and TP53 mutant status.

8. The method of claim 7, wherein the BRAF mutation status comprises mutation status at codon 600 in exon 15 (e.g., BRAFV600E), wherein the KRAS mutation status comprises mutation status at codon 12 and/or 13 in exon 2, and wherein the TP53 mutation status comprises mutation status at exons 4 through 8.

9. The method of claim 8, wherein a positive mutation status comprises at least one of missense mutations, nonsense mutations, splice-site mutations, frame-shift mutations, and in-frame deletions.

10. The method of claim 1, further comprising determining a MLH1 gene methylation status, wherein MLH1 hypermethylation is strongly associated with CIMP-H CRC.

11. The method of claim 1, wherein determining methylation status comprises treating the genomic DNA, or a fragment thereof, with one or more reagents (e.g., bisulfite, hydrogen sulfite, disulfite, and combinations thereof) to convert cytosine bases that are unmethylated in the 5-position thereof to uracil or to another base that is detectably dissimilar to cytosine in terms of hybridization properties.

12. A method of at least one of diagnosing, detecting and classifying a colorectal cancer belonging to a distinct colorectal cancer (CRC) subgroup having frequent CpG island hypermethylation (CIMP CRC), comprising:
determining, by analyzing a human subject biological sample comprising colorectal cancer (CRC) cell genomic DNA using a suitable assay, a CpG methylation status of at least one CpG dinucleotide from each gene of the gene marker panel of FAM78A, FSTL1, KCNC1, MYOCD, and SLC6A4 (CIMP-H marker panel); wherein CpG hypermethylation, relative to normal control values, of at least three genes of the CIMP-H marker gene panel is indicative of a CIMP-H subgroup of CIMP CRC, and wherein a method of at least one of diagnosing, detecting and classifying a colorectal cancer belonging to the CIMP-H subgroup of CIMP CRC is afforded.

13. The method of claim 12 wherein CIMP-H tumors are identified with about 100% sensitivity and about 100% specificity (about 0% misclassification) using conditions that three or more markers show DNA methylation β-value threshold of ≧0.1. as defined herein.

14. The method of claim 12, further comprising determination of at least one of KRAS, BRAF and TP53 mutant status.

15. The method of claim 14, wherein the BRAF mutation status comprises mutation status at codon 600 in exon 15 (e.g., BRAFV600E), wherein the KRAS mutation status comprises mutation status at codon 12 and/or 13 in exon 2, and wherein the TP53 mutation status comprises mutation status at exons 4 through 8.

16. The method of claim 15, wherein a positive mutation status comprises at least one of missense mutations, nonsense mutations, splice-site mutations, frame-shift mutations, and in-frame deletions.

17. The method of claim 12, wherein determining a CpG methylation status of at least one CpG dinucleotide from each gene of the gene marker panel of FAM78A, FSTL1, KCNC1, MYOCD, and SLC6A4 (CIMP-H marker panel), comprises determining a CpG methylation status of at least one CpG dinucleotide from each of: at least one of SEQ ID NOS:50, 51 and 247 (FAM78A promoter, CpG island and amplicon, respectively); at least one of SEQ ID NOS:65, 66, 259, 263 and 265 (FSTL1 promoter, CpG island and amplicons, respectively); at least one of SEQ ID NOS:60, 61 and 255 (KCNC1 promoter, CpG island and amplicon, respectively); at least one of SEQ ID NOS:55, 56 and 251 (MYOCD promoter, CpG island and amplicon, respectively); and at least one of SEQ ID NOS:70, 71, and 269 (SLC6A4 promoter, CpG island and amplicon, respectively), respectively.

18. The method of claim 12, further comprising determining a MLH1 gene methylation status, wherein MLH1 hypermethylation is strongly associated with CIMP-H CRC.

19. The method of claim 12, wherein determining methylation status comprises treating the genomic DNA, or a fragment thereof, with one or more reagents (e.g., bisulfite, hydrogen sulfite, disulfite, and combinations thereof) to convert cytosine bases that are unmethylated in the 5-position thereof to uracil or to another base that is detectably dissimilar to cytosine in terms of hybridization properties.

20. A kit suitable for performing the methods according to claim 1, comprising, for each gene of the gene marker panel of B3GAT2, FOXL2, KCNK13, RAB31 and SLIT1, at least two oligonucleotides whose sequences in each case are identical, are complementary, or hybridize under stringent or highly stringent conditions to the respective marker gene; and optionally comprising a bisulfite reagent (e.g., bisulfite, hydrogen sulfite, disulfite, and combinations thereof).

21. The kit of claim 20, wherein the respective marker gene sequences comprise at least one sequence from each of: at least one of SEQ ID NOS:45, 46 and 278 (B3GAT2 promoter, CpG island and amplicon, respectively); at least one of SEQ ID NOS:40, 41 and 240 (FOXL2 promoter, CpG island and amplicon, respectively); at least one of SEQ ID NOS:25, 26 and 224 (KCNK13 promoter, CpG island and amplicon, respectively); at least one of SEQ ID NOS:35, 36 and 236 (RAB31 promoter, CpG island and amplicon, respectively); and at least one of SEQ ID NOS:30, 31, 228 and 232 (SLIT1 promoter, CpG island and amplicons, respectively), respectively.

22. A kit suitable for performing the method according to claim 12, comprising, for each gene of the gene marker panel of FAM78A, FSTL1, KCNC1, MYOCD, and SLC6A4, at least two oligonucleotides whose sequences in each case are identical, are complementary, or hybridize under stringent or highly stringent conditions to the respective marker gene; and optionally comprising a bisulfite reagent (e.g., bisulfite, hydrogen sulfite, disulfite, and combinations thereof).

23. The method of claim 22, wherein the respective marker gene sequences comprise at least one sequence from each of: at least one of SEQ ID NOS:50, 51 and 247 (FAM78A promoter, CpG island and amplicon, respectively); at least one of SEQ ID NOS:65, 66, 259, 263 and 265 (FSTL1 promoter, CpG island and amplicons, respectively); at least one of SEQ ID NOS:60, 61 and 255 (KCNC1 promoter, CpG island and amplicon, respectively); at least one of SEQ ID NOS:55, 56 and 251 (MYOCD promoter, CpG island and amplicon, respectively); and at least one of SEQ ID NOS:70, 71, and 269 (SLC6A4 promoter, CpG island and amplicon, respectively), respectively.
